(19) United States
(12) Patent Application Publication
(10) Pub. No.: US 2026/0241013 A1
(43) Pub. Date: Aug. 20, 2026

(54) TARGETED PROTEIN DEGRADATION USING PRODRUGS OF BIFUNCTIONAL COMPOUNDS THAT BIND UBIQUITIN LIGASE AND A TARGET MCL-1 PROTEIN

(71) Applicant: Captor Therapeutics S.A., Wroclaw (PL)

(72) Inventors: Sylvain COTTENS, Witterswil (CH); Andrzej TRACZ, Wilczyce (PL); Michal Jerzy WALCZAK, Wroclaw (PL); Karolina WÓJCIK, Wroclaw (PL)

(21) Appl. No.: 19/135,307

(22) PCT Filed: Dec. 6, 2023
(86) PCT No.: PCT/EP2023/084604

§ 371 Date: Jun. 3, 2025

(30) Foreign Application Priority Data
Dec. 6, 2022 (WO) PCT/PL2022/000067

(51) Int. Cl. A61K 47/55; A61K 31/357; A61K 31/553; A61K 31/565; A61K 31/635; A61K 31/7068; A61K 38/07; A61K 39/395; A61K 45/06; A61K 47/54; A61P 35/00

(52) U.S. Cl. CPC A61K 47/55; A61K 31/357; A61K 31/553; A61K 31/565; A61K 31/635; A61K 31/7068; A61K 38/07; A61K 39/3955; A61K 45/06; A61K 47/545; A61P 35/00

(57) ABSTRACT

A compound of formula (I) [MCL-1 ligand prodrug moiety]-[linker]-[ligase ligand moiety](I) or a salt, solvate, hydrate or isomer thereof, wherein [MCL-1 ligand prodrug moiety] is e.g. and its use in the treatment of cancer.

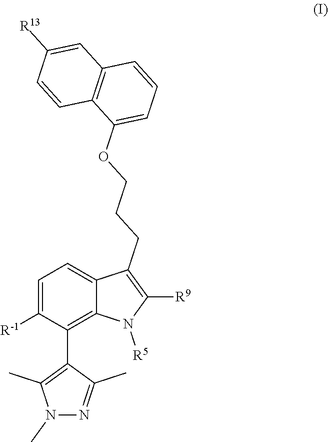

### FIELD OF THE INVENTION

The present invention relates to prodrugs of bifunctional compounds which can bind to a ubiquitin ligase and also to a target protein, such that the target protein is placed in proximity to the ubiquitin ligase in order to induce its degradation.

### BACKGROUND

The Ubiquitin-Proteasome System (UPS) is responsible for the maintenance of healthy and well-balanced proteome. In the process of ubiquitination, ubiquitin units are covalently attached to the protein, forming a polyubiquitin chain, which marks the protein for degradation via the proteasome. Ubiquitination is central to the regulation of nearly all cellular processes and is also tightly regulated itself. Ubiquitin ligases facilitate ubiquitination of different proteins in vivo and contribute to precise regulation of the system. Upon recognition, the ubiquitin ligases mediate the attachment of ubiquitin moieties to the target protein, which label it for degradation by the proteasome.

The idea of selective target protein degradation (TPD) by modulation of UPS was first described in 1999 (US2002173049 A1 (PROTEINIX INC) 21 Nov. 2002). One approach to TPD is by the use of bifunctional molecules that bind the ubiquitin ligase and the target protein simultaneously, allowing for efficient ubiquitin transfer to the latter. This concept was first described by Sakamoto K M et al. (Proc Natl Acad Sci USA. 2001 Jul. 17; 98(15):8554-9) and more recently reviewed by Burslem G M and Crews C M (Cell. 2020 Apr. 2; 181(1):102-114).

Oncogenic stress, such as DNA damage, may result in programmed cell death, the cellular response meant to prevent the oncogenic transformation. This mechanism depends on an interplay between pro-apoptotic and anti-apoptotic Bcl-2 proteins, and the balance of these proteins is essential for the proper functioning of the cell.

BCL-2, BCL-xL and MCL-1 are BH3-domain-containing anti-apoptotic proteins. These proteins bind to effector Bcl-2 proteins Bak and Bax (via their BH3 domains), preventing their pro-apoptotic activity.

Inhibition of BH3 domain—BH3 pocket binding interface is a well-known approach to cancer therapy (Leber B, Kale J, Andrews D W. Cancer Discov. 2018 December; 8(12):1511-1514).

High expression of induced myeloid leukaemia cell differentiation protein (MCL-1) is observed in many human cancers and is associated with resistance to cytotoxic drugs. Research shows that inhibition of MCL-1 protein in some malignancies leads to the release of pro-apoptotic proteins and induction of apoptosis. Therefore, targeting MCL-1 can be applied as a therapeutic strategy in these types of cancer which are MCL-1 dependent, such as multiple myeloma, acute myeloid leukaemia, chronic myeloid leukaemia, B-cell acute lymphoblastic leukaemia, hepatocellular carcinoma and non-small cell lung cancers. This concept was confirmed in vitro and in vivo (Tron A E et al. Nat Commun. 2018 Dec. 17; 9(1):5341). Also, treatment with Bcl-2 inhibitors and MEK inhibitors often elicits MCL-1 dependence and subsequent inactivation of MCL-1 results in synthetic lethality (Leber B, Kale J, Andrews D W. Cancer Discov. 2018 December; 8(12):1511-1514). As demonstrated by Montero, J. et al. (Nat. Commun. 10, 5157 (2019)) and Sale, M. J. et al. (Nat. Commun. 10, 5167 (2019)), MCL-1 is a driver of adaptive survival in tumor cells treated with oncogene targeted therapies, therefore MCL-1 targeting drugs are likely to overcome cancer resistance to these therapeutics.

In parallel to the efforts focused on inhibition of MCL-1, targeted degradation appears as an attractive therapeutic alternative. Both Papatzimas et al. (J. Med. Chem. 2019, 62, 11, 5522-5540) and Wang Z et al. (J. Med. Chem. 2019, 62, 17, 8152-8163) have demonstrated degradation of the MCL-1 protein. However, the potency of reported compounds in terms of cellular degradation and the ability to induce apoptosis remains suboptimal. Therefore, alternative chemotypes with improved potency are needed to develop therapeutically applicable MCL-1 degraders.

One of the challenges in the development of MCL-1 targeted therapeutics is related to safety, as MCL-1 has been shown to be essential for cardiac homeostasis in adult murine models, and the absence of MCL-1 led to loss of cardiomyocytes. Some clinical trials involving MCL-1 inhibitors are currently on clinical hold to evaluate a safety signal for cardiac toxicity (Wei A H et al. Blood Rev. 2020 November; 44: 100672).

It would also be desirable to develop prodrugs of MCL-1 targeted therapeutics. Some potential advantages of prodrugs may be that the bioavailability of the active therapeutic compounds could be increased, thereby allowing administration of a lower dose. The risk of adverse side effects of the active therapeutic compounds may also be reduced.

### SUMMARY OF INVENTION

In accordance with a first aspect of the invention, there is provided a compound of formula (I)

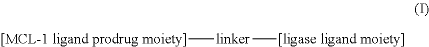

or a salt, solvate, hydrate or isomer thereof,
wherein [ligase ligand moiety] is:

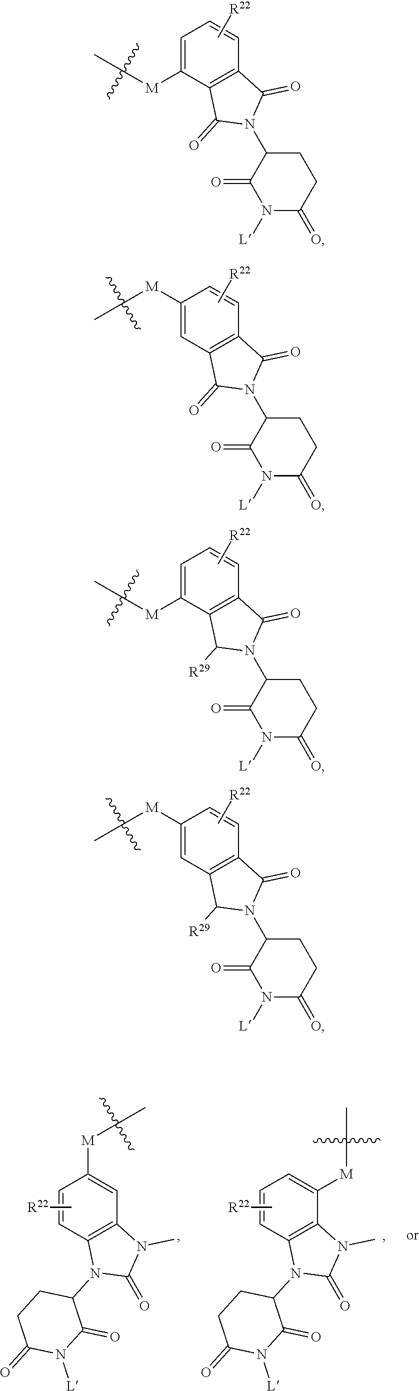

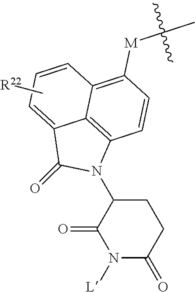

wherein
M is O, S or NH, or is absent;
[symbol] indicates attachment to R¹⁸ of the linker;
R²² is hydrogen, halogen, —OMe, an amino group, heterocycloalkyl, or unsubstituted C₁-C₆ alkyl; and
R²⁹ is hydrogen or Me
L′ is hydrogen, alkyl, benzyl, acetyl or pivaloyl;
   wherein [MCL-1 ligand prodrug moiety] is a compound of Formula (A1), (A2), (A3) or (A4):

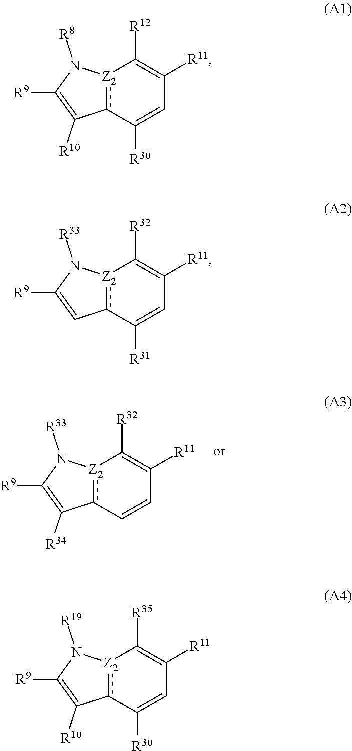

wherein
[symbol] is a single bond or a double bond;
each Z₂ is independently N or C, wherein when Z₂ is N, then [symbol] is a single bond; and when Z₂ is C, then [symbol] is a double bond;
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is

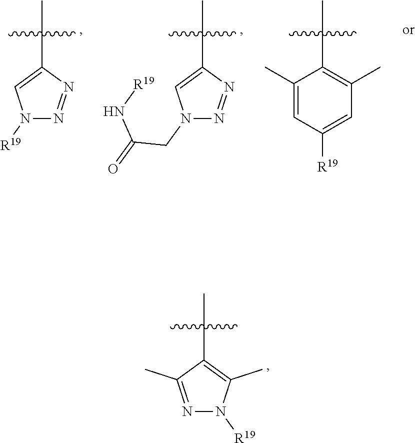

R³¹ is —C₂₋₅alkyl-O—R¹³, —O—C₂₋₅alkyl-R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, R

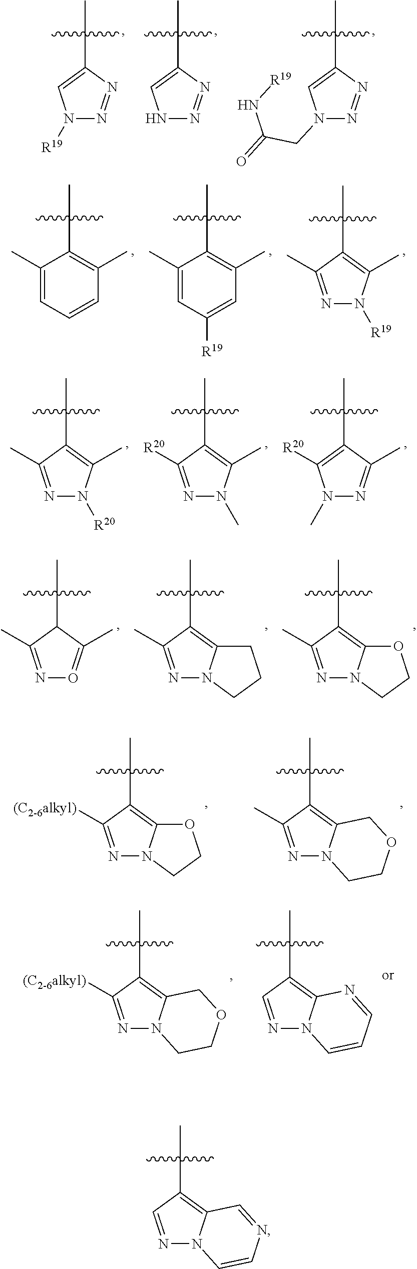

each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is

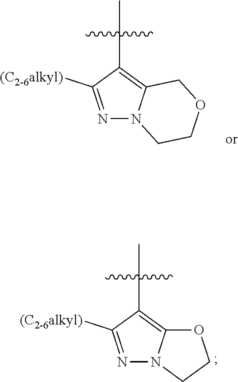

R¹⁹ is a bond connected to R¹⁴ of the linker; wherein each of Formula (A1), Formula (A2), Formula (A3) and Formula (A4) contains a single R¹⁹;
   and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O) m(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂) mS(O)(unsubstituted C₁-C₆alkyl), —OC(O)O(CH₂) mS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, —C(O)NMe₂;
each R^{p} is independently —(CH₂)ₘOC(O)(unsubstituted C₁-C₆alkyl), and each m is independently 1, 2, 3 or 4;
or
(ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me wherein each p is independently 2, 3, 4, 5, or 6; and wherein [linker] has the following formula

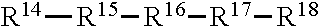

wherein
R¹⁴ is —C₁-C₆ alkyl, —C₂₋₆ alkenyl, —C₂₋₆ alkynyl, —C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)ₓ, —(C₃H₆—O)ₓ, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
wherein at least one of R¹⁴—R¹⁸ is present.

In some embodiments, [ligase ligand moiety] is:

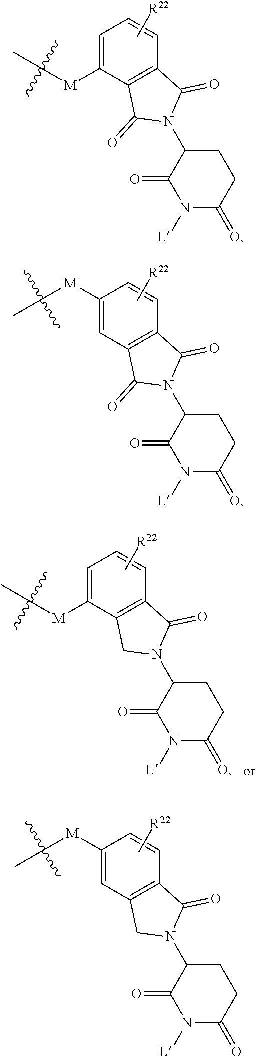

In some embodiments, [ligase ligand moiety] is:

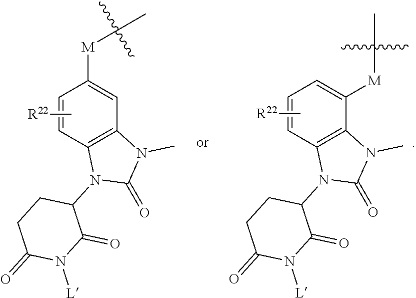

In some embodiments, [ligase ligand moiety] is:

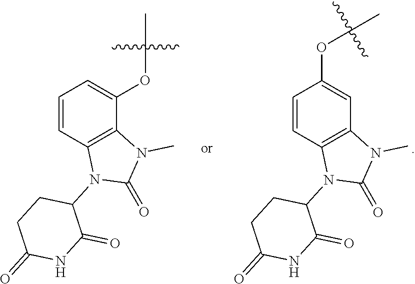 [SMILES: *Oc1cccc2c1n(C)c(=O)n2C1CCC(=O)NC1=O ; *Oc1ccc2c(c1)n(C)c(=O)n2C1CCC(=O)NC1=O]

In some embodiments, R²² is hydrogen.

In some embodiments, L′ is hydrogen.

In some embodiments, M is O or NH, or is absent.

In some embodiments, [ligase ligand moiety] is

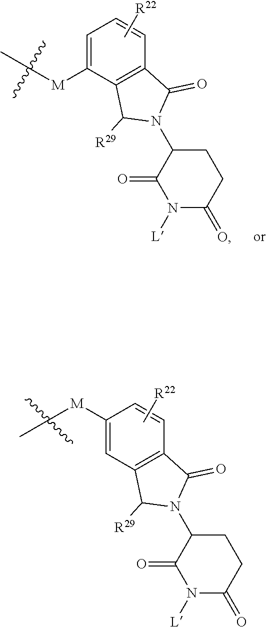

In some embodiments, [ligase ligand moiety] is

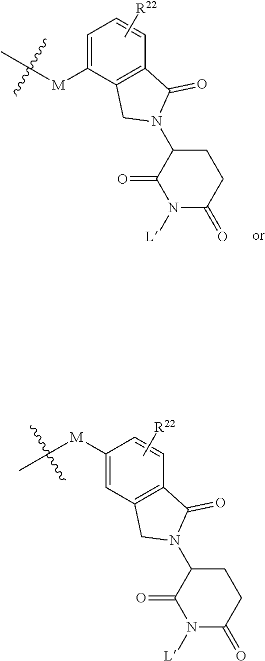

In some embodiments, [ligase ligand moiety] is:

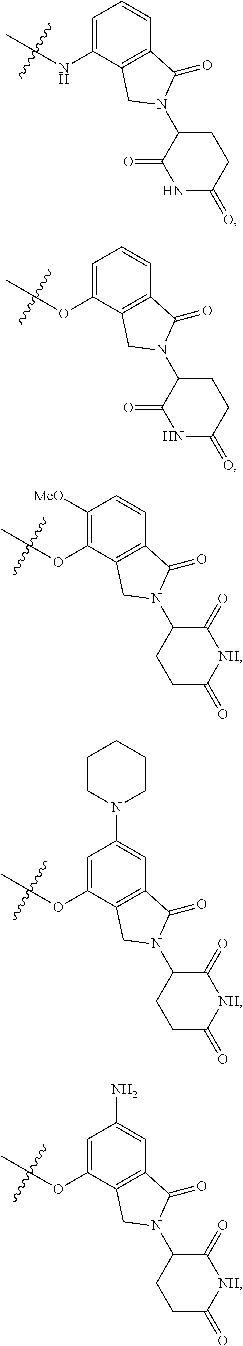 [SMILES: *Nc1cccc2c1CN(C1CCC(=O)NC1=O)C2=O ; *Oc1cccc2c1CN(C1CCC(=O)NC1=O)C2=O ; *Oc1c(OC)ccc2c1CN(C1CCC(=O)NC1=O)C2=O ; *Oc1cc(N2CCCCC2)cc2c1CN(C1CCC(=O)NC1=O)C2=O ; *Oc1cc(N)cc2c1CN(C1CCC(=O)NC1=O)C2=O]

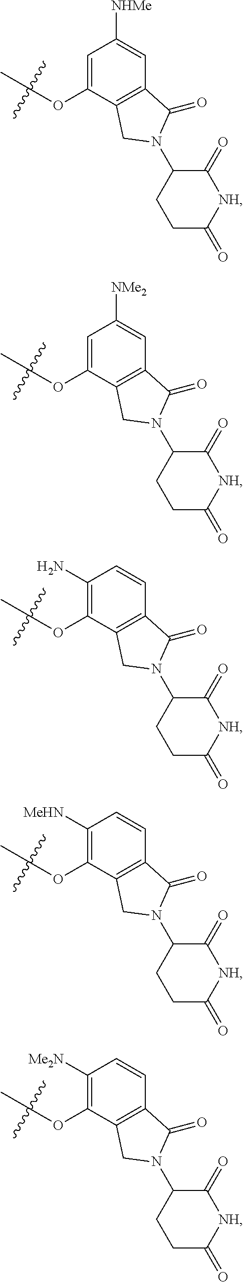 [SMILES: *Oc1cc(NC)cc2c1CN(C1CCC(=O)NC1=O)C2=O ; *Oc1cc(N(C)C)cc2c1CN(C1CCC(=O)NC1=O)C2=O ; *Oc1c(N)ccc2c1CN(C1CCC(=O)NC1=O)C2=O ; *Oc1c(NC)ccc2c1CN(C1CCC(=O)NC1=O)C2=O ; *Oc1c(N(C)C)ccc2c1CN(C1CCC(=O)NC1=O)C2=O]

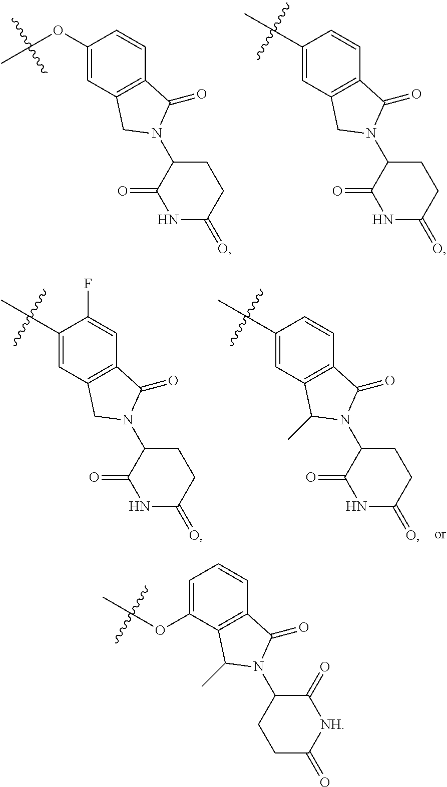

In some embodiments, [ligase ligand moiety] is

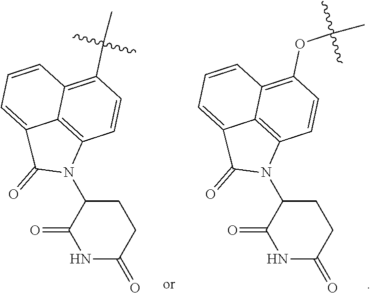 [SMILES: *c1ccc2c3c(cccc13)C(=O)N2C1CCC(=O)NC1=O ; *Oc1ccc2c3c(cccc13)C(=O)N2C1CCC(=O)NC1=O]

In some embodiments, [ligase ligand moiety] is

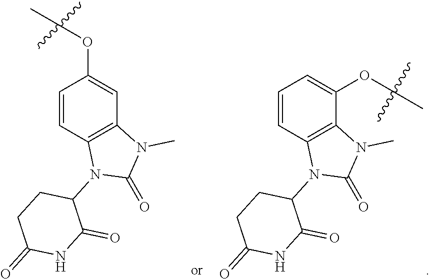 [SMILES: *Oc1ccc2c(c1)n(C)c(=O)n2C1CCC(=O)NC1=O ; *Oc1cccc2c1n(C)c(=O)n2C1CCC(=O)NC1=O]

In some embodiments, [ligase ligand moiety] is:

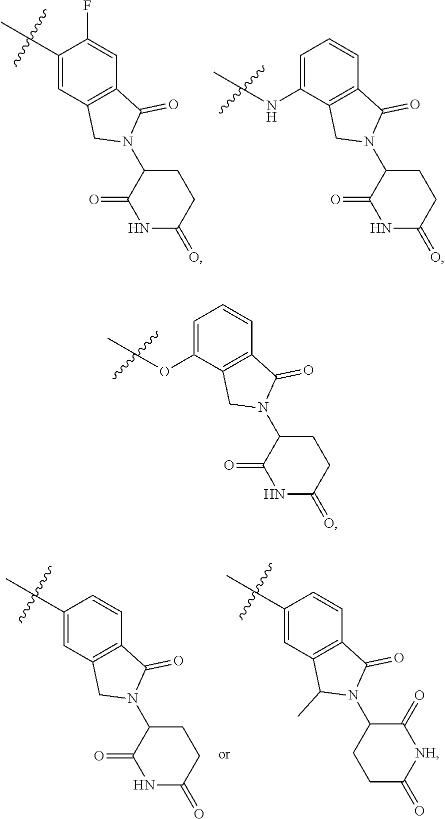 [SMILES: *c1cc2c(cc1F)C(=O)N(C1CCC(=O)NC1=O)C2 ; *Nc1cccc2c1CN(C1CCC(=O)NC1=O)C2=O ; *Oc1cccc2c1CN(C1CCC(=O)NC1=O)C2=O ; *c1ccc2c(c1)CN(C1CCC(=O)NC1=O)C2=O ; *c1ccc2c(c1)C(C)N(C1CCC(=O)NC1=O)C2=O]

In some embodiments, [ligase ligand moiety] is:

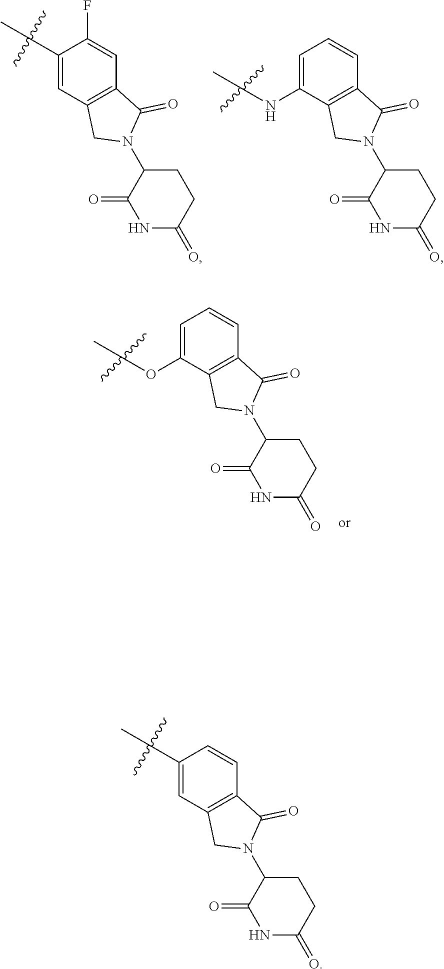 [SMILES: *c1cc2c(cc1F)C(=O)N(C1CCC(=O)NC1=O)C2 ; *Nc1cccc2c1CN(C1CCC(=O)NC1=O)C2=O ; *Oc1cccc2c1CN(C1CCC(=O)NC1=O)C2=O ; *c1ccc2c(c1)CN(C1CCC(=O)NC1=O)C2=O]

In some embodiments:
R¹⁶ is —C₁-C₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(C₂H₄—O)ₓ, —(C₃H₆—O)ₓ, or is absent
R¹⁸ is —C₁₋₆ alkyl, cycloalkyl, —CH₂—NH—C(O)—, heterocycloalkyl, or is absent.

In some embodiments, R¹⁴ is —C₁-C₆ alkyl, —C₂₋₆alkenyl, —C₂₋₆alkynyl, —C(O)—, —SO₂— or is absent. In some such embodiments, R¹⁴ is —C₁-C₆ alkyl.

In some embodiments, R¹⁵ is cycloalkyl, heterocycloalkyl, aryl, heteroaryl, —C₁₋₆ alkyl-NH—, -cycloalkyl-NH— or is absent. In some embodiments, R¹⁵ is heterocycloalkyl. In some such embodiments, R¹⁵ is piperazine, bridged piperazine, piperazine N-oxide, piperazine cation,

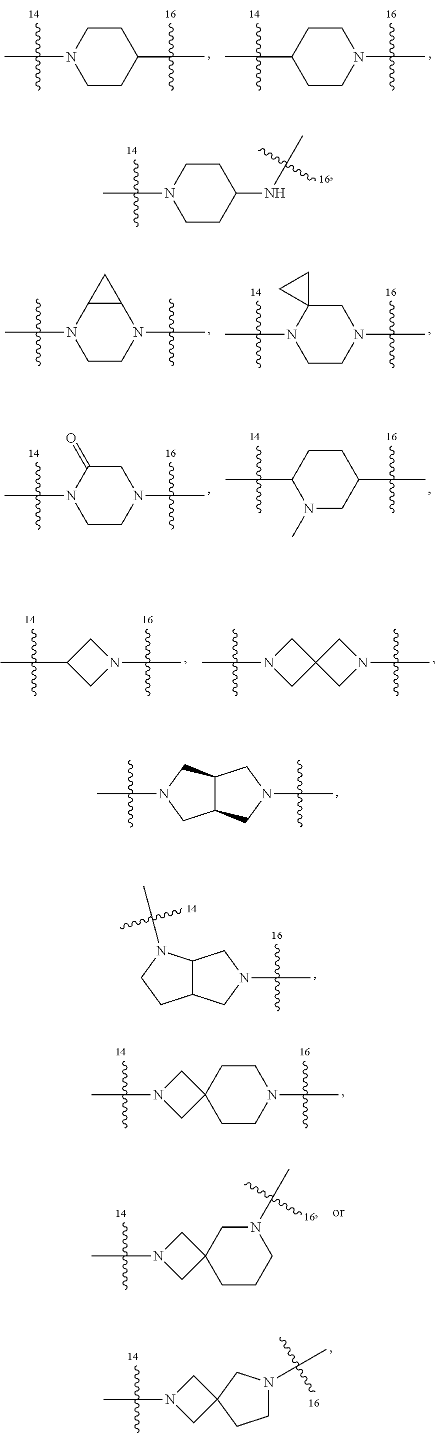

wherein [symbol] indicates attachment to R¹⁴ and [symbol] indicates attachment to R¹⁶. In some such embodiments, R¹⁵ is piperazine or

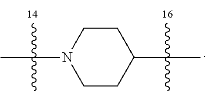 [SMILES: *C1CCN(*)CC1]

In other embodiments, R¹⁵ is absent.

In some embodiments, R¹⁶ is —C₁-C₆ alkyl, —CH₂—C(O)—NH— or —C(O)—. In some such embodiments, R¹⁶ is —C₁-C₆ alkyl or —C(O)—.

In some embodiments, R¹⁷ is absent.

In some embodiments, R¹⁸ is —C₁₋₆ alkyl, heterocycloalkyl, or is absent. In some embodiments, R¹⁸ is —C₁-C₆ alkyl or heterocycloalkyl. In some such embodiments, R¹⁸ is —C₁₋₆ alkyl or piperazine.

In some embodiments, R¹⁸ is absent.

In some embodiments, [linker] is selected from

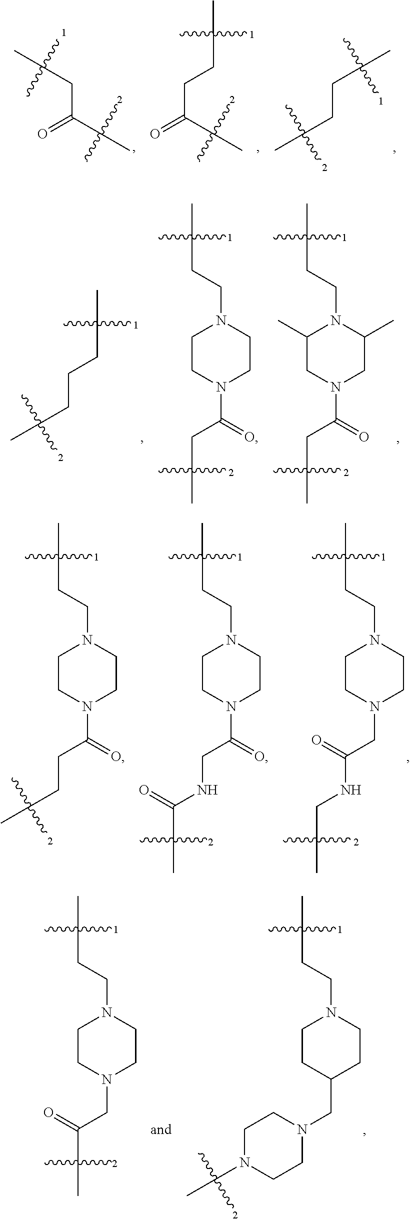

wherein
[symbol] indicates attachment to [MCL-1 ligand prodrug moiety] and
[symbol] indicates attachment to [ligase ligand moiety];

In some embodiments, [linker] is selected from

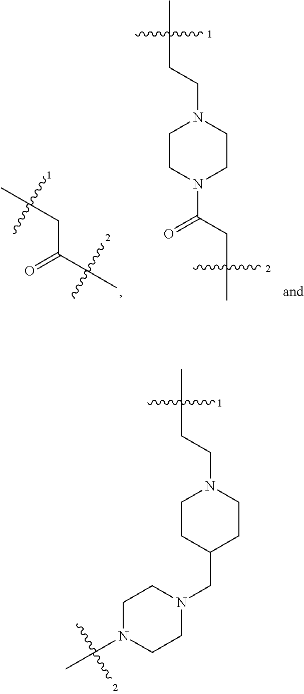 [SMILES: *CC(*)=O ; *CCN1CCN(C(=O)C*)CC1 ; *CCN1CCC(CN2CCN(*)CC2)CC1]

wherein
[symbol] indicates attachment to [MCL-1 ligand prodrug moiety] and
[symbol] indicates attachment to [ligase ligand moiety].

In some embodiments, in each of Formula (A1) and Formula (A4), one of R¹⁰ and R³⁰ is H and the other of R¹⁰ and R³⁰ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments, in Formula (A1) R¹² is

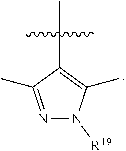

In some embodiments, in Formula (A4) R³⁵ is

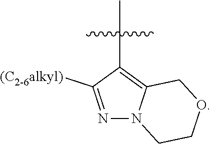

In some embodiments, in Formula (A2) R³¹ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments, in Formula (A3) R³⁴ is C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments, Z₂ is C and [symbol] is a double bond.

In some embodiments, [MCL-1 ligand prodrug moiety] is:

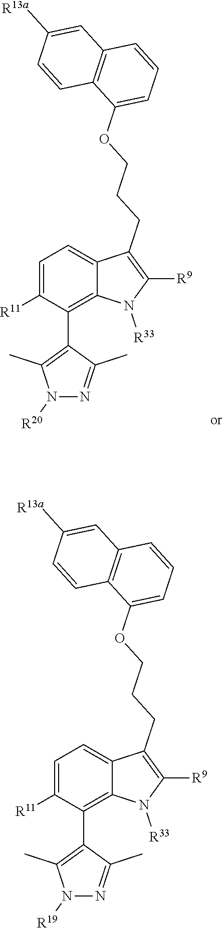

wherein
R¹¹ is halogen;
R^{13a} is halogen; and
R²⁰ is Me, —CH₂—OMe, or —(CH₂CH₂O)ₚ(C₁-C₆ alkyl), wherein p is 1-5.

In some embodiments, R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂) mS(O)(unsubstituted C₁-C₆alkyl), —OC(O)O(CH₂) mS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolyl cation.

In some embodiments, R⁹ is —C(O)O(C₁-C₆alkyl), —C(O)Ocycloalkyl, —C(O)Oheterocycloalkyl, —C(O)Oaryl, or —P(O)(OR^{p})₂, wherein said C₁-C₆alkyl is substituted by one or more groups selected from —OC(O)Me, —OC(O)Et, —OC(O)ⁱPr, —OC(O)C(CH₃)₃, —OC(O)CH₂OH, —OC(O)CH(CH₃)OH, —OC(O)C(CH₃)₂OH, —OC(O)CH₂CH₂OH, —OC(O)CH(OH)CHMe₂, —OC(O)CH(OH)CH₂CHMe₂, —OC(O)CH₂CH(OH)Me, —OC(O)CH₂C(OH)Me₂, —OC(O)CH(CH₂OH)(NHC(O)Me), —OC(O)CH(NHC(O)Me)(isobutyl), —OC(O)CH(OH)aryl, —OC(O)CH(OH)CH₂aryl, —OC(O)heterocycloalkyl, —OC(O)cycloalkyl, —OC(O)aryl, —OC(O)heteroaryl, —OC(O)CH₂CH₂aryl, —OC(O)CH(NH₂)ⁱPr, —OC(O)CH(ⁱPr)NHC(O)O^{t}Bu, —OC(O)CH₂OCH₂CH₂OMe, —OC(O)CH₂(OCH₂CH₂)₂OMe, —OC(O)OMe, —OC(O)OEt, —OC(O)OⁱPr, —OC(O)Ocycloalkyl, —OC(O)OCH₂CH₂OEt, —OC(O)O(CH₂CH₂O)₂Et, —OC(O)O(CH₂CH₂O)₃Et, —OC(O)O(CH₂CH₂O)₃CH₂CH₂OP(O)(OH)₂, —OC(O)OCH₂CH₂SMe, —OC(O)OCH₂CH₂S(O)Me, —OC(O)OCH₂CH₂S(O)₂Me, —OC(O)OCH₂heteroaryl, —OC(O)OCH₂heterocycloalkyl, —OC(O)OCH₂CH₂OCH₂aryl —OC(O)OCH₂CH₂C(O)NMe₂, —OC(O)O(CH₂)₆NH₂, —OC(O)O(CH₂)₆NMe₂, —OC(O)OCH(CH₂OEt)₂, —OC(O)NHCH₂CH₂OMe, —OC(O)NMeCH₂CH₂OMe, —SMe, —S(O)Me, —P(O)(OH)₂, —OP(O)(OH)₂, —C(O)NMe₂, —C(O)heterocycloalkyl, heteroaryl, heterocycloalkyl, —N⁺(Me)₃, —N⁺(Me)₂cycloalkyl, N-methyl morpholinium cation, N-methyl piperidinium cation and N-methyl imidazolium cation. In some such embodiments, R⁹ is —C(O)O(C₁-C₆alkyl) wherein said C₁-C₆alkyl is substituted by one or more groups selected from —OC(O)C(CH₃)₃, —OC(O)CH₂CH₂OH, —OC(O)C(CH₃)₂OH, —OC(O)heterocycloalkyl, —OC(O)CH₂OCH₂CH₂OMe, —OC(O)OiPr, —OC(O)Ocycloalkyl, —OC(O)O(CH₂CH₂O)₂Et, —OP(O)(OH)₂, heteroaryl or heterocycloalkyl.

In other embodiments, R⁹ is —C(O)OCH₂OC(O)C(CH₃)₃, —C(O)OCH₂OC(O)Me, —C(O)OCH(Me)OC(O)Me, —(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂OMe, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, —C(O)OCH₂CH₂CH₂piperazine, or —P(O)(OCH₂OC(O)C(CH₃)₃)₂. In some such embodiments, R⁹ is —C(O)OCH₂OC(O)C(CH₃)₃, —C(O)OCH₂OC(O)Me, —C(O)OCH(Me)OC(O)Me, —(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂OMe, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, or —C(O)OCH₂CH₂CH₂piperazine.

In some embodiments, R⁹ is —C(O)O(CH₂)ₚNMe₂, —C(O)O(CH₂)ₚNHMe, —C(O)OCH₂CH(OH)CH₂OH, C(O)OCH₂CH₂CMe₂OH, or —C(O)OCH₂CH₂SO₂Me wherein each p is independently 2, 3, 4, 5, or 6. In some such embodiments, R⁹ is —C(O)OCH₂CH₂NMe₂, —C(O)OCH₂CH(OH)CH₂OH or —C(O)OCH₂CH₂SO₂Me.

In some embodiments, R²⁰ is Me or —(CH₂CH₂O)₂Me.

In some embodiments, R³³ is R¹⁹. In other embodiments, R³³ is C₁-C₆ alkyl substituted with morpholine or a piperazine.

In some embodiments, the C₁-C₆ alkyl substituted with morpholine or a piperazine is

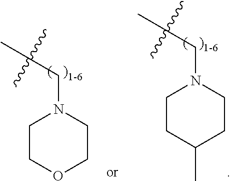

In some embodiments, R¹¹ is Cl.

In some embodiments, R¹¹ is F.

In some embodiments, the [MCL-1 ligand prodrug moiety] is:

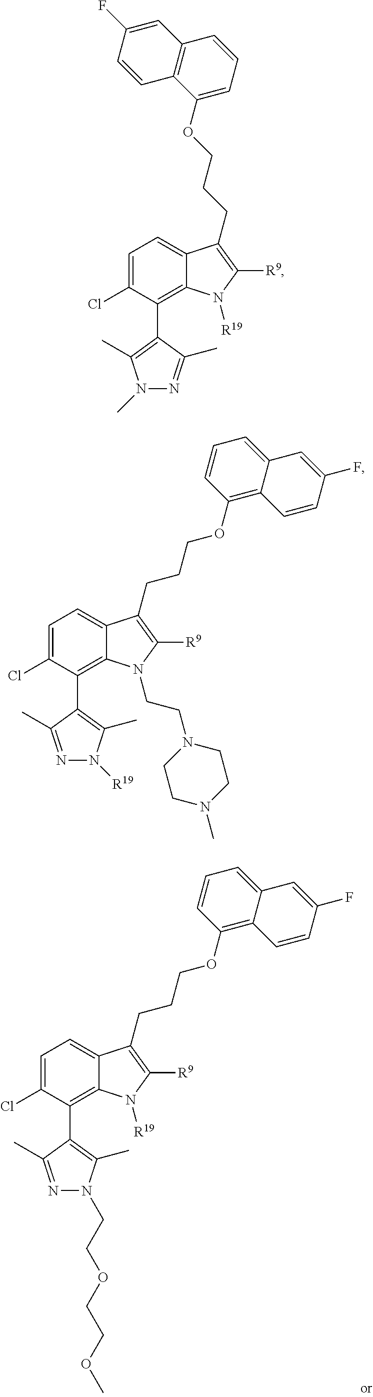

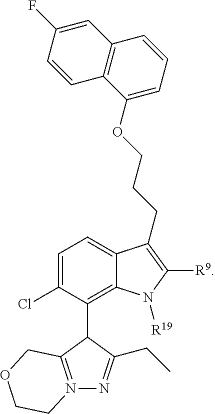

In some embodiments, [MCL-1 ligand prodrug moiety] is:

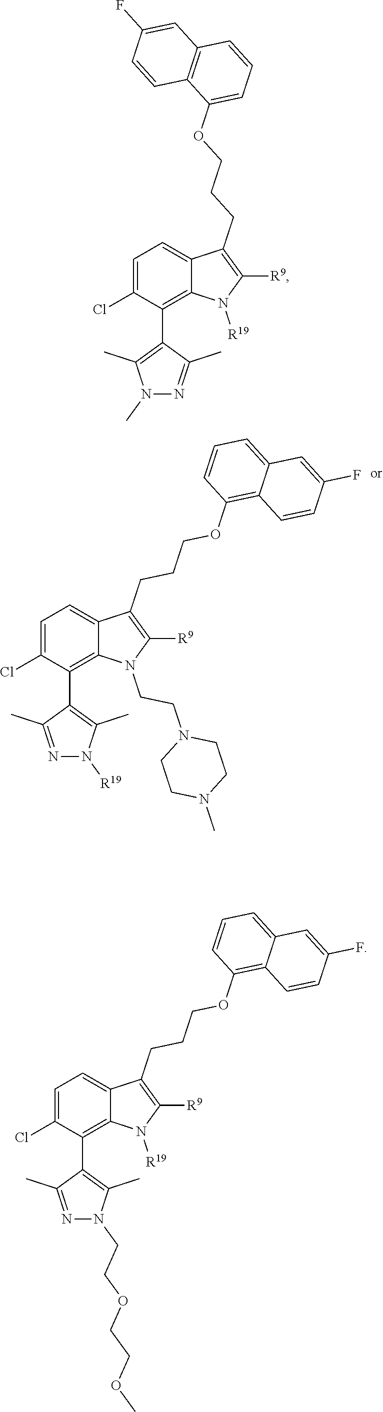

In some embodiments:
[linker] is

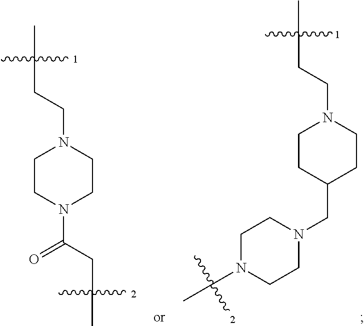 [SMILES: *CCN1CCN(C(=O)C*)CC1 ; *CCN1CCC(CN2CCN(*)CC2)CC1]

[ligase ligand moiety] is

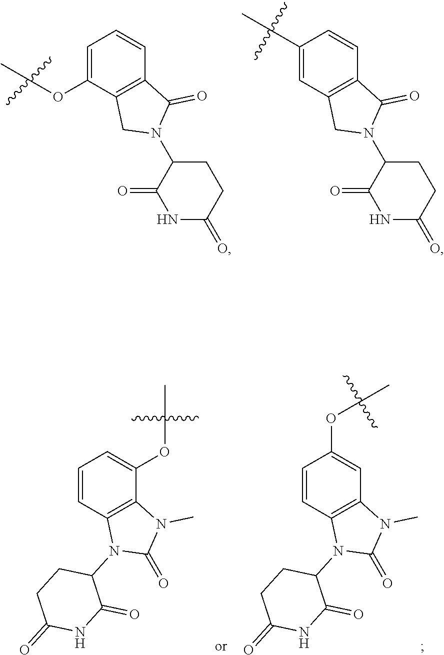 [SMILES: *Oc1cccc2c1CN(C1CCC(=O)NC1=O)C2=O ; *c1ccc2c(c1)CN(C1CCC(=O)NC1=O)C2=O ; *Oc1cccc2c1n(C)c(=O)n2C1CCC(=O)NC1=O ; *Oc1ccc2c(c1)n(C)c(=O)n2C1CCC(=O)NC1=O]

and
[MCL-1 ligand prodrug moiety] is a compound of Formula (A3), wherein
   R³² is

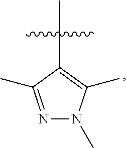

and
R⁹ is C(O)O(linear C₁-C₆alkyl)OC(O)C(CH₃)₃, C(O)O(linear C₁-C₆alkyl)OC(O)heterocyloalkyl, C(O)O(linear C₁-C₆alkyl)OC(O)CH₂CH₂OH, C(O)O(linear C₁-C₆alkyl)OC(O)CH₂OCH₂CH₂OMe, C(O)O(linear C₁-C₆alkyl)OC(O)Me, C(O)O(branched C₁-C₆alkyl)OC(O)OiPr, C(O)O(branched C₁-C₆alkyl)OC(O)Ocycloalkyl, C(O)O(branched C₁-C₆alkyl)OC(O)O(CH₂CH₂O)₂Et, or C(O)O(branched C₁-C₆alkyl)OC(O)Me.

In some embodiments:
[linker] is

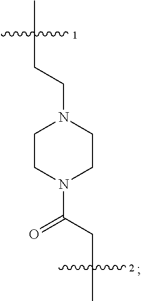 [SMILES: *CCN1CCN(C(=O)C*)CC1]

[ligase ligand moiety] is

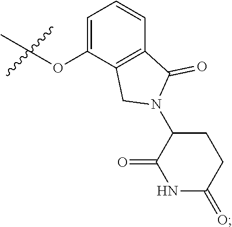 [SMILES: *Oc1cccc2c1CN(C1CCC(=O)NC1=O)C2=O]

and
[MCL-1 ligand prodrug moiety] is a compound of Formula (A3),
wherein
   R³² is

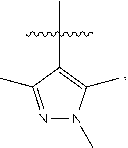

and
R⁹ is C(O)O(linear C₁-C₆alkyl)OC(O)C(CH₃)₃, C(O)O(branched C₁-C₆alkyl)OC(O)OiPr, C(O)O(branched C₁-C₆alkyl)OC(O)Ocycloalkyl, C(O)O(branched C₁-C₆alkyl)OC(O)O(CH₂CH₂O)₂Et, or C(O)O(branched C₁-C₆alkyl)OC(O)Me.

In some embodiments:
[linker] is

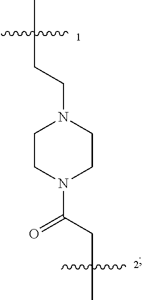 [SMILES: *CCN1CCN(C(=O)C*)CC1]

[ligase ligand moiety] is

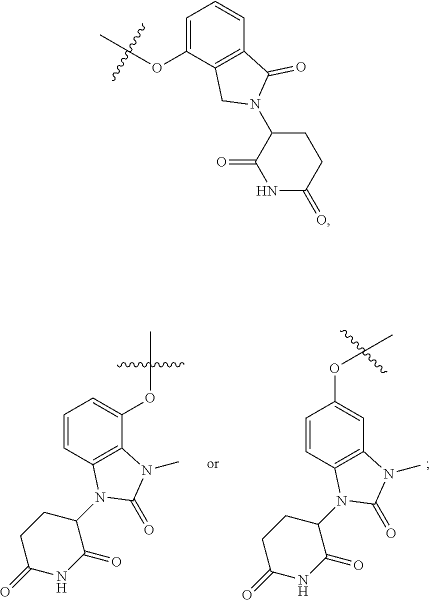 [SMILES: *Oc1cccc2c1CN(C1CCC(=O)NC1=O)C2=O ; *Oc1cccc2c1n(C)c(=O)n2C1CCC(=O)NC1=O ; *Oc1ccc2c(c1)n(C)c(=O)n2C1CCC(=O)NC1=O]

and
[MCL-1 ligand prodrug moiety] is a compound of Formula (A3), wherein
R³² is

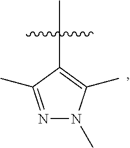

and
R⁹ is C(O)O(linear C₁-C₆alkyl)OC(O)C(CH₃)₃, C(O)O(linear C₁-C₆alkyl)OC(O)CH₂CH₂OH, C(O)O(linear C₁-C₆alkyl)OC(O)Me, C(O)OCH₂CH₂heterocycloalkyl, or C(O)O(branched C₁-C₆alkyl)OC(O)O(CH₂CH₂O)₂Et.

In some embodiments:
[linker] is

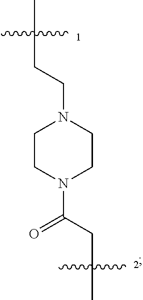 [SMILES: *CCN1CCN(C(=O)C*)CC1]

[ligase ligand moiety] is

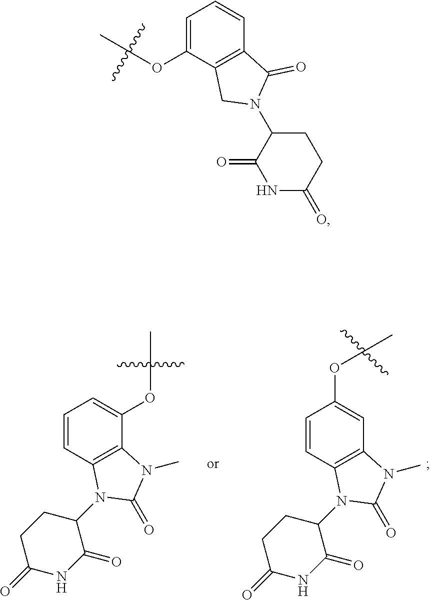 [SMILES: *Oc1cccc2c1CN(C1CCC(=O)NC1=O)C2=O ; *Oc1cccc2c1n(C)c(=O)n2C1CCC(=O)NC1=O ; *Oc1ccc2c(c1)n(C)c(=O)n2C1CCC(=O)NC1=O]

and
[MCL-1 ligand prodrug moiety] is a compound of Formula (A3), wherein
R³² is

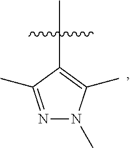

and
R⁹ is C(O)O(linear C₁-C₆alkyl)OC(O)C(CH₃)₃, C(O)O(linear C₁-C₆alkyl)OC(O)CH₂CH₂OH, C(O)O(linear C₁-C₆alkyl)OC(O)Me, or C(O)OCH₂CH₂heterocycloalkyl;
wherein when R⁹ is C(O)O(linear C₁-C₆alkyl)OC(O)C(CH₃)₃ then [ligase ligand moiety] is

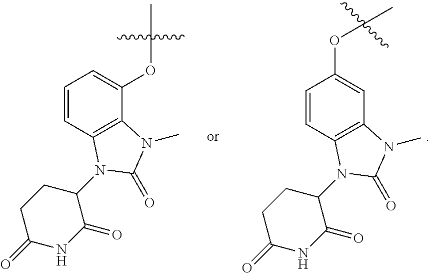 [SMILES: *Oc1cccc2c1n(C)c(=O)n2C1CCC(=O)NC1=O ; *Oc1ccc2c(c1)n(C)c(=O)n2C1CCC(=O)NC1=O]

In some embodiments, the compound is selected from:

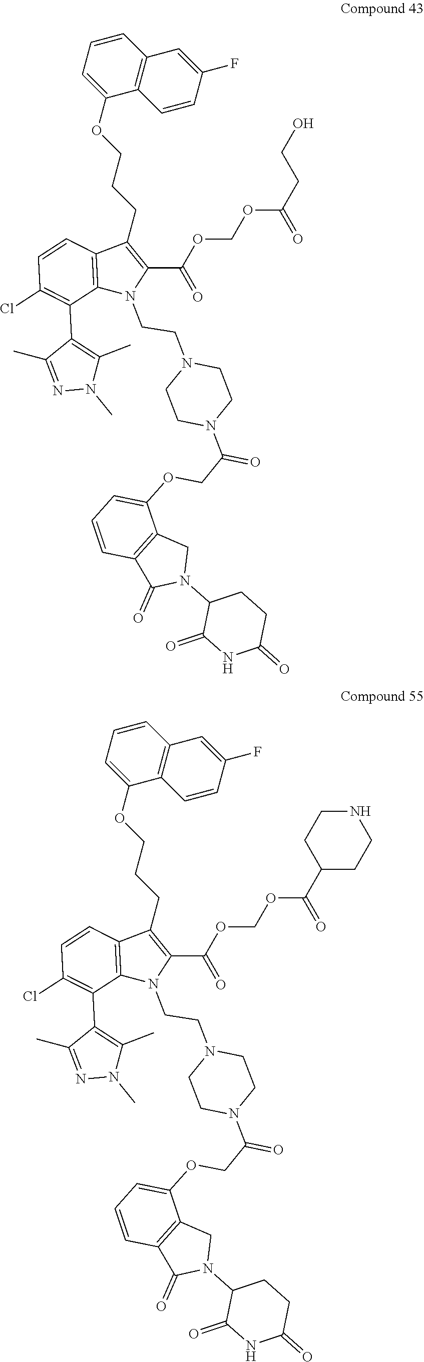 [SMILES: Cc1nn(C)c(C)c1-c1c(Cl)ccc2c(CCCOc3cccc4cc(F)ccc34)c(C(=O)OCOC(=O)CCO)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C3CCNCC3)n(CCN3CCN(C(=O)COc4cccc5c4CN(C4CCC(=O)NC4=O)C5=O)CC3)c12]

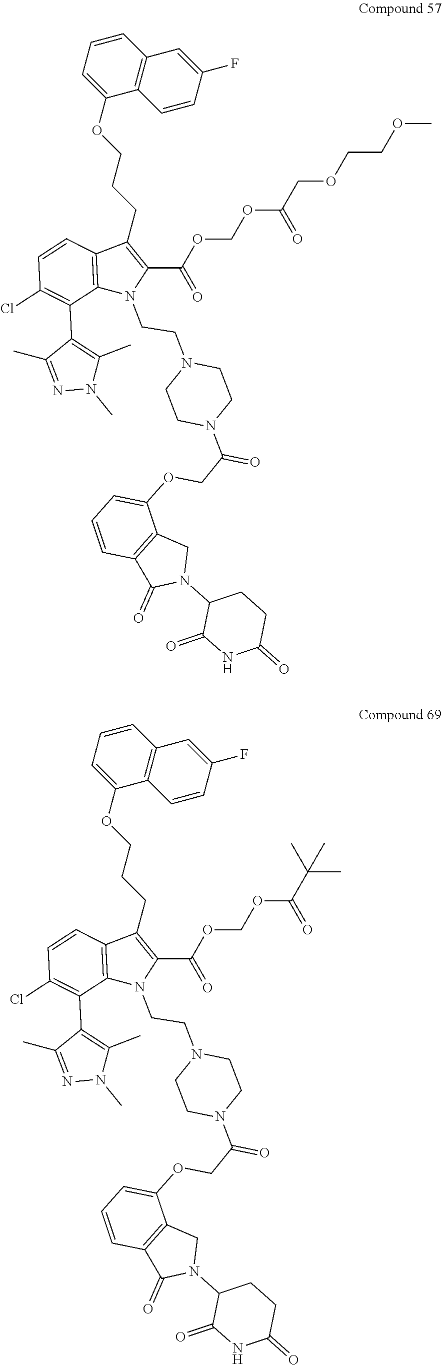 [SMILES: COCCOCC(=O)OCOC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1 ; Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4cccc5c4CN(C4CCC(=O)NC4=O)C5=O)CC3)c12]

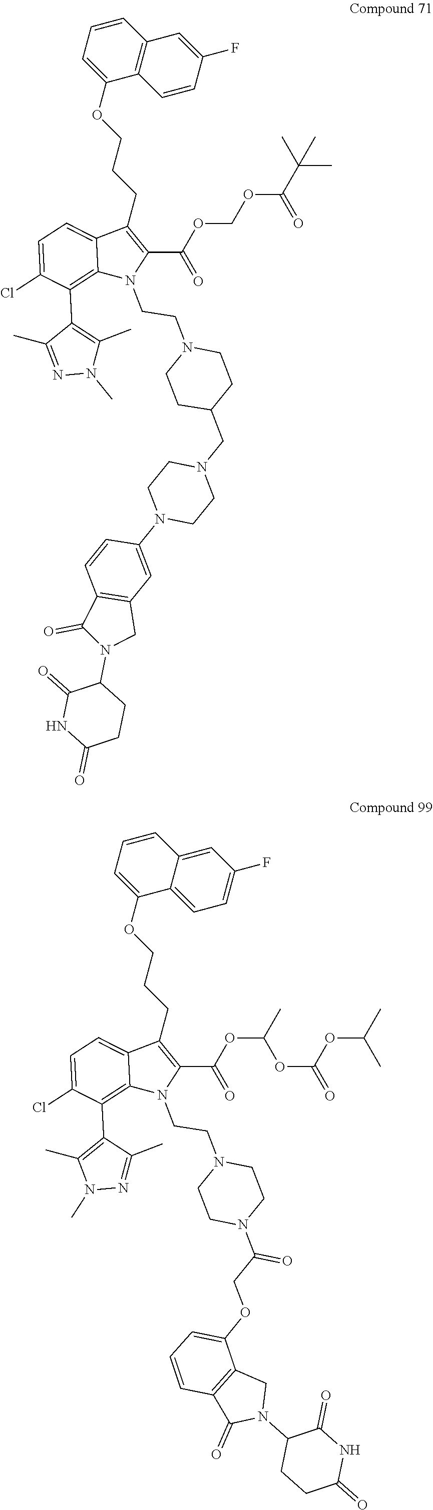 [SMILES: Cc1nn(C)c(C)c1-c1c(Cl)ccc2c(CCCOc3cccc4cc(F)ccc34)c(C(=O)OCOC(=O)C(C)(C)C)n(CCN3CCC(CN4CCN(c5ccc6c(c5)CN(C5CCC(=O)NC5=O)C6=O)CC4)CC3)c12 ; Cc1nn(C)c(C)c1-c1c(Cl)ccc2c(CCCOc3cccc4cc(F)ccc34)c(C(=O)OC(C)OC(=O)OC(C)C)n(CCN3CCN(C(=O)COc4cccc5c4CN(C4CCC(=O)NC4=O)C5=O)CC3)c12]

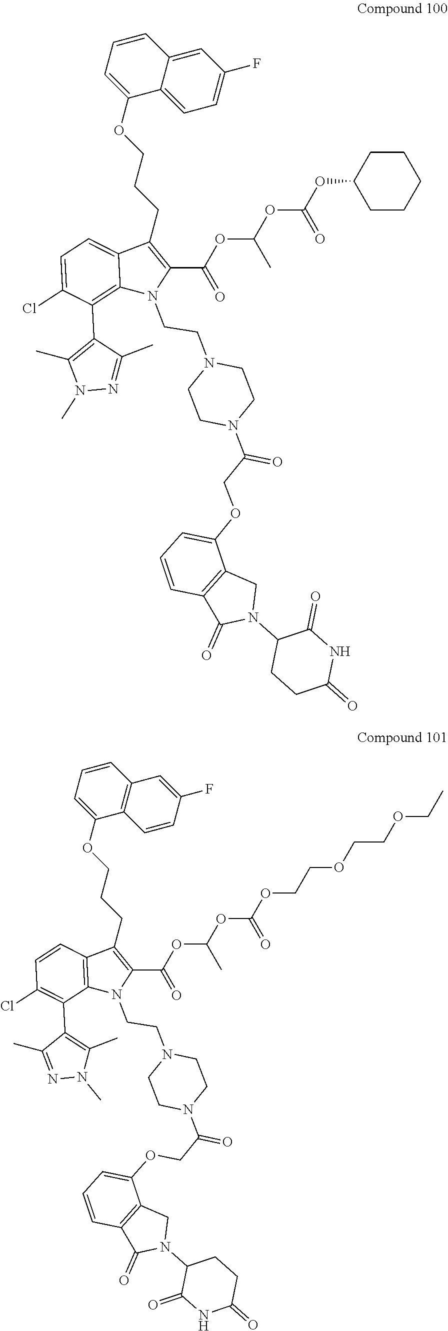

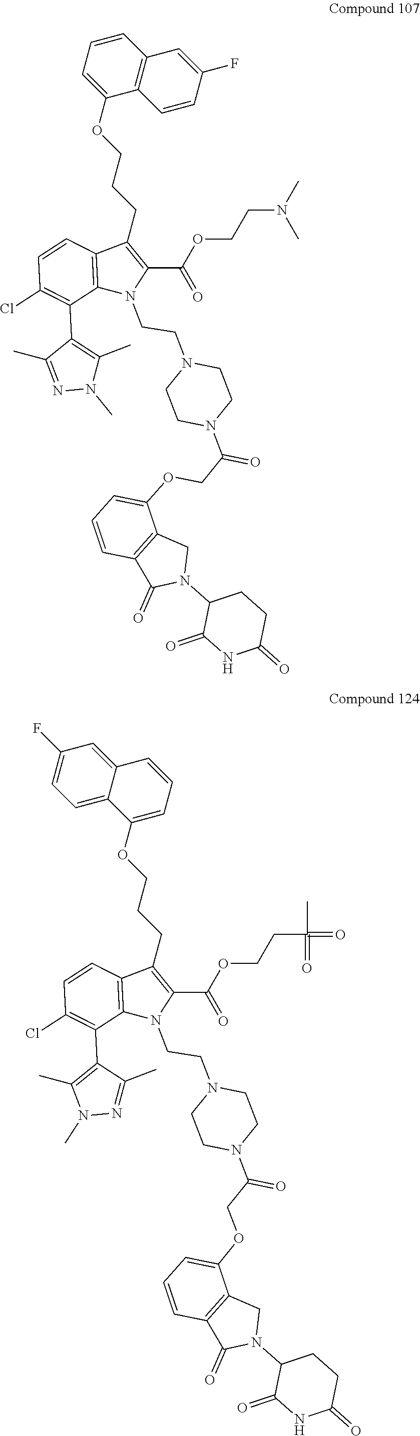

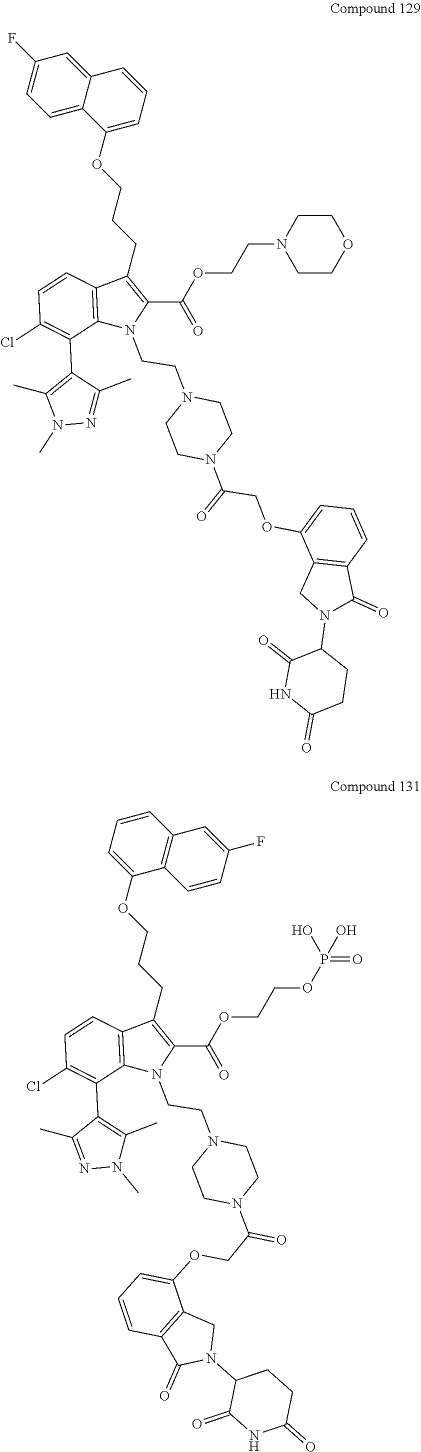 [SMILES: Cc1nn(C)c(C)c1-c1c(Cl)ccc2c(CCCOc3cccc4cc(F)ccc34)c(C(=O)OCCN3CCOCC3)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCOP(=O)(O)O)n(CCN3CCN(C(=O)COc4cccc5c4CN(C4CCC(=O)NC4=O)C5=O)CC3)c12]

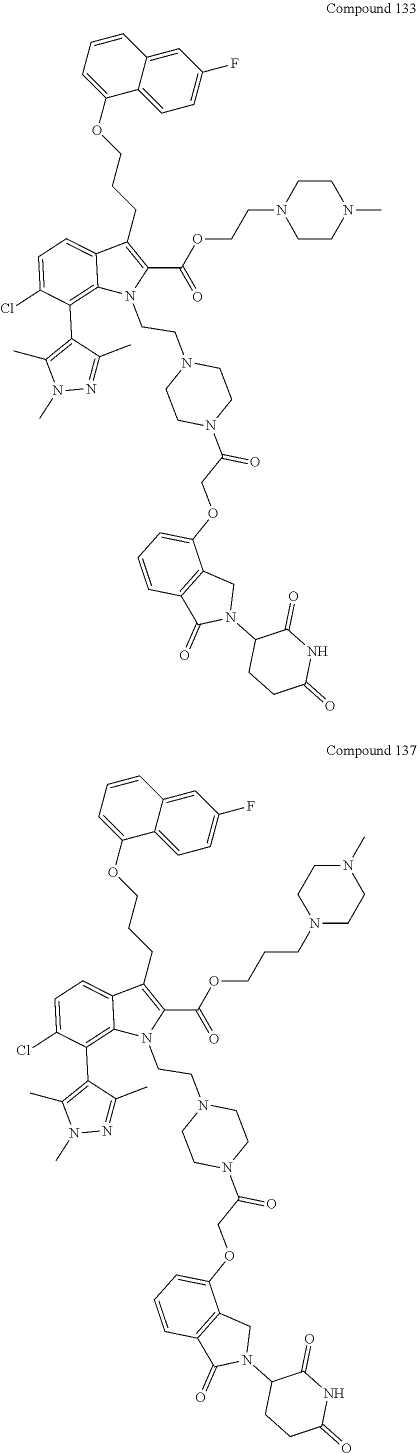 [SMILES: Cc1nn(C)c(C)c1-c1c(Cl)ccc2c(CCCOc3cccc4cc(F)ccc34)c(C(=O)OCCN3CCN(C)CC3)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCCN3CCN(C)CC3)n(CCN3CCN(C(=O)COc4cccc5c4CN(C4CCC(=O)NC4=O)C5=O)CC3)c12]

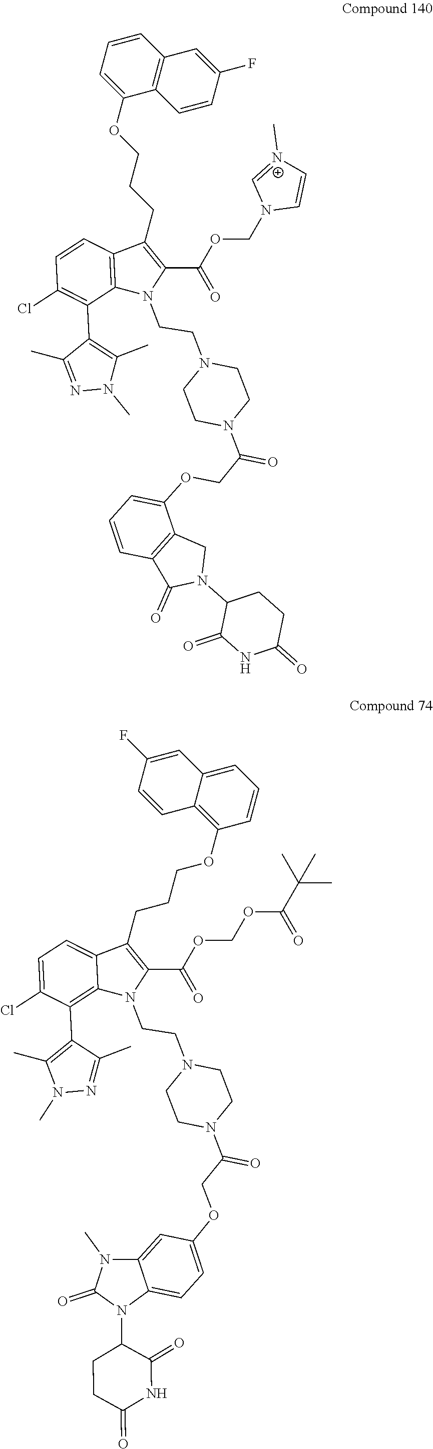 [SMILES: Cc1nn(C)c(C)c1-c1c(Cl)ccc2c(CCCOc3cccc4cc(F)ccc34)c(C(=O)OCn3cc[n+](C)c3)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4ccc5c(c4)n(C)c(=O)n5C4CCC(=O)NC4=O)CC3)c12]

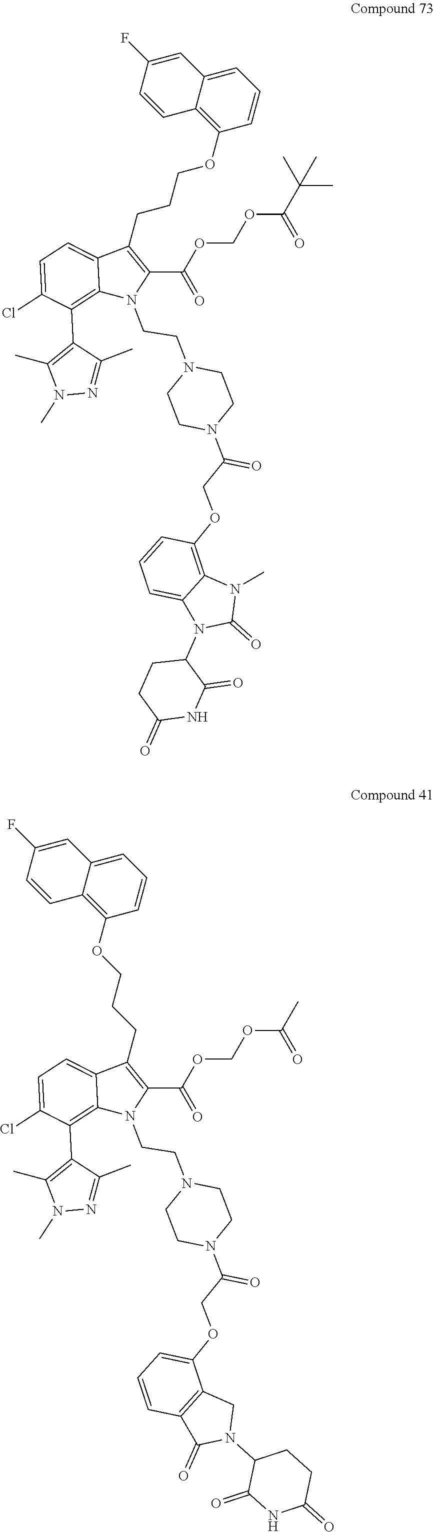 [SMILES: Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4cccc5c4n(C)c(=O)n5C4CCC(=O)NC4=O)CC3)c12 ; CC(=O)OCOC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1]

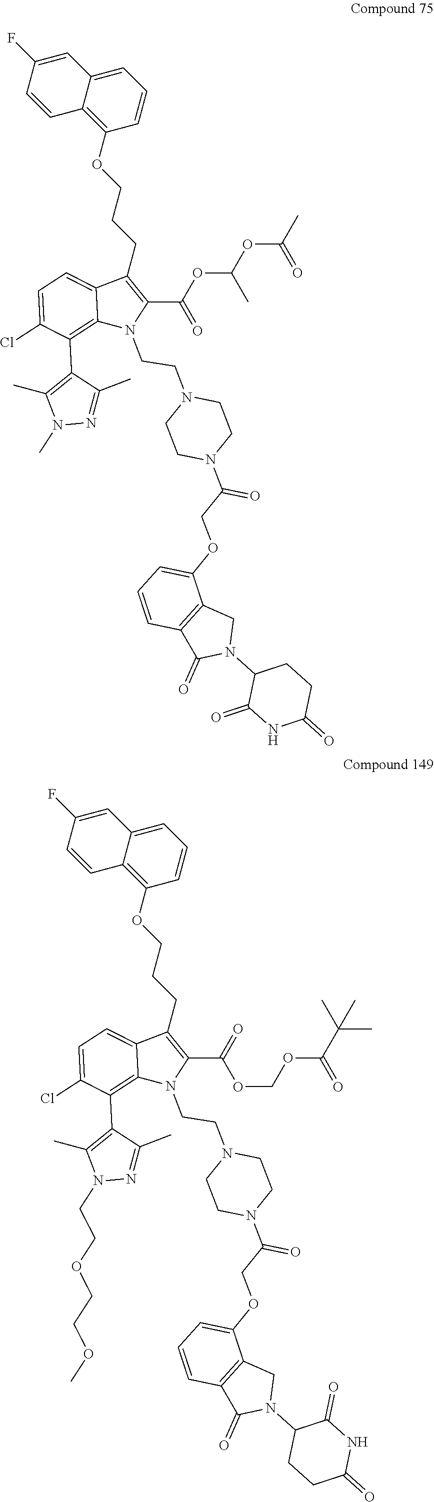 [SMILES: CC(=O)OC(C)OC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1 ; COCCOCCn1nc(C)c(-c2c(Cl)ccc3c(CCCOc4cccc5cc(F)ccc45)c(C(=O)OCOC(=O)C(C)(C)C)n(CCN4CCN(C(=O)COc5cccc6c5CN(C5CCC(=O)NC5=O)C6=O)CC4)c23)c1C]

In some embodiments, the compound is selected from Compounds 43, 55, 57, 69, 71, 99, 100, 101, 129, 131, 133, 137 and 140. In other embodiments, the compound is selected from Compound 107 and Compound 124.

In some embodiments, the compound is selected from Compounds 69, 99, 100, 107, 124 and 140.

In some embodiments, the compound is selected from Compounds 41, 43, 55, 57, 69, 71, 73, 74, 75, 99, 100, 101 and 124. In some embodiments, the compound is selected from Compounds 41, 43, 55, 57, 69, 71, 73, 74, 75, 99, 100 and 101. In some embodiments, the compound is selected from Compounds 69, 75, 99, 100, 101 and 124. In some embodiments, the compound is selected from Compounds 69, 75, 99, 100 and 101.

In some embodiments, the compound is selected from Compounds 41, 43, 69, 73, 74, 101, 129 and 133.

In some embodiments, the compound is selected from Compounds 41, 43, 73, 74, 129 and 133.

In some embodiments, wherein unless otherwise specified each alkyl, alkenyl, alkynyl, aryl, heteroaryl and benzyl is unsubstituted.

In accordance with a second aspect of the invention, there is provided a compound of formula (I)

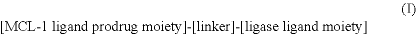

or a salt, solvate, hydrate or isomer thereof,
wherein [ligase ligand moiety] is:
(a) Formula (IV) or (IVa)

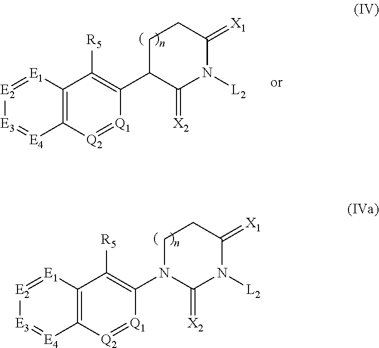

wherein:
each of X₁ and X₂ is independently O or S;
each of Q₁ and Q₂ is independently N or CRS, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —N₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
each Rⁱ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹;
and
each R^{b} is independently hydrogen, alkyl, alkenyl, aryl, heteroaryl, or benzyl;
   wherein R²¹ is a bond connected to R¹⁸ of the linker, and wherein Formula (IV) contains a single R²¹;
(b) Formula (VIIa), (VIIb), (VIIc) or (VIId):

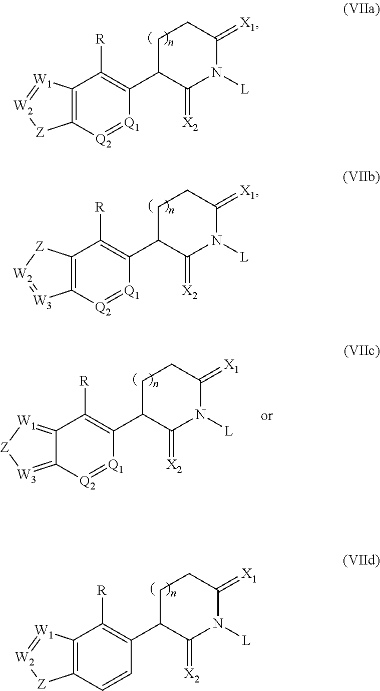

wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR^{e};
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹;
each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII):

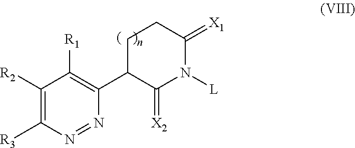

wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX):

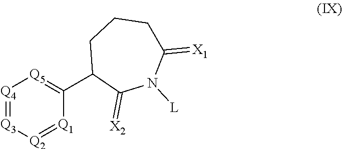

wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single R²¹;
   wherein [MCL-1 ligand prodrug moiety] is a compound of Formula (A1), (A2), (A3) or (A4):

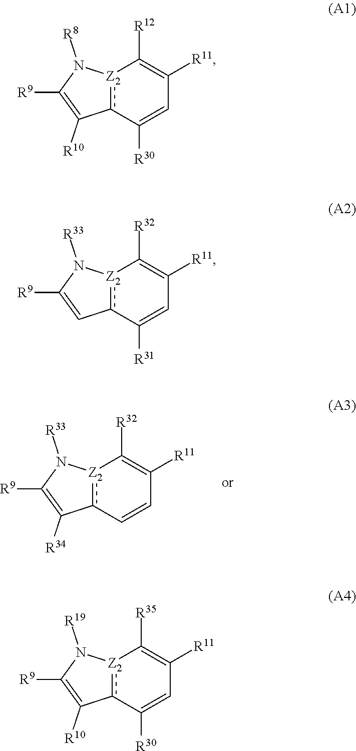

wherein
[symbol] is a single bond or a double bond;
each Z₂ is independently N or C, wherein when Z₂ is N, then [symbol] is a single bond; and when Z₂ is C, then [symbol] is a double bond;
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is

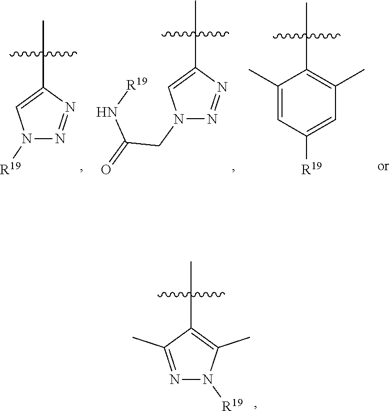

R³¹ is —C₂₋₅alkyl-O—R¹³, —O—C₂₋₅alkyl-R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

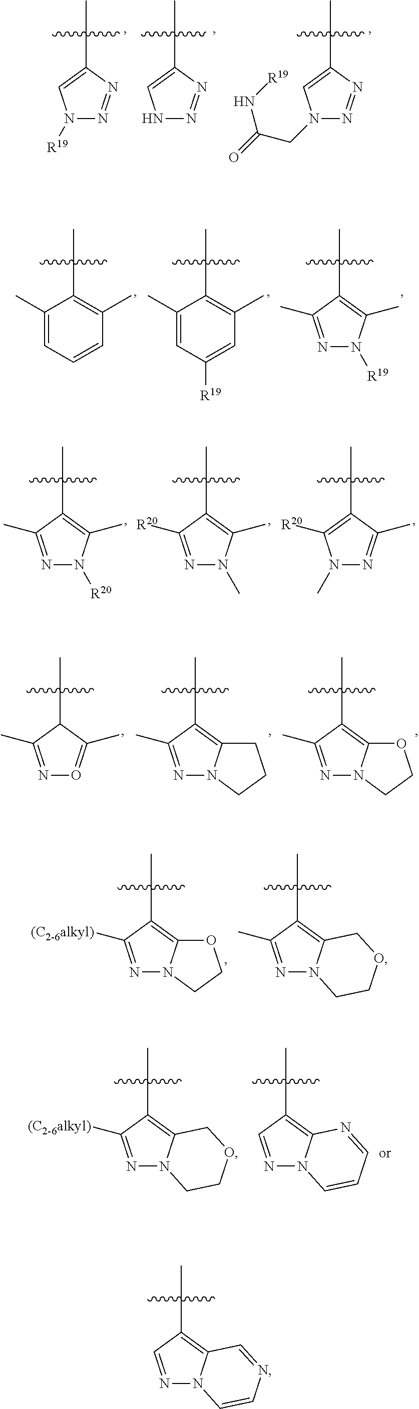

each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is

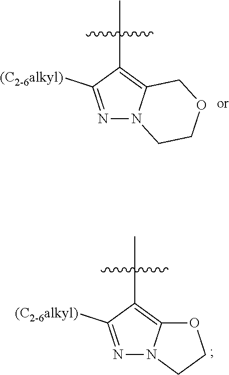

R¹⁹ is a bond connected to R¹⁴ of the linker; wherein each of Formula (A1), Formula (A2), Formula (A3) and Formula (A4) contains a single R¹⁹; and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, C(O)NMe₂;
each R^{p} is independently —(CH₂)ₘOC(O)(unsubstituted C₁-C₆alkyl), and
each m is independently 1, 2, 3 or 4;
or
(ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me, wherein each p is independently 2, 3, 4, 5, or 6;
   and wherein [linker] has the following formula

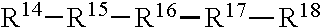

wherein
R¹⁴ is —C₁-C₆ alkyl, —C₂₋₆ alkenyl, —C₂₋₆ alkynyl, —C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)ₓ, —(C₃H₆—O)ₓ, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
   wherein at least one of R¹⁴—R¹⁸ is present.

In some embodiments of the second aspect, unless otherwise specified each alkyl, alkenyl, alkynyl, aryl, heteroaryl and benzyl groups is unsubstituted.

In some embodiments of the second aspect, each Rⁱ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹. In some such embodiments, each R^{b} is independently hydrogen, alkyl, cycloalkyl, or aryl. In some such embodiments, the aryl is substituted with one or more groups selected from halogen, alkyl and O-haloalkyl, optionally wherein the halogen is Cl, the alkyl is methyl and the O-haloalkyl is O—CF₃.

In some embodiments of the second aspect, one of E₁, E₂, E₃ and E₄ is N, and the remaining three of E₁, E₂, E₃ and E₄ are each CR′. In some such embodiments, E₁ is N, and E₂, E₃ and E₄ are CR′. In other such embodiments, E₂ is N, and E₁, E₃ and E₄ are CR′. In other such embodiments, E₃ is N, and E₁, E₂ and E₄ are CR′. In other such embodiments, E₄ is N, and E₁, E₂ and E₃ are CR′.

In some embodiments of the second aspect, E₁, E₂, E₃ and E₄ are each CR′, optionally wherein E₁, E₂, E₃ and E₄ are each CH. In some such embodiments, three of E₁, E₂, E₃ and E₄ are CH, and one of E₁, E₂, E₃ and E₄ is C-halogen, C-alkyl, C-alkenyl, C-alkynyl, C-aryl, C-heteroaryl, C-benzyl, C-haloalkyl, C-haloalkenyl, C—NH₂, C—NHR^{b}, C—NR^{b}₂, C—NR^{b}C(O)R^{b}, C—NR^{b}C(O)OR^{b}, C—NO₂, C—CN, C—C(O)R^{b}, C—C(O)OR^{b}, C—C(O)NH₂, C—C(O)NHR^{b}, C—C(O)NR^{b}₂, C—C(O)NHCHR^{b}₂, C—CHR^{b}NHC(O)NHR^{b}, C—CHR^{b}NHC(O)C(halogen)₂R^{b}, C—OR^{b}, C—OC(O)R^{b}, C—OC(O)OR^{b}, C—OC(O)NH₂, C—OC(O)NHR^{b}, C—OC(O)NR^{b}₂, C—SR^{b}, C—S(O)₂R^{b}, C—S(O)₂OR^{b}, C—S(O)₂NH₂, C—S(O)₂NHR^{b}, C—S(O)₂NR^{b}₂, C—NHS(O)₂R^{b}, —R²¹, —O—R²¹, —NH—R²¹, —C(O)—R²¹, —C(O)—NH—R²¹, or —CH₂—NH—C(O)—R²¹.

In some embodiments of the second aspect, E₂, E₃ and E₄ are each CH. In other embodiments, E₁, E₃ and E₄ are each CH. In other embodiments, E₁, E₂ and E₄ are each CH.

In some embodiments of the second aspect, two of E₁, E₂, E₃ and E₄ are N, and the remaining two of E₁, E₂, E₃ and E₄ are each CR′.

In some embodiments of the second aspect, three of E₁, E₂, E₃ and E₄ are N, and the remaining one of E₁, E₂, E₃ and E₄ is CR′.

In some embodiments of the second aspect, one of W₁, W₂ and W₃ is N, and the other of W₁, W₂ and W₃ is CR^{a}. In some such embodiments, R^{a} is H.

In some embodiments of the second aspect, W₁, W₂ and W₃ are each CR^{a}.

In some embodiments of the second aspect, W₁ is C—NH₂, C—NHR^{b} or C—NR^{b}₂; optionally C—NH₂.

In some embodiments of the second aspect, W₁, W₂ and W₃ are each N.

In some embodiments of the second aspect, Z is NH or N-alkyl, optionally NH or N-Me.

In some embodiments of the second aspect, in Formula (IV), Formula (VIIa), Formula (VIIb) and Formula (VIIc), Q₁ is N and Q₂ is CR. In other embodiments, Q₁ is N and Q₂ is N. In other embodiments, Q₁ is CR and Q₂ is N. In some such embodiments, Q₁ is C—H or C-alkyl. In some such embodiments, Q₁ is C—H or C-Me.

In some embodiments of the second aspect, in Formula (IX) one of Q₁, Q₂, Q₃, Q₄ and Q₅ is N, and the remaining four of Q₁, Q₂, Q₃, Q₄ and Q₅ are each CR. In some such embodiments, Q₁ is N, In other embodiments, Q₂ is N. In other embodiments, Q₃ is N.

In some embodiments of the second aspect, in Formula (IX) two of Q₁, Q₂, Q₃, Q₄ and Q₅ are N, and the remaining three of Q₁, Q₂, Q₃, Q₄ and Q₅ are each CR. In some such embodiments, Q₁ and Q₂ are N, and Q₃, Q₄ and Q₅ are each CR. In other embodiments, Q₂ and Q₃ are N, and Q₁, Q₄ and Q₅ are each CR. In other embodiments, Q₁ and Q₃ are N, and Q₂, Q₄ and Q₅ are each CR. In other embodiments, Q₂ and Q₄ are N, and Q₁, Q₃ and Q₅ are each CR. In other embodiments, Q₁ and Q₄ are N, and Q₂, Q₃ and Q₅ are each CR.

In some embodiments of the second aspect, in Formula (IX) three of Q₁, Q₂, Q₃, Q₄ and Q₅ are N, and the remaining two of Q₁, Q₂, Q₃, Q₄ and Q₅ are each CR.

In some embodiments of the second aspect, [ligase ligand moiety] is:

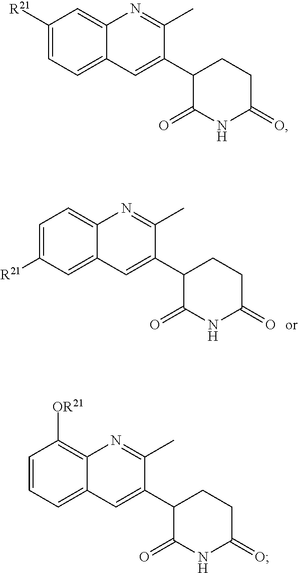

In some embodiments of the second aspect, [ligase ligand moiety] is:

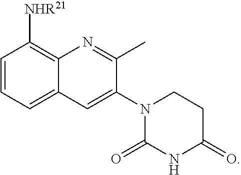

In other embodiments of the second aspect, [ligase ligand moiety] is:

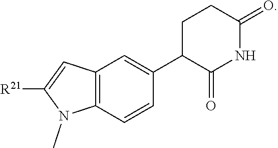

In accordance with a third aspect of the invention, there is provided a compound of formula (I)

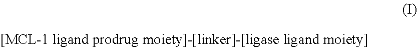

or a salt, solvate, hydrate or isomer thereof,
wherein [ligase ligand moiety] is:
(a) Formula (Va) or (Vb):

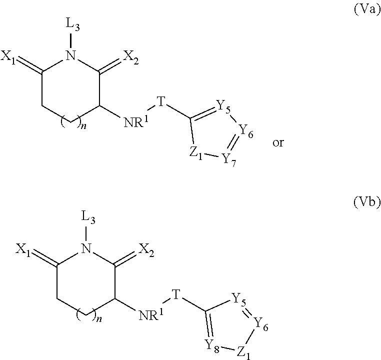

or a pharmaceutically acceptable salt or tautomer thereof,
wherein
each of X₁ and X₂ is independently O or S;
Z₁ is O, S or NR⁶;
T is is C═O or SO₂;
R¹ is hydrogen, alkyl, cycloalkyl, alkenyl, cycloalkenyl, aryl, heteroaryl, or benzyl;
each of Y₅, Y₆, Y₇, and Y₈ is independently N or CR⁷,
wherein at least one of Y₅, Y₆ and Y₇ in Formula (Va) is CR⁷, and at least one of Y₅, Y₅ and Y₈ in Formula (Vb) is CR⁷;
n is 0, 1 or 2;
L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″₂, —OR″″, —NR″″₂, or —S(O)₂R″″;
each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
each R″″ is independently hydrogen, alkyl, cycloalkyl, alkenyl, cycloalkenyl, aryl, heteroaryl, or benzyl;
R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
wherein R²¹ is a bond connected to R¹⁸ of the linker, and wherein formula (Va) and formula (Vb) each contain a single R²¹;
wherein when Z₁ is O, then Y₆ is CR⁷ and
   wherein when the compound is of Formula (Va), then
(i) when each of Y₅, Y₆ and Y₇ is CR⁷, then at least one of R⁷ is not H;
(ii) when Z₁ is NR⁶, then Y₆ and Y₇ are CR⁷;
(iii) when Z₁ is S, then Y₅ is not C—OMe and Y₆ is not C—OMe;
(iv) when Z₁ is S and Y₅ is C—NHCOMe, then Y₇ is not C—CH₂NR″″C(O)OR″″;
(v) when Z₁ is S and Y₅ is N, then Y₆ is not C—H, C-aryl or C—C(O)OR″″; and
(vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
   and when the compound is of Formula (Vb), then:
(vii) when each of Y₅, Y₆ and Y₈ is CR⁷, then at least one of R⁷ is not H;
(viii) when Z₁ is S, then Y₅ is not C—COOH or C—NHC(O)Me, and Y₈ is not C—Br;
(ix) when Z₁ is S and Y₆ is C—Br, then Y₈ is C—OR″″
(x) when Z₁ is S, Y₅ is N and Y₆ is C—H or C—NH₂, then Y₈ is not C—H
(xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷.
   or
(b) Formula (IIa) or (IIb):

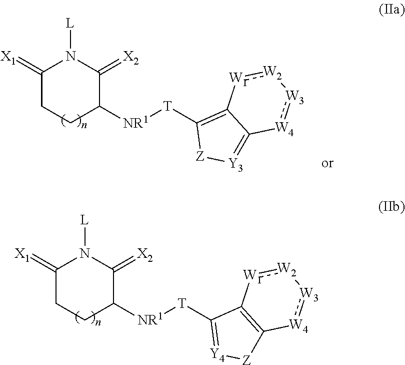

wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or CR^{a}, wherein at least one of W₁, W₂, W₃ and W₄ is N, and
   when each [symbol] is a single bond, W₁, W₂, W₃ and W₄ are each CR^{a}₂ and Y₄ is CR;
n is 0, 1 or 2; [symbol]
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
   wherein [MCL-1 ligand prodrug moiety] is a compound of Formula (A1), (A2), (A3) or (A4):

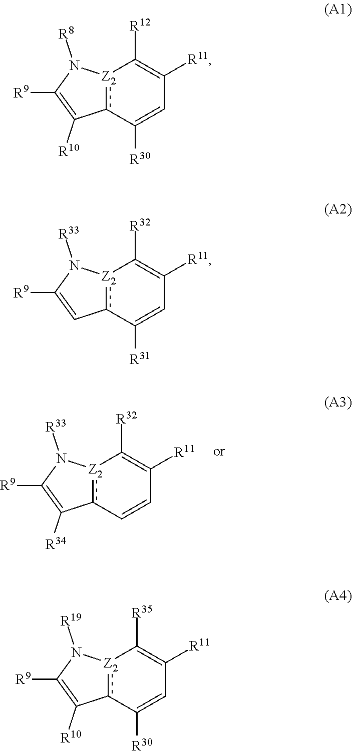

wherein
[symbol] is a single bond or a double bond;
each Z₂ is independently N or C, wherein when Z₂ is N, then [symbol] is a single bond; and when Z₂ is C, then [symbol] is a double bond;
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is

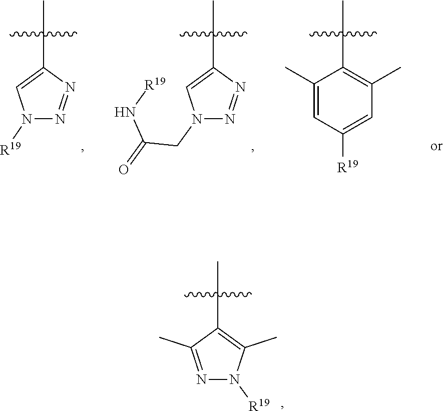

R³¹ is —C₂₋₅alkyl-O—R¹³, —O—C₂₋₅alkyl-R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

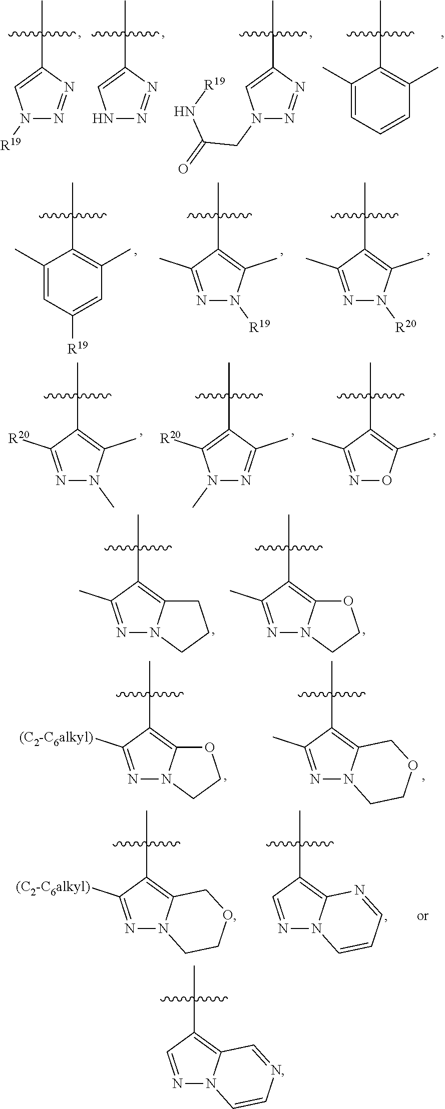

each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is

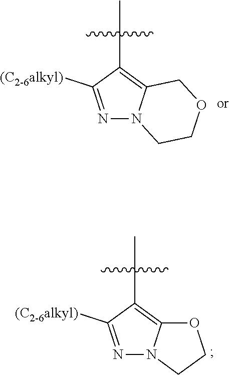

R¹⁹ is a bond connected to R¹⁴ of the linker; wherein each of Formula (A1), Formula (A2), Formula (A3) and Formula (A4) contains a single R¹⁹; and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, —C(O)NMe₂;
each R^{p} is independently —(CH₂)ₘOC(O)(unsubstituted C₁-C₆alkyl), and
each m is independently 1, 2, 3 or 4;
or
(ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me wherein each p is independently 2, 3, 4, 5, or 6;
   and wherein [linker] has the following formula

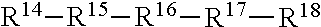

wherein
R¹⁴ is —C₁-C₆ alkyl, —C₂₋₆ alkenyl, —C₂₋₆ alkynyl, —C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)ₓ, —(C₃H₆—O)ₓ, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
wherein at least one of R¹⁴—R¹⁸ is present.

In some embodiments of the third aspect, unless otherwise specified each alkyl, alkenyl, alkynyl, aryl, heteroaryl and benzyl groups is unsubstituted.

In some embodiments of the third aspect, when Z₁ is S in Formula (Vb), then Y₅ is not C— NHC(O)R″″ or —C(O)OR″″.

In some embodiments of the third aspect, Z₁ is NR⁶.

In some embodiments of the third aspect, each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —NR″″C(O)CH(OH)R″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹.

In some embodiments of the third aspect, R¹ is hydrogen.

In some embodiments of the third aspect, R⁶ is hydrogen.

In some embodiments of the third aspect, [ligase ligand moiety] is of Formula (Va) and Y₅, Y₆ and Y₇ are each CR⁷. In some such embodiments, Y₅ is —C—NHC(O)R″″; Y₆ is CH; and Y₇ is CH or CCl. In some such embodiments, L₃ is hydrogen; Z₁ is S; R¹ is hydrogen; T is C═O; and Y₇ is CH.

In some embodiments of the third aspect, the compound is of Formula (Vb) and Y₅, Y₆ and Y₈ are each CR⁷. In some such embodiments:
L₃ is hydrogen;
Z₁ is S;
R¹ is H;
T is C═O;
Y₅ is CH, C—OR″″, CCI, C—CN, or C—NHC(O)R″″;
Y₆ is CH, CCI, C-alkyl, C-cycloalkyl, or C-haloalkyl; and
Y₈ is CH, C—OR″″, C—NHC(O)R″″, C—NHC(O)OR″″, C—NHR″″, C—NH₂, or C—NHSO₂R″″; wherein, when Y₅ is CCI, then Y₆ is CH, C-alkyl, C-cycloalkyl, or C-haloalkyl. In some such embodiments, each R″″ is independently alkyl, cycloalkyl, aryl or benzyl. In some embodiments, Y₅ is CH; Y₆ is CH or CCI; and Y₈ is C—OR″″ or C—NH₂, optionally C—OMe or C—NH₂.

In some embodiments of the third aspect, Z is NR². In other embodiments, Z is S.

In some embodiments of the third aspect, each [symbol] is a double bond.

In some embodiments of the third aspect, L is hydrogen.

In some embodiments of the third aspect, one of W₁, W₂, W₃ and W₄ is N, and the remaining three of W₁, W₂, W₃ and W₄ are each CR^{a}. In some such embodiments W₄ is CR^{a}.

In some embodiments of the third aspect, two of W₁, W₂, W₃ and W₄ is N, and the remaining two of W₁, W₂, W₃ and W₄ are each CR^{a}.

In some embodiments of the third aspect, one of W₁, W₂, W₃ and W₄ is CR^{a}, and the remaining three of W₁, W₂, W₃ and W₄ are each N.

In some embodiments of the third aspect, each R is independently hydrogen, halogen or —NR^{h}C(O)R^{h}.

In accordance with a fourth aspect of the invention, there is provided a compound of formula (I)

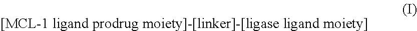

or a salt, solvate, hydrate or isomer thereof,
wherein [ligase ligand moiety] is:
(a) Formula (VIa) or (Vib):

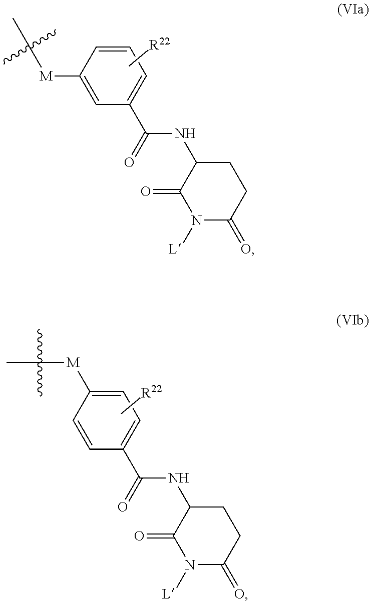

wherein
M is O, S or NH, or is absent;
[symbol] indicates attachment to R¹⁸ of the linker;
R²² is hydrogen, halogen, —OMe, an amino group, heterocycloalkyl, or unsubstituted C₁-C₆ alkyl; and
L′ is hydrogen, alkyl, benzyl, acetyl or pivaloyl
(b) Formula (II):

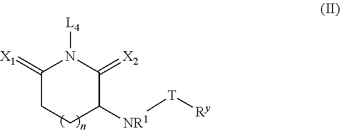

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{y} is selected from

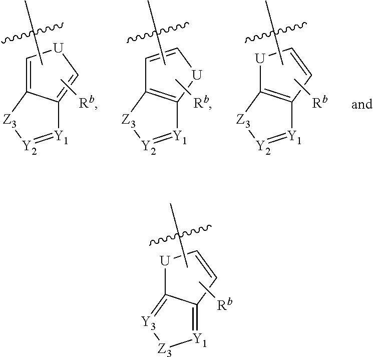

wherein [symbol] indicates attachment to T,
Z₃ is O, S or NR³;
U is O, S, NR^{b} or CRⁱ₂;
each of Y₁, Y₂ and Y₃ is independently N or CR^{d};
each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
each R^{b} is independently alkyl, cycloalkyl, alkenyl, cycloalkenyl, aryl, heteroaryl, or benzyl;
R²¹ is a bond connected to R¹⁸ of the linker, wherein Formula (II) contains a single R²¹;
wherein,
(i) when R^{y} is

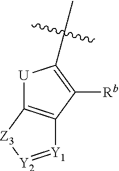

then Y₂ is CR^{d}; and
(ii) when R^{y} is

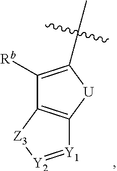

then Rⁱ in CRⁱ₂ is not hydrogen or
(c) Formula (III):

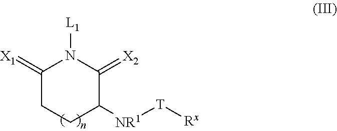

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from

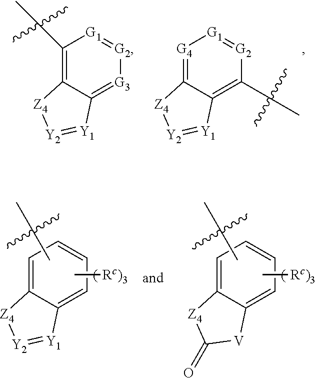

wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f}then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
each R^{b} is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{c}, then C═X₁ may be replaced by CH;
   and wherein:
(i) when R^{x} is

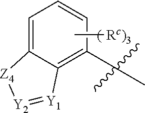

and Z₄ is NH, then L₁ is hydrogen, —CH₂C(O)OR^{b}, or —OR^{b};
(ii) when R^{x} is

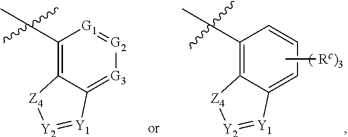

Z₄ is NR⁴, Y₁ is CR^{f}, and Y₂ is N, then R⁴ is not alkyl and at least one of R² and R is not H;
(iii) when R^{x} is

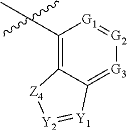

Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not

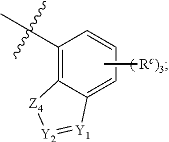

(v) when R^{x} is

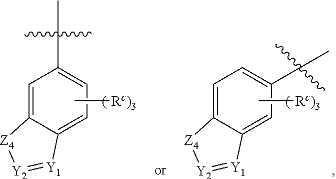

Z₄ is NR⁴, and Y₁ or Y₂ is N, then R⁴ is not alkyl;
(vi) when R^{x} is

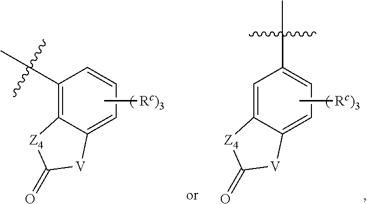

then n=1 or 2; and
(vii) when R^{x} is

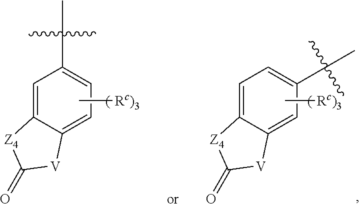

then Z₄=O or S
   wherein [MCL-1 ligand prodrug moiety] is a compound of Formula (A1), (A2), (A3) or (A4):

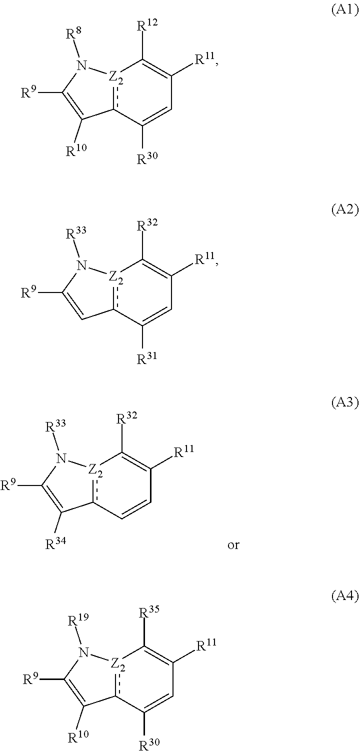

wherein
[symbol] is a single bond or a double bond;
each Z₂ is independently N or C, wherein when Z₂ is N, then [symbol] is a single bond; and when Z₂ is C, then [symbol] is a double bond;
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is

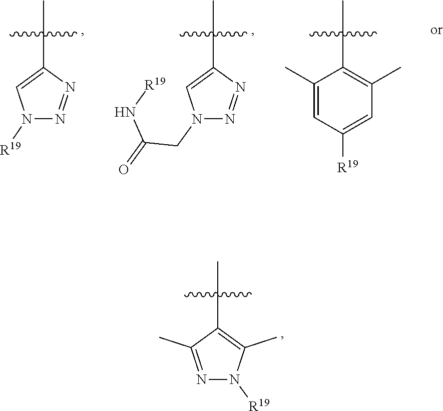

R³¹ is —C₂₋₅alkyl-O—R¹³, —O—C₂₋₅alkyl-R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

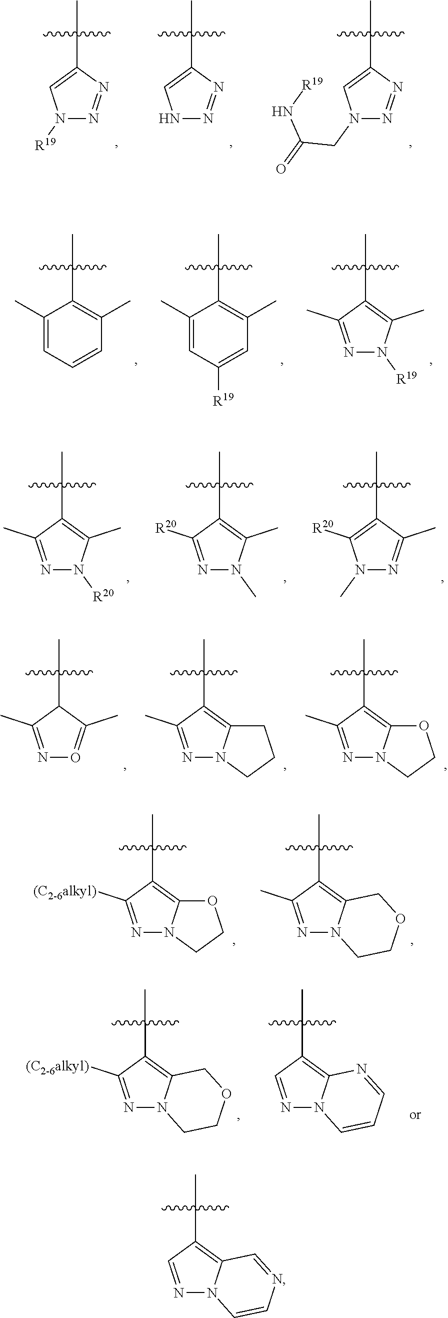

each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is

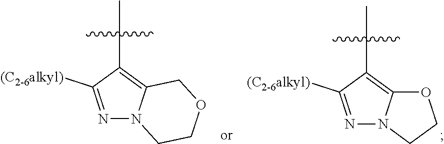

R¹⁹ is a bond connected to R¹⁴ of the linker; wherein each of Formula (A1), Formula (A2), Formula (A3) and Formula (A4) contains a single R¹⁹; and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation; wherein each R¹⁰ is independently —OH, —O(unsubstituted C₁-C₆alkyl), —Obn, —NH₂, —Nme₂, —NHC(O)(unsubstituted C₁-C₆alkyl), —NHC(O)O(unsubstituted C₁-C₆alkyl), heterocycloalkyl, heteroaryl, aryl, —C(O)Nme₂;
each R^{p} is independently —(CH₂)ₘOC(O)(unsubstituted C₁-C₆alkyl), and
each m is independently 1, 2, 3 or 4;
or
(ii) R⁹ is —C(O)O(CH₂)ₚNme₂, —C(O)O(CH₂)ₚNHMe, —C(O)OCH₂CH(OH)CH₂OH, —C(O)OCH₂CH₂CMe₂OH, or —C(O)OCH₂CH₂SO₂Me, wherein each p is independently 2, 3, 4, 5, or 6;
   and wherein [linker] has the following formula

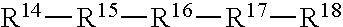

wherein
R¹⁴ is —C₁-C₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)ₓ, —(C₃H₆—O)ₓ, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
wherein at least one of R¹⁴—R¹⁸ is present

In some embodiments of the fourth aspect, unless otherwise specified each alkyl, alkenyl, alkynyl, aryl, heteroaryl and benzyl is unsubstituted.

In some embodiments of the fourth aspect, in Formula (III):
each of X₁ and X₂ is O;
T is C═O;
R¹ is hydrogen,
L₁ is hydrogen,
R^{x} is

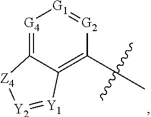

Z₄ is NR⁴;
each of G₁, G₂ and G₄ is CR^{c},
Y₁ is N, and
Y₂ is CR^{f}, wherein R^{f} is not hydrogen.

In some embodiments of the fourth aspect, [ligase ligand moiety] is Formula (III):

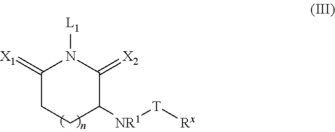

In some embodiments of the fourth aspect, one of R^{c} is —O—R²¹, —NH—R²¹, —C(O)—NH—R²¹, or —CH₂—NH—C(O)—R²¹. In other embodiments, G₁ is C—O—R²¹, C—NH—R²¹, C—C(O)—NH—R²¹, or C—CH₂—NH—C(O)—R²¹. In other embodiments, G₂ is C—O—R²¹, C—NH—R²¹, C—C(O)—NH—R²¹, or C—CH₂—NH—C(O)—R²¹. In other embodiments, R⁴ is R²¹, —C(O)—NH—R²¹, or —CH₂—NH—C(O)—R²¹. In other embodiments, one of R^{f} is —R²¹, —O—R²¹, —NH—R²¹, —C(O)—NH—R²¹, or —CH₂—NH—C(O)—R²¹.

In some embodiments of the fourth aspect, Y₂ is C—R²¹, CO—R²¹, C—NH—R²¹, C—C(O)—NH—R²¹, or C—CH₂—NH—C(O)—R²¹.

In some embodiments of the fourth aspect, [ligase ligand moiety] is selected from:

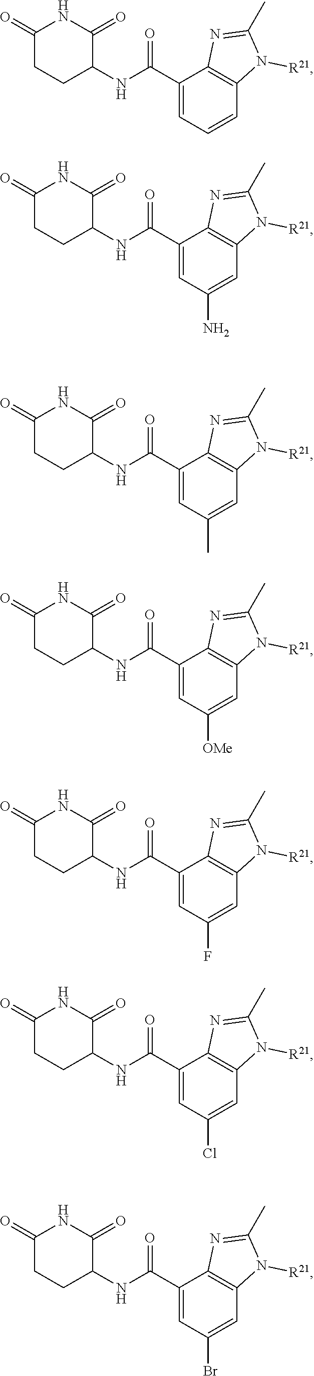

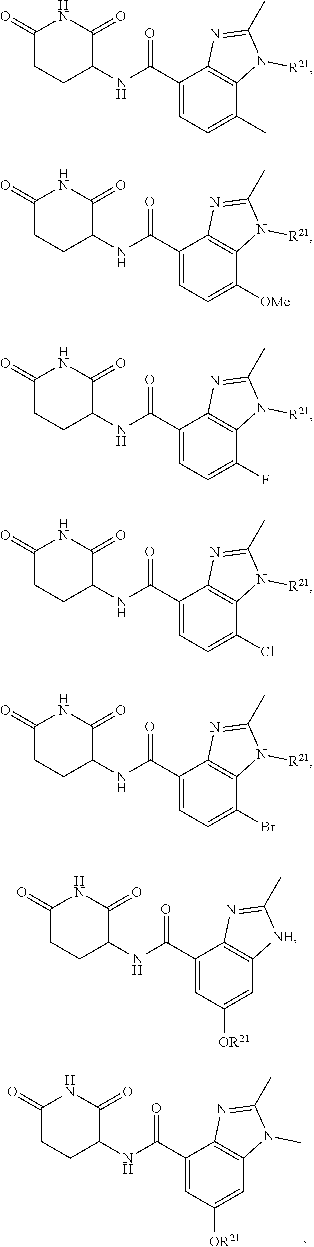

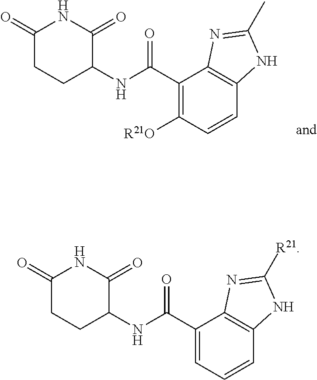

In some embodiments of the fourth aspect, [ligase ligand moiety] is selected from:

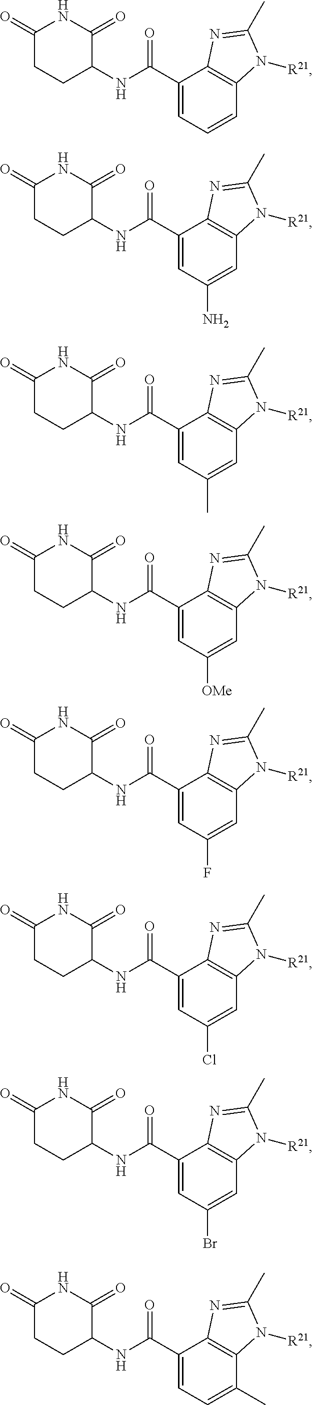

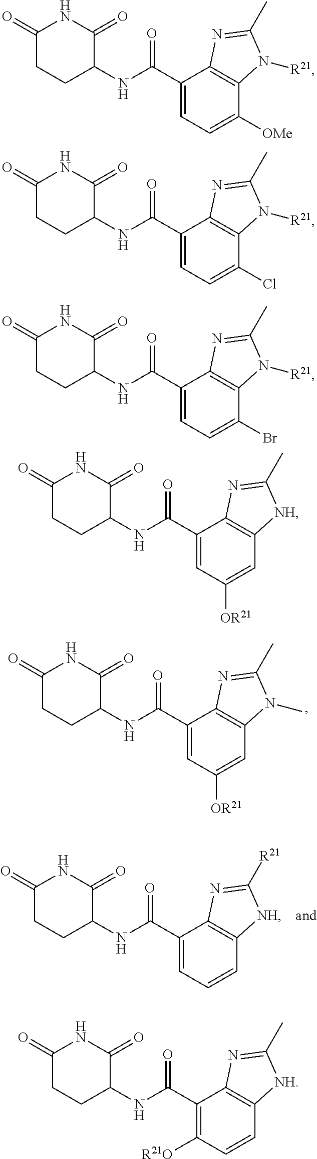

In some embodiments of the fourth aspect, [ligase ligand moiety] is

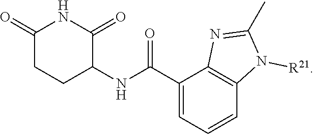

In some embodiments of the fourth aspect, [ligase ligand moiety] is

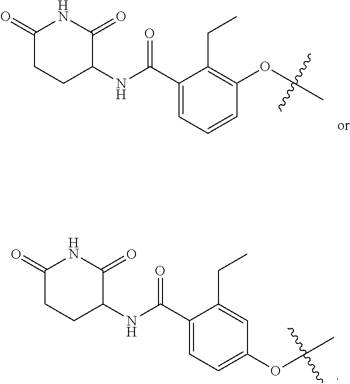 [SMILES: *Oc1cccc(C(=O)NC2CCC(=O)NC2=O)c1CC ; *Oc1ccc(C(=O)NC2CCC(=O)NC2=O)c(CC)c1]

In some embodiments of any of the second, third and fourth aspects:
R¹⁶ is —C₁-C₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(C₂H₄—O)ₓ, —(C₃H₆—O)ₓ, or is absent
R¹⁸ is —C₁₋₆ alkyl, cycloalkyl, —CH₂—NH—C(O)—, heterocycloalkyl, or is absent.

In some embodiments of any of the second, third and fourth aspects, R¹⁴ is —C₁-C₆ alkyl, —C₂₋₆alkenyl, —C₂₋₆ alkynyl, —C(O)—, —SO₂— or is absent. In some embodiments, R¹⁴ is —C₁₋₆alkyl.

In some embodiments of the second, third and fourth aspects, R¹⁵ is cycloalkyl, heterocycloalkyl, aryl, heteroaryl, —C₁₋₆ alkyl-NH—, -cycloalkyl-NH— or is absent. In some such embodiments, R¹⁵ is heterocycloalkyl. In some such embodiments, R¹⁵ is piperazine, bridged piperazine, piperazine N-oxide, piperazine cation,

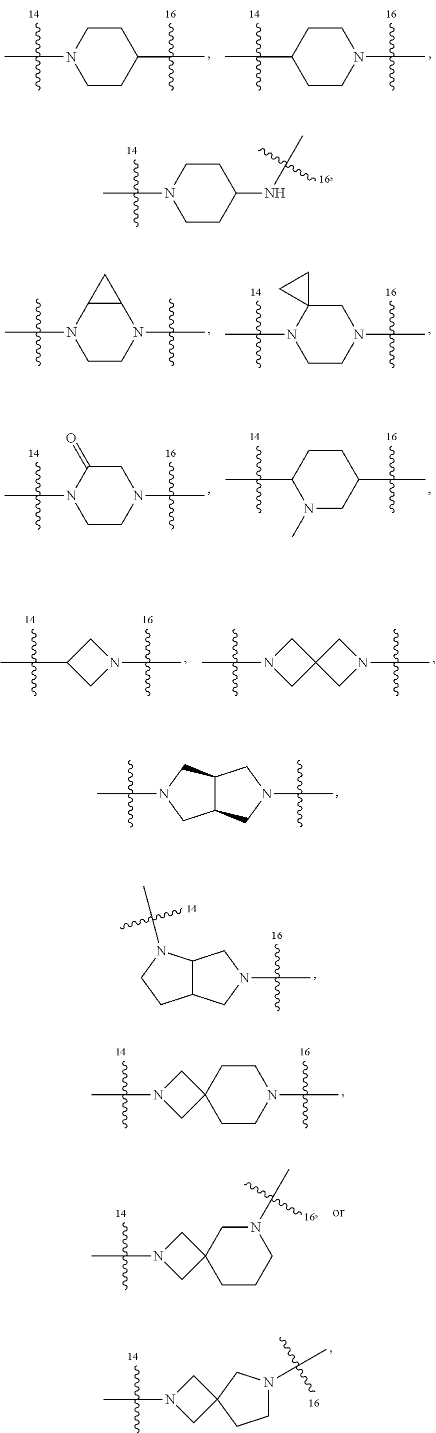

wherein [symbol] indicates attachment to R¹⁴ and [symbol] indicates attachment to R¹⁶. In some such embodiments, R¹⁵ is piperazine or

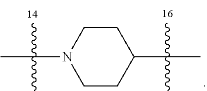 [SMILES: *C1CCN(*)CC1]

In other embodiments, R¹⁵ is absent.

In some embodiments of the second, third and fourth aspects, R¹⁶ is —C₁₋₆alkyl, —CH₂—C(O)—NH— or —C(O)—. In some such embodiments, R¹⁶ is —C₁₋₆alkyl or —C(O)—.

In some embodiments of any of the second, third and fourth aspects, R¹⁷ is absent.

In some embodiments of any of the second, third and fourth aspects, R¹⁸ is —C₁₋₆ alkyl, heterocycloalkyl, or is absent. In some such embodiments, R¹⁸ is —C₁₋₆ alkyl or heterocycloalkyl. In some such embodiments, R¹⁸ is —C₁₋₆ alkyl or piperazine. In other embodiments, R¹⁸ is absent.

In some embodiments of any of the second, third and fourth aspects, [linker] is selected from

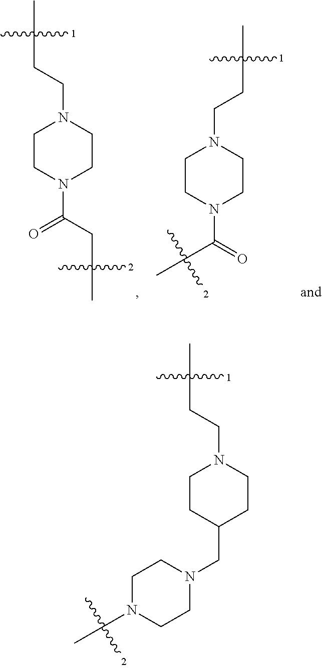 [SMILES: *CCN1CCN(C(=O)C*)CC1 ; *CCN1CCN(C(*)=O)CC1 ; *CCN1CCC(CN2CCN(*)CC2)CC1]

wherein
[symbol] indicates attachment to [MCL-1 ligand prodrug moiety] and
[symbol] indicates attachment to [ligase ligand moiety].

In some embodiments of any of the second, third and fourth aspects, in each of Formula (A1) and Formula (A4), one of R¹⁰ and R³⁰ is H and the other of R¹⁰ and R³⁰ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments of any of the second, third and fourth aspects, in Formula (A1) R¹² is

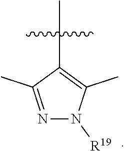

In some embodiments of any of the second, third and fourth aspects, in Formula (A4) R³⁵ is

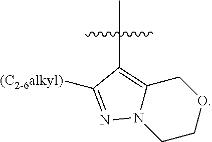

In some embodiments of any of the second, third and fourth aspects, in Formula (A2) R³¹ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments of any of the second, third and fourth aspects, in Formula (A3) R³⁴ is C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

In some embodiments any of of the second, third and fourth aspects, Z₂ is C and [symbol] is a double bond.

In some embodiments of any of the second, third and fourth aspects, [MCL-1 ligand prodrug moiety] is:

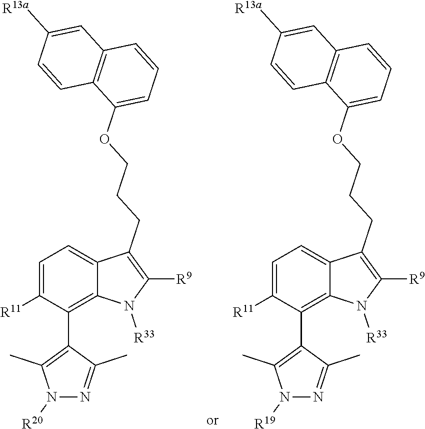

wherein
R¹¹ is halogen;
R¹³, is halogen; and
R²⁰ is Me, —CH₂—OMe, or —(CH₂CH₂O)ₚ(C₁-C₆ alkyl), wherein p is 1-5.

In some embodiments of any of the second, third and fourth aspects, R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), -oC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolyl cation.

In some embodiments of any of the second, third and fourth aspects, R⁹ is —C(O)O(C₁-C₆alkyl), —C(O)Ocycloalkyl, —C(O)Oheterocycloalkyl, —C(O)Oaryl, or —P(O)(OR^{p})₂, wherein said C₁-C₆alkyl is substituted by one or more groups selected from —OC(O)Me, —OC(O)Et, —OC(O)ⁱPr, —OC(O)C(CH₃)₃, —OC(O)CH₂OH, —OC(O)CH(CH₃)OH, —OC(O)C(CH₃)₂OH, —OC(O)CH₂CH₂OH, —OC(O)CH(OH)CHMe₂, —OC(O)CH(OH)CH₂CHMe₂, —OC(O)CH₂CH(OH)Me, —OC(O)CH₂C(OH)Me₂, —OC(O)CH(CH₂OH)(NHC(O)Me), —OC(O)CH(NHC(O)Me)(isobutyl), —OC(O)CH(OH)aryl, —OC(O)CH(OH)CH₂aryl, —OC(O)heterocycloalkyl, —OC(O)cycloalkyl, —OC(O)aryl, —OC(O)heteroaryl, —OC(O)CH₂CH₂aryl, —OC(O)CH(NH₂)ⁱPr, —OC(O)CH(ⁱPr)NHC(O)O^{t}Bu, —OC(O)CH₂OCH₂CH₂OMe, —OC(O)CH₂(OCH₂CH₂)₂OMe, —OC(O)OMe, —OC(O)OEt, —OC(O)OiPr, —OC(O)Ocycloalkyl, —OC(O)OCH₂CH₂OEt, —OC(O)O(CH₂CH₂O)₂Et, —OC(O)O(CH₂CH₂O)₃Et, —OC(O)O(CH₂CH₂O)₃CH₂CH₂OP(O)(OH)₂, —OC(O)OCh₂CH₂SMe, —OC(O)OCH₂CH₂S(O)Me, —OC(O)OCH₂CH₂S(O)₂Me, —OC(O)OCH₂heteroaryl, —OC(O)OCH₂heterocycloalkyl, —OC(O)OCH₂CH₂OCH₂aryl —OC(O)OCH₂CH₂C(O)NMe₂, —OC(O)O(CH₂)₆NH₂, —OC(O)O(CH₂)₆NMe₂, —OC(O)OCH(CH₂OEt)₂, —OC(O)NHCH₂CH₂OMe, -oC(O)NMeCH₂CH₂OMe, —SMe, —S(O)Me, —P(O)(OH)₂, —OP(O)(OH)₂, —C(O)NMe₂, —C(O)heterocycloalkyl, heteroaryl, heterocycloalkyl, —N⁺(Me)₃, —N⁺(Me)₂cycloalkyl, N-methyl morpholinium cation, N-methyl piperidinium cation and N-methyl imidazolium cation.

In some embodiments of any of the second, third and fourth aspects, R⁹ is —C(O)O(C₁-C₆alkyl) wherein said C₁-C₆alkyl is substituted by one or more groups selected from —OC(O)C(CH₃)₃, —OC(O)CH₂CH₂OH, —OC(O)C(CH₃)₂OH, —OC(O)heterocycloalkyl, —OC(O)CH₂OCH₂CH₂OMe, —OC(O)OiPr, —OC(O)Ocycloalkyl, —OC(O)O(CH₂CH₂O)₂Et, —OP(O)(OH)₂, heteroaryl or heterocycloalkyl. In some such embodiments, R⁹ is —C(O)O(C₁-C₆alkyl) wherein said C₁-C₆alkyl is substituted by —OC(O)C(CH₃)₃. In some embodiments, R⁹ is —C(O)OCH₂OC(O)C(CH₃)₃.

In some embodiments of any of the second, third and fourth aspects, R⁹ is —C(O)OCH₂OC(O)C(CH₃)₃, —C(O)OCH₂OC(O)Me, —C(O)OCH(Me)OC(O)Me, —C(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂OMe, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, —C(O)OCH₂CH₂CH₂piperazine or —P(O)(OCH₂OC(O)C(CH₃)₃)₂.

In some embodiments of any of the second, third and fourth aspects, R²⁰ is Me or —(CH₂CH₂O)₂Me.

In some embodiments of any of the second, third and fourth aspects, R³³ is R¹⁹. In other embodiments, R³³ is C₁-C₆ alkyl substituted with morpholine or a piperazine.

In some embodiments of any of the second, third and fourth aspects, the C₁-C₆ alkyl substituted with morpholine or a piperazine is

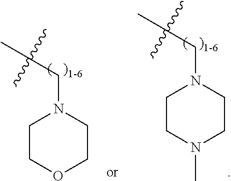

In some embodiments of any of the second, third and fourth aspects, [MCL-1 ligand prodrug moiety] is:

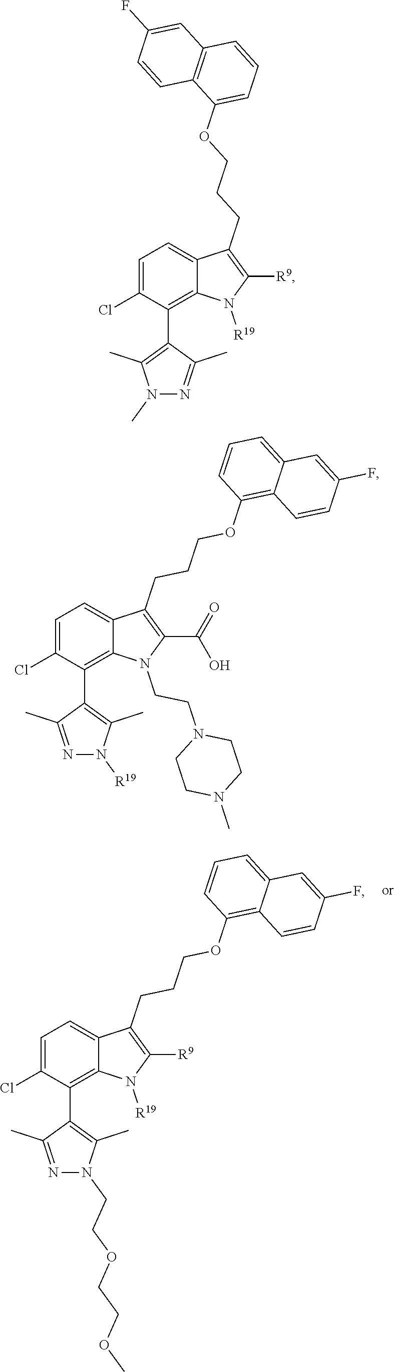

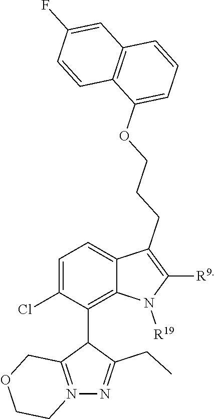

In some embodiments of any of the second, third and fourth aspects, [MCL-1 ligand prodrug moiety] is:

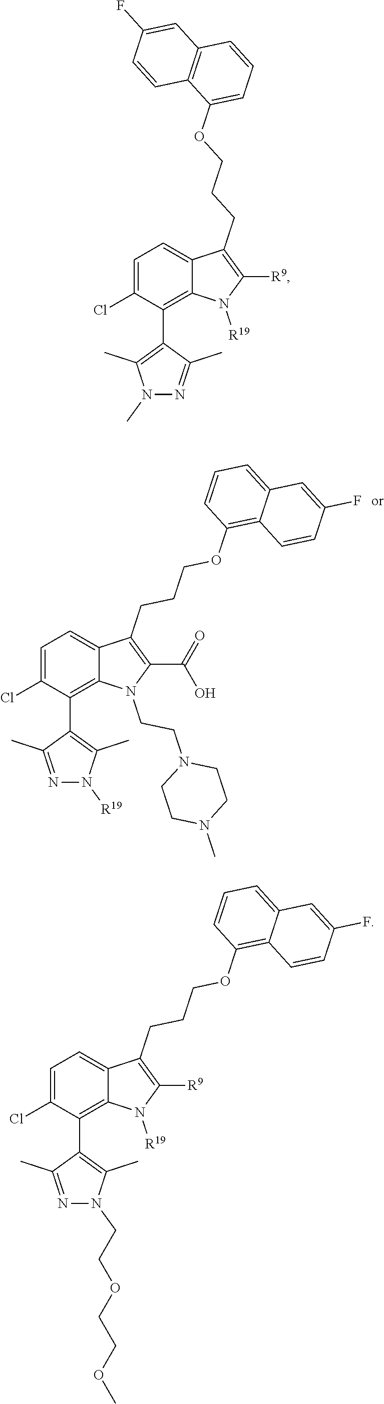

In some embodiments of the fourth aspect, the compound is:

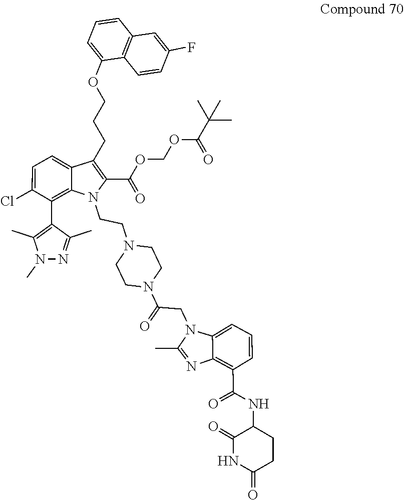 [SMILES: Cc1nn(C)c(C)c1-c1c(Cl)ccc2c(CCCOc3cccc4cc(F)ccc34)c(C(=O)OCOC(=O)C(C)(C)C)n(CCN3CCN(C(=O)Cn4c(C)nc5c(C(=O)NC6CCC(=O)NC6=O)cccc54)CC3)c12]

In some embodiments of any of the above aspects, T is C═O.

In some embodiments of any of the above aspects, X₁ and X₂ are O. In other embodiments, X₁ is O and X₂ is S. In other embodiments, X₁ is S and X₂ is O. In other embodiments, X₁ and X₂ are S.

In some embodiments of any of the above aspects, n is 0. In other embodiments, n is 1 or 2. In some embodiments, n is 1. In other embodiments, n is 2.

In accordance with a fifth aspect of the invention, there is provided a pharmaceutical composition comprising a compound of any embodiment of any one of the first, second, third or fourth aspects.

In accordance with a sixth aspect of the invention, there is provided a compound of any embodiment of any one of the first, second, third or fourth aspects or a pharmaceutical composition of the fifth aspect, for use in medicine.

In accordance with a seventh aspect of the invention, there is provided a compound of any embodiment of any one of the first, second, third or fourth aspects or a pharmaceutical composition of the fifth aspect, for use in the treatment of cancer. In some embodiments, the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer. In certain embodiments, the cancer is multiple myeloma or acute myeloid leukaemia.

In accordance with an eighth aspect of the invention, there is provided a method of treating cancer in a subject in need thereof, the method comprising administering to the subject an effective amount of a compound a of any embodiment of any one of the first, second, third or fourth aspects or a pharmaceutical composition of the fifth aspect. In some embodiments, the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer. In certain embodiments, the cancer is multiple myeloma acute myeloid leukaemia.

In some embodiments of the eighth aspect, the administration does not result in cytotoxicity in cardiomyocytes in the subject.

In some embodiments of the eighth aspect, the method further comprises administering at least one additional active agent to the subject. In some embodiments, the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

In accordance with a ninth aspect of the invention, there is provided a compound of any embodiment of any one of the first, second, third or fourth aspects or a pharmaceutical composition of the fifth aspect, for use in reversing resistance to chemotherapy or targeted cancer therapies.

In accordance with a tenth aspect of the invention, there is provided a method of reversing resistance to chemotherapy or targeted cancer therapies in a subject in need thereof, the method comprising administering to the subject an effective amount of a compound according to any one of claims 1-147, or a pharmaceutical composition according to claim 148.

In accordance with an eleventh aspect of the invention, there is provided a combined preparation of a compound of any embodiment of any one of the first, second, third or fourth aspects and at least one additional active agent, for simultaneous, separate or sequential use in therapy.

In some embodiments of the eleventh aspect, the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

In some embodiments of the eleventh aspect, the therapy is the treatment of cancer.

In accordance with a twelfth aspect of the invention, there is provided a method of reducing the cardiac cytotoxicity of an MCL-1 inhibitor, comprising coupling a cereblon binding moiety to the MCL-1 inhibitor, wherein the cereblon binding moiety is a [ligase ligand moiety] as defined in any embodiment of any one of the first, second, third and fourth aspects, and the MCL-1 inhibitor is an [MCL-1 ligand prodrug moiety] as defined in any embodiment of any one of the first, second, third and fourth aspects.

As used herein the term “alkyl” is intended to include both linear and branched alkyl groups, both of which either may be unsubstituted, or may be substituted by one or more additional groups. In some embodiments, the alkyl group is an unsubstituted alkyl group. In some embodiments, the alkyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkyl group is a C₁-C₁₂ alkyl, a C₁-C₁₀ alkyl, a C₁-C₈ alkyl, a C₁-C₆ alkyl, or a C₁-C₄ alkyl group. In some embodiments the alkyl group is a linear alkyl group. In some embodiments the alkyl group is an unsubstituted linear alkyl group. In some embodiments the alkyl group is a linear alkyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkyl group is a branched alkyl group. In some embodiments the alkyl group is an unsubstituted branched alkyl group. In some embodiments the alkyl group is a branched alkyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “alkenyl” is intended to include both unsubstituted alkenyl groups, and alkenyl groups which are substituted by one or more additional groups. In some embodiments, the alkenyl group is an unsubstituted alkenyl group. In some embodiments, the alkenyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkenyl group is a C₂-C₁₂ alkenyl, a C₂-C₁₀ alkenyl, a C₂-C₈ alkenyl, a C₂-C₆ alkenyl, or a C₂-C₄ alkenyl group. In some embodiments the alkenyl group is a linear alkenyl group. In some embodiments the alkenyl group is an unsubstituted linear alkenyl group. In some embodiments the alkenyl group is a linear alkenyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkenyl group is a branched alkenyl group. In some embodiments the alkenyl group is an unsubstituted branched alkenyl group. In some embodiments the alkenyl group is a branched alkenyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “alkynyl” is intended to include both unsubstituted alkynyl groups, and alkynyl groups which are substituted by one or more additional groups. In some embodiments, the alkynyl group is an unsubstituted alkynyl group. In some embodiments, the alkynyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the alkynyl group is a C₂-C₁₂ alkynyl, a C₂-C₁₀ alkynyl, a C₂-C₈ alkynyl, a C₂-C₆ alkynyl, or a C₂-C₄ alkynyl group. In some embodiments the alkynyl group is a linear alkynyl group. In some embodiments the alkynyl group is an unsubstituted linear alkynyl group. In some embodiments the alkynyl group is a linear alkynyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments the alkynyl group is a branched alkynyl group. In some embodiments the alkynyl group is an unsubstituted branched alkynyl group. In some embodiments the alkynyl group is a branched alkynyl group which is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “cycloalkyl” is intended to include both unsubstituted cycloalkyl groups, and cycloalkyl groups which are substituted by one or more additional groups. The term “cycloalkyl” is also intended to include monocyclic and bicyclic ring systems (including spirocyclic ring systems, in which the two rings share a single atom; fused bicyclic ring systems, in which the two rings share two adjacent atoms; and bridged bicyclic ring systems, in which the two rings share three or more atoms). In some embodiments, the cycloalkyl group is an unsubstituted cycloalkyl group. In some embodiments, the cycloalkyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the cycloalkyl group is a C₃-C₁₂ cycloalkyl, a C₄-C₁₂ cycloalkyl, a C₅-C₁₂ cycloalkyl, a C₃-C₁₀ cycloalkyl, a C₄-C₁₀ cycloalkyl, a C₅-C₁₀ cycloalkyl, a C₃-C₈ cycloalkyl, a C₄-C₈ cycloalkyl, a C₅-C₈ cycloalkyl, a C₃-C₆ cycloalkyl, a C₄-C₆ cycloalkyl, a C₅-C₆ cycloalkyl, a C₃-C₄ cycloalkyl, or a C₄-C₅ cycloalkyl group.

As used herein the term “cycloalkenyl” is intended to include both unsubstituted cycloalkenyl groups, and cycloalkenyl groups which are substituted by one or more additional groups. In some embodiments, the cycloalkenyl group is an unsubstituted cycloalkenyl group. In some embodiments, the cycloalkenyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the cycloalkenyl group is a C₄-C₁₂ cycloalkenyl, a C₅-C₁₂ cycloalkenyl, a C₄-C₁₀ cycloalkenyl, a C₅-C₁₀ cycloalkenyl, a C₄-C₈ cycloalkenyl, a C₅-C₈ cycloalkenyl, a C₄-C₆ cycloalkenyl, a C₅-C₆ cycloalkenyl, or a C₄-C₅ cycloalkenyl group.

As used herein the term “heterocycloalkyl” is intended to include both unsubstituted heterocycloalkyl groups, and heterocycloalkyl groups which are substituted by one or more additional groups. The term “heterocycloalkyl” is also intended to include monocyclic and bicyclic ring systems (including spirocyclic ring systems, in which the two rings share a single atom; fused bicyclic ring systems, in which the two rings share two adjacent atoms; and bridged bicyclic ring systems, in which the two rings share three or more atoms). In some embodiments, the heterocycloalkyl group is a monocyclic ring system, a spirocyclic ring system, or a fused bicyclic ring system. In some embodiments, the heterocycloalkyl group is an unsubstituted heterocycloalkyl group. In some embodiments, the heterocycloalkyl group is substituted by one or more groups selected from —R^{W}, —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, one or more —CH₂— groups of the heterocycloalkyl ring may be replaced with a —C(O)— group, In some embodiments, the heterocycloalkyl group is a C₃-C₁₂ heterocycloalkyl, a C₄-C₁₂ heterocycloalkyl, a C₅-C₁₂ heterocycloalkyl, a C₃-C₁₀ heterocycloalkyl, a C₄-C₁₀ heterocycloalkyl, a C₅-C₁₀ heterocycloalkyl, a C₃-C₈ heterocycloalkyl, a C₄-C₈ heterocycloalkyl, a C₅-C₈ heterocycloalkyl, a C₃-C₆ heterocycloalkyl, a C₄-C₆ heterocycloalkyl, a C₅-C₆ heterocycloalkyl, a C₃-C₄ heterocycloalkyl, or a C₄-C₅ heterocycloalkyl group.

As used herein the term “aryl” is intended to include both unsubstituted aryl groups, and aryl groups which are substituted by one or more additional groups. In some embodiments, the aryl group is an unsubstituted aryl group. In some embodiments, the aryl group is substituted by one or more groups selected from —OH, —OR^{W}, —NH₂, —NHR^{W}, —NR^{W}₂, —SO₂R^{W}, —C(O)R^{W}, —OC(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, the aryl group is a C₆-C₁₀ aryl, a C₆-C₈ aryl, or a C₆ aryl.

As used herein the term “heteroaryl” is intended to include both unsubstituted heteroaryl groups, and heteroaryl groups which are substituted by one or more additional groups. In some embodiments, the heteroaryl group is an unsubstituted heteroaryl group. In some embodiments, the heteroaryl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl. In some embodiments, one or more —NH— groups of the heteroaryl ring may be replaced with a —NR^{W}— group. In some embodiments, the heteroaryl group is a C₆-C₁₀ heteroaryl, a C₆-C₉ heteroaryl, a C₆-C₈ heteroaryl, or a C₆ heteroaryl.

As used herein the term “fused heterocycloalkyl-heteroaryl” is intended to mean a bicyclic ring system in which one ring is a heterocycloalkyl ring and the other is a heteroaryl ring, and in which the two rings share two adjacent atoms. Of the two adjacent atoms shared by the two rings, both may be carbon atoms; both may be heteroatoms (e. g. independently O, N or S); or one may be a carbon atom and the other a heteroatom (e. g. O, N or S). The fused heterocycloalkyl-heteroaryl may be unsubstituted or may be substituted by one or more additional groups. In some embodiments, the fused heterocycloalkyl-heteroaryl group is an unsubstituted cycloalkenyl group. In some embodiments, the fused heterocycloalkyl-heteroaryl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

As used herein the term “benzyl” is intended to include both unsubstituted benzyl groups, and benzyl groups which are substituted by one or more additional groups. In some embodiments, the benzyl group is an unsubstituted benzyl group. In some embodiments, the benzyl group is substituted by one or more groups selected from —OH, —OR^{W}, —NH₂, —NHR^{W}, —NR^{W}₂, —SO₂R^{W}, —C(O)R^{W}, —CN, and —NO₂, wherein each R^{W} is unsubstituted and is independently alkyl, cycloalkyl, alkenyl, cycloalkenyl, aryl, heteroaryl, or benzyl.

In some embodiments of any of the above aspects of the invention, all alkyl, alkenyl, alkynyl, cycloalkyl, cycloalkenyl, heterocycloalkyl, aryl, heteroaryl and benzyl groups in the compounds are unsubstituted.

### BRIEF DESCRIPTION OF THE FIGURES

FIG. 1 shows a representative Western blotting membrane demonstrating MCL-1 protein degradation in MV4-11 cells treated for 6 hours with Compound 69 of the present invention. Loading control: total protein (SF—stain free).

FIG. 2 shows a representative Western blotting membrane demonstrating MCL-1 protein degradation in MV4-11 cells treated for 6 hours with Compound of WO2022/253713A1, and Compound 41 of the present invention, in the presence of apoptosis inhibitor. Loading control: total protein (SF—stain free).

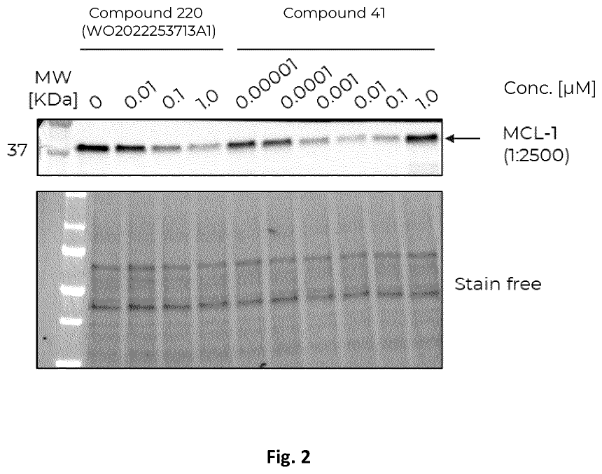

### DETAILED DESCRIPTION OF THE INVENTION

As discussed above, the present invention provides a compound of formula (I)

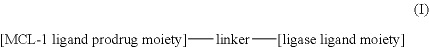

or a salt, solvate, hydrate or isomer thereof.

Prodrugs of bifunctional compounds that bind to a ubiquitin ligase and target MCL-1 protein according to the present invention could potentially have improved bioavailability and thus require less frequent administration while maintaining favorable efficacy thanks to an increase in half-life. Reduction of the therapeutic dose may also have an impact in terms of reduction of side effects and toxicity as compared to administration of the drug itself as an active compound. Furthermore, some prodrugs of the invention may have improved aqueous solubility as compared to the active compounds.

### Ligase Ligand Moieties

Ligase Ligand Moieties with Thalidomide-Type Structure

In the first aspect of the present invention, [ligase ligand moiety] is:

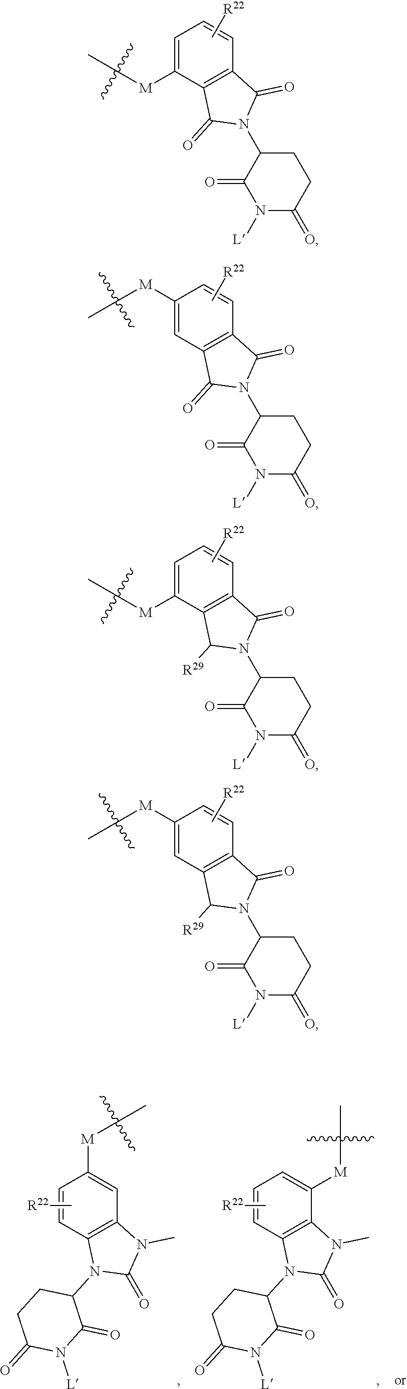

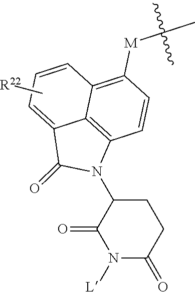

wherein
M is O, S or NH, or is absent;
[symbol] indicates attachment to R¹⁸ of the linker;
R²² is hydrogen, halogen, —OMe, an amino group, heterocycloalkyl, or unsubstituted C₁-C₆ alkyl;
R²⁹ is hydrogen or Me; and
L′ is hydrogen, alkyl, benzyl, acetyl or pivaloyl.

In some embodiments of the first aspect of the present invention, M is O or NH, or is absent.

In some embodiments of the first aspect of the present invention, R²² is hydrogen.

In some embodiments of the first aspect of the present invention, L′ is hydrogen or methyl. In some embodiments, L′ is hydrogen.

In some embodiments of the first aspect of the present invention, R²⁹ is hydrogen.

In some embodiments of the first aspect of the present invention, [ligase ligand moiety] is:

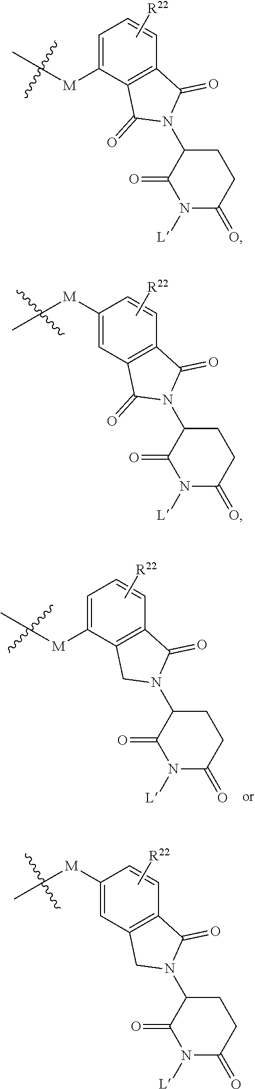

In some embodiments of the first aspect of the present invention, [ligase ligand moiety] is:

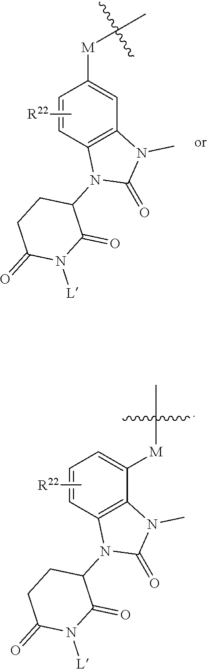

In some embodiments of the first aspect of the present invention, [ligase ligand moiety] is:

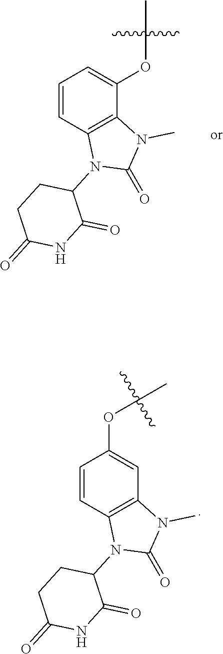 [SMILES: *Oc1cccc2c1n(C)c(=O)n2C1CCC(=O)NC1=O ; *Oc1ccc2c(c1)n(C)c(=O)n2C1CCC(=O)NC1=O]

In some embodiments of the first aspect of the present invention, [ligase ligand moiety] is:

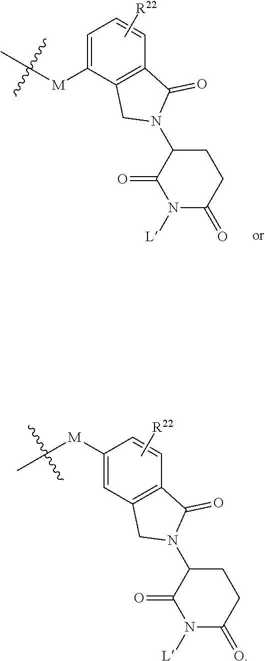

In some embodiments of the first aspect of the present invention, [ligase ligand moiety] is

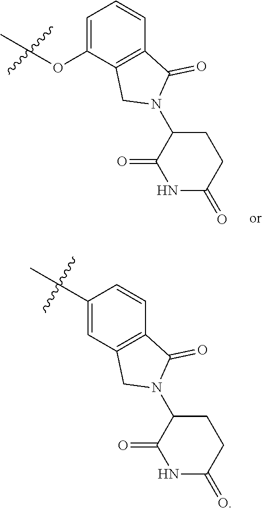 [SMILES: *Oc1cccc2c1CN(C1CCC(=O)NC1=O)C2=O ; "or" ; *c1ccc2c(c1)CN(C1CCC(=O)NC1=O)C2=O]

Examples of the above ligase ligand moieties are shown in Table 1 below:

TABLE 1

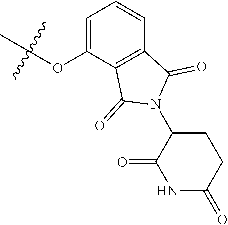 [SMILES: *Oc1cccc2c1C(=O)N(C1CCC(=O)NC1=O)C2=O]

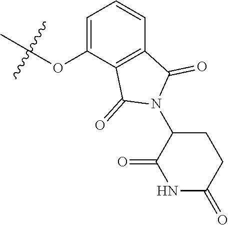 [SMILES: *Oc1cccc2c1C(=O)N(C1CCC(=O)NC1=O)C2=O]

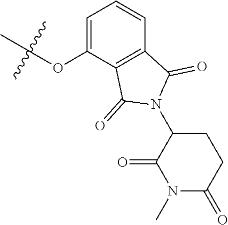 [SMILES: *Oc1cccc2c1C(=O)N(C1CCC(=O)N(C)C1=O)C2=O]

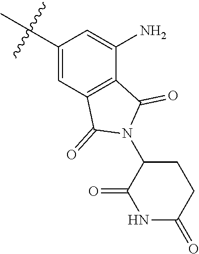 [SMILES: *c1cc(N)c2c(c1)C(=O)N(C1CCC(=O)NC1=O)C2=O]

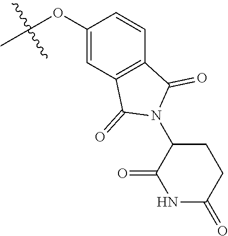 [SMILES: *Oc1ccc2c(c1)C(=O)N(C1CCC(=O)NC1=O)C2=O]

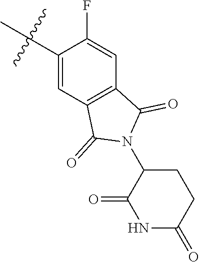 [SMILES: *c1cc2c(cc1F)C(=O)N(C1CCC(=O)NC1=O)C2=O]

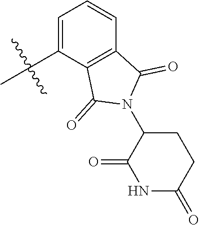 [SMILES: *c1cccc2c1C(=O)N(C1CCC(=O)NC1=O)C2=O]

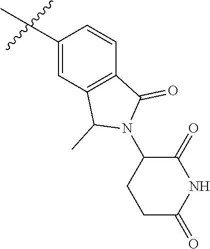 [SMILES: *c1ccc2c(c1)C(C)N(C1CCC(=O)NC1=O)C2=O]

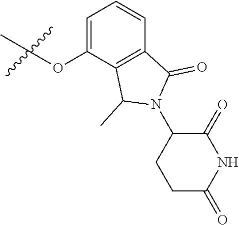 [SMILES: *Oc1cccc2c1C(C)N(C1CCC(=O)NC1=O)C2=O]

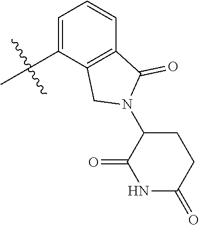 [SMILES: *c1cccc2c1CN(C1CCC(=O)NC1=O)C2=O]

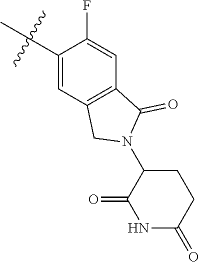 [SMILES: *c1cc2c(cc1F)C(=O)N(C1CCC(=O)NC1=O)C2]

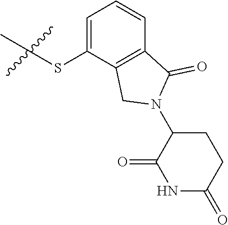 [SMILES: *Sc1cccc2c1CN(C1CCC(=O)NC1=O)C2=O]

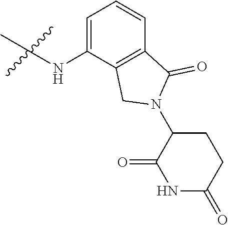 [SMILES: *Nc1cccc2c1CN(C1CCC(=O)NC1=O)C2=O]

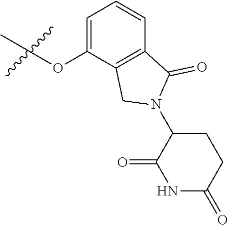 [SMILES: *Oc1cccc2c1CN(C1CCC(=O)NC1=O)C2=O]

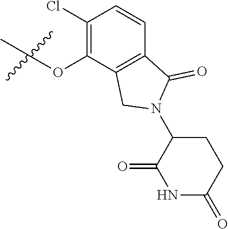 [SMILES: *Oc1c(Cl)ccc2c1CN(C1CCC(=O)NC1=O)C2=O]

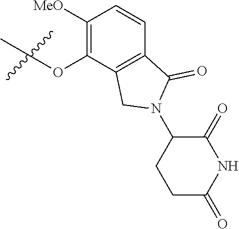 [SMILES: *Oc1c(OC)ccc2c1CN(C1CCC(=O)NC1=O)C2=O]

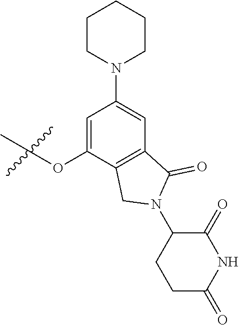 [SMILES: *Oc1cc(N2CCCCC2)cc2c1CN(C1CCC(=O)NC1=O)C2=O]

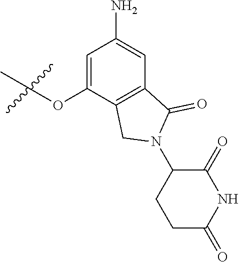 [SMILES: *Oc1cc(N)cc2c1CN(C1CCC(=O)NC1=O)C2=O]

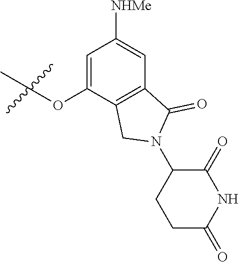 [SMILES: *Oc1cc(NC)cc2c1CN(C1CCC(=O)NC1=O)C2=O]

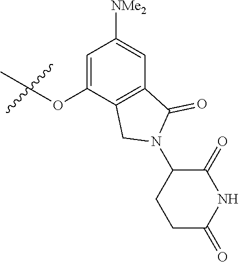 [SMILES: *Oc1cc(N(C)C)cc2c1CN(C1CCC(=O)NC1=O)C2=O]

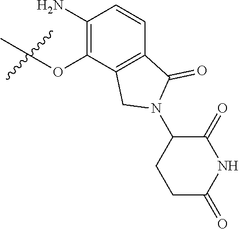 [SMILES: *Oc1c(N)ccc2c1CN(C1CCC(=O)NC1=O)C2=O]

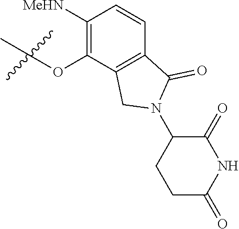 [SMILES: *Oc1c(NC)ccc2c1CN(C1CCC(=O)NC1=O)C2=O]

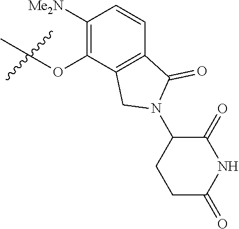 [SMILES: *Oc1c(N(C)C)ccc2c1CN(C1CCC(=O)NC1=O)C2=O]

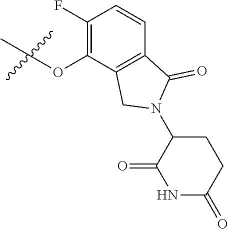 [SMILES: *Oc1c(F)ccc2c1CN(C1CCC(=O)NC1=O)C2=O]

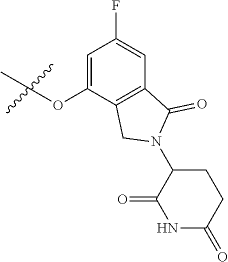 [SMILES: *Oc1cc(F)cc2c1CN(C1CCC(=O)NC1=O)C2=O]

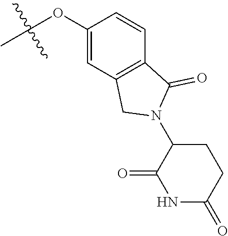 [SMILES: *Oc1ccc2c(c1)CN(C1CCC(=O)NC1=O)C2=O]

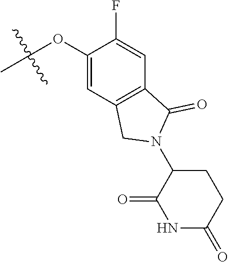 [SMILES: *Oc1cc2c(cc1F)C(=O)N(C1CCC(=O)NC1=O)C2]

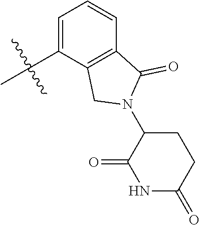 [SMILES: *c1cccc2c1CN(C1CCC(=O)NC1=O)C2=O]

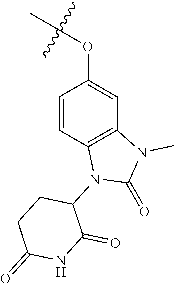 [SMILES: *Oc1ccc2c(c1)n(C)c(=O)n2C1CCC(=O)NC1=O]

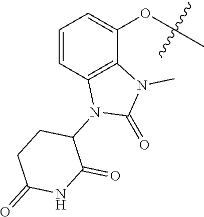 [SMILES: *Oc1cccc2c1n(C)c(=O)n2C1CCC(=O)NC1=O]

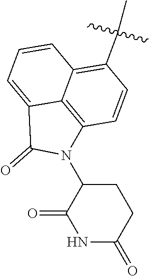 [SMILES: *c1ccc2c3c(cccc13)C(=O)N2C1CCC(=O)NC1=O]

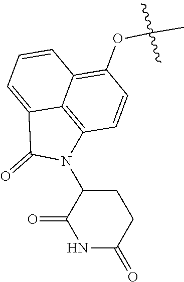 [SMILES: *Oc1ccc2c3c(cccc13)C(=O)N2C1CCC(=O)NC1=O]

Many of the compounds in Table 1 above are commercially available in the forms shown below.

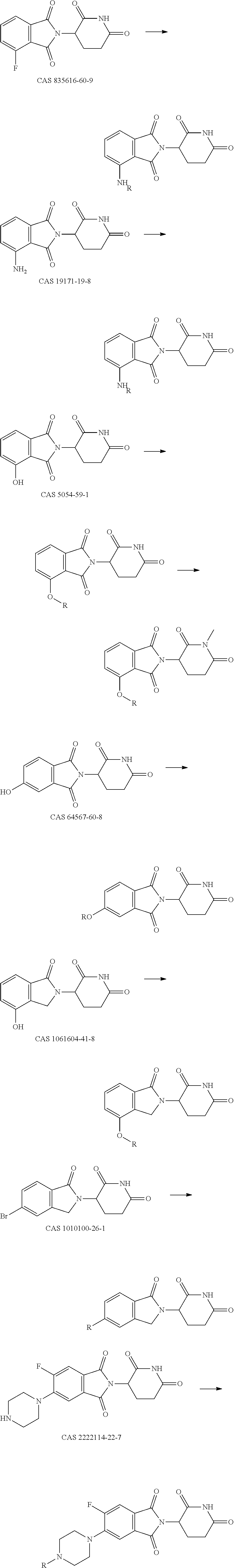

### Ligase Ligand Moieties of Formulae (IV) and (IVa); Formulae (VIIa), (VIIb), (VIIc) and (VIId); Formula (VIII) and Formula (IX)

In the second aspect of the present invention, the [ligase ligand moiety] may be of Formula (IV) or (IVa); of Formulae (VIIa), (VIIb), (VIIc) or (Vild); or Formula (VIII); or of Formula (IX). The synthesis of the ligase ligand moieties of Formula (IV) (as defined above) can be summarized as follows:

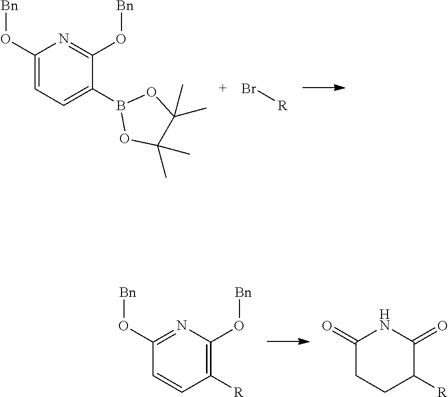

Example ligase ligand moieties of Formula (IV) and (IVa) are shown in Table 2 below. For example,

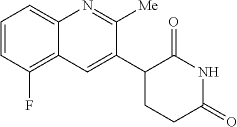 [SMILES: Cc1nc2cccc(F)c2cc1C1CCC(=O)NC1=O]

could be modified to allow attachment to the [linker](e.g. by nucleophilic aromatic substitution; or by exchange of fluorine for bromine followed by attachment—by palladium coupling—of either the [linker] itself, or of a functional group to which the [linker] could be attached).

TABLE 2

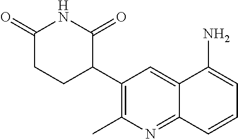 [SMILES: Cc1nc2cccc(N)c2cc1C1CCC(=O)NC1=O]

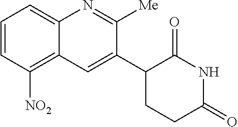 [SMILES: Cc1nc2cccc([N+](=O)[O-])c2cc1C1CCC(=O)NC1=O]

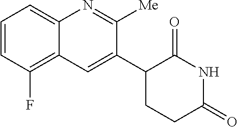 [SMILES: Cc1nc2cccc(F)c2cc1C1CCC(=O)NC1=O]

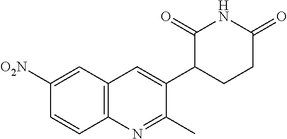 [SMILES: Cc1nc2ccc([N+](=O)[O-])cc2cc1C1CCC(=O)NC1=O]

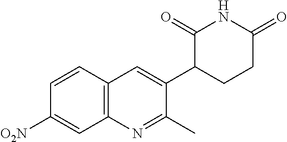 [SMILES: Cc1nc2cc([N+](=O)[O-])ccc2cc1C1CCC(=O)NC1=O]

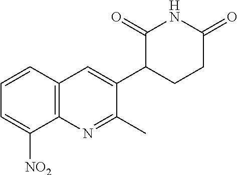 [SMILES: Cc1nc2c([N+](=O)[O-])cccc2cc1C1CCC(=O)NC1=O]

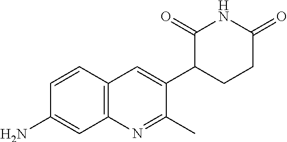 [SMILES: Cc1nc2cc(N)ccc2cc1C1CCC(=O)NC1=O]

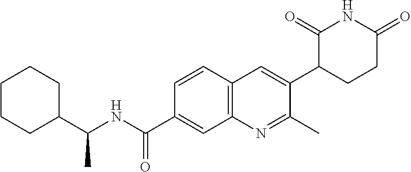 [SMILES: Cc1nc2cc(C(=O)N[C@@H](C)C3CCCCC3)ccc2cc1C1CCC(=O)NC1=O]

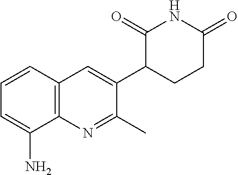 [SMILES: Cc1nc2c(N)cccc2cc1C1CCC(=O)NC1=O]

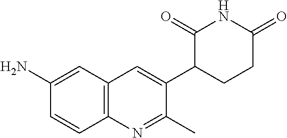 [SMILES: Cc1nc2ccc(N)cc2cc1C1CCC(=O)NC1=O]

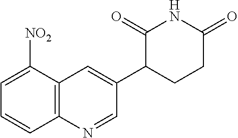 [SMILES: O=C1CCC(c2cnc3cccc([N+](=O)[O-])c3c2)C(=O)N1]

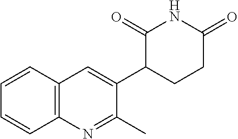 [SMILES: Cc1nc2ccccc2cc1C1CCC(=O)NC1=O]

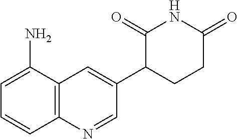 [SMILES: Nc1cccc2ncc(C3CCC(=O)NC3=O)cc12]

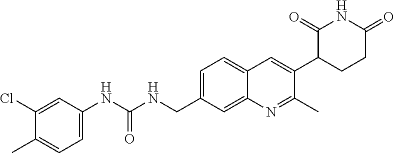 [SMILES: Cc1ccc(NC(=O)NCc2ccc3cc(C4CCC(=O)NC4=O)c(C)nc3c2)cc1Cl]

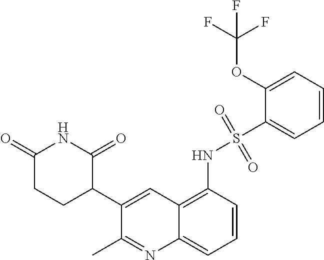 [SMILES: Cc1nc2cccc(NS(=O)(=O)c3ccccc3OC(F)(F)F)c2cc1C1CCC(=O)NC1=O]

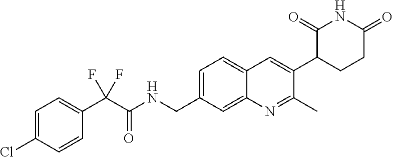 [SMILES: Cc1nc2cc(CNC(=O)C(F)(F)c3ccc(Cl)cc3)ccc2cc1C1CCC(=O)NC1=O]

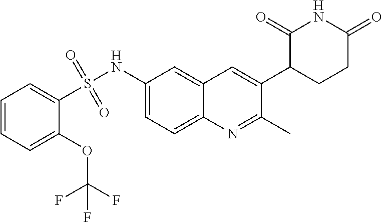 [SMILES: Cc1nc2ccc(NS(=O)(=O)c3ccccc3OC(F)(F)F)cc2cc1C1CCC(=O)NC1=O]

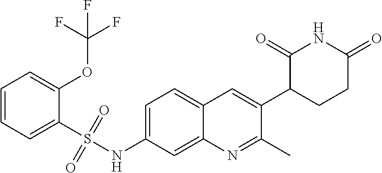 [SMILES: Cc1nc2cc(NS(=O)(=O)c3ccccc3OC(F)(F)F)ccc2cc1C1CCC(=O)NC1=O]

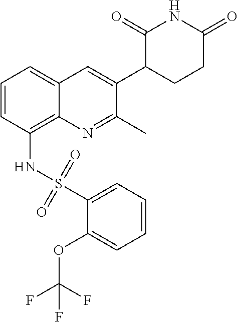 [SMILES: Cc1nc2c(NS(=O)(=O)c3ccccc3OC(F)(F)F)cccc2cc1C1CCC(=O)NC1=O]

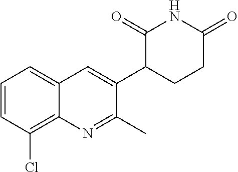 [SMILES: Cc1nc2c(Cl)cccc2cc1C1CCC(=O)NC1=O]

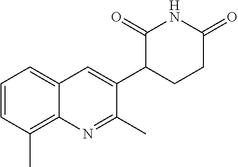 [SMILES: Cc1nc2c(C)cccc2cc1C1CCC(=O)NC1=O]

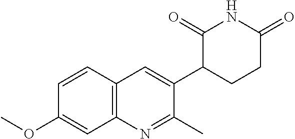 [SMILES: COc1ccc2cc(C3CCC(=O)NC3=O)c(C)nc2c1]

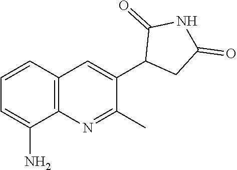 [SMILES: Cc1nc2c(N)cccc2cc1C1CC(=O)NC1=O]

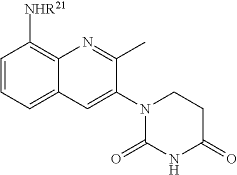

Example ligase ligand moieties of Formula (VIIa), (VIIb), (VIIc) and (Vild) are shown in Table 3 below:

TABLE 3

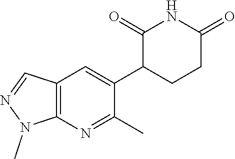 [SMILES: Cc1nc2c(cnn2C)cc1C1CCC(=O)NC1=O]

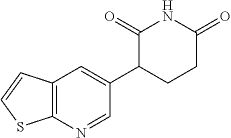 [SMILES: O=C1CCC(c2cnc3sccc3c2)C(=O)N1]

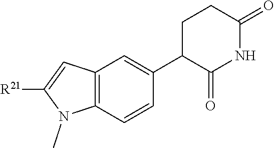

The synthesis of these compounds is summarized in Steps 1-4 below:

### Step 1:

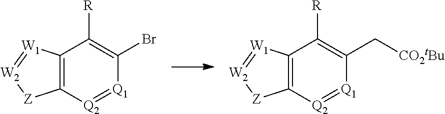

To a solution of bromoarene (1 equiv) in dioxane were added KOAc (2 equiv), ((1-(tert-butoxy)vinyl)oxy)(tert-butyl)dimethylsilane (4 equiv) and Pd[P(o-Tol)₃]₂Cl₂ (0.2 equiv) under inert gas and the reaction mixture was stirred at 130° C. for 48 h. The reaction mixture was filtered through Celite, concentrated under reduced pressure and purified by flash column chromatography to give appropriate tert-butyl arylacetate.

### Step 2:

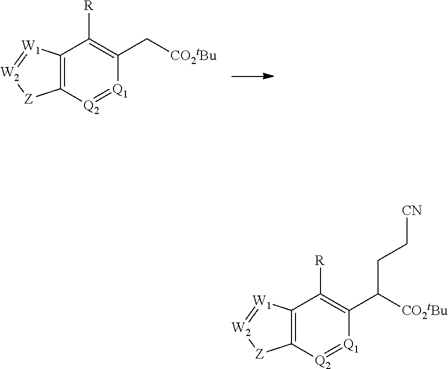

To a solution of appropriate tert-butyl 2-(quinolin-3-yl)acetate (1 equiv) in DMF were added K₂CO₃ (1 equiv), benzyltriethylammonium chloride (1 equiv) and acrylonitrile (1 equiv) and the reaction mixture was stirred at RT for 16 h. The reaction mixture was diluted with water and the product was extracted with ethyl acetate. Combined organic phases were dried over Na₂SO₄, concentrated under reduced pressure and purified by flash column chromatography.

### Step 3:

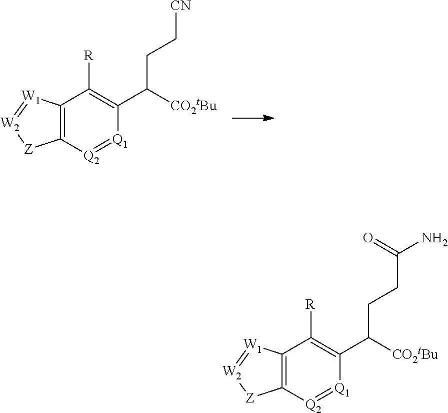

To an ice cold solution of appropriate tert-butyl 4-cyano-2-(quinolin-3-yl)butanoate (1 equiv) in DMSO were added H₂O₂(5 equiv) and K₂CO₃ (0.1 equiv). The reaction mixture was warmed to RT and stirred for 16 h. The reaction mixture was diluted with water and the product was extracted with ethyl acetate. Combined organic phases were dried over Na₂SO₄, concentrated under reduced pressure and purified by flash column chromatography.

### Step 4:

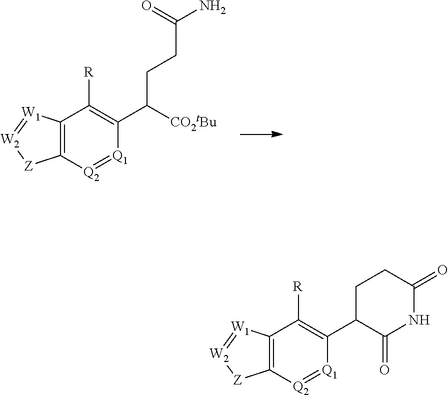

In a vial were placed appropriate tert-butyl 5-amino-2-(2-methylquinolin-3-yl)-5-oxopentanoate (1 equiv), p-toluenesulfonic acid (5-10 equiv) and ACN and the reaction mixture was stirred at 80° C. for 2-48 h. The mixture was concentrated under reduced pressure and purified by flash column chromatography or preparative HPLC.

### Ligase Ligand Moieties of Formula (Va) and Formula (Vb), and Formula (IIa) and Formula (IIb)

In the third aspect of the present invention, the [ligase ligand moiety] may be of Formulae (Va) or (Vb), or may be of Formulae (Ila) or (IIb). The synthesis of the ligase ligand moieties of Formulae (Va), (Vb), (Ila) and (IIb) (as defined above) can be summarized in the following general procedure (carried out under Synthetic Conditions D, E, F or G, as set out below:

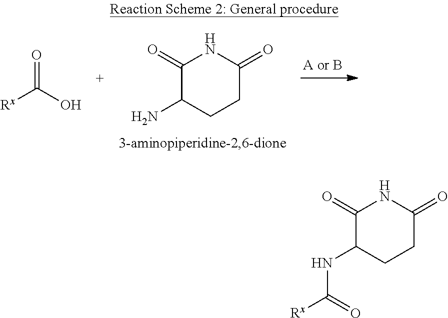

### Synthetic Conditions D

An appropriate acid (R^{x}COOH in the above reaction scheme) (1.1 eq), DMAP (0.04 eq), and EDC (1.2 eq) were added to a solution of 3-aminopiperidine-2,6-dione (1 eq) and N-hydroxybenzotriazole (1.2 eq) in DMF (0.5 M). The reaction mixture was stirred overnight at room temperature (20-25° C.). Water (2×DMF volume) was added and the obtained solution was extracted with dichloromethane (3×DMF volume). The combined organic layers were washed with water, dried over Na₂SO₄, and concentrated under reduced pressure. The crude product was purified by preparative HPLC or by column chromatography.

### Synthetic Conditions E

An appropriate acid R^{x}COOH in the above reaction scheme) (1 eq) and EDC (1.2 eq) were added to a solution of 3-aminopiperidine-2,6-dione (hydrochloride salt, 1.1 eq), triethylamine (1.2 eq) and N-hydroxybenzotriazole (1.2 eq) in DMA (0.5 M). The reaction mixture was stirred overnight at rt. Water (2×DMA volume) was added and obtained mixture was extracted with dichloromethane (3×DMA volume). The combined organic layers were washed with water, dried over Na₂SO₄, and concentrated under reduced pressure. The crude product was isolated by preparative HPLC or by column chromatography.

### Synthetic Conditions F

To a solution of appropriate acid (R^{x}COOH in the above reaction scheme) (1 eq) and HATU (1.5 eq) in dry DMF were added 3-aminopiperidine-2,6-dione (hydrochloride salt, 1.2 eq) and DIPEA (3 eq). The reaction mixture was stirred overnight at rt. The crude product was purified by preparative HPLC or/and by preparative TLC.

### Synthetic Conditions G

To a solution of appropriate acid (R^{x}COOH in the above reaction scheme) (1 eq) 3-aminopiperidine-2,6-dione (hydrochloride salt, 1.2 eq) and DMAP (0.1 eq.) in an inert atmosphere in dry DMF were added DIPEA (2.2 eq.) and HATU (1.5 eq) in dry DMF. The reaction mixture was stirred overnight at rt. The crude product was purified by preparative HPLC or/and by preparative TLC.

### Example Method 1: Formation of Chlorinated R^{x} Group of R^{x}COOH (or its Ester R^{x}COOR^{y})

NCS (1.1 eq) was added to a solution of an appropriate starting material (1 eq) in DMF (0.5 M) and the reaction mixture was stirred for 2 h at room temperature (20-25° C.). The reaction mixture was poured into water (2×DMF volume) and occurred precipitate was filtered. The solids were washed with water and dried in vacuum to give the acid, ROOH.

### Example method 2: synthesis of R^{x}COOH from corresponding ester R^{x}COOR^{y})

LiOH (1.1 eq) was added to a solution of an appropriate ester (1 eq) in THF:water mixture (3:1 or 5:1, 85 mM) and the resulting mixture was stirred overnight at room temperature (20-25° C.). The mixture was concentrated under reduced pressure, diluted with water, and acidified with concentrated HCl to pH=2-3. The precipitate was filtered, washed with water, and dried in vacuum to give the target carboxylic acid.

### Example Method 3: Formation of Acetylated R^{x} Group of R^{x}COOR^{y}

A mixture of an appropriate amine (1 eq.), Ac₂O (3 eq.), and DMAP (0.2 eq.) in dioxane (0.2 M) was heated to 80° C. for 2 h. Upon completion, the mixture was cooled down to room temperature (20-25° C.) and concentrated under reduced pressure. The residue was diluted with water (1×dioxane volume) and extracted with EtOAc (3×dioxane volume). The organic layers were washed with water, brine, dried over Na₂SO₄, and evaporated to dryness to afford an acylated product typically used without further purification.

Example ligase ligand moieties of Formula (Va) and Formula (Vb) are shown in Table 4 below. Many of these compounds could be modified to allow attachment to the [linker](e.g. by C—H bond activation).

TABLE 4

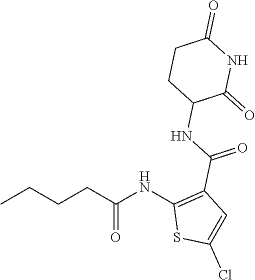 [SMILES: CCCCC(=O)Nc1sc(Cl)cc1C(=O)NC1CCC(=O)NC1=O]

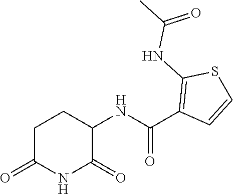 [SMILES: CC(=O)Nc1sccc1C(=O)NC1CCC(=O)NC1=O]

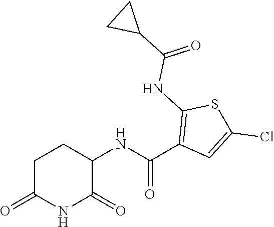 [SMILES: O=C1CCC(NC(=O)c2cc(Cl)sc2NC(=O)C2CC2)C(=O)N1]

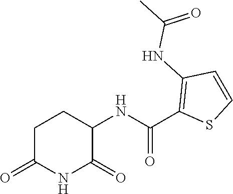 [SMILES: CC(=O)Nc1ccsc1C(=O)NC1CCC(=O)NC1=O]

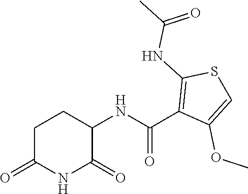 [SMILES: COc1csc(NC(C)=O)c1C(=O)NC1CCC(=O)NC1=O]

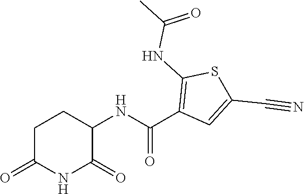 [SMILES: CC(=O)Nc1sc(C#N)cc1C(=O)NC1CCC(=O)NC1=O]

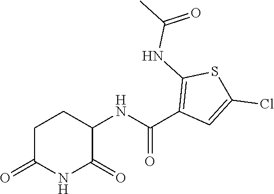 [SMILES: CC(=O)Nc1sc(Cl)cc1C(=O)NC1CCC(=O)NC1=O]

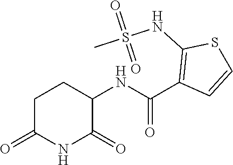 [SMILES: CS(=O)(=O)Nc1sccc1C(=O)NC1CCC(=O)NC1=O]

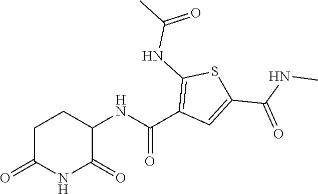 [SMILES: CNC(=O)c1cc(C(=O)NC2CCC(=O)NC2=O)c(NC(C)=O)s1]

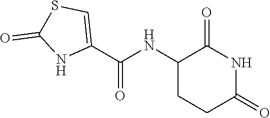 [SMILES: O=C1CCC(NC(=O)c2csc(=O)[nH]2)C(=O)N1]

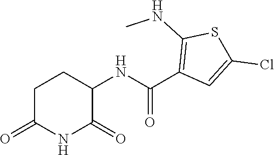 [SMILES: CNc1sc(Cl)cc1C(=O)NC1CCC(=O)NC1=O]

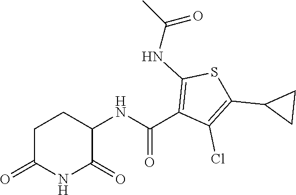 [SMILES: CC(=O)Nc1sc(C2CC2)c(Cl)c1C(=O)NC1CCC(=O)NC1=O]

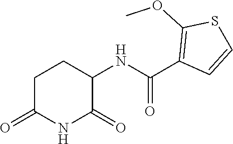 [SMILES: COc1sccc1C(=O)NC1CCC(=O)NC1=O]

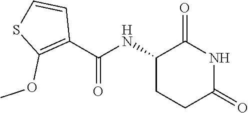 [SMILES: COc1sccc1C(=O)N[C@H]1CCC(=O)NC1=O]

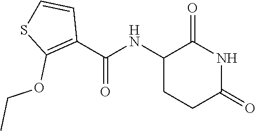 [SMILES: CCOc1sccc1C(=O)NC1CCC(=O)NC1=O]

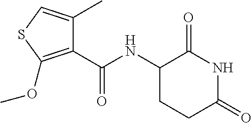 [SMILES: COc1scc(C)c1C(=O)NC1CCC(=O)NC1=O]

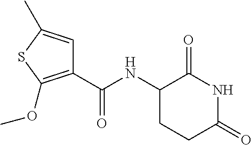 [SMILES: COc1sc(C)cc1C(=O)NC1CCC(=O)NC1=O]

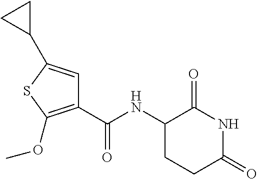 [SMILES: COc1sc(C2CC2)cc1C(=O)NC1CCC(=O)NC1=O]

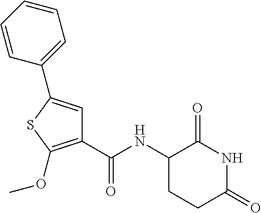 [SMILES: COc1sc(-c2ccccc2)cc1C(=O)NC1CCC(=O)NC1=O]

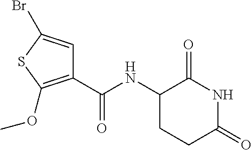 [SMILES: COc1sc(Br)cc1C(=O)NC1CCC(=O)NC1=O]

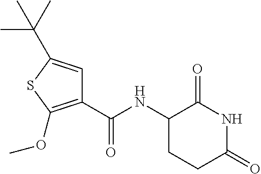 [SMILES: COc1sc(C(C)(C)C)cc1C(=O)NC1CCC(=O)NC1=O]

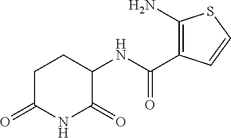 [SMILES: Nc1sccc1C(=O)NC1CCC(=O)NC1=O]

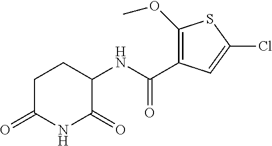 [SMILES: COc1sc(Cl)cc1C(=O)NC1CCC(=O)NC1=O]

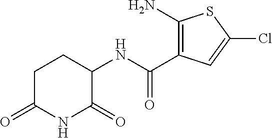 [SMILES: Nc1sc(Cl)cc1C(=O)NC1CCC(=O)NC1=O]

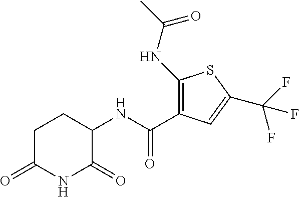 [SMILES: CC(=O)Nc1sc(C(F)(F)F)cc1C(=O)NC1CCC(=O)NC1=O]

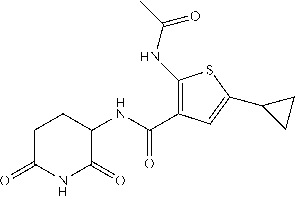 [SMILES: CC(=O)Nc1sc(C2CC2)cc1C(=O)NC1CCC(=O)NC1=O]

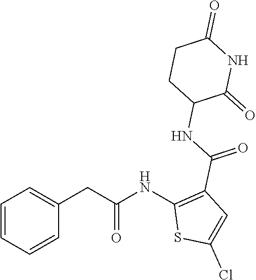 [SMILES: O=C1CCC(NC(=O)c2cc(Cl)sc2NC(=O)Cc2ccccc2)C(=O)N1]

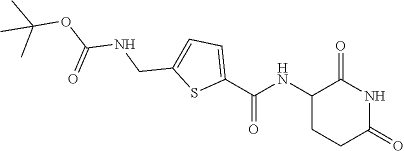 [SMILES: CC(C)(C)OC(=O)NCc1ccc(C(=O)NC2CCC(=O)NC2=O)s1]

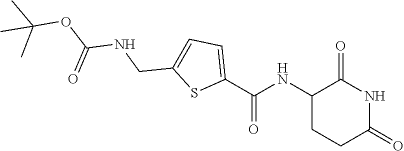 [SMILES: CC(C)(C)OC(=O)NCc1ccc(C(=O)NC2CCC(=O)NC2=O)s1]

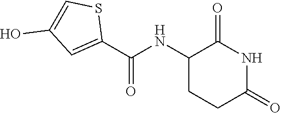 [SMILES: O=C1CCC(NC(=O)c2cc(O)cs2)C(=O)N1]

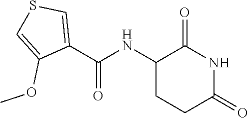 [SMILES: COc1cscc1C(=O)NC1CCC(=O)NC1=O]

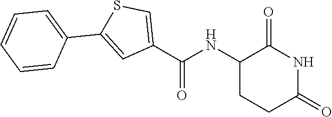 [SMILES: O=C1CCC(NC(=O)c2csc(-c3ccccc3)c2)C(=O)N1]

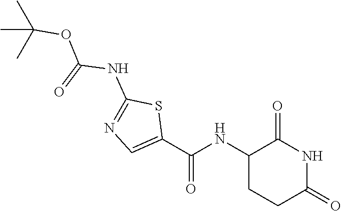 [SMILES: CC(C)(C)OC(=O)Nc1ncc(C(=O)NC2CCC(=O)NC2=O)s1]

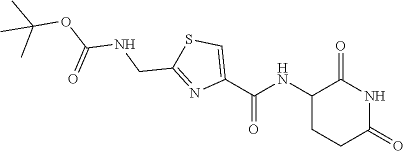 [SMILES: CC(C)(C)OC(=O)NCc1nc(C(=O)NC2CCC(=O)NC2=O)cs1]

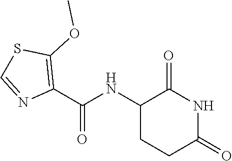 [SMILES: COc1scnc1C(=O)NC1CCC(=O)NC1=O]

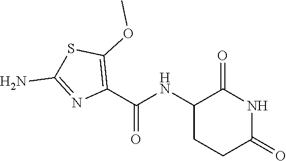 [SMILES: COc1sc(N)nc1C(=O)NC1CCC(=O)NC1=O]

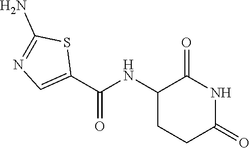 [SMILES: Nc1ncc(C(=O)NC2CCC(=O)NC2=O)s1]

Example ligand moieties of Formula (IIa) and Formula (IIb) are shown in Table 5 below. Many of these compounds could be modified to allow attachment to the [linker] (e.g. by C—H bond activation).

TABLE 5

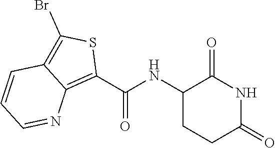 [SMILES: O=C1CCC(NC(=O)c2sc(Br)c3cccnc23)C(=O)N1]

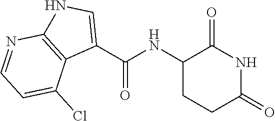 [SMILES: O=C1CCC(NC(=O)c2c[nH]c3nccc(Cl)c23)C(=O)N1]

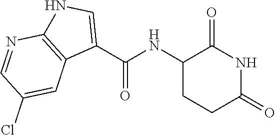 [SMILES: O=C1CCC(NC(=O)c2c[nH]c3ncc(Cl)cc23)C(=O)N1]

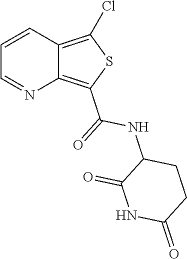 [SMILES: O=C1CCC(NC(=O)c2sc(Cl)c3cccnc23)C(=O)N1]

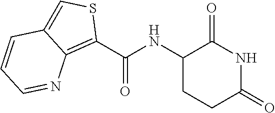 [SMILES: O=C1CCC(NC(=O)c2scc3cccnc23)C(=O)N1]

Ligase ligand moieties of Formula (VIa) and Formula (VIb)

In the fourth aspect of the present invention, the [ligase ligand moiety] may be of Formula (VIa) or (VIb):

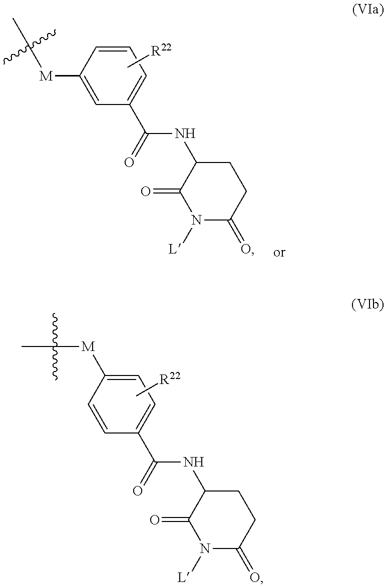

wherein
M is O, S or NH, or is absent;
[symbol] indicates attachment to R¹⁸ of the linker;
R²² is hydrogen, halogen, —OMe, an amino group, heterocycloalkyl, or unsubstituted C₁-C₆ alkyl; and
L′ is hydrogen, alkyl, benzyl, acetyl or pivaloyl.

In some embodiments, L′ is hydrogen. In some embodiments, M is O. In some embodiments, R²² is unsubstituted C₁-C₆ alkyl.

The synthesis of the ligase ligand moieties of Formula (VIa) and (VIb) is set out in the Examples section, below.

Example ligase ligand moieties of Formula (VIa) and Formula (VIb) are shown in Table 6 below, where [symbol] indicates attachment to R¹⁸ of the linker:

TABLE 6

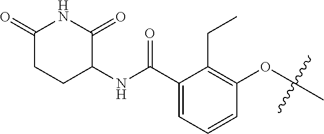 [SMILES: *Oc1cccc(C(=O)NC2CCC(=O)NC2=O)c1CC]

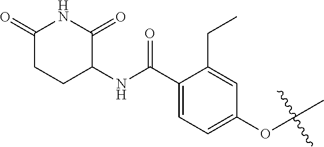 [SMILES: *Oc1ccc(C(=O)NC2CCC(=O)NC2=O)c(CC)c1]

### Ligase Ligand Moieties of Formula (II) and Formula (III)

In the fourth aspect of the present invention, the [ligase ligand moiety] may alternatively be of Formula (II) or of Formula (III). The synthesis of the ligase ligand moieties of Formula (II) and Formula (III) (as defined above) can be summarized as follows:

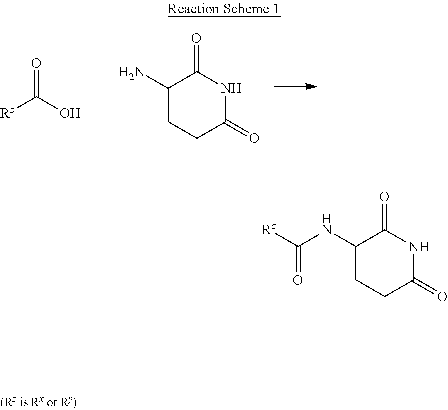

Example ligase ligand moieties of Formula (II) and Formula (III) are shown in Table 7 below. Many of these compounds could be modified to allow attachment to the [linker] (for example, by bromination of the aromatic ring followed by attachment—by palladium coupling—of either the [linker] itself, or of a functional group to which the [linker] could be attached).

TABLE 7

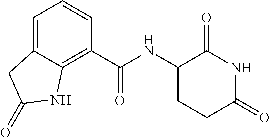 [SMILES: O=C1CCC(NC(=O)c2cccc3c2NC(=O)C3)C(=O)N1]

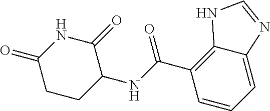 [SMILES: O=C1CCC(NC(=O)c2cccc3nc[nH]c23)C(=O)N1]

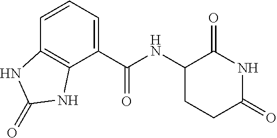 [SMILES: O=C1CCC(NC(=O)c2cccc3[nH]c(=O)[nH]c23)C(=O)N1]

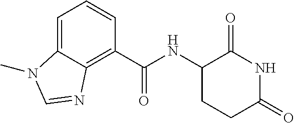 [SMILES: Cn1cnc2c(C(=O)NC3CCC(=O)NC3=O)cccc21]

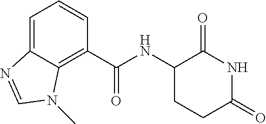 [SMILES: Cn1cnc2cccc(C(=O)NC3CCC(=O)NC3=O)c21]

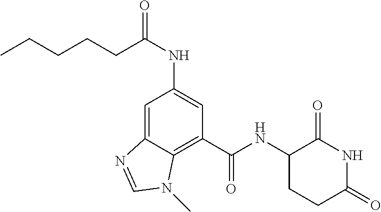 [SMILES: CCCCCC(=O)Nc1cc(C(=O)NC2CCC(=O)NC2=O)c2c(c1)ncn2C]

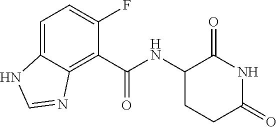 [SMILES: O=C1CCC(NC(=O)c2c(F)ccc3[nH]cnc23)C(=O)N1]

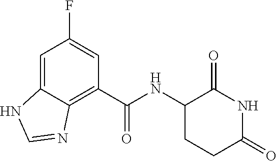 [SMILES: O=C1CCC(NC(=O)c2cc(F)cc3[nH]cnc23)C(=O)N1]

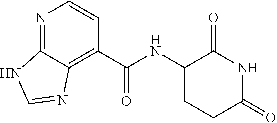 [SMILES: O=C1CCC(NC(=O)c2ccnc3[nH]cnc23)C(=O)N1]

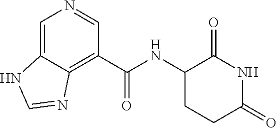 [SMILES: O=C1CCC(NC(=O)c2cncc3[nH]cnc23)C(=O)N1]

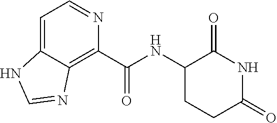 [SMILES: O=C1CCC(NC(=O)c2nccc3[nH]cnc23)C(=O)N1]

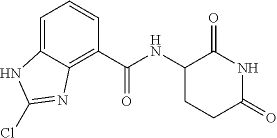 [SMILES: O=C1CCC(NC(=O)c2cccc3[nH]c(Cl)nc23)C(=O)N1]

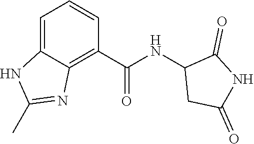 [SMILES: Cc1nc2c(C(=O)NC3CC(=O)NC3=O)cccc2[nH]1]

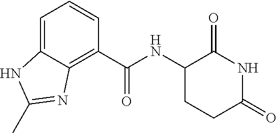 [SMILES: Cc1nc2c(C(=O)NC3CCC(=O)NC3=O)cccc2[nH]1]

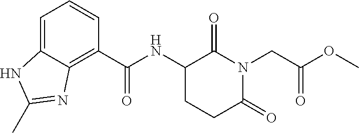

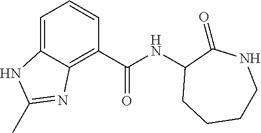 [SMILES: Cc1nc2c(C(=O)NC3CCCCNC3=O)cccc2[nH]1]

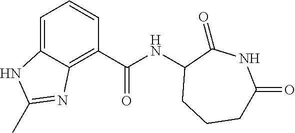 [SMILES: Cc1nc2c(C(=O)NC3CCCC(=O)NC3=O)cccc2[nH]1]

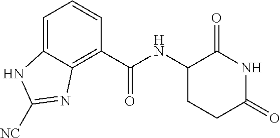 [SMILES: N#Cc1nc2c(C(=O)NC3CCC(=O)NC3=O)cccc2[nH]1]

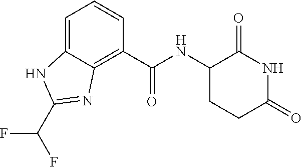 [SMILES: O=C1CCC(NC(=O)c2cccc3[nH]c(C(F)F)nc23)C(=O)N1]

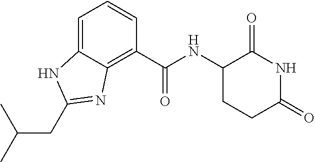 [SMILES: CC(C)Cc1nc2c(C(=O)NC3CCC(=O)NC3=O)cccc2[nH]1]

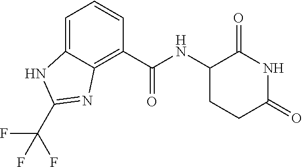 [SMILES: O=C1CCC(NC(=O)c2cccc3[nH]c(C(F)(F)F)nc23)C(=O)N1]

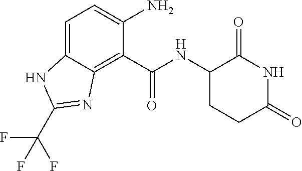 [SMILES: Nc1ccc2[nH]c(C(F)(F)F)nc2c1C(=O)NC1CCC(=O)NC1=O]

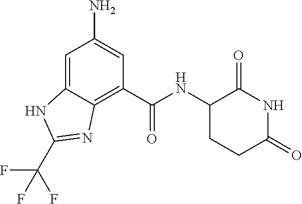 [SMILES: Nc1cc(C(=O)NC2CCC(=O)NC2=O)c2nc(C(F)(F)F)[nH]c2c1]

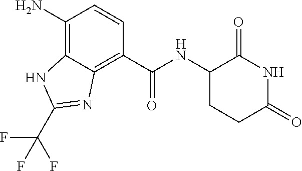 [SMILES: Nc1ccc(C(=O)NC2CCC(=O)NC2=O)c2nc(C(F)(F)F)[nH]c12]

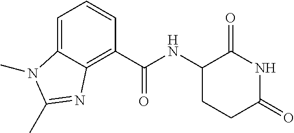 [SMILES: Cc1nc2c(C(=O)NC3CCC(=O)NC3=O)cccc2n1C]

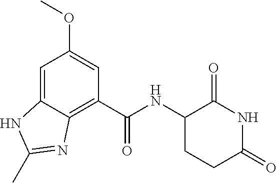 [SMILES: COc1cc(C(=O)NC2CCC(=O)NC2=O)c2nc(C)[nH]c2c1]

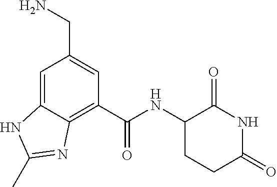 [SMILES: Cc1nc2c(C(=O)NC3CCC(=O)NC3=O)cc(CN)cc2[nH]1]

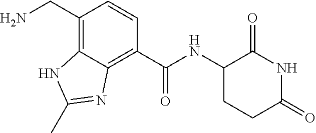 [SMILES: Cc1nc2c(C(=O)NC3CCC(=O)NC3=O)ccc(CN)c2[nH]1]

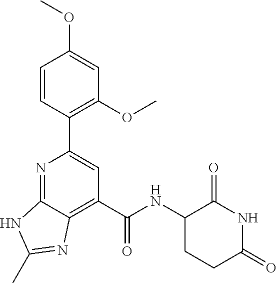 [SMILES: COc1ccc(-c2cc(C(=O)NC3CCC(=O)NC3=O)c3nc(C)[nH]c3n2)c(OC)c1]

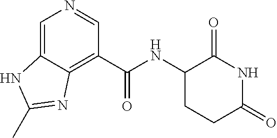 [SMILES: Cc1nc2c(C(=O)NC3CCC(=O)NC3=O)cncc2[nH]1]

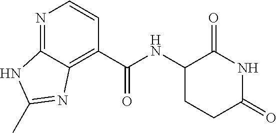 [SMILES: Cc1nc2c(C(=O)NC3CCC(=O)NC3=O)ccnc2[nH]1]

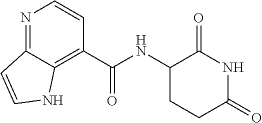 [SMILES: O=C1CCC(NC(=O)c2ccnc3cc[nH]c23)C(=O)N1]

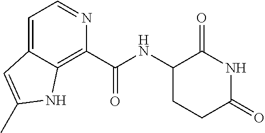 [SMILES: Cc1cc2ccnc(C(=O)NC3CCC(=O)NC3=O)c2[nH]1]

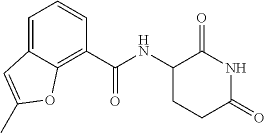 [SMILES: Cc1cc2cccc(C(=O)NC3CCC(=O)NC3=O)c2o1]

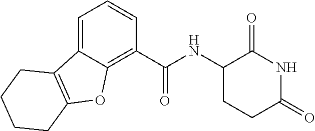 [SMILES: O=C1CCC(NC(=O)c2cccc3c4c(oc23)CCCC4)C(=O)N1]

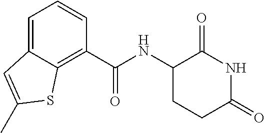 [SMILES: Cc1cc2cccc(C(=O)NC3CCC(=O)NC3=O)c2s1]

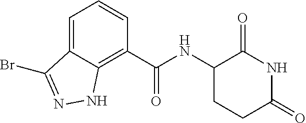 [SMILES: O=C1CCC(NC(=O)c2cccc3c(Br)n[nH]c23)C(=O)N1]

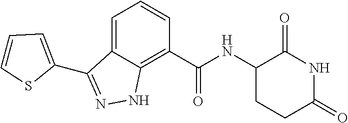 [SMILES: O=C1CCC(NC(=O)c2cccc3c(-c4cccs4)n[nH]c23)C(=O)N1]

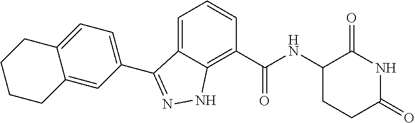 [SMILES: O=C1CCC(NC(=O)c2cccc3c(-c4ccc5c(c4)CCCC5)n[nH]c23)C(=O)N1]

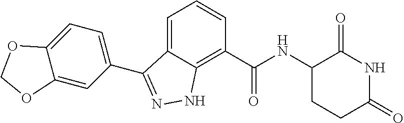 [SMILES: O=C1CCC(NC(=O)c2cccc3c(-c4ccc5c(c4)OCO5)n[nH]c23)C(=O)N1]

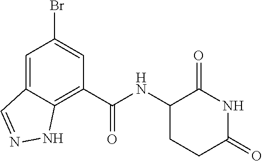 [SMILES: O=C1CCC(NC(=O)c2cc(Br)cc3cn[nH]c23)C(=O)N1]

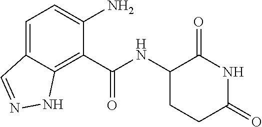 [SMILES: Nc1ccc2cn[nH]c2c1C(=O)NC1CCC(=O)NC1=O]

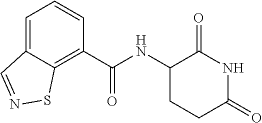 [SMILES: O=C1CCC(NC(=O)c2cccc3cnsc23)C(=O)N1]

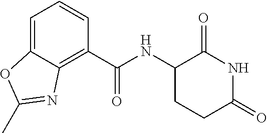 [SMILES: Cc1nc2c(C(=O)NC3CCC(=O)NC3=O)cccc2o1]

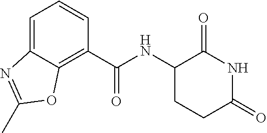 [SMILES: Cc1nc2cccc(C(=O)NC3CCC(=O)NC3=O)c2o1]

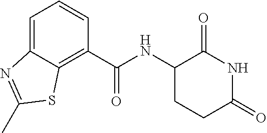 [SMILES: Cc1nc2cccc(C(=O)NC3CCC(=O)NC3=O)c2s1]

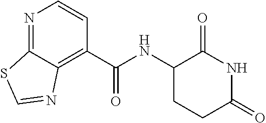 [SMILES: O=C1CCC(NC(=O)c2ccnc3scnc23)C(=O)N1]

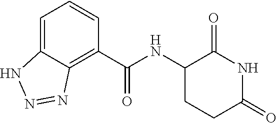 [SMILES: O=C1CCC(NC(=O)c2cccc3[nH]nnc23)C(=O)N1]

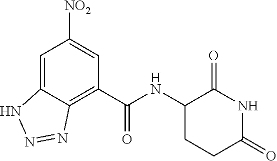 [SMILES: O=C1CCC(NC(=O)c2cc([N+](=O)[O-])cc3[nH]nnc23)C(=O)N1]

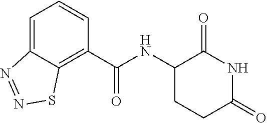 [SMILES: O=C1CCC(NC(=O)c2cccc3nnsc23)C(=O)N1]

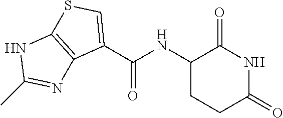 [SMILES: Cc1nc2c(C(=O)NC3CCC(=O)NC3=O)csc2[nH]1]

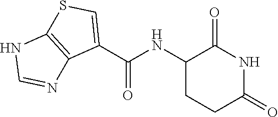 [SMILES: O=C1CCC(NC(=O)c2csc3[nH]cnc23)C(=O)N1]

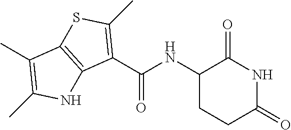 [SMILES: Cc1[nH]c2c(C(=O)NC3CCC(=O)NC3=O)c(C)sc2c1C]

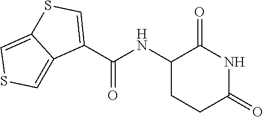 [SMILES: O=C1CCC(NC(=O)c2csc3cscc23)C(=O)N1]

Example ligase ligand moieties of Formula (III) in which the attachment to the linker is indicated (R²¹ being a bond connected to R¹⁸ of the linker) are shown in Table 8 below:

TABLE 8

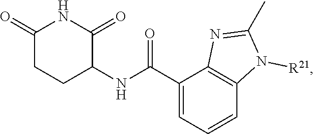

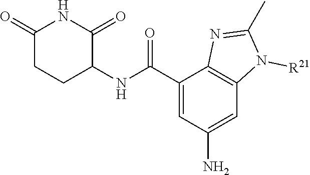

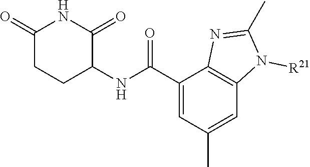

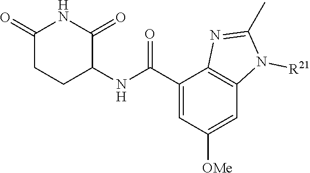

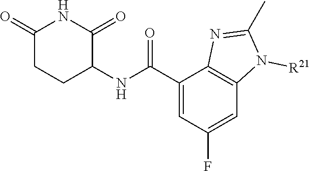

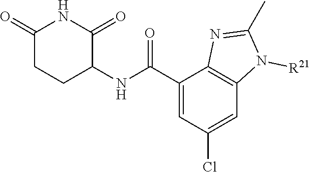

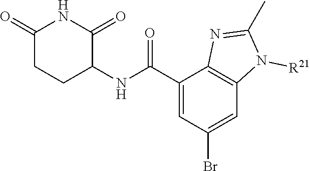

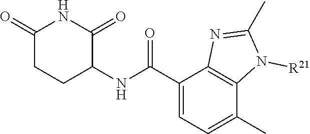

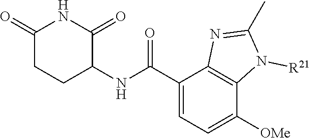

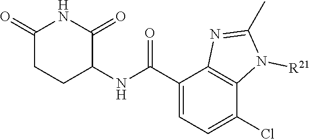

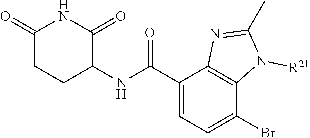

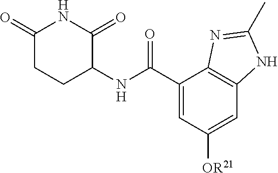

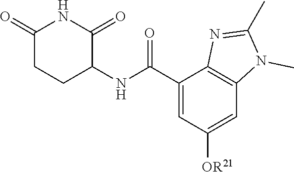

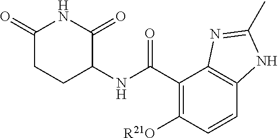

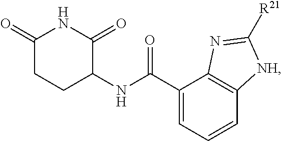

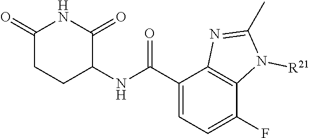

### Linkers

In the first, second, third and fourth aspects of the invention, the [linker] has the following formula:

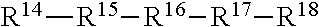

wherein
R¹⁴ is —C₁₋₆alkyl, —C₂₋₆alkenyl, —C₂₋₆alkynyl, —C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)₁, —(C₃H₆—O),, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
   wherein at least one of R¹⁴—R¹⁸ is present.

In some embodiments, R¹⁴ is —C₁-C₆ alkyl, —C₂₋₆alkenyl, —C₂₋₆alkynyl, —C(O)—, —SO₂— or is absent. In some such embodiments, R¹⁴ is —C₁-C₆ alkyl.

In some embodiments, R¹⁵ is cycloalkyl, heterocycloalkyl, aryl, heteroaryl, —C₁₋₆ alkyl-NH—, -cycloalkyl-NH— or is absent. In some such embodiments, R¹⁵ is heterocycloalkyl or is absent. In some embodiments, R¹⁵ is heterocycloalkyl. In some such embodiments, R¹⁵ is piperazine, bridged piperazine, piperazine N-oxide, piperazine cation,

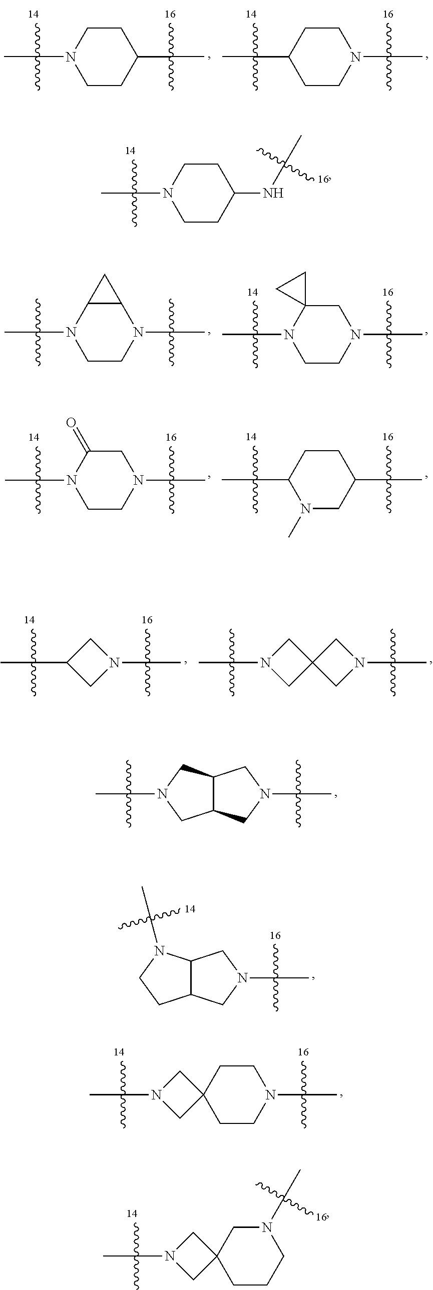

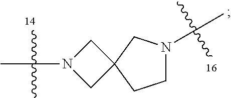 [SMILES: *N1CCC2(C1)CN(*)C2]

wherein [symbol] indicates attachment to R¹⁴ and [symbol] indicates attachment to R¹⁶.

In some embodiments, R¹⁵ is piperazine or

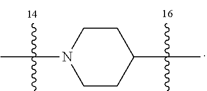 [SMILES: *C1CCN(*)CC1]

In some embodiments, R¹⁶ is —C₁-C₆ alkyl, —CH₂—C(O)—NH—, —CH₂—C(O)—, —C(O)— or is absent. In some embodiments, R¹⁶ is C₁-C₆ alkyl, —CH₂—C(O)—NH— or —C(O)—. In some embodiments, R¹⁶ is —C₁-C₆ alkyl or —C(O)—.

In some embodiments, R¹⁷ is absent.

In some embodiments, R¹⁸ is —C₁₋₆ alkyl, heterocycloalkyl, or is absent. In some embodiments, R¹⁸ is —C₁-C₆ alkyl or heterocycloalkyl. In some embodiments, R¹⁸ is —C₁₋₆ alkyl or piperazine.

In some embodiments,
R¹⁴ is —C₁-C₆ alkyl, —C₁₋₆ alkyl-N(Me)-, —SO₂— or is absent;
R¹⁵ is piperazine, bridged piperazine, piperazine N-oxide, piperazine cation,

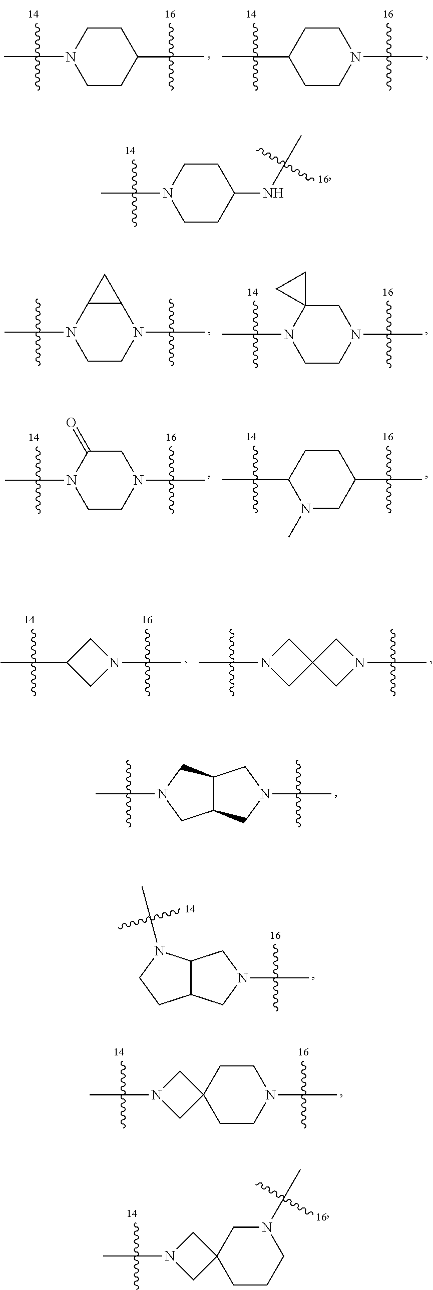

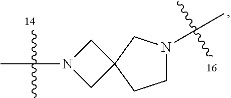 [SMILES: *N1CCC2(C1)CN(*)C2]

or is absent,
   wherein [symbol] indicates attachment to R¹⁴ and [symbol] indicates attachment to R¹⁶,
R¹⁶ is —C₁-C₆ alkyl, —CH₂—C(O)—NH—, —CH₂—C(O)—, —C(O)— or is absent;
R¹⁷ is —CH₂(C₂H₄—O)_{y}, (C₂H₄—O)ₓ, (C₃H₆—O)ₓ, or is absent, wherein x is 1-6 and y is 2-6; and
R¹⁸ is —C₁₋₆ alkyl, piperazine,

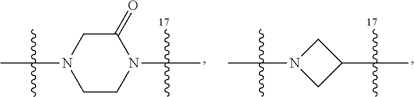 [SMILES: *N1CCN(*)C(=O)C1 ; *C1CN(*)C1]

or is absent, wherein [symbol] indicates attachment to R¹⁷,
and wherein at least one of R¹⁴—R¹⁸ is present.

In some embodiments,
R¹⁴ is —C₁-C₆ alkyl,
R¹⁵ is piperazine or

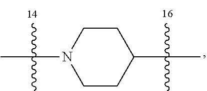 [SMILES: *C1CCN(*)CC1]

R¹⁶ is —C₁-C₆ alkyl or —C(O)—
R¹⁷ is absent
R¹⁸ is —C₁₋₆ alkyl or piperazine.

In some embodiments, [linker] is selected from

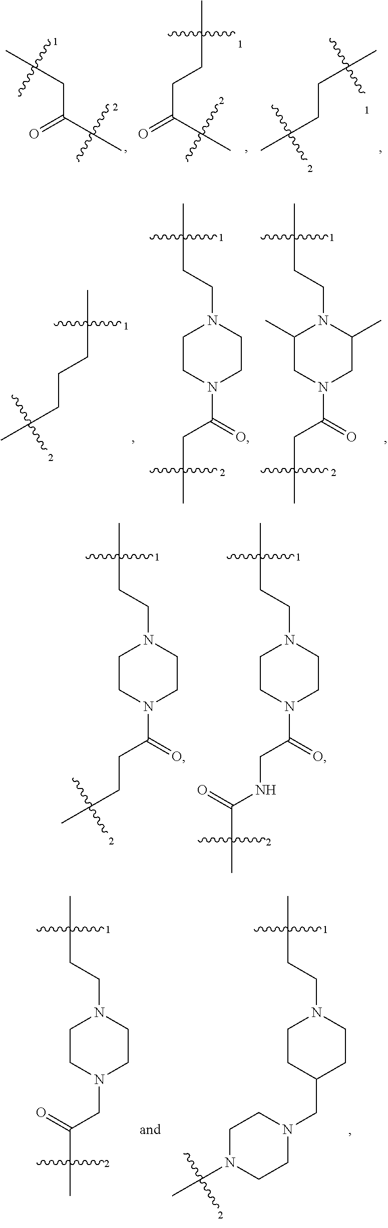

wherein
[symbol] indicates attachment to [MCL-1 ligand prodrug moiety] and
[symbol] indicates attachment to [ligase ligand moiety].

In some embodiments, [linker] is selected from

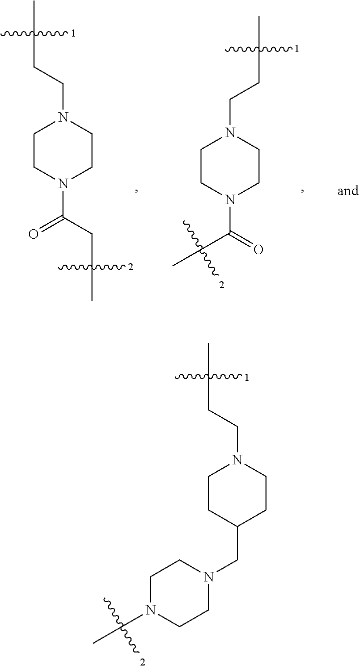 [SMILES: *CCN1CCN(C(=O)C*)CC1 ; *CCN1CCN(C(*)=O)CC1 ; *CCN1CCC(CN2CCN(*)CC2)CC1]

wherein
[symbol] indicates attachment to [MCL-1 ligand prodrug moiety] and
[symbol] indicates attachment to [ligase ligand moiety].

Linkers as used in the compounds of the present invention may be synthesized according to standard methods.

Most of the alkyl and polyethylene glycol (PEG) linkers were commercially available, or prepared due to procedures described in literature.

Examples of commercially available linkers include:

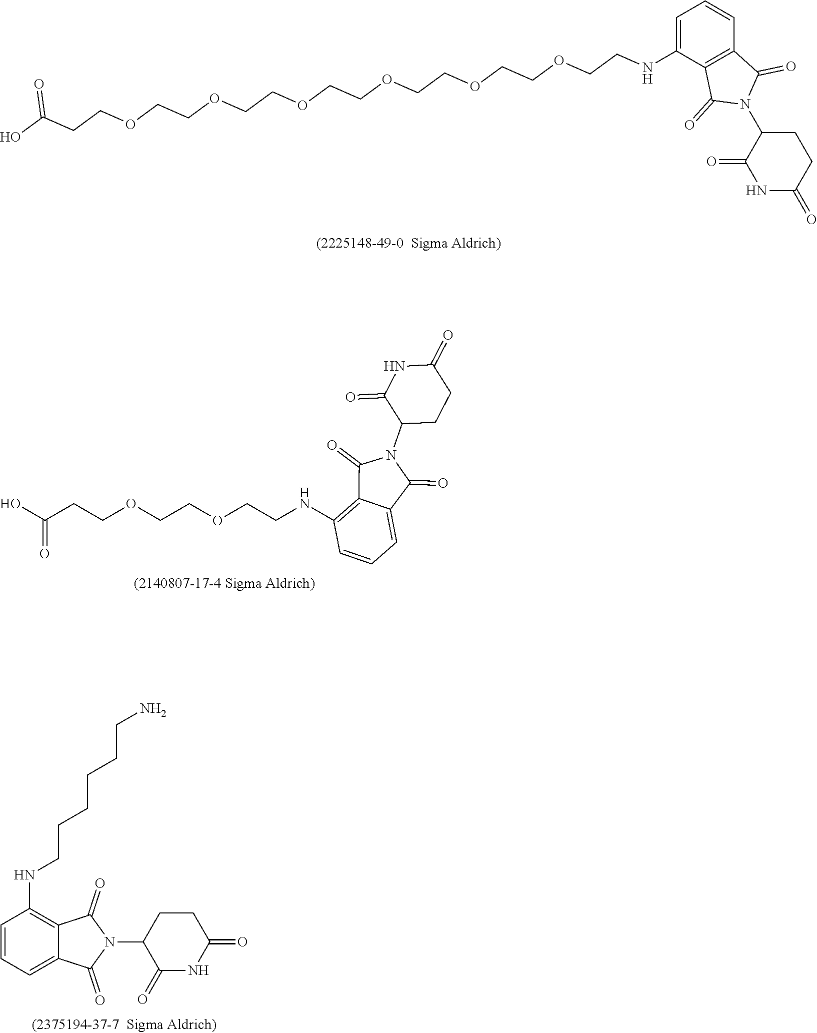 [SMILES: O=C(O)CCOCCOCCOCCOCCOCCOCCNc1cccc2c1C(=O)N(C1CCC(=O)NC1=O)C2=O ; O=C(O)CCOCCOCCNc1cccc2c1C(=O)N(C1CCC(=O)NC1=O)C2=O ; NCCCCCCNc1cccc2c1C(=O)N(C1CCC(=O)NC1=O)C2=O]

Synthesis of linkers which are not commercially available are described in the examples.

Linkers containing modifications of piperazine were prepared according to the following scheme:

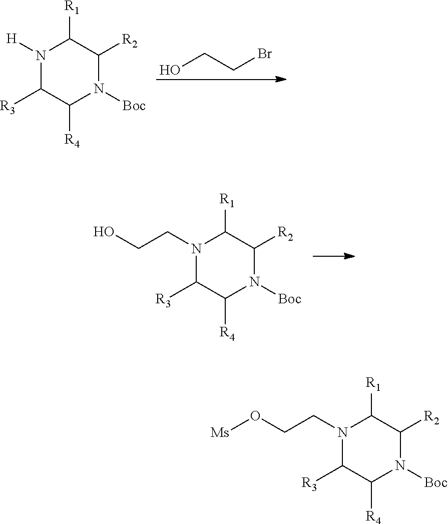

### MCL-1 Ligand Prodrug Moiety

In the first, second, third and fourth aspects of the invention, the [MCL-1 ligand prodrug moiety] is a compound of Formula (A1), (A2), (A3) or (A4):

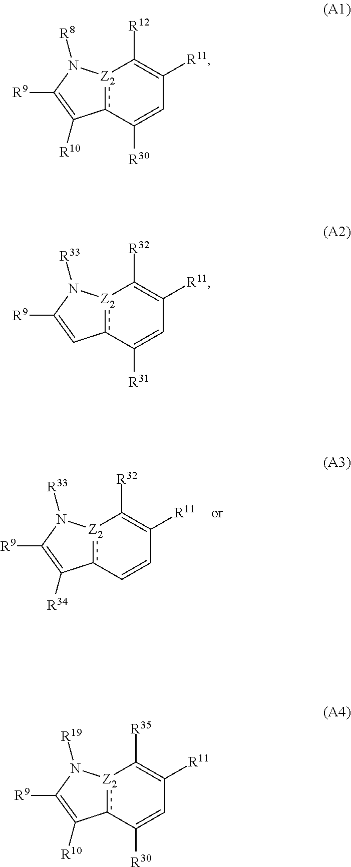

wherein
[symbol] is a single bond or a double bond;
each Z₂ is independently N or C, wherein when Z₂ is N, then [symbol] is a single bond; and when Z₂ is C, then [symbol] is a double bond;
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is

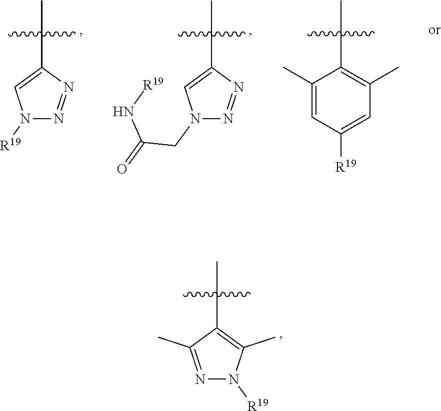

R³¹ is —C₂₋₅alkyl-O—R¹³, —O—C₂₋₅alkyl-R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H,

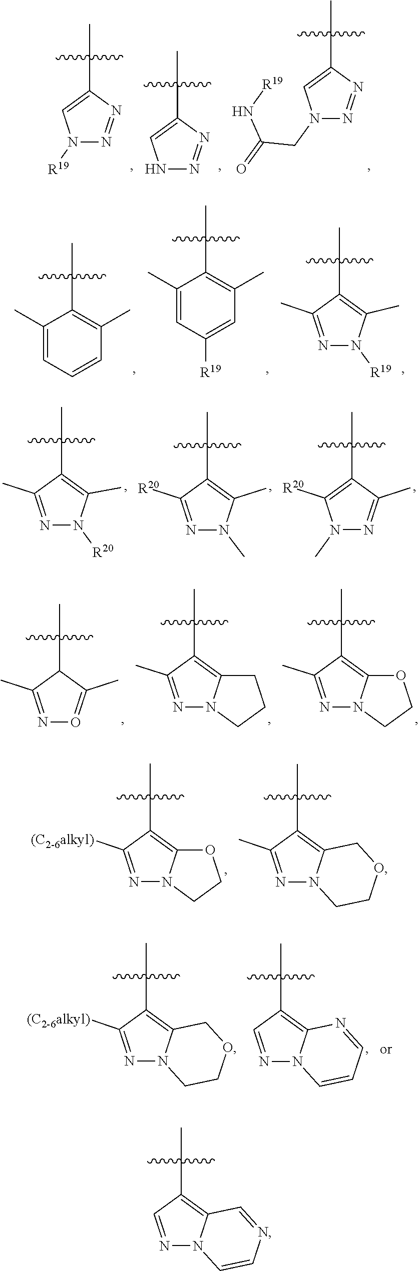

each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is

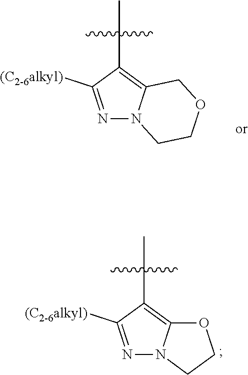

R¹⁹ is a bond connected to R¹⁴ of the linker; wherein each of Formula (A1), Formula (A2), Formula (A3) and Formula (A4) contains a single R¹⁹; and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, —C(O)NMe₂;
each R^{p} is independently —(CH₂)ₘOC(O)(unsubstituted C₁-C₆alkyl), and
each m is independently 1, 2, 3 or 4;
or
(ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me wherein each p is independently 2, 3, 4, 5, or 6.

In some embodiments, [MCL-1 ligand prodrug moiety] is:

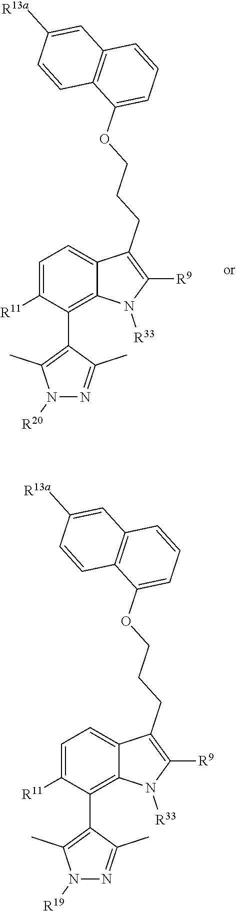

wherein
R¹¹ is halogen;
R^{13a} is halogen; and
R²⁰ is Me, —CH₂—OMe, or —(CH₂CH₂O)ₚ(C₁-C₆ alkyl), wherein p is 1-5.

In some embodiments, R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolyl cation. In some such embodiments, R⁹ is —C(O)O(C₁-C₆alkyl), —C(O)Ocycloalkyl, —C(O)Oheterocycloalkyl, —C(O)Oaryl, or —P(O)(OR^{p})₂, wherein said C₁-C₆alkyl is substituted by one or more groups selected from —OC(O)Me, —OC(O)Et, —OC(O)ⁱPr, —OC(O)C(CH₃)₃, —OC(O)CH₂OH, —OC(O)CH(CH₃)OH, —OC(O)C(CH₃)₂OH, —OC(O)CH₂CH₂OH, —OC(O)CH(OH)CHMe₂, —OC(O)CH(OH)CH₂CHMe₂, —OC(O)CH₂CH(OH)Me, —OC(O)CH₂C(OH)Me₂, —OC(O)CH(CH₂OH)(NHC(O)Me), —OC(O)CH(NHC(O)Me)(isobutyl), —OC(O)CH(OH)aryl, —OC(O)CH(OH)CH₂aryl, —OC(O)heterocycloalkyl, —OC(O)cycloalkyl, —OC(O)aryl, —OC(O)heteroaryl, —OC(O)CH₂CH₂aryl, —OC(O)CH(NH₂)ⁱPr, —OC(O)CH(ⁱPr)NHC(O)O^{t}Bu, —OC(O)CH₂OCH₂CH₂OMe, —OC(O)CH₂(OCH₂CH₂)₂OMe, —OC(O)OMe, —OC(O)OEt, —OC(O)OiPr, —OC(O)Ocycloalkyl, —OC(O)OCH₂CH₂OEt, —OC(O)O(CH₂CH₂O)₂Et, —OC(O)O(CH₂CH₂O)₃Et, —OC(O)O(CH₂CH₂O)₃CH₂CH₂OP(O)(OH)₂, —OC(O)OCH₂CH₂SMe, —OC(O)OCH₂CH₂S(O)Me, —OC(O)OCH₂CH₂S(O)₂Me, —OC(O)OCH₂heteroaryl, —OC(O)OCH₂heterocycloalkyl, —OC(O)OCH₂CH₂OCH₂aryl —OC(O)OCH₂CH₂C(O)NMe₂, —OC(O)O(CH₂)₆NH₂, —OC(O)O(CH₂)₆NMe₂, —OC(O)OCH(CH₂OEt)₂, —OC(O)NHCH₂CH₂OMe, —OC(O)NMeCH₂CH₂OMe, —SMe, —S(O)Me, —P(O)(OH)₂, —OP(O)(OH)₂, —C(O)NMe₂, —C(O)heterocycloalkyl, heteroaryl, heterocycloalkyl, —N⁺(Me)₃, —N⁺(Me)₂cycloalkyl, N-methyl morpholinium cation, N-methyl piperidinium cation and N-methyl imidazolium cation.

In some embodiments, R⁹ is —C(O)O(C₁-C₆alkyl) wherein said C₁-C₆alkyl is substituted by one or more groups selected from —OC(O)C(CH₃)₃, —OC(O)CH₂CH₂OH, —OC(O)C(CH₃)₂OH, —OC(O)heterocycloalkyl, —OC(O)CH₂OCH₂CH₂OMe, —OC(O)OiPr, —OC(O)Ocycloalkyl, —OC(O)O(CH₂CH₂O)₂Et, —OP(O)(OH)₂, heteroaryl or heterocycloalkyl. In some such embodiments, R⁹ is —C(O)O(C₁-C₆alkyl) wherein said C₁-C₆alkyl is substituted by —OC(O)C(CH₃)₃

In some embodiments, R⁹ is —C(O)OCH₂OC(O)C(CH₃)₃, —C(O)OCH₂OC(O)Me, —C(O)OCH(Me)OC(O)Me —C(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂OMe, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, —C(O)OCH₂CH₂CH₂piperazine or —P(O)(OCH₂OC(O)C(CH₃)₃)₂.

In some embodiments, R⁹ is —C(O)OCH₂OC(O)C(CH₃)₃.

In some embodiments, [MCL-1 ligand prodrug moiety] is:

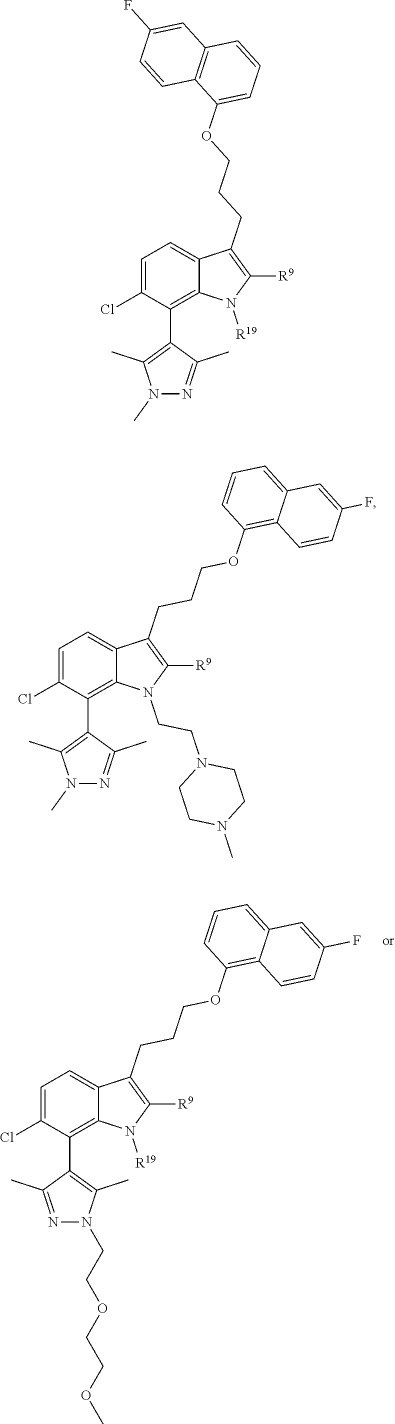

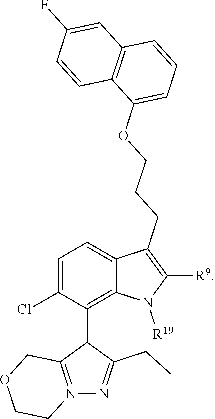

### EXAMPLES

There are a number of ways in which the bifunctional compounds [MCL-1 ligand prodrug moiety]-[linker]-[ligase ligand prodrug moiety] of the present application may be synthesized:

1. [Mcl-1 Ligand Prodrug Moiety] is Coupled with linkerA, Followed by Coupling with [Ligase Ligand Moiety]-linkerB

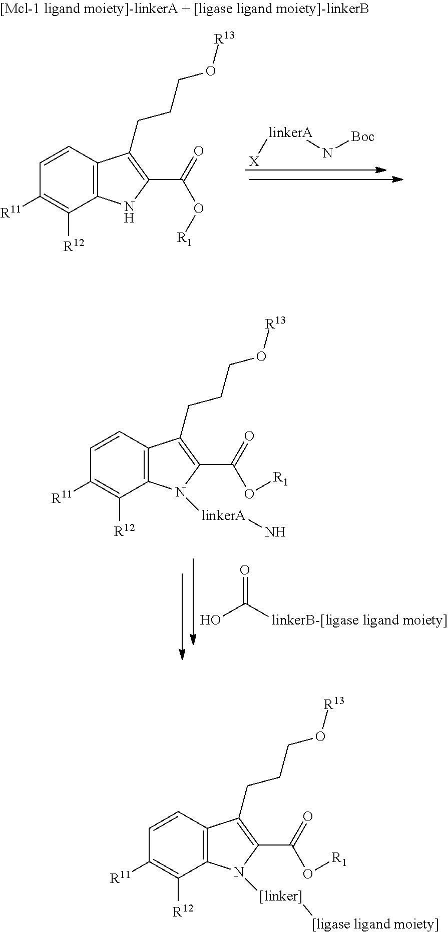

wherein X is halogen or OMs, OTs; linkerA-N-Boc corresponds to linkerA terminating with a Boc-protected primary or secondary amine; linkerA-NH corresponds to linkerA terminating with a primary or secondary amine; and R¹¹—R¹³ and [linker] are as defined herein; wherein [linker] is formed in the above synthesis by the reaction of -linkerA-NHR^{w} with -linkerB-COOH.
2. [MCL-1 Ligand Prodrug Moiety] is Coupled with linkerA, Followed by Coupling with linkerB, Followed Coupling with [Ligase Ligand Moiety]

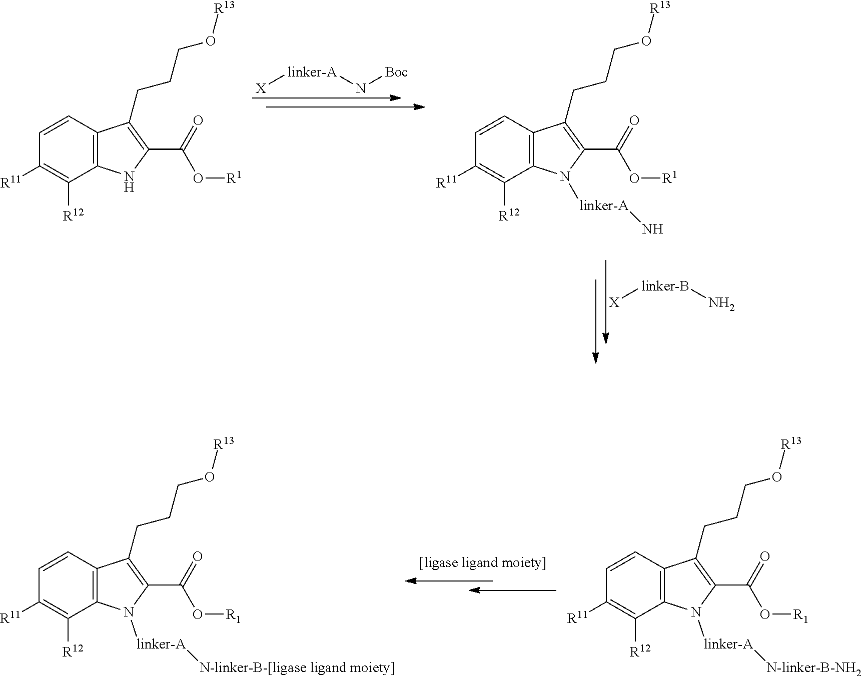

wherein X is halogen or OMs, OTs; linkerA-N-Boc corresponds to linkerA terminating with a Boc-protected primary or secondary amine; linkerA-NH corresponds to linkerA terminating with a primary or secondary amine; and R¹¹—R¹³ are as defined herein; and wherein linkerA-N-linkerB corresponds to [linker].
3. [MCL-1 Ligand Prodrug Moiety]-[Linker] is Coupled with [Ligase Ligand Moiety]

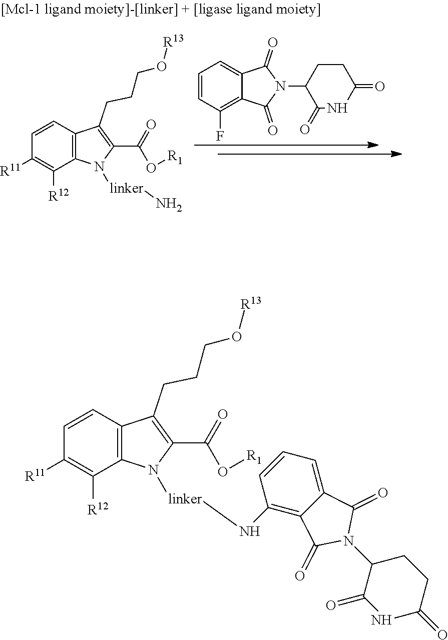

4. [MCL-1 Ligand Prodrug Moiety] is Coupled with [Linker]-[Ligase Ligand Moiety]

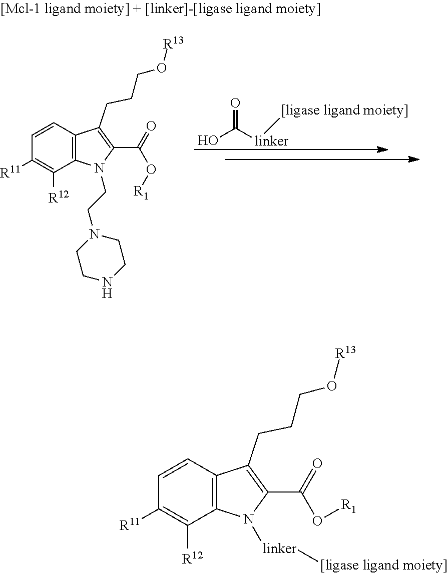

5. [MCL-1 Ligand Prodrug Moiety]-[Linker]-R^{v} is Coupled with 3-Aminopiperidine-2,6-Dione

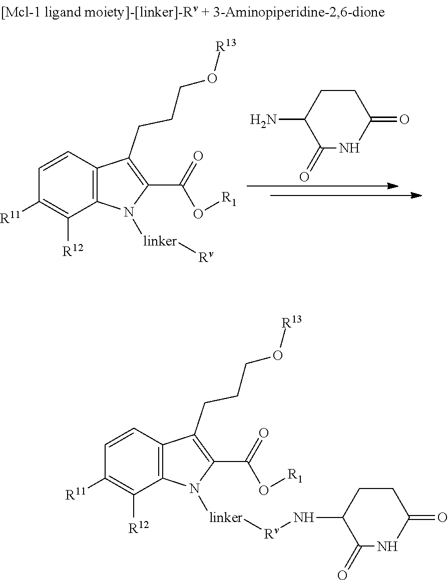

The bifunctional compounds of the present invention were prepared as follows:

### Example 1: (2-(2-Methoxyethoxy)acetoxy)methyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 57)

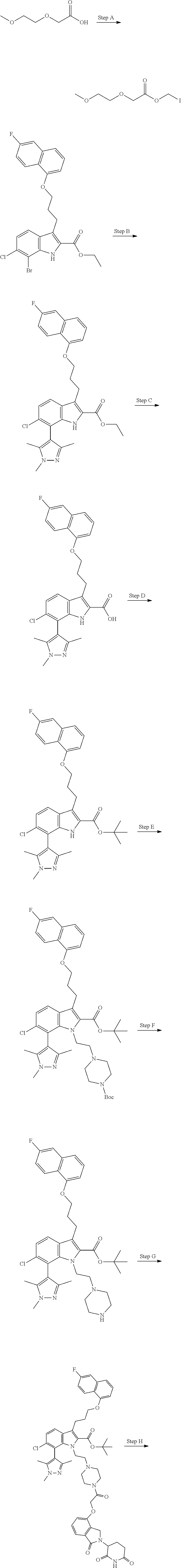

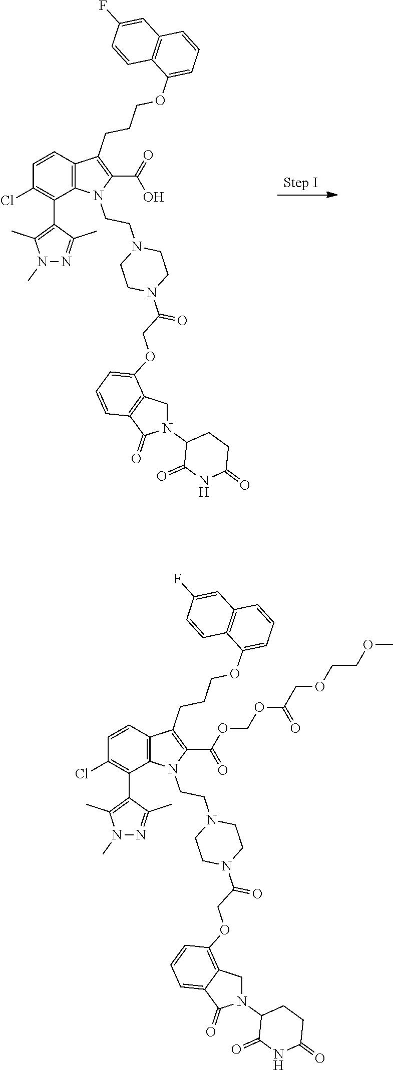

### Step A

To a solution of 2-(2-methoxyethoxy)acetic acid (250 mg, 1.86 mmol) in DMF (4 mL) was added chloroiodomethane (0.172 mL, 2.052 mmol), followed by KHCO₃ (554 mg, 5.6 mmol) at room temperature under nitrogen. The resulting mixture was allowed to stir at 60° C. for 1 h under nitrogen. After complete consumption of the starting material (monitored by TLC) the reaction mixture was cooled to room temperature and filtered through a sintered funnel. The filtrate of chloromethyl 2-(2-methoxyethoxy)acetate in DMF was used without further purification.

### Step B

To a stirred solution of ethyl 7-bromo-6-chloro-3-(3-((6-fluoronaphthalen-1-yl)oxy)propyl)-1H-indole-2-carboxylate (1.7 g, 3.368 mmol) in dioxane (20 mL) and water (5 mL) were added 1,3,5-trimethyl-4-(4,4,5,5-tetramethyl-1,3,2-dioxaborolan-2-yl)-1H-pyrazole (2.386 g, 10.103 mmol) and K₂CO₃ (1.859 g, 13.471 mmol). The mixture was deoxygenated with argon and to it was added Pd(dppf)Cl₂ (0.369 g, 0.505 mmol) under argon atmosphere. Then the reaction mixture was heated under reflux for 16 h. After complete consumption of the starting material (monitored by TLC and LCMS) the reaction mixture was filtered through celite pad and the solvents were evaporated under reduced pressure get the crude material. It was then diluted with EtOAc, washed successively with water and brine, the organic layer was dried over Na₂SO₄ and evaporated under reduced pressure to get the crude compound, which was then purified by column chromatography (SiO₂, 50% EtOAc in hexane) to get ethyl 6-chloro-3-(3-((6-fluoronaphthalen-1-yl)oxy)propyl)-7-(1,3,5-trimethyl-1H-pyrazol-4-yl)-1H-indole-2-carboxylate (1.2 g, 2.247 mmol, 66.72%) as brown solid.

LCMS (ESI): 534.2 m/z [M+H]⁺

### Step C

Ethyl 6-chloro-3-(3-((6-fluoronaphthalen-1-yl)oxy)propyl)-7-(1,3,5-trimethyl-1H-pyrazol-4-yl)-1H-indole-2-carboxylate (1.2 g, 2.251 mmol) was dissolved in EtOH (20 mL) and solution of NaOH (0.315 g, 7.88 mmol) in water (10 mL) was added to it. The mixture was heated under reflux for 16 h. After complete consumption of the starting material (monitored by TLC and LCMS) the reaction mixture was cooled down to room temperature, solvents were evaporated under reduced pressure to get the crude reaction mixture. It was then diluted with water, washed with EtOAc. Aqueous layer was carefully acidified using 1M HCl to pH=3 and extracted with EtOAc (×3). The organic layer was dried over Na₂SO₄, filtered, and concentrated in vacuo to afford 6-chloro-3-(3-((6-fluoronaphthalen-1-yl)oxy)propyl)-7-(1,3,5-trimethyl-1H-pyrazol-4-yl)-1H-indole-2-carboxylic acid (0.9 g, crude) as brown liquid which was used for the next step without further purification.

LCMS (ESI): 506.3 m/z [M+H]⁺

### Step D

6-Chloro-3-(3-((6-fluoronaphthalen-1-yl)oxy)propyl)-7-(1,3,5-trimethyl-1H-pyrazol-4-yl)-1H-indole-2-carboxylic acid (1.2 g, 2.376 mmol) was suspended in toluene (20 mL) and the mixture was heated to reflux under nitrogen. N,N-dimethylformamide di-tert-butyl acetal (4.547 mL, 19.01 mmol) was added drop-wise to the refluxing mixture. The mixture was heated under reflux for 16 h under nitrogen. After complete consumption of the starting material (monitored by TLC and LCMS) the reaction mixture was then diluted with EtOAc, washed successively with sodium bicarbonate (aqueous, saturated), water and brine. Organic layer was dried over Na₂SO₄ and evaporated under reduced pressure to get the crude compound, which was then purified by column chromatography (SiO₂, 70% EtOAc in hexane) to get tert-butyl 6-chloro-3-(3-((6-fluoronaphthalen-1-yl)oxy)propyl)-7-(1,3,5-trimethyl-1H-pyrazol-4-yl)-1H-indole-2-carboxylate (0.772 g, 1.373 mmol, 61% over two steps) as brownish liquid.

LCMS (ESI): 561.9 m/z [M+H]⁺

### Step E

To solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (0.555 g, 0.989 mmol) in DMF (10 mL) was added tert-butyl 4-(2-chloroethyl)piperazine-1-carboxylate (0.492 g, 1.979 mmol) followed by cesium carbonate (1.607 g, 4.947 mmol) in DMF (5 mL) and the mixture was allowed to stir at 90° C. for 16 h under nitrogen. After complete consumption of the starting material (monitored by TLC and LCMS) the reaction mixture was diluted with EtOAc, washed successively with water and brine. The organic layer was dried over Na₂SO₄ and evaporated under reduced pressure to get the crude compound, which was then purified by column chromatography (SiO₂, 30% EtOAc in hexane) to get tert-butyl 1-(2-(4-(tert-butoxycarbonyl)piperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (0.5 g, 0.645 mmol, 65.27%) as off white solid.

LCMS (ESI): 774.6 m/z [M+H]⁺

### Step F

tert-Butyl 1-(2-(4-(tert-butoxycarbonyl)piperazin-1-yl)ethyl)-6-chloro-3-(3-((6-fluoronaphthalen-1-yl)oxy)propyl)-7-(1,3,5-trimethyl-1H-pyrazol-4-yl)-1H-indole-2-carboxylate (0.3 g, 0.388 mmol) dissolved in 20 mL of 4M HCl in dioxan at 0° C. and the mixture was stirred for 2 h under nitrogen at the same temperature. After complete consumption of the starting material (monitored by TLC and LCMS) the reaction mixture was poured in to cold 1M NaOH solution and extracted several times with DCM. The combined organics were dried over Na₂SO₄ and concentrated in vacuo to get the crude compound which was then purified by column chromatography (amine SiO₂, 10% MeOH in DCM) to get tert-butyl 6-chloro-3-(3-((6-fluoronaphthalen-1-yl)oxy)propyl)-1-(2-(piperazin-1-yl)ethyl)-7-(1,3,5-trimethyl-1H-pyrazol-4-yl)-1H-indole-2-carboxylate (0.165 g, 0.244 mmol, 62.98%) as off white solid.

LCMS (ESI): 674.4 m/z [M+H]⁺

### Step G

2-{[2-(2,6-Dioxopiperidin-3-yl)-1-oxo-2,3-dihydro-1H-isoindol-4-yl]oxy}acetic acid (18.3 mg, 0.058 mmol) was dissolved in dry DMF (1.0 mL) under argon atmosphere and DIPEA (0.025 mL, 0.144 mmol) was added, followed by tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (32.3 mg, 0.048 mmol). The solution was cooled down to 0° C. and HATU (21.9 mg, 0.058 mmol) was added dropwise as a solution in 0.2 mL of dry DMF. The reaction was stirred under argon atmosphere, allowing to warm to room temperature. After 15 min full conversion was observed (monitored with LCMS). The solution was diluted with DCM and washed with brine, NaHCO₃ (sat.), again brine and water. Organic layer was dried over anhydrous MgSO₄, filtered, concentrated and dried under reduced pressure to give tert-butyl 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (44.7 mg, crude) as orange oil. The product was used in the next step without further purification.

LCMS (ESI): 974.6 m/z [M+H]⁺

### Step H

tert-Butyl 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (44.7 mg, crude) was dissolved in dry DCM (0.351 mL) under argon atmosphere. TFA (0.351 mL, 4.584 mmol) was added and the reaction was stirred for 18 h at room temperature. DCM and TFA were evaporated under reduced pressure and the resulting residue dissolved in DMSO and purified with preparative HPLC (H₂O:MeCN+0.1% FA) to give 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (12.6 mg, 0.014 mmol, 29% over two steps) as white solid.

LCMS (ESI): 917.6 m/z [M+H]⁺

### Step I

To a solution of 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (120 mg, 0.13 mmol) in DMF (3 mL), at room temperature and under nitrogen atmosphere, was added KHCO₃ (26 mg, 0.26 mmol), followed by the solution of chloromethyl 2-(2-methoxyethoxy)acetate in DMF obtained in the previous step. The resulting mixture was allowed to stir at 60° C. for 2 h under nitrogen. After complete consumption of the starting material the reaction mixture was diluted with EtOAc and washed with water and brine. The organic layer was dried over sodium sulfate and evaporated under reduced pressure to get the crude compound which was purified twice by preparative TLC (SiO₂, 10% MeOH in DCM, then 50% acetone in DCM) to afford (2-(2-methoxyethoxy)acetoxy)methyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (20 mg, 0.0188 mmol, 14.4%) as white solid.

LCMS (ESI): 1065.0 m/z [M+H]⁺.

¹H NMR (500 MHz, DMSO) δ=10.67 (s, 1H), 8.23 (dd, J=9.2, 5.8, 1H), 7.77 (d, J=8.6, 1H), 7.59 (dd, J=10.4, 2.7, 1H), 7.49-7.40 (m, 3H), 7.37-7.30 (m, 2H), 7.26 (d, J=8.6, 1H), 7.14 (d, J=8.1, 1H), 6.88 (dd, J=5.6, 3.0, 1H), 6.03 (d, J=0.9, 2H), 5.05 (dd, J=13.1, 5.2, 1H), 4.89 (s, 2H), 4.40 (d, J=17.1, 1H), 4.32 (dd, J=17.2, 1.6, 1H), 4.29-4.11 (m, 6H), 3.76 (d, J=1.9, 3H), 3.60-3.57 (m, 2H), 3.44-3.40 (m, 2H), 3.38-3.30 (m, 4H), 3.29-3.24 (m, 2H), 3.20 (s, 2H), 2.88 (ddd, J=17.4, 13.4, 5.5, 1H), 2.64-2.59 (m, 1H), 2.46-2.39 (m, 2H), 2.26-2.18 (m, 2H), 2.15-2.02 (m, 7H), 2.01 (s, 3H), 1.88 (s, 3H).

### Example 2: ((Piperidine-4-carbonyl)oxy)methyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (TFA salt) (Compound 55)

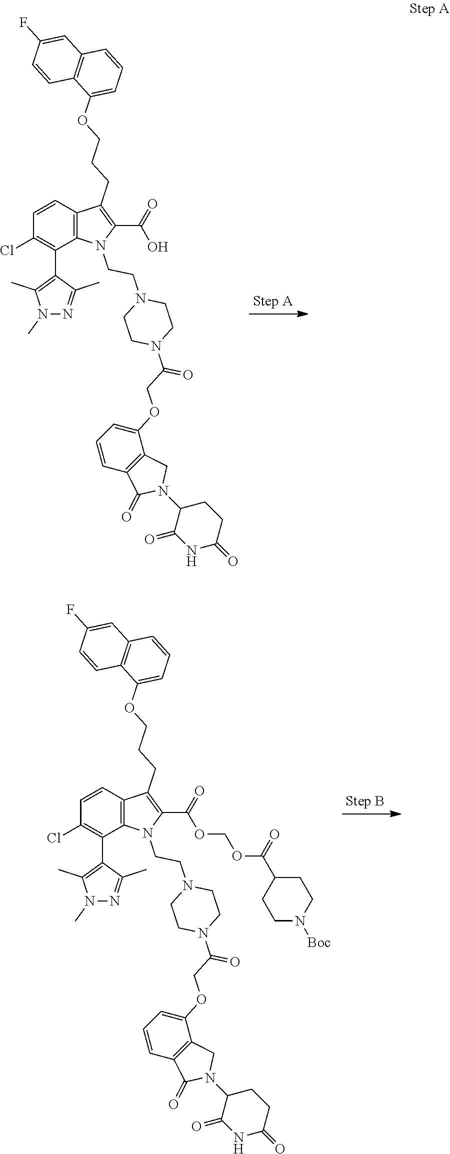 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C3CCN(C(=O)OC(C)(C)C)CC3)n(CCN3CCN(C(=O)COc4cccc5c4CN(C4CCC(=O)NC4=O)C5=O)CC3)c12]

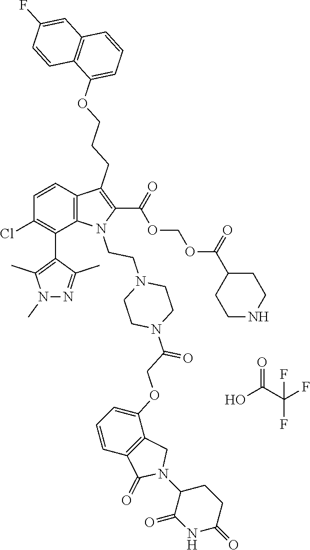 [SMILES: Cc1nn(C)c(C)c1-c1c(Cl)ccc2c(CCCOc3cccc4cc(F)ccc34)c(C(=O)OCOC(=O)C3CCNCC3)n(CCN3CCN(C(=O)COc4cccc5c4CN(C4CCC(=O)NC4=O)C5=O)CC3)c12 ; O=C(O)C(F)(F)F]

To a solution of 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (150 mg, 0.163 mmol) in DMF (2 mL) was added K₂CO₃ (45 mg, 0.327 mmol), followed by the solution of crude 1-(tert-butyl) 4-(chloromethyl) piperidine-1,4-dicarboxylate in DMF at room temperature under nitrogen. The resulting mixture was allowed to stir at 70° C. for 2 h under nitrogen. After complete consumption of the starting material, (monitored by TLC and LCMS), the mixture was concentrated under reduced pressure. The resulting residue was diluted with EtOAc and washed with water and brine. The organic layer was dried over sodium sulfate and concentrated under reduced pressure to get the crude product, which was purified by column chromatography (SiO₂, 3-4% MeOH in DCM). 1-(tert-Butyl) 4-(((6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carbonyl)oxy)methyl) piperidine-1,4-dicarboxylate (110 mg, 0.095 mmol, 58%) was obtained as white solid.

LCMS (ESI): 1157.1 m/z [M−H]⁻.

### Step B

To a solution of 1-(tert-butyl) 4-(((6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy) propyl)-7-(1,3,5-trimethyl-1H-pyrazol-4-yl)-1H-indole-2-carbonyl)oxy)methyl) piperidine-1,4-dicarboxylate (110 mg, 0.095 mmol) in DCM (3 mL) was added dropwise TFA (2 mL) at 0° C. under nitrogen. The reaction mixture was allowed to stir at ambient temperature for 3 h under nitrogen. After complete consumption of the starting material, the volatiles were removed under reduced pressure, and crude was purified by triturating with diethyl ether and n-pentane to afford ((piperidine-4-carbonyl)oxy)methyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (100 mg, 0.085 mmol, 89%) as TFA salt (white solid).

LCMS (ESI): 1060.1 m/z [M+H]⁺

¹H NMR (500 MHz, DMSO, 353 K) δ 10.69 (s, 1H), 8.42 (bs, 1H), 8.25 (dd, J=9.3, 5.9 Hz, 1H), 8.14 b(s, 1H), 7.80 (d, J=8.6 Hz, 1H), 7.63 (dd, J=10.4, 2.5 Hz, 1H), 7.50-7.42 (m, 3H), 7.40-7.32 (m, 2H), 7.29 (d, J=8.6 Hz, 1H), 7.16 (d, J=8.2 Hz, 1H), 6.90 (dd, J=6.2, 2.5 Hz, 1H), 6.04 (d, J=5.9 Hz, 1H), 6.03 (d, J=5.9 Hz, 1H), 5.08 (dd, J=13.0, 5.2 Hz, 1H), 4.92 (s, 2H), 4.42 (d, J=17.2 Hz, 1H), 4.33 (dd, J=17.1, 1.6 Hz, 1H), 4.31-4.23 (m, 3H), 4.23-4.14 (m, 1H), 3.78 (d, J=2.0 Hz, 3H), 3.39-3.35 (m, 4H), 3.31-3.26 (m, 2H), 3.26-3.19 (m, 3H), 2.96-2.85 (m, 2H), 2.78 (tt, J=10.8, 4.0 Hz, 1H), 2.68-2.62 (m, 1H), 2.49-2.41 (m, 1H), 2.28-2.20 (m, 2H), 2.15 (s, 6H), 2.10-1.98 (m, 6H), 1.89 (s, 3H), 1.77 (dtd, J=14.7, 11.1, 4.0 Hz, 2H).

### Example 3: (6-Chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carbonyloxy)methyl 2,2-dimethylpropanoate (Compound 70)

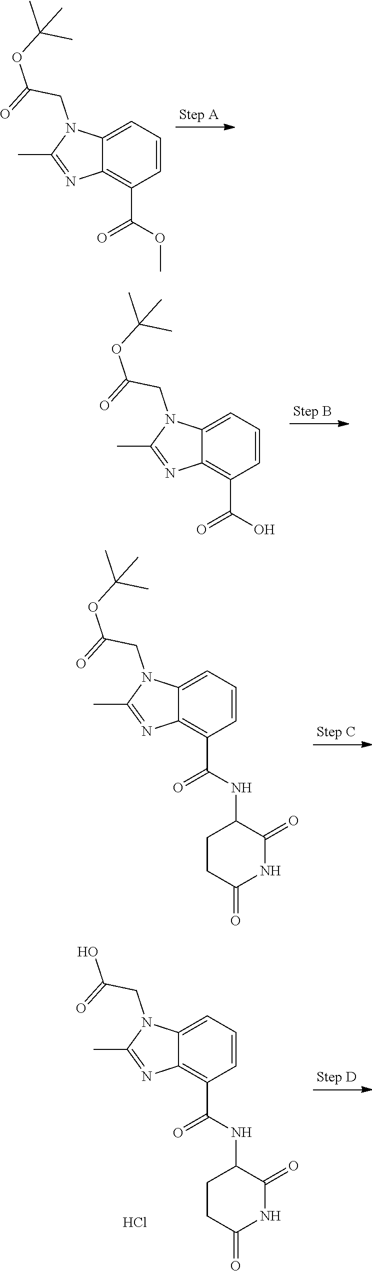

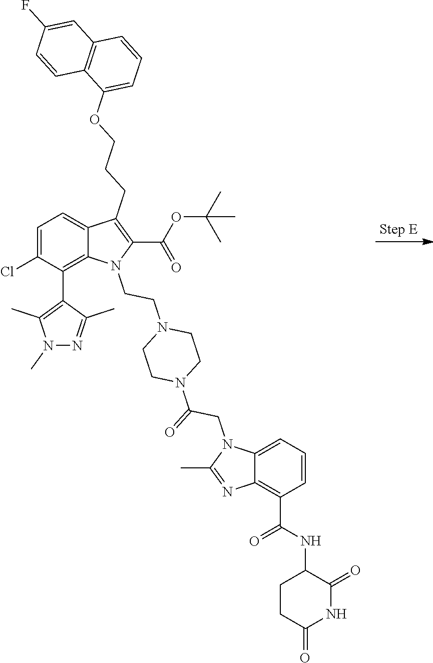 [SMILES: Cc1nn(C)c(C)c1-c1c(Cl)ccc2c(CCCOc3cccc4cc(F)ccc34)c(C(=O)OC(C)(C)C)n(CCN3CCN(C(=O)Cn4c(C)nc5c(C(=O)NC6CCC(=O)NC6=O)cccc54)CC3)c12]

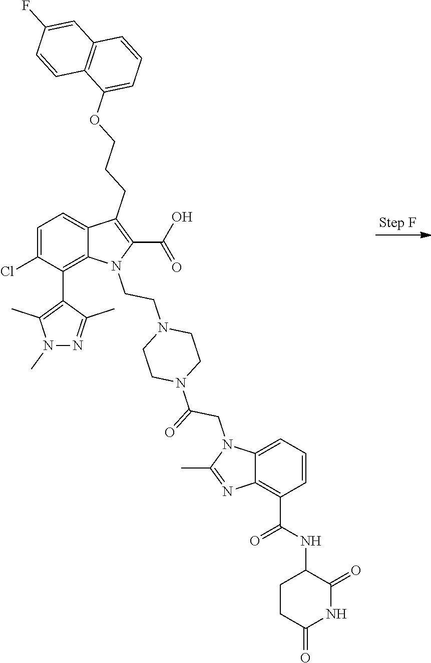 [SMILES: Cc1nn(C)c(C)c1-c1c(Cl)ccc2c(CCCOc3cccc4cc(F)ccc34)c(C(=O)O)n(CCN3CCN(C(=O)Cn4c(C)nc5c(C(=O)NC6CCC(=O)NC6=O)cccc54)CC3)c12]

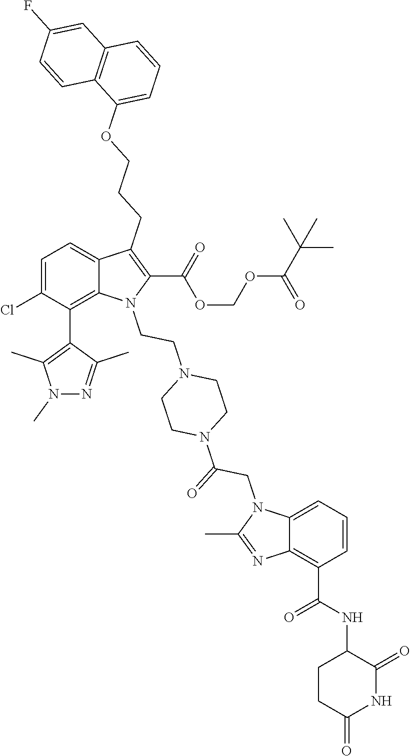 [SMILES: Cc1nn(C)c(C)c1-c1c(Cl)ccc2c(CCCOc3cccc4cc(F)ccc34)c(C(=O)OCOC(=O)C(C)(C)C)n(CCN3CCN(C(=O)Cn4c(C)nc5c(C(=O)NC6CCC(=O)NC6=O)cccc54)CC3)c12]

### Step A

To a solution of methyl 1-[2-(tert-butoxy)-2-oxoethyl]-2-methyl-1H-1,3-benzodiazole-4-carboxylate (152.0 mg, 0.499 mmol) in H₂O (0.500 mL) and MeCN (5.0 mL) was added LiBr (867.4 mg, 9.989 mmol) and Et₃N (0.695 mL, 4.994 mmol). The mixture was stirred for 4 days at RT. The crude was concentrated in vacuo and purified by reverse phase flash chromatography (H₂O:MeCN+0.1% FA) to get 1-[2-(tert-butoxy)-2-oxoethyl]-2-methyl-1H-1,3-benzodiazole-4-carboxylic acid (75.0 mg, 0.233 mmol, 47%) as a yellow solid.

LCMS (ESI): 291.0 m/z [M+H]⁺

### Step B

To a solution of 1-[2-(tert-butoxy)-2-oxoethyl]-2-methyl-1H-1,3-benzodiazole-4-carboxylic acid (100.0 mg, 0.344 mmol) and 3-aminopiperidine-2,6-dione hydrochloride (68.0 mg, 0.413 mmol) in dry DMF (2.0 mL) was added DIPEA (0.300 mL, 1.722 mmol) and HATU (196.5 mg, 0.517 mmol). The mixture was stirred for 18 h at RT. After this time, the crude was concentrated in vacuo and the residues were dissolved in DCM and purified by flash chromatography (SiO₂, MeOH:DCM, 0-50%) to isolate tert-butyl 2-{4-[(2,6-dioxopiperidin-3-yl)carbamoyl]-2-methyl-1H-1,3-benzodiazol-1-yl}acetate (74.0 mg, 0.185 mmol, 54%) as a yellow solid.

LCMS (ESI): 401.1 m/z [M+H]⁺

### Step C

To a solution of tert-butyl 2-{4-[(2,6-dioxopiperidin-3-yl)carbamoyl]-2-methyl-1H-1,3-benzodiazol-1-yl}acetate (64.5 mg, 0.161 mmol) in DCM (2.0 mL) was added TFA (1.0 mL, 13.059 mmol). The mixture was stirred for 18 h at RT. The crude was concentrated in vacuo and dissolved in H₂O. To the solution was added 1M HCl in water and it was evaporated. The product, 2-{4-[(2,6-dioxopiperidin-3-yl)carbamoyl]-2-methyl-1H-1,3-benzodiazol-1-yl}acetic acid hydrochloride (58.8 mg, 0.154 mmol, 96%) was isolated as beige solid.

LCMS (ESI): 344.8 m/z [M+H]⁺

### Step D

2-{4-[(2,6-Dioxopiperidin-3-yl)carbamoyl]-2-methyl-1H-1,3-benzodiazol-1-yl}acetic acid hydrochloride (37.3 mg, 0.098 mmol), tert-butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (55.0 mg, 0.082 mmol) and HATU (62.0 mg, 0.163 mmol) were dissolved in dry DMF (2.0 mL) and to the mixture was added DIPEA (0.071 mL, 0.408 mmol). The reaction was stirred at RT for 2 h. The solvent was evaporated. Residues were dissolved in DCM and washed with brine. The organic layer was dried over Na₂SO₄, filtered and concentrated in vacuo to get tert-butyl 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (102.2 mg, crude) which was used without a further purification to next step.

LCMS (ESI): 1001.3 m/z [M+H]⁺

### Step E

To a solution of tert-butyl 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (102.2 mg, crude) in DCM (1.0 mL) was added TFA (0.500 mL, 6.529 mmol). The mixture was stirred for 18 h at RT. The crude was concentrated in vacuo and purified by preparative HPLC (H₂O:MeCN+0.1% FA) to give 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carboxylic acid (32.3 mg, 0.034 mmol, 35%) as a white solid.

LCMS (ESI): 944.3 m/z [M+H]⁺

### Step F

To a solution of 6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (20.0 mg, 0.021 mmol) in dry DMF (1.2 mL) were added Et₃N (0.030 mL, 0.212 mmol) and chloromethyl 2,2-dimethylpropanoate (0.009 mL, 0.064 mmol). The reaction mixture was stirred at 60° C. for next 18 h. Then, the crude was purified by preparative HPLC (C18, H₂O:MeCN+0.1% FA) to obtain (6-chloro-1-{2-[4-(2-{4-[(2,6-dioxopiperidin-3-yl)carbamoyl]-2-methyl-1H-1,3-benzodiazol-1-yl}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carbonyloxy)methyl 2,2-dimethylpropanoate (16.6 mg, 0.016 mmol, 74%) as a white solid.

LCMS (ESI): 1058.2 m/z [M+H]⁺

¹H NMR (500 MHz, DMSO) δ 10.91 (s, 1H), 10.23 (d, J=7.3 Hz, 1H), 8.22 (dd, J=9.2, 5.9 Hz, 1H), 7.84 (dd, J=7.6, 1.1 Hz, 1H), 7.81 (d, J=8.6 Hz, 1H), 7.67-7.62 (m, 2H), 7.46-7.43 (m, 2H), 7.36 (td, J=8.9, 2.6 Hz, 1H), 7.31-7.27 (m, 2H), 6.90-6.86 (m, 1H), 6.01 (s, 2H), 5.29 (s, 2H), 4.88 (ddd, J=12.6, 7.4, 5.3 Hz, 1H), 4.29-4.20 (m, 3H), 4.19-4.12 (m, 1H), 3.77 (s, 3H), 3.48-3.43 (m, 2H), 3.28-3.20 (m, 4H), 2.82 (ddd, J=17.2, 13.5, 5.6 Hz, 1H), 2.61-2.52 (m, 1H), 2.47 (s, 3H), 2.27-2.02 (m, 13H), 1.88 (s, 3H), 1.14 (s, 9H).

### Example 4: (Pivaloyloxy)methyl (S)-6-chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 71)

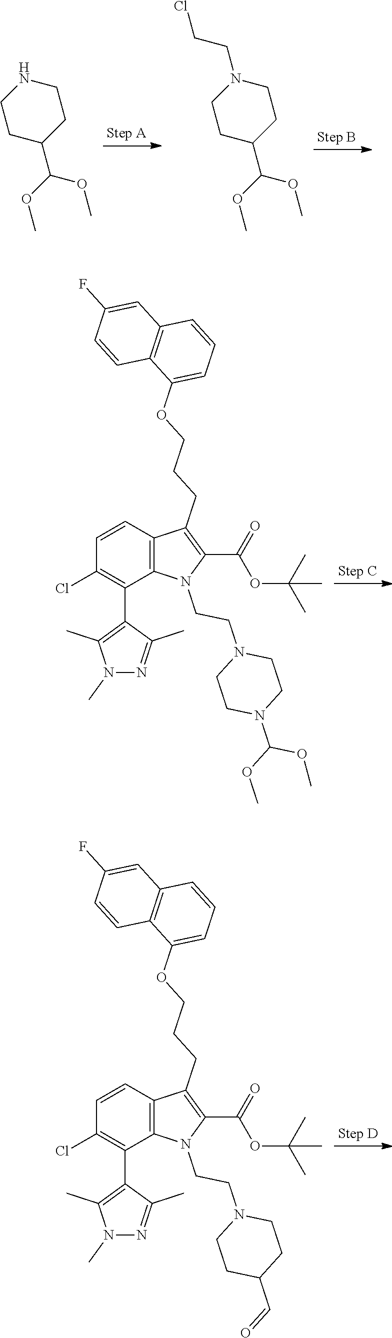

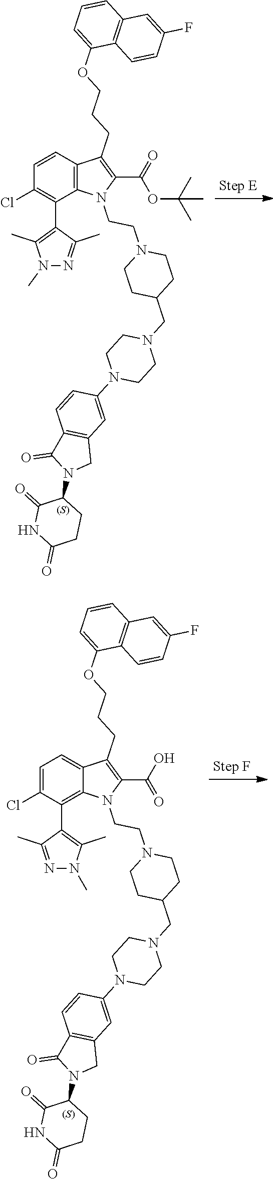

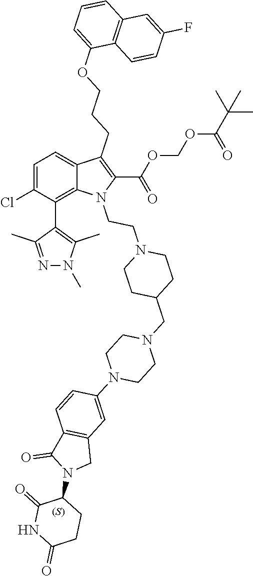

### Step A

To a stirred solution of 4-(dimethoxymethyl)piperidine (0.4 g, 2.51 mmol) in DMF (10 mL) was added dry (washed with n-hexane) sodium hydride (0.18 g, 7.5 mmol) portionwise at 0° C. under nitrogen. The reaction mixture was allowed to stir at room temperature for 30 min. Next, the reaction mixture was again cooled to 0° C. and 1-bromo-2-chloroethane (2.08 mL, 25.1 mmol) was added dropwise. The resulting reaction mixture was allowed to stir at room temperature under nitrogen for 4 h. The excess sodium hydride was then quenched by addition of ice water and diluted with EtOAc. The organic layer was washed with ice water and brine, dried over Na₂SO₄ and evaporated under reduced pressure. Crude compound was purified with column chromatography (SiO₂, 30-40% EtOAc in hexane) to afford 1-(2-chloroethyl)-4-(dimethoxymethyl)piperidine (300 mg, 1.35 mmol, 54%) as light brown sticky solid.

LCMS (ESI): 222.2 m/z [M+H]⁺

### Step B

To a well stirred solution of tert-butyl 6-chloro-3-(3-((6-fluoronaphthalen-1-yl)oxy)propyl)-7-(1,3,5-trimethyl-1H-pyrazol-4-yl)-1H-indole-2-carboxylate (350 mg, 0.62 mmol) in DMF (3 mL) was added 1-(2-chloroethyl)-4-(dimethoxymethyl)piperidine (276 mg, 1.24 mmol) followed by Cs₂CO₃ (304 mg, 0.93 mmol) and the mixture was allowed to stir at 100° C. for 16 h under nitrogen. After complete consumption of the starting material, (monitored by TLC and LCMS), the reaction mixture was diluted with EtOAc and washed with water and brine. The organic layer was dried over Na₂SO₄ and evaporated under reduced pressure to get the crude compound, which was then purified by column chromatography (SiO₂, 60-70% EtOAc in DCM) to get tert-butyl 6-chloro-1-(2-(4-(dimethoxymethyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (120 mg, 0.16 mmol, 26%) as light pink solid.

LCMS (ESI): 747.6 m/z [M+H]⁺

### Step C

tert-Butyl 6-chloro-1-{2-[4-(dimethoxymethyl)piperidin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (10.0 mg, 0.013 mmol) was dissolved in THF (0.535 mL) and a 2M aqueous solution of H₂SO₄ (0.268 mL, 0.535 mmol) was added. Reaction was stirred in a sealed vial at 70° C. for 1 h. After complete conversion of the starting material, a solution of 1M NaOH_{aq} was added to pH=7 and reaction product was extracted with DCM. Organic layer was washed with brine, dried over anhydrous MgSO₄, filtered and dried under reduced pressure. Crude tert-butyl 6-chloro-3-{3-[(6-fluoronaphthalen-1-yl)oxy]propyl}-1-[2-(4-formylpiperidin-1-yl)ethyl]-7-(1,3,5-trimethyl-1H-pyrazol-4-yl)-1H-indole-2-carboxylate (7.5 mg) was obtained as off white solid and used directly in the next step.

LCMS (ESI): 701.4 m/z [M+H]⁺

### Step D

Crude tert-butyl 6-chloro-3-{3-[(6-fluoronaphthalen-1-yl)oxy]propyl}-1-[2-(4-formylpiperidin-1-yl)ethyl]-7-(1,3,5-trimethyl-1H-pyrazol-4-yl)-1H-indole-2-carboxylate (7.5 mg) and (3S)-3-[1-oxo-5-(piperazin-1-yl)-2,3-dihydro-1H-isoindol-2-yl]piperidine-2,6-dione (4.8 mg, 0.015 mmol) were dissolved in dry DCM (0.531 mL) and MeOH (0.133 mL) under argon atmosphere. To it, STAB (14.2 mg, 0.067 mmol) and AcOH (0.001 mL, 0.017 mmol) were added, and the reaction was stirred for 16 h under argon at room temperature. After complete consumption of the starting material, the mixture was diluted with DCM and washed with saturated aqueous NaHCO₃, brine and water. Organic layer was dried over anhydrous MgSO₄, filtered and dried under reduced pressure to give tert-butyl (S)-6-chloro-1-(2-(4-((4-(2-(2,6-dioxopiperidin-3-yl)-1-oxoisoindolin-5-yl)piperazin-1-yl)methyl) piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (8.4 mg, crude) as beige film. Crude product was submitted to the subsequent step without further purification.

LCMS (ESI): 1013.3 m/z [M+H]⁺

### Step E

tert-Butyl (S)-6-chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (8.4 mg, crude) was dissolved under argon atmosphere in dry DCM (0.300 mL) and TFA (0.300 mL, 3.918 mmol) was added. Reaction was stirred for 16 h at room temperature under argon. After complete consumption of the starting material, the solution was concentrated under reduced pressure to dryness and dissolved in DMSO. Crude product was purified with preparative HPLC (C18, H₂O:MeCN+0.1% FA) to get (S)-6-chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (2.8 mg, 0.003 mmol, 23% over 3 steps) as white solid.

LCMS (ESI): 957.25 m/z [M+H]⁺

### Step F

To a well stirred solution of (S)-6-chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (150 mg, 0.157 mmol) in DMF (3 ml) was added chloromethyl pivalate (0.027 ml, 0.188 mmol) followed by potassium carbonate (21 mg, 0.157 mmol) and the mixture was allowed to stir at 60° C. for 2 h under nitrogen. After complete consumption of the starting material the reaction mixture was diluted with ethyl acetate and washed with water and brine. The organic layer was dried over sodium sulphate and concentrated under reduced pressure to get the crude compound, which was then purified by preparative HPLC (10 mM ammonium acetate in H₂O:MeCN) to get (pivaloyloxy)methyl (S)-6-chloro-1-(2-(4-((4-(2-(2,6-dioxopiperidin-3-yl)-1-oxoisoindolin-5-yl)piperazin-1-yl)methyl)piperidin-1-yl)ethyl)-3-(3-((6-fluoronaphthalen-1-yl)oxy)propyl)-7-(1,3,5-trimethyl-1H-pyrazol-4-yl)-1H-indole-2-carboxylate (15 mg, 0.014 mmol, 9%) as white solid.

LCMS (ESI): 1071.2 m/z [M+H]⁺

¹H NMR (500 MHz, DMSO) δ=10.93 (s, 1H), 8.20 (dd, J=9.3, 5.9, 1H), 7.80 (d, J=8.6, 1H), 7.65 (dd, J=10.3, 2.7, 1H), 7.59-7.48 (m, 1H), 7.48-7.40 (m, 2H), 7.36 (td, J=8.9, 2.6, 1H), 7.27 (d, J=8.5, 1H), 7.15-6.96 (m, 2H), 6.94-6.80 (m, 1H), 5.98 (s, 2H), 5.04 (dd, J=13.3, 5.2, 1H), 4.32 (d, J=16.9, 1H), 4.28-4.14 (m, 4H), 4.14-4.01 (m, 1H), 3.75 (s, 3H), 3.26-3.14 (m, 6H), 2.90 (ddd, J=17.8, 13.6, 5.4, 1H), 2.62-2.55 (m, 1H), 2.47-2.24 (m, 6H), 2.24-2.15 (m, 2H), 2.14-1.92 (m, 7H), 1.91-1.80 (m, 5H), 1.80-1.60 (m, 3H), 1.60-1.42 (m, 2H), 1.14 (s, 9H), 1.01-0.78 (m, 2H).

### Example 5: (6-Chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carbonyloxy)methyl 2,2-dimethylpropanoate (Compound 69)

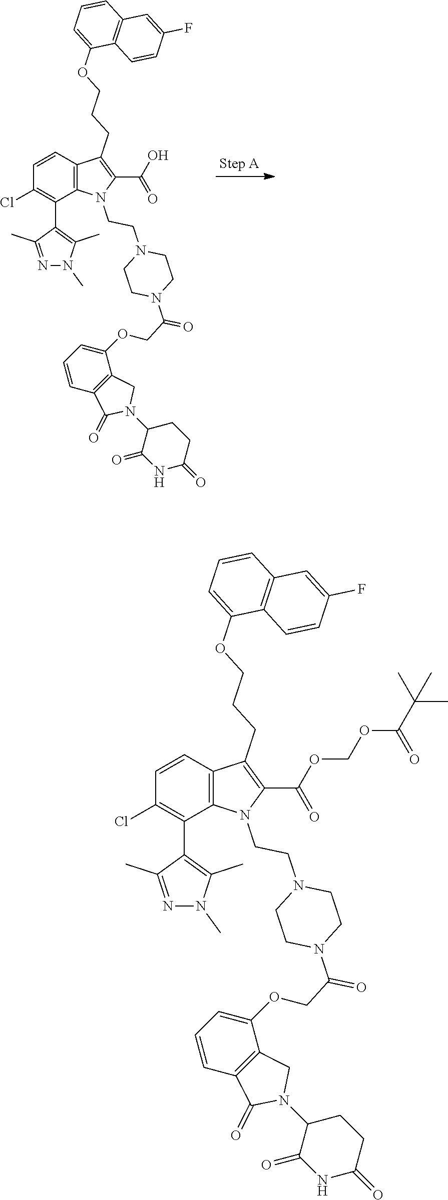 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4cccc5c4CN(C4CCC(=O)NC4=O)C5=O)CC3)c12]

### Step A

6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (100.0 mg, 0.109 mmol) was dissolved in DMF (6.3 mL) and triethylamine (0.076 mL, 0.544 mmol) was added, followed by chloromethyl pivalate (0.031 mL, 0.218 mmol). Mixture was stirred overnight at room temperature, and the next 2 days at 80° C. Solvents were removed under reduced pressure and the desired product was purified using flash chromatography (SiO₂, 5% of MeOH in DCM) to give a corresponding (6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carbonyloxy)methyl 2,2-dimethylpropanoate (66.0 mg, 0.064 mmol, 58.7%) as a white solid.

LCMS (ESI): 1032.2 m/z [M+H]⁺

¹H NMR (500 MHz, DMSO, 353 K) δ=10.64 (s, 1H), 8.21 (dd, J=9.3, 5.8, 1H), 7.77 (d, J=8.6, 1H), 7.58 (dd, J=10.4, 2.6, 1H), 7.47-7.38 (m, 3H), 7.35-7.28 (m, 2H), 7.26 (d, J=8.6, 1H), 7.14 (d, J=8.1, 1H), 6.87 (dd, J=6.1, 2.6, 1H), 5.99 (s, 2H), 5.05 (dd, J=13.0, 5.2, 1H), 4.89 (s, 2H), 4.40 (d, J=17.1, 1H), 4.32 (d, J=16.9, 1H), 4.28-4.19 (m, 3H), 4.20-4.11 (m, 1H), 3.76 (d, J=1.9, 3H), 3.37-3.30 (m, 4H), 3.30-3.23 (m, 2H), 2.88 (ddd, J=17.2, 13.4, 5.5, 1H), 2.64-2.56 (m, 1H), 2.48-2.38 (m, 1H), 2.27-2.18 (m, 2H), 2.14-2.02 (m, 7H), 2.01 (s, 3H), 1.88 (s, 3H), 1.16 (s, 9H).

### Example 6: 4-Oxo-3,5,8,11-tetraoxatridecan-2-yl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 101)

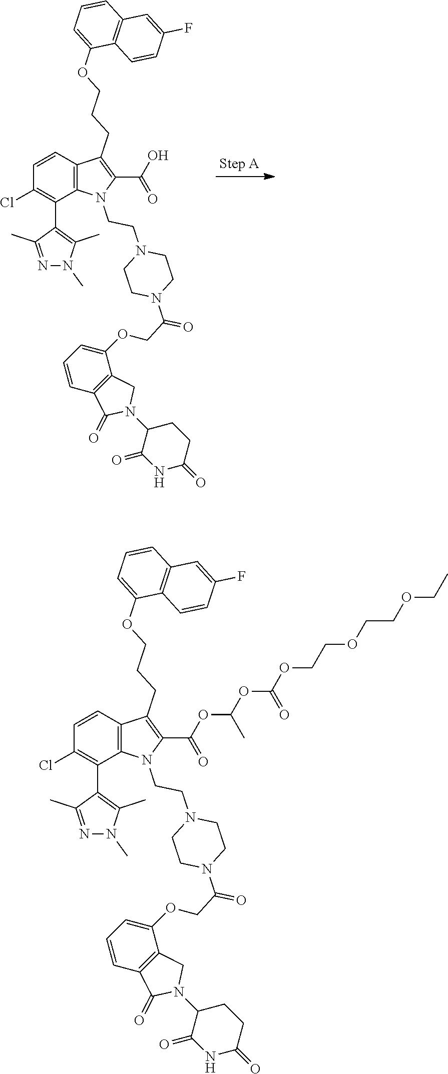 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; CCOCCOCCOC(=O)OC(C)OC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1]

### Step A

To a well stirred solution of 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (100 mg, 0.109 mmol) in DMF (1 mL) was added K₂CO₃ (45 mg, 0.327 mmol) followed by 1-chloroethyl (2-(2-ethoxyethoxy)ethyl) carbonate (100 mg, 0.417 mmol) and the reaction mixture was allowed to stir at 60° C. for 2 h under nitrogen. After complete consumption of the starting material the solvent was concentrated under reduced pressure and the target molecule was purified by preparative HPLC (10 mM ammonium acetate buffer:MeCN) to afford 4-oxo-3,5,8,11-tetraoxatridecan-2-yl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (45 mg, 0.04 mmol, 37%) as white solid.

LCMS (ESI): 1120.84 m/z [M−H]−

¹H NMR (500 MHz, DMSO, 353 K) δ 10.66 (s, 1H), 8.24 (dd, J=9.2, 5.9 Hz, 1H), 7.77 (d, J=8.6 Hz, 1H), 7.59 (dd, J=10.4, 2.6 Hz, 1H), 7.48-7.38 (m, 3H), 7.37-7.30 (m, 2H), 7.26 (d, J=8.6 Hz, 1H), 7.17-7.11 (m, 1H), 7.00-6.93 (m, 1H), 6.89 (dd, J=5.8, 2.9 Hz, 1H), 5.05 (dd, J=13.0, 5.2 Hz, 1H), 4.89 (s, 2H), 4.40 (d, J=17.1 Hz, 1H), 4.31 (dd, J=17.0, 1.6 Hz, 1H), 4.29-4.11 (m, 6H), 3.76 (d, J=2.2 Hz, 3H), 3.63-3.58 (m, 2H), 3.51-3.45 (m, 2H), 3.43-3.40 (m, 2H), 3.38 (q, J=7.0 Hz, 2H), 3.35-3.31 (m, 4H), 3.30-3.25 (m, 2H), 2.88 (ddd, J=17.4, 13.5, 5.5 Hz, 1H), 2.64-2.59 (m, 1H), 2.47-2.39 (m, 1H), 2.28-2.20 (m, 2H), 2.18-2.02 (m, 7H), 2.01 (d, J=1.7 Hz, 3H), 1.88 (d, J=1.5 Hz, 3H), 1.63 (dd, J=5.4, 1.6 Hz, 3H), 1.04 (t, J=7.0 Hz, 3H).

### Example 7: 1-{[(Propan-2-yloxy)carbonyl]oxy}ethyl 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylate (Compound 99)

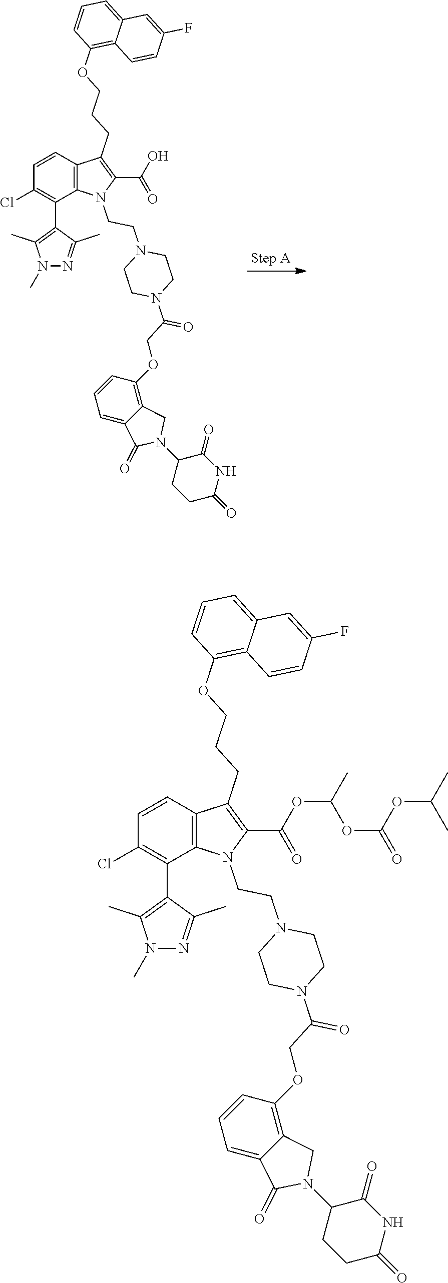 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OC(C)OC(=O)OC(C)C)n(CCN3CCN(C(=O)COc4cccc5c4CN(C4CCC(=O)NC4=O)C5=O)CC3)c12]

### Step A

To a solution of 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (40.0 mg, 0.044 mmol) in DMF (2.5 mL), triethylamine (17.6 mg, 0.174 mmol) and 1-chloroethyl isopropyl carbonate (7.6 mg, 0.046 mmol) were added. Reaction was stirred for 18 h at room temperature. Solvents were removed under reduced pressure and the desired product was purified using flash chromatography (SiO₂, DCM: MeOH gradient from 5 to 20% of MeOH) to give 1-{[(propan-2-yloxy)carbonyl]oxy}ethyl 6-chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carboxylate (21.1 mg, 0.020 mmol, 46.2%) as a white solid.

LCMS (ESI): 1046.1 m/z [M−H]⁻.

¹H NMR (500 MHz, DMSO, 353 K) δ=10.67 (s, 1H), 8.24 (dd, J=9.2, 5.9, 1H), 7.77 (d, J=8.6, 1H), 7.59 (dd, J=10.3, 2.6, 1H), 7.47-7.39 (m, 3H), 7.36-7.29 (m, 2H), 7.26 (d, J=8.6, 1H), 7.16-7.11 (m, 1H), 6.96-6.91 (m, 1H), 6.88 (dd, J=6.1, 2.5, 1H), 5.05 (dd, J=13.0, 5.2, 1H), 4.89 (s, 2H), 4.84-4.75 (m, 1H), 4.40 (d, J=17.2, 1H), 4.31 (dd, J=17.0, 1.6, 1H), 4.26 (t, J=6.3, 2H), 4.24-4.20 (m, 1H), 4.20-4.12 (m, 1H), 3.76 (d, J=2.0, 3H), 3.38-3.31 (m, 4H), 3.31-3.21 (m, 2H), 2.88 (ddd, J=17.4, 13.5, 5.5, 1H), 2.65-2.59 (m, 1H), 2.47-2.38 (m, 1H), 2.27-2.20 (m, 2H), 2.16-2.02 (m, 7H), 2.01 (s, 3H), 1.88 (d, J=1.1, 3H), 1.62 (dd, J=5.4, 1.7, 3H), 1.22 (d, J=6.2, 3H), 1.19 (dd, J=6.2, 1.4, 3H).

### Example 8: 1-(((Cyclohexyloxy)carbonyl)oxy)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 100)

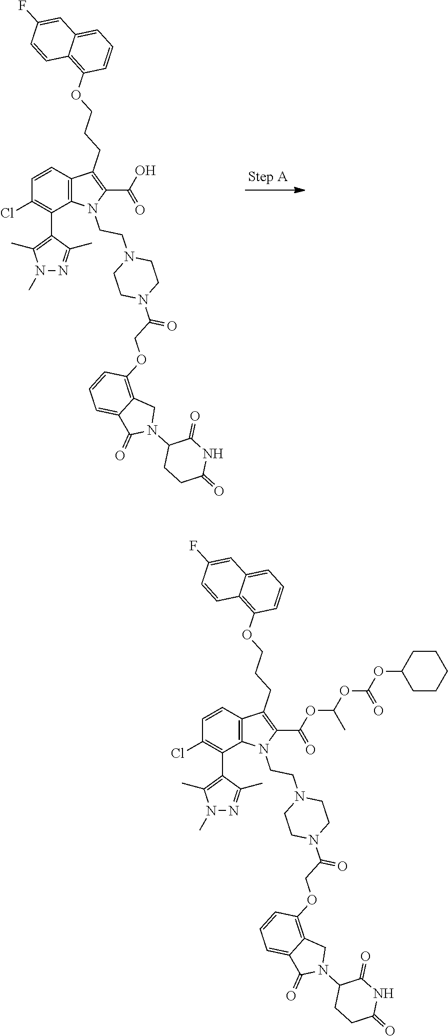 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OC(C)OC(=O)OC3CCCCC3)n(CCN3CCN(C(=O)COc4cccc5c4CN(C4CCC(=O)NC4=O)C5=O)CC3)c12]

### Step A

6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (30.0 mg, 0.033 mmol) was dissolved in dry MeCN (1.0 mL) and Et₃N (0.046 mL, 0.327 mmol), followed by 1-chloroethyl cyclohexyl carbonate (8.1 mg, 0.039 mmol). Reaction mixture (monitored with LCMS) was stirred in a sealed vial at 60° C. for 96 h. After reaction completion the mixture was concentrated under reduced pressure to dryness and dissolved in DCM. Organic phase was washed with brine and water, dried over anhydrous magnesium sulphate, filtered and concentrated. Crude product was purified with column chromatography (SiO₂, 5% to 20% MeOH in DCM) to give 1-(((cyclohexyloxy)carbonyl)oxy)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (17.2 mg, 0.016 mmol, 48.5%) as white solid.

LCMS (ESI): 1088.2 m/z [M+H]⁺.

¹H NMR (500 MHz, DMSO, 353 K) δ 10.67 (s, 1H), 8.24 (dd, J=9.2, 5.9 Hz, 1H), 7.78 (d, J=8.6 Hz, 1H), 7.59 (dd, J=10.4, 2.6 Hz, 1H), 7.50-7.38 (m, 3H), 7.36-7.29 (m, 2H), 7.26 (d, J=8.6 Hz, 1H), 7.14 (dt, J=8.2, 0.7 Hz, 1H), 6.94 (q, J=5.4 Hz, 1H), 6.88 (dd, J=6.1, 2.5 Hz, 1H), 5.05 (dd, J=13.1, 5.2 Hz, 1H), 4.89 (s, 2H), 4.62-4.52 (m, 1H), 4.40 (d, J=17.1 Hz, 1H), 4.31 (dd, J=17.2, 1.5 Hz, 1H), 4.29-4.11 (m, 4H), 3.76 (d, J=2.0 Hz, 3H), 3.38-3.31 (m, 4H), 3.31-3.20 (m, 2H), 2.88 (ddd, J=17.4, 13.5, 5.5 Hz, 1H), 2.67-2.59 (m, 1H), 2.44 (qd, J=13.1, 4.9 Hz, 1H), 2.28-2.19 (m, 2H), 2.17-2.03 (m, 7H), 2.01 (d, J=1.3 Hz, 3H), 1.88 (d, J=1.6 Hz, 3H), 1.85-1.73 (m, 2H), 1.62 (dd, J=5.4, 1.7 Hz, 3H), 1.61-1.49 (m, 2H), 1.45-1.30 (m, 4H), 1.26-1.10 (m, 2H).

### Example 9: (2-(Phosphonooxy)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 131)

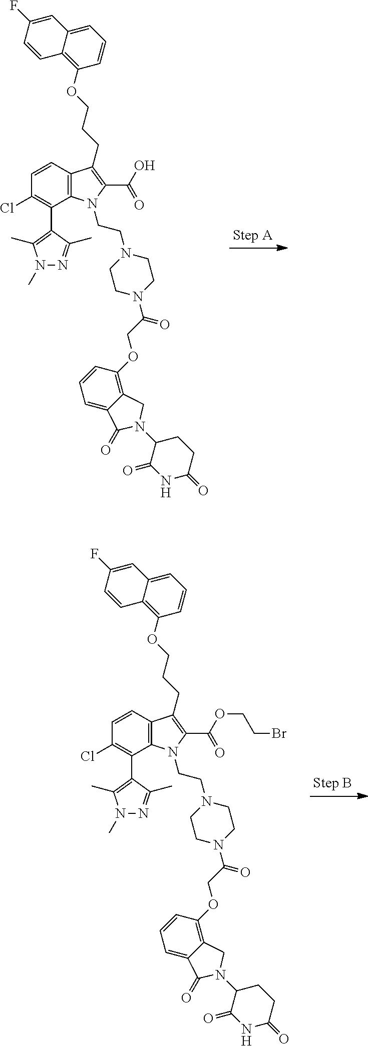 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCBr)n(CCN3CCN(C(=O)COc4cccc5c4CN(C4CCC(=O)NC4=O)C5=O)CC3)c12]

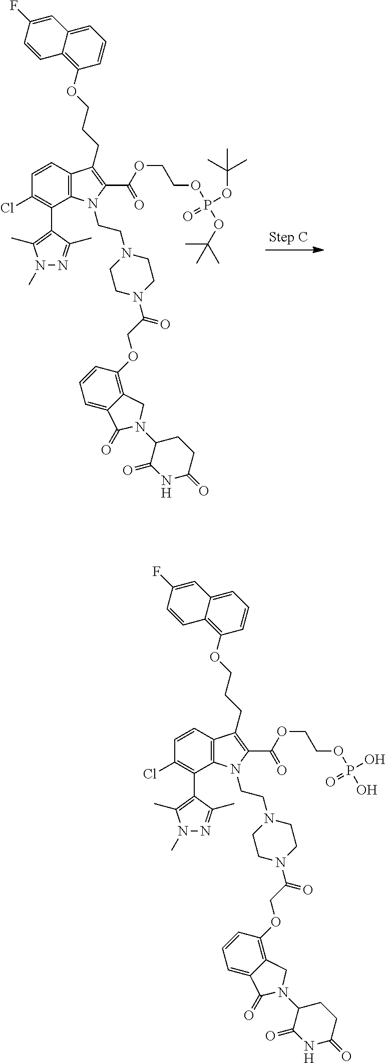

### Step A

To a solution of 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (200 mg, 0.218 mmol) in DMF (1 mL) was added HATU (165.5 mg, 0.436 mmol) and Et₃N (0.092 mL, 0.654 mmol) at room temperature under nitrogen, and the reaction was allowed to stir for 15 min. To it, 2-bromoethan-1-ol (0.08 ml, 1.089 mmol) was added and then the resulting mixture was stirred for another 4 h at room temperature. After complete consumption of starting material the reaction was quenched by adding ice-water. The mixture was concentrated under vacuum, diluted with DCM and washed with saturated sodium bi carbonate solution (2×50 mL), water (50 mL) and brine (50 mL). Organic layer was dried over anhydrous Na₂SO₄, filtered and concentrated. Crude compound was purified with column chromatography (SiO₂, 3-5% MeOH in DCM) to get 2-bromoethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (150 mg, 0.146 mmol, 67%) as white solid.

LCMS (ESI): 1026.1 m/z [M+H]⁺.

### Step B

To a solution of 2-bromoethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (150 mg, 0.146 mmol) in DMF (1 mL) was added potassium di-tert-butyl phosphate (72.5 mg, 0.293 mmol) at room temperature under nitrogen atmosphere. The resulting mixture was allowed to stir at 100° C. for 16 h under nitrogen. After complete consumption of the starting material the solvent was concentrated under reduced pressure, diluted with EtOAc and washed with cold water and brine. The organic layer was dried over sodium sulfate and evaporated under reduced pressure to get the crude 2-((di-tert-butoxyphosphoryl)oxy)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (120 mg) as off white solid which was directly used in the next step without further purification.

LCMS (ESI): 1154.4 m/z [M+H]⁺

### Step C

To a solution of 2-((di-tert-butoxyphosphoryl)oxy)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (120 mg, 0.104 mmol) in DCM (3 mL) was added TFA (2 mL) at 0° C. dropwise under nitrogen and the reaction mixture was allowed to stir at ambient temperature for 3 h. After complete consumption of the starting material the volatiles were removed under reduced pressure to get the crude product, which was then purified by preparative HPLC (10 mM ammonium acetate buffer:MeCN) to afford 2-(phosphonooxy)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (13 mg, 0.0124 mmol, 8.4% over 2 steps) of as white solid.

LCMS (ESI): 1040.1 m/z [M−H]−

¹H NMR (400 MHz, DMSO, 372 K) δ 10.57 (s, 1H), 8.22 (dd, J=9.3, 5.9 Hz, 1H), 7.73 (d, J=8.5 Hz, 1H), 7.57 (dd, J=10.3, 2.8 Hz, 1H), 7.47-7.38 (m, 3H), 7.37-7.29 (m, 2H), 7.23 (dd, J=8.6, 1.6 Hz, 1H), 7.15 (d, J=8.1 Hz, 1H), 6.93-6.85 (m, 1H), 5.04 (dd, J=13.5, 5.2 Hz, 1H), 4.89 (s, 2H), 4.53-4.44 (m, 2H), 4.41 (d, J=17.1 Hz, 1H), 4.32 (d, J=16.9 Hz, 1H), 4.30-4.12 (m, 6H), 3.80-3.72 (m, 3H), 3.38-3.35 (m, 4H), 3.31-3.27 (m, 2H), 2.87 (ddd, J=18.2, 13.7, 5.8 Hz, 1H), 2.68-2.59 (m, 1H), 2.47-2.38 (m, 1H), 2.29-2.21 (m, 2H), 2.19-2.03 (m, 7H), 2.01 (s, 3H), 1.88 (s, 3H).

### Example 10: 3-(4-Methylpiperazin-1-yl)propyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 137)

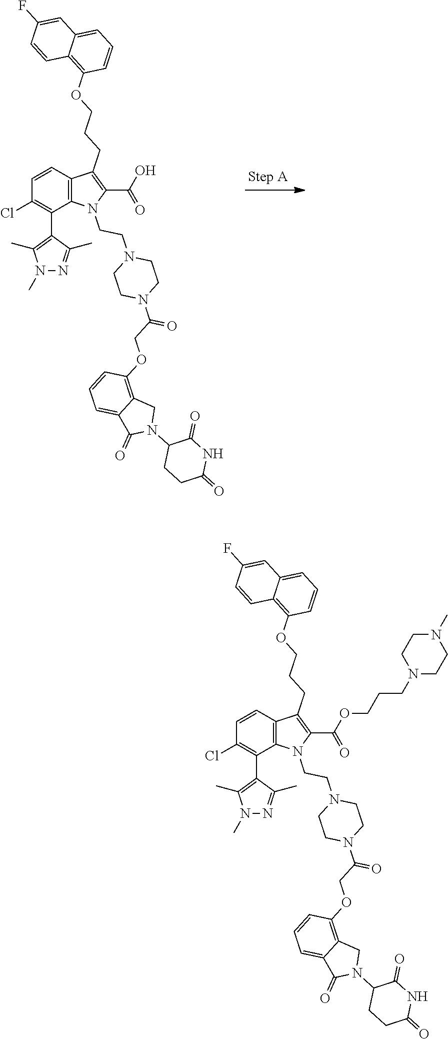

### Step A

6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (20.0 mg, 0.022 mmol) and HATU (9.1 mg, 0.024 mmol) were dissolved in dry DMF (1.0 mL) under argon atmosphere and DIPEA (0.011 mL, 0.065 mmol) was added. Reaction was stirred at room temperature under argon for 20 min and 3-(4-methylpiperazin-1-yl)propan-1-ol (4.1 mg, 0.026 mmol) was added. Reaction, (monitored with LCMS) was stirred at room temperature for the next 16 h. After that time, additional portion of HATU (9.1 mg, 0.024 mmol), DIPEA (0.011 mL, 0.065 mmol) and 3-(4-methylpiperazin-1-yl)propan-1-ol (4.1 mg, 0.026 mmol) was added. Reaction was continued for 2 h under argon at room temperature. After complete consumption of the starting material the solution was concentrated under reduced pressure, diluted with DMSO and purified with reverse phase preparative HPLC (C18, H₂O:MeCN+0.1% FA). 3-(4-Methylpiperazin-1-yl)propyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (6.5 mg, 0.006 mmol, 28.3%) was obtained as white solid.

LCMS (ESI): 1058.2 m/z [M+H]⁺.

¹H NMR (500 MHz, DMSO, 353 K) δ 10.68 (s, 1H), 8.22 (dd, J=9.2, 5.9 Hz, 1H), 7.75 (d, J=8.6 Hz, 1H), 7.60 (dd, J=10.4, 2.6 Hz, 1H), 7.47-7.40 (m, 3H), 7.36-7.30 (m, 2H), 7.25 (d, J=8.6 Hz, 1H), 7.16-7.12 (m, 1H), 6.88 (dd, J=6.4, 2.3 Hz, 1H), 5.05 (dd, J=13.1, 5.1 Hz, 1H), 4.89 (s, 2H), 4.40 (d, J=17.1 Hz, 1H), 4.35-4.28 (m, 3H), 4.28-4.10 (m, 4H), 3.76 (d, J=2.1 Hz, 3H), 3.39-3.30 (m, 4H), 3.30-3.24 (m, 2H), 2.88 (ddd, J=17.3, 13.4, 5.5 Hz, 1H), 2.66-2.59 (m, 1H), 2.44 (qd, J=13.3, 4.6 Hz, 1H), 2.39-2.34 (m, 2H), 2.34-2.29 (m, 4H), 2.28-2.19 (m, 6H), 2.15-2.10 (m, 1H), 2.10 (s, 3H), 2.09-2.02 (m, 6H), 2.01 (s, 3H), 1.88 (s, 3H), 1.85 (p, J=6.8 Hz, 2H).

### Example 11: 2-(Dimethylamino)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 107)

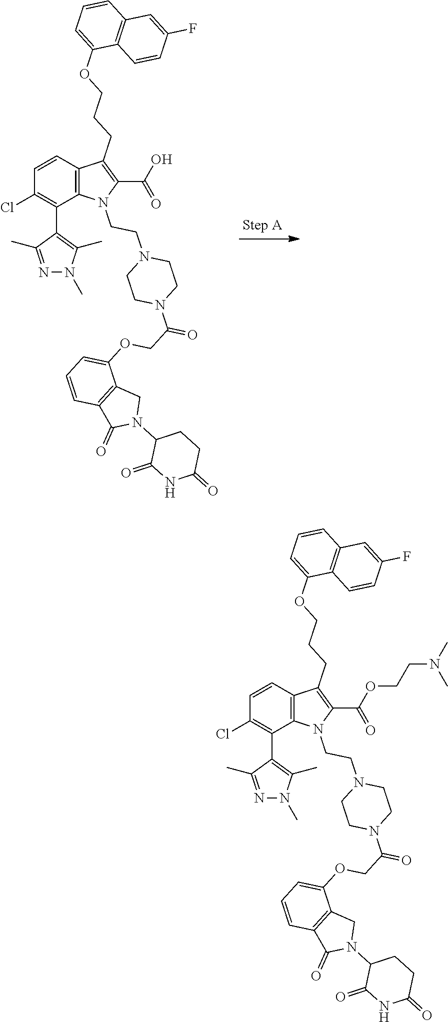

### Step A

6-Chloro-1-{2-[4-(2-{[2-(2,6-dioxopiperidin-3-yl)-1-oxo-2,3-dihydro-1H-isoindol-4-yl]oxy}acetyl)piperazin-1-yl]ethyl}-3-{3-[(6-fluoronaphthalen-1-yl)oxy]propyl}-7-(1,3,5-trimethyl-1H-pyrazol-4-yl)-1H-indole-2-carboxylic acid (40.0 mg, 0.044 mmol) was dissolved in DMF (0.8 mL). DIPEA (0.023 mL, 0.131 mmol), HATU (19.9 mg, 0.052 mmol) and 2-Dimethylaminoethanol (7.8 mg, 0.087 mmol) were added. The mixture was stirred for 18 h at room temperature. Solvents were removed under reduced pressure, and desired product was purified using flash chromatography (SiO₂, DCM:MeOH gradient from 0 to 20% of MeOH), to give 2-(dimethylamino)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (33.0 mg, 0.033 mmol, 76.6%) as a white solid.

LCMS (ESI): 989.25 m/z [M+H]⁺

¹H NMR (500 MHz, DMSO, 353 K) δ=10.70 (s, 1H), 8.22 (dd, J=9.2, 5.8, 1H), 7.74 (d, J=8.5, 1H), 7.60 (dd, J=10.4, 2.7, 1H), 7.48-7.39 (m, 3H), 7.37-7.29 (m, 2H), 7.24 (d, J=8.5, 1H), 7.14 (d, J=8.1, 1H), 6.88 (dd, J=5.6, 3.0, 1H), 5.05 (dd, J=13.1, 5.2, 1H), 4.89 (s, 2H), 4.39 (d, J=17.2, 1H), 4.37-4.28 (m, 3H), 4.28-4.20 (m, 3H), 4.19-4.11 (m, 1H), 3.75 (d, J=2.1, 3H), 3.36-3.31 (m, 4H), 3.30-3.26 (m, 2H), 2.93-2.84 (m, 1H), 2.62-2.57 (m, 2H), 2.46-2.38 (m, 1H), 2.28-2.21 (m, 2H), 2.18 (s, 6H), 2.13-2.02 (m, 7H), 2.01 (s, 3H), 1.88 (s, 3H). one proton in the aliphatic area overlaps with solvents.

### Example 12: 2-(Methylsulfonyl)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 124)

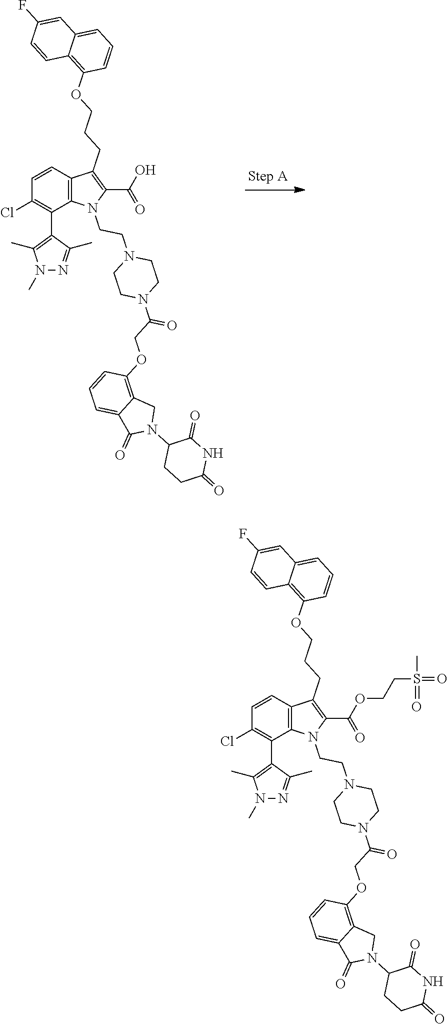 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCS(C)(=O)=O)n(CCN3CCN(C(=O)COc4cccc5c4CN(C4CCC(=O)NC4=O)C5=O)CC3)c12]

### Step A

6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (20.0 mg, 0.022 mmol) and HATU (9.1 mg, 0.024 mmol) were dissolved in dry DMF (2.0 mL) under argon atmosphere. To it, Et₃N (0.011 mL, 0.065 mmol) was added and the reaction was stirred at room temperature for 20 min. Then 2-(methylsulfonyl)ethan-1-ol (3.2 mg, 0.026 mmol) was added and the reaction (monitored with LCMS) was stirred for 3.5 h at room temperature under argon. Then, second portion of HATU (9.1 mg, 0.024 mmol) with Et₃N (0.011 mL, 0.065 mmol) in DMF (1.0 mL) were added and the reaction was continued for the next 16 h at room temperature under argon. After complete consumption of the starting material the solution was diluted with DCM (up to 10 mL) and washed with brine and water. Organic layer was dried over anhydrous sodium sulphate, filtrated and dried under reduced pressure. The resulting residue was dissolved in DCM and purified with column chromatography (SiO₂, 1% to 40% MeOH in DCM) to give 2-(methylsulfonyl)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (11.4 mg, 0.011 mmol, 51.1%) as white solid.

LCMS (ESI): 1024.1 m/z [M+H]⁺

¹H NMR (500 MHz, DMSO, 353 K) δ 10.64 (s, 1H), 8.23 (dd, J=9.2, 5.9 Hz, 1H), 7.74 (d, J=8.6 Hz, 1H), 7.59 (dd, J=10.4, 2.6 Hz, 1H), 7.47-7.39 (m, 3H), 7.38-7.30 (m, 2H), 7.25 (d, J=8.6 Hz, 1H), 7.14 (d, J=8.1 Hz, 1H), 6.89 (dd, J=5.5, 3.2 Hz, 1H), 5.05 (dd, J=13.1, 5.2 Hz, 1H), 4.89 (s, 2H), 4.69 (t, J=6.1 Hz, 2H), 4.40 (d, J=17.1 Hz, 1H), 4.32 (d, J=17.0 Hz, 1H), 4.30-4.20 (m, 3H), 4.20-4.10 (m, 1H), 3.76 (d, J=2.1 Hz, 3H), 3.64 (t, J=6.1 Hz, 2H), 3.36-3.32 (m, 4H), 3.32-3.28 (m, 2H), 3.02 (s, 3H), 2.88 (ddd, J=17.2, 13.5, 5.5 Hz, 1H), 2.67-2.60 (m, 1H), 2.48-2.40 (m, 1H), 2.29-2.21 (m, 2H), 2.15-2.02 (m, 7H), 2.01 (s, 3H), 1.88 (s, 3H).

### Example 13: 2-(4-Methylpiperazin-1-yl)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 133)

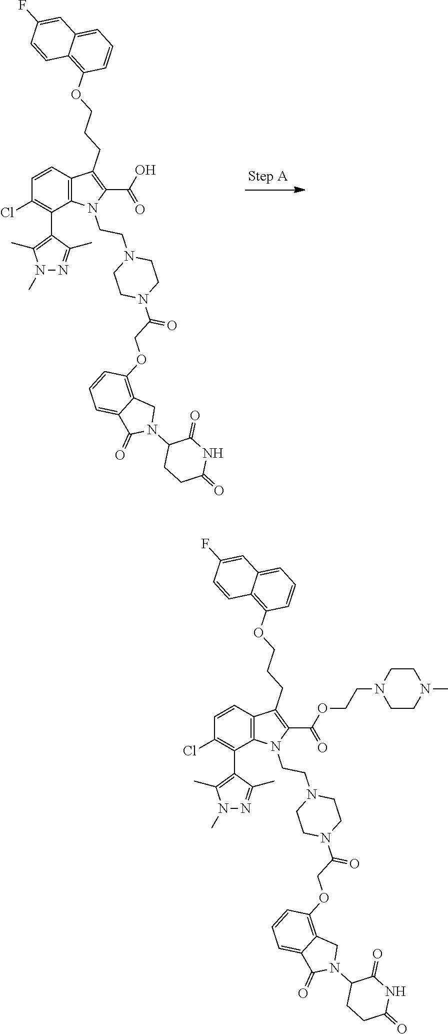

### Step A

6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (20.0 mg, 0.022 mmol), EDC—HCl (6.3 mg, 0.033 mmol) and DMAP (0.8 mg, 0.007 mmol) were dissolved in dry DMF (1.0 mL) under argon atmosphere and DIPEA (0.011 mL, 0.065 mmol), followed by 2-(4-methylpiperazin-1-yl)ethan-1-ol (3.8 mg, 0.026 mmol) were added. Reaction was stirred for 16 h at room temperature under argon. After that HATU (8.3 mg, 0.022 mmol) was added and the reaction was continued for the next 45 min. Then, the mixture was diluted with DCM and the solution was washed with brine and water. Organic layer was dried over anhydrous magnesium sulphate, filtered and dried under reduced pressure. The resulting residue was dissolved in DCM and purified with flash column chromatography (SiO₂, 1% to 40% MeOH in DCM) to give 2-(4-methylpiperazin-1-yl)ethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (5.8 mg, 0.006 mmol, 25.6%) as white powder.

LCMS (ESI): 1044.2 m/z [M+H]⁺

¹H NMR (500 MHz, DMSO, 353 K) δ 10.65 (s, 1H), 8.22 (dd, J=9.3, 5.9 Hz, 1H), 7.75 (d, J=8.6 Hz, 1H), 7.59 (dd, J=10.4, 2.6 Hz, 1H), 7.47-7.39 (m, 3H), 7.37-7.30 (m, 2H), 7.25 (d, J=8.9 Hz, 1H), 7.14 (d, J=8.1 Hz, 1H), 6.87 (dd, J=6.3, 2.3 Hz, 1H), 5.05 (dd, J=13.0, 5.2 Hz, 1H), 4.89 (s, 2H), 4.43-4.35 (m, 3H), 4.32 (d, J=17.0 Hz, 1H), 4.29-4.20 (m, 3H), 4.19-4.09 (m, 1H), 3.76 (d, J=2.0 Hz, 3H), 3.37-3.31 (m, 4H), 3.31-3.27 (m, 2H), 2.88 (ddd, J=18.9, 13.5, 5.6 Hz, 1H), 2.68 (t, J=5.8 Hz, 2H), 2.66-2.53 (m, 2H), 2.49-2.44 (m, 4H), 2.43-2.37 (m, 4H), 2.30-2.23 (m, 2H), 2.19 (s, 3H), 2.14-2.03 (m, 7H), 2.01 (s, 3H), 1.88 (s, 3H).

### Example 14: 2-Morpholinoethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 129)

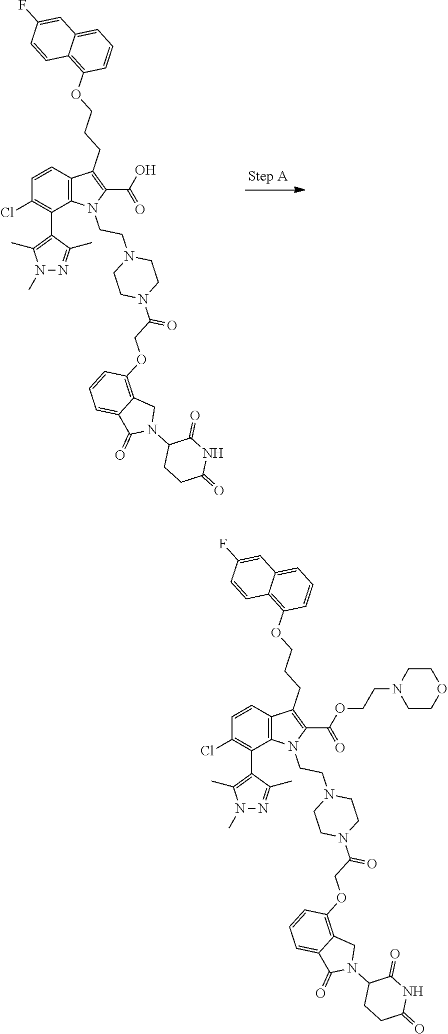 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCN3CCOCC3)n(CCN3CCN(C(=O)COc4cccc5c4CN(C4CCC(=O)NC4=O)C5=O)CC3)c12]

### Step A

6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (30.0 mg, 0.033 mmol) was suspended in MeCN (1.0 mL) and 4-(2-bromoethyl)morpholine hydrobromide (18.0 mg, 0.065 mmol), followed by Et₃N (0.046 mL, 0.327 mmol) was added. Reaction (monitored with LCMS) was stirred at 60° C. for 96 h. The solution was concentrated to dryness under reduced pressure and purified with flash column chromatography (SiO₂, 0% to 40% MeOH in DCM) and reverse phase preparative HPLC (C18, H₂O:MeCN+0.1% FA) to give 2-morpholinoethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (17.5 mg, 0.017 mmol, 51.8%) as white solid.

LCMS (ESI): 1031.2 m/z [M+H]³⁰

¹H NMR (500 MHz, DMSO, 353K) δ 10.65 (s, 1H), 8.21 (dd, J=9.2, 5.8 Hz, 1H), 7.75 (d, J=8.7 Hz, 1H), 7.59 (dd, J=10.4, 2.6 Hz, 1H), 7.48-7.37 (m, 3H), 7.37-7.28 (m, 2H), 7.24 (d, J=8.5 Hz, 1H), 7.14 (d, J=8.1 Hz, 1H), 6.87 (dd, J=6.3, 2.3 Hz, 1H), 5.05 (dd, J=13.0, 5.2 Hz, 1H), 4.89 (s, 2H), 4.46-4.35 (m, 3H), 4.32 (dd, J=17.1, 1.6 Hz, 1H), 4.29-4.12 (m, 4H), 3.76 (d, J=2.0 Hz, 3H), 3.57-3.48 (m, 4H), 3.38-3.31 (m, 4H), 3.31-3.27 (m, 2H), 2.88 (ddd, J=17.2, 13.5, 5.5 Hz, 1H), 2.67 (t, J=5.8 Hz, 2H), 2.65-2.60 (m, 1H), 2.47-2.39 (m, 5H), 2.30-2.23 (m, 2H), 2.15-2.02 (m, 7H), 2.01 (s, 3H), 1.88 (s, 3H).

### Example 15: 3-(((6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carbonyl)oxy)methyl)-1-methyl-1H-imidazol-3-ium iodide (Compound 140)

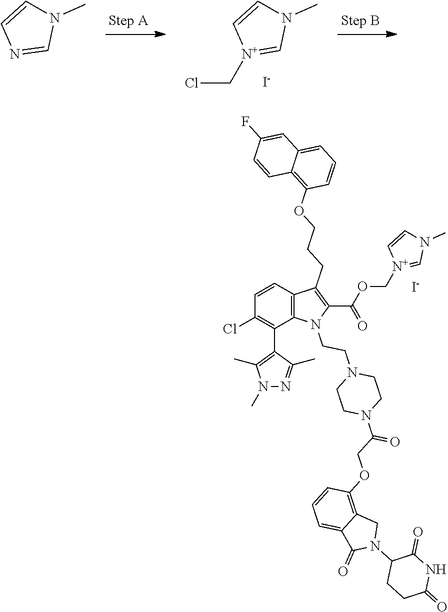

### Step A

1-Methyl-1H-imidazole (0.097 mL, 1.218 mmol) and chloroiodomethane (0.093 mL, 1.279 mmol) were placed in a vial and stirred for 48 h at room temperature. After that time reaction product was washed 3 times with Et₂O and dried under reduced pressure to give crude 3-(chloromethyl)-1-methyl-1H-imidazol-3-ium iodide (135.1 mg) as white solid.

### Step B

6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (5.0 mg, 0.005 mmol), crude 3-(chloromethyl)-1-methyl-1H-imidazol-3-ium iodide (3.1 mg), KI (4.5 mg, 0.027 mmol) and Et₃N (0.004 mL, 0.027 mmol) were dissolved in dry DMF and stirred for 3 h at 60° C. The mixture was diluted with DCM and washed with water. Organic layer was dried over anhydrous magnesium sulphate, filtered and dried under reduced pressure. The resulting residue was purified with preparative TLC (Al₂O₃, 20% MeOH in DCM) to give 3-(((6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carbonyl)oxy)methyl)-1-methyl-1H-imidazol-3-ium iodide (2.6 mg, 0.002 mmol, 41.9%) as beige solid.

LCMS (ESI): 1012.2 m/z [M+H]⁺

¹H NMR (500 MHz, DMSO, 353 K) δ 10.68 (s, 1H), 9.43-9.38 (m, 1H), 8.19 (dd, J=9.3, 5.9 Hz, 1H), 7.93 (t, J=1.9 Hz, 1H), 7.78 (d, J=8.7 Hz, 1H), 7.72 (t, J=1.8 Hz, 1H), 7.61 (dd, J=10.3, 2.6 Hz, 1H), 7.47-7.41 (m, 3H), 7.38-7.31 (m, 2H), 7.27 (d, J=8.6 Hz, 1H), 7.15 (d, J=8.1 Hz, 1H), 6.87 (dd, J=6.3, 2.4 Hz, 1H), 6.43 (d, J=10.7 Hz, 1H), 6.40 (d, J=10.7 Hz, 1H), 5.06 (dd, J=13.0, 5.3 Hz, 1H), 4.96-4.85 (m, 2H), 4.40 (d, J=17.1 Hz, 1H), 4.32 (dd, J=17.1, 1.6 Hz, 1H), 4.29-4.19 (m, 3H), 4.19-4.10 (m, 1H), 3.88 (s, 3H), 3.76 (d, J=2.2 Hz, 3H), 3.35-3.28 (m, 4H), 3.28-3.24 (m, 2H), 2.89 (ddd, J=17.3, 13.4, 5.5 Hz, 1H), 2.67-2.59 (m, 1H), 2.46-2.38 (m, 1H), 2.23-2.15 (m, 2H), 2.15-2.02 (m, 7H), 2.01 (s, 3H), 1.87 (s, 3H).

### Example 16: (Pivaloyloxy)methyl 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 74)

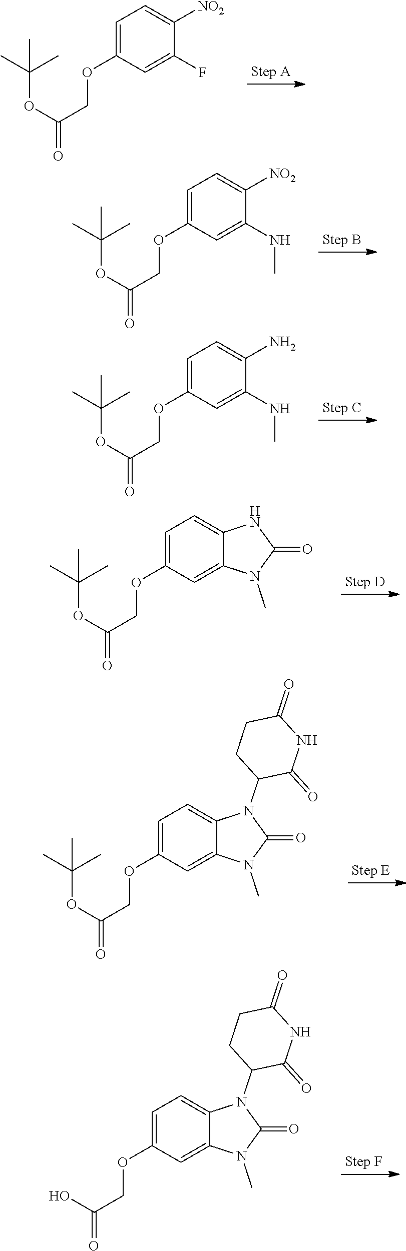

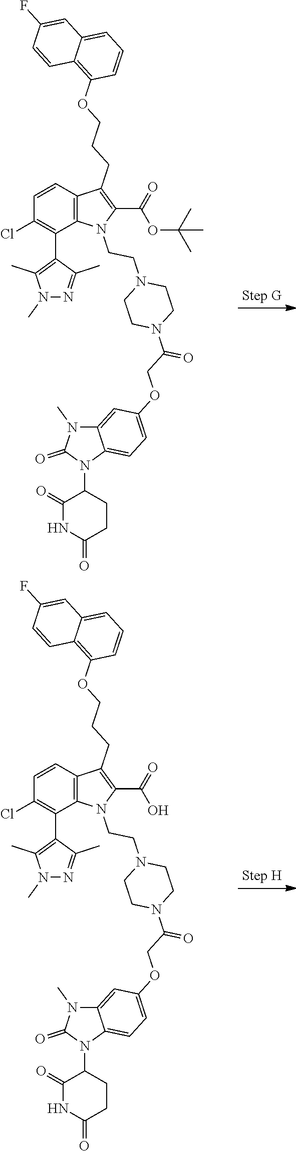

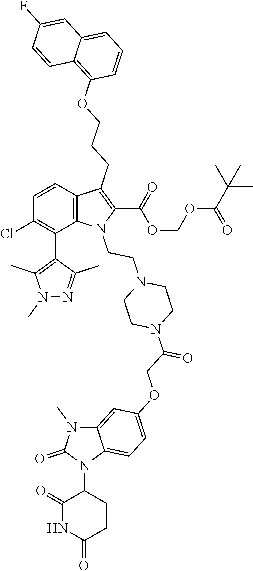 [SMILES: Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4ccc5c(c4)n(C)c(=O)n5C4CCC(=O)NC4=O)CC3)c12]

### Step A

To a solution of tert-butyl 2-(3-fluoro-4-nitrophenoxy)acetate (500 mg, 1.844 mmol) in EtOH (10 mL), an aqueous solution of methyl amine (0.41 ml, 9.22 mmol, 40% in H₂O) was added at room temperature and the reaction mixture was stirred for 2 h at 50° C. After complete consumption of the starting material (monitored by TLC and LCMS), the volatiles were removed and crude was purified by flash column chromatography (SiO₂, 50% EtOAc in hexane) to afford tert-butyl 2-(3-(methylamino)-4-nitrophenoxy)acetate (400 mg, 1.418 mmol, 77%) as brown liquid.

tert-Butyl 2-(3-fluoro-4-nitrophenoxy)acetate was prepared as described in US2009/163552A1.

LCMS (ESI+): 283.2 m/z [M+H]⁺.

### Step B

A solution of tert-butyl 2-(3-(methylamino)-4-nitrophenoxy)acetate (400 mg, 1.418 mmol) in MeOH (10 mL) was degassed with argon for 15 min. Pd/C (400 mg, 10% w/w) was added slowly at room temperature under nitrogen. The reaction mixture was then stirred for 4 h at room temperature under H₂ balloon pressure. After complete consumption of starting material, the reaction mixture was filtered through celite pad and the filtrate was concentrated under reduced pressure to get the crude material. The product was purified by column chromatography (SiO₂, 70% EtOAc in hexane) to get tert-butyl 2-(4-amino-3-(methylamino)phenoxy)acetate (250 mg, 0.992 mmol, 70%) as brown liquid.

LCMS (ESI+): 253.1 m/z [M+H]⁺.

### Step C

To a solution of tert-butyl 2-(4-amino-3-(methylamino)phenoxy)acetate (250 mg, 0.992 mmol) in THF (6 mL) was added CDI (290 mg, 1.785 mmol) at room temperature under nitrogen atmosphere. The reaction mixture was stirred at 70° C. for 4 h and monitored with LCMS and TLC. After completion, the volatiles were evaporated and crude material was purified by column chromatography (SiO₂, 50-60% EtOAc in hexane) to afford tert-butyl 2-((3-methyl-2-oxo-2,3-dihydro-1H-benzo[d]imidazol-5-yl)oxy)acetate (150 mg, 0.539 mmol, 55%) as white solid.

LCMS (ESI+): 278.9 m/z [M+H]⁺.

### Step D

To a solution of tert-butyl 2-((3-methyl-2-oxo-2,3-dihydro-1H-benzo[d]imidazol-5-yl)oxy)acetate (150 mg, 0.54 mmol) in DMF (4 mL) was added NaH (388 mg, 8.1 mmol, 60% in mineral oil) portionwise at 0° C. under nitrogen. The reaction mixture was stirred at the same temperature for 30 min. DMF (2 mL) solution of 3-bromopiperidine-2,6-dione (1.026 g, 5.4 mmol) was then added dropwise to the reaction at 0° C., over the period of 5 min. The mixture was then heated at 80° C. for 2 h under nitrogen. After complete consumption of the starting material (monitored by LCMS) the reaction mixture was cooled to −78° C., diluted with ethyl acetate and stirred for the next 5 min. A saturated solution of NH₄Cl was then added dropwise to the reaction mixture to quench the excess sodium hydride. Organic layer was separated from the solidified aqueous layer and the solid was washed with ethyl acetate. The combined organic phases were washed with brine and evaporated under reduced pressure to get the crude. Product was purified with preparative TLC (SiO₂, 60% EtOAc in hexane) to afford tert-butyl 2-((1-(2,6-dioxopiperidin-3-yl)-3-methyl-2-oxo-2,3-dihydro-1H-benzo[d]imidazol-5-yl)oxy)acetate (60 mg, 0.154 mmol, 28%) as off white solid.

LCMS (ESI+): 390.1 m/z [M+H]⁺.

### Step E

To a solution of tert-butyl 2-((1-(2,6-dioxopiperidin-3-yl)-3-methyl-2-oxo-2,3-dihydro-1H-benzo[d]imidazol-5-yl)oxy)acetate (60 mg, 0.154 mmol) in DCM (2 mL) was added dropwise TFA (1 mL) at 0° C. and the reaction mixture was stir at room temperature for 16 h under nitrogen. After complete consumption of the starting material (reaction monitored by TLC and LCMS) the volatiles were evaporated under reduced pressure to get the crude compound which was then purified by triturating with diethyl ether and n-pentane to get 2-((1-(2,6-dioxopiperidin-3-yl)-3-methyl-2-oxo-2,3-dihydro-1H-benzo[d]imidazol-5-yl)oxy)acetic acid (45 mg, 0.135 mmol, 87%) as off white solid.

LCMS (ESI+): 334.2 m/z [M+H]⁺.

### Step F

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol) and 2-((1-(2,6-dioxopiperidin-3-yl)-3-methyl-2-oxo-2,3-dihydro-1H-benzo[d]imidazol-5-yl)oxy)acetic acid (10.9 mg, 0.033 mmol) were dissolved under argon atmosphere in dry DMF (0.989 mL) and DIPEA (0.016 mL, 0.089 mmol) was added followed by HATU (11.8 mg, 0.031 mmol). The reaction (monitored by LCMS) was stirred for 20 min under argon. After complete consumption of the starting material the mixture was diluted with DCM and washed with brine and water. Organic layer was dried over anhydrous MgSO₄, filtered and dried under reduced pressure to give crude tert-butyl 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate as yellow oil, that was directly used in the next step.

LCMS (ESI+): 989.0 m/z [M+H]⁺.

### Step G

Crude tert-butyl 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate was dissolved in dry DCM (0.5 mL) under argon atmosphere and TFA (0.5 mL) was added. The reaction (monitored with LCMS) was stirred under argon for 16 h. After complete consumption of the starting material the solution was concentrated under reduced pressure and the resulting residue was purified by preparative HPLC (C18, H₂O:MeCN+0.1% FA) to give 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (10.5 mg, 0.011 mmol, 36.7% over 2 steps) as white solid.

LCMS (ESI+): 933.15 m/z [M+H]⁺.

### Step H

To a solution of 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (13.0 mg, 0.014 mmol) in MeCN (500 μL) was added Et₃N (10 μL, 0.070 mmol). The reaction was stirred for 15 min and chloromethyl pivalate (2 μL, 0.017 mmol) was added. The reaction was carried out overnight at room temperature. The second portion of chloromethyl pivalate (2 μL, 0.017 mmol) was added and the mixture was stirred at 60° C. for 48 h. The solvents were removed under reduced pressure, the mixture was diluted with DCM and brine. The aqueous layer was extracted with DCM. The combined organic layers were dried over Na₂SO₄ and concentrated under vacuum to give the corresponding (pivaloyloxy)methyl 6-chloro-1-(2-(4-(2-((1-(2,6-dioxopiperidin-3-yl)-3-methyl-2-oxo-2,3-dihydro-1H-benzo[d]imidazol-5-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (13.5 mg, 0.013 mmol, 92%) as a white solid.

LCMS (ESI): 1047.25 m/z [M+H]⁺.

¹H NMR (500 MHz, DMSO, 353 K) δ 10.76 (s, 1H), 8.21 (dd, J=9.2, 5.9 Hz, 1H), 7.77 (d, J=8.6 Hz, 1H), 7.59 (dd, J=10.4, 2.6 Hz, 1H), 7.45-7.38 (m, 2H), 7.32 (td, J=8.9, 2.6 Hz, 1H), 7.26 (d, J=8.6 Hz, 1H), 6.96 (d, J=8.6 Hz, 1H), 6.86 (dd, J=6.1, 2.6 Hz, 1H), 6.82 (d, J=2.4 Hz, 1H), 6.62 (dd, J=8.6, 2.5 Hz, 1H), 5.99 (s, 2H), 5.25 (dd, J=12.5, 5.4 Hz, 1H), 4.69 (s, 2H), 4.28-4.20 (m, 3H), 4.20-4.12 (m, 1H), 3.75 (s, 3H), 3.38-3.32 (m, 4H), 3.30 (s, 3H), 3.28-3.23 (m, 2H), 2.91-2.84 (m, 1H), 2.72-2.63 (m, 1H), 2.54-2.51 (m, 1H), 2.26-2.19 (m, 2H), 2.14-2.01 (m, 7H), 2.00 (s, 3H), 1.87 (s, 3H), 1.15 (s, 9H).

### Example 17: (Pivaloyloxy)methyl 6-chloro-1-(2-(4-(2-((1-(2,6-dioxopiperidin-3-yl)-3-methyl-2-oxo-2,3-dihydro-1H-benzo[d]imidazol-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 73)

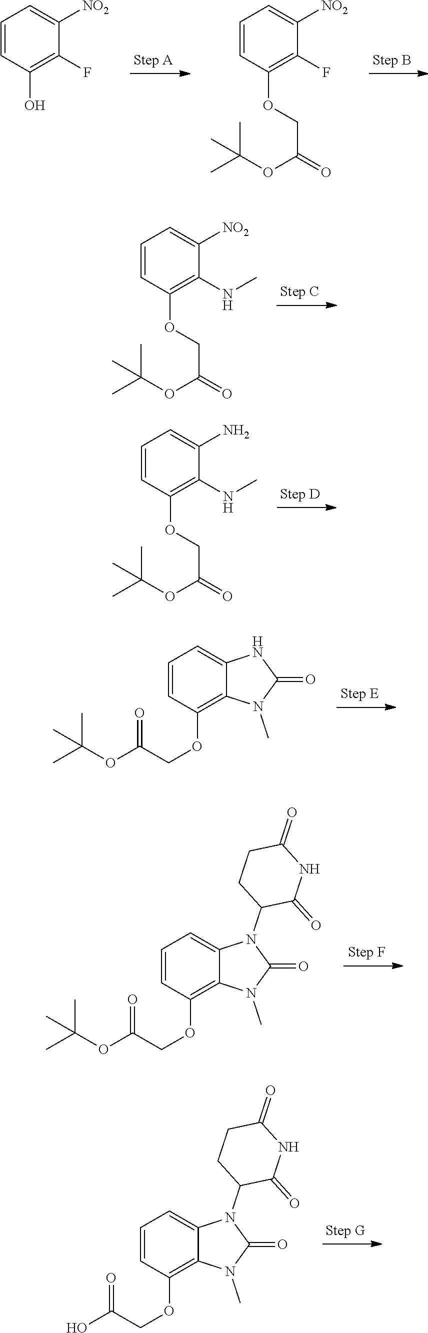

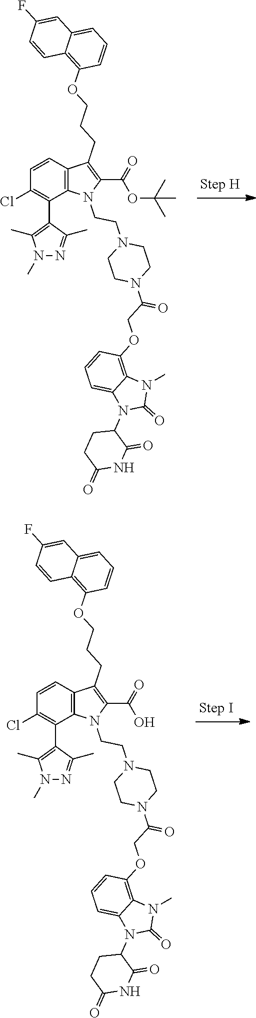

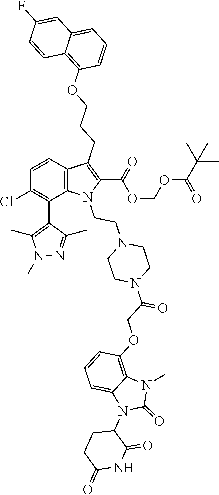 [SMILES: Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4cccc5c4n(C)c(=O)n5C4CCC(=O)NC4=O)CC3)c12]

### Step A

2-Fluoro-3-nitrophenol (1 g, 6.365 mmol) was dissolved in DMF (20 mL) and KI (317 mg, 1.91 mmol) and KHCO₃ (764 mg, 7.638 mmol) were added. Then, tert-butyl bromoacetate (1.691 ml, 11.458 mmol) was added dropwise and the reaction mixture was stirred at 60° C. for 5 h under nitrogen. After complete consumption of the starting material the reaction mixture was diluted with ethyl acetate and washed with water and brine. The organic layer was dried over Na₂SO₄ and evaporated to get the crude compound, which was then purified by column chromatography (SiO₂, 0-10% EtOAc in hexane) to afford tert-butyl 2-(2-fluoro-3-nitrophenoxy)acetate (1.6 g, 5.90 mmol, 92%) as a yellow solid.

### Step B

To a solution of tert-butyl 2-(2-fluoro-3-nitrophenoxy)acetate (400 mg, 1.476 mmol) in EtOH (8 mL) an aqueous solution of methylamine (0.33 mL, 7.378 mmol, 40% in H₂O) was added at room temperature and the reaction mixture was stirred for 2 h at 50° C. After complete consumption of the starting material, the volatiles were removed under reduced pressure and crude was purified by column chromatography (SiO₂, 50% EtOAc in hexane) to afford tert-butyl 2-(2-(methylamino)-3-nitrophenoxy)acetate (370 mg, 1.31 mmol, 88%) as brown liquid.

LCMS (ESI+): 283.2 m/z [M+H]⁺.

### Step C

A solution of tert-butyl 2-(2-(methylamino)-3-nitrophenoxy)acetate (370 mg, 1.312 mmol) in MeOH (10 mL) was degassed with argon for 15 minutes. Pd/C (370 mg, 10% w/w) was added slowly under argon. The reaction mixture was then stirred for 4 h at room temperature under H₂ balloon pressure. After complete consumption of the starting material, the reaction mixture was filtered through celite pad and the filtrate was concentrated under reduced pressure. Crude material was purified with column chromatography (SiO₂, 70% EtOAc in hexane) to get tert-butyl 2-(3-amino-2-(methylamino)phenoxy)acetate (225 mg, 0.892 mmol, 68%) of as brown liquid.

LCMS (ESI+): 253.0 m/z [M+H]⁺.

### Step D

To a solution of tert-butyl 2-(3-amino-2-(methylamino)phenoxy)acetate (225 mg, 0.892 mmol) in THF (12 mL) was added CDI (261 mg, 1.606 mmol) at room temperature under nitrogen atmosphere. The reaction mixture was stirred at 60° C. for 4 h. The volatiles were removed under reduced pressure and crude material was purified by column chromatography (SiO₂, 50-60% EtOAc in hexane) to afford tert-butyl 2-((3-methyl-2-oxo-2,3-dihydro-1H-benzo[d]imidazol-4-yl)oxy)acetate (150 mg, 0.539 mmol, 60%) of as a white solid.

LCMS (ESI+): 279.2 m/z [M+H]⁺.

### Step E

To a solution of tert-butyl 2-((3-methyl-2-oxo-2,3-dihydro-1H-benzo[d]imidazol-4-yl)oxy)acetate (150 mg, 0.54 mmol) in DMF (4 mL) was added NaH (388 mg, 8.1 mmol, 60% in mineral oil) portionwise at 0° C. under nitrogen and the reaction mixture was stirred at the same temperature for 30 min. The solution of 3-bromopiperidine-2,6-dione (1.026 g, 5.4 mmol) in DMF (2 mL) was then added dropwise to the reaction mixture at 0° C. over a period of 5 min. The mixture was then heated to 80° C. for 2 h under nitrogen. After complete consumption of the starting material (monitored by LCMS) the reaction mixture was cooled to −78° C., diluted with EtOAc and stirred for the 5 min. A saturated solution of ammonium chloride was then added dropwise to the reaction mixture to quench the excess sodium hydride. Organic layer was separated from the solidified aqueous layer and the solid was washed with EtOAc. The combined organic phases were washed with brine and evaporated. Crude compound was purified with preparative TLC (SiO₂, 60% EtOAc in hexane) to afford 60 mg (0.154 mmol, 28%) of tert-butyl 2-((1-(2,6-dioxopiperidin-3-yl)-3-methyl-2-oxo-2,3-dihydro-1H-benzo[d]imidazol-4-yl)oxy)acetate as off white solid.

LCMS (ESI+): 390.2 m/z [M+H]⁺.

### Step F

To a solution of tert-butyl 2-((1-(2,6-dioxopiperidin-3-yl)-3-methyl-2-oxo-2,3-dihydro-1H-benzo[d]imidazol-4-yl)oxy)acetate (60 mg, 0.154 mmol) in DCM (2 mL) was added TFA (1 mL) at 0° C. dropwise and the reaction mixture was stirred at room temperature for 16 h under nitrogen. After complete consumption of the starting material the volatiles were removed under reduced pressure to get the crude compound which was then purified by triturating with diethyl ether and n-pentane to get 2-((1-(2,6-dioxopiperidin-3-yl)-3-methyl-2-oxo-2,3-dihydro-1H-benzo[d]imidazol-4-yl)oxy)acetic acid (30 mg, 0.09 mmol, 58%) of as off white solid.

LCMS (ESI+): 334.2 m/z [M+H]⁺.

### Step G

tert-Butyl 6-chloro-3-{3-[(6-fluoronaphthalen-1-yl)oxy]propyl}-1-[2-(piperazin-1-yl)ethyl]-7-(1,3,5-trimethyl-1H-pyrazol-4-yl)-1H-indole-2-carboxylate (20.0 mg, 0.030 mmol), 2-((1-(2,6-dioxopiperidin-3-yl)-3-methyl-2-oxo-2,3-dihydro-1H-benzo[d]imidazol-4-yl)oxy)acetic acid (10.9 mg, 0.033 mmol) and DIPEA (0.016 mL, 0.089 mmol) were dissolved in dry DMF (1.0 mL) under argon atmosphere. HATU (11.8 mg, 0.031 mmol) in DMF (1.0 mL) was added. The reaction was stirred for 15 min at room temperature under argon. After complete conversion, the solution was diluted with DCM up to 10 mL. The resulting solution was washed with brine and water, dried over anhydrous MgSO₄, filtered and dried under reduced pressure. Crude tert-butyl 6-chloro-1-{2-[4-(2-{[1-(2,6-dioxopiperidin-3-yl)-3-methyl-2-oxo-2,3-dihydro-1H-1,3-benzodiazol-4-yl]oxy}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carboxylate (41.8 mg) was obtained as yellow oil.

LCMS (ESI+): 989.2 m/z [M+H]⁺.

### Step H

Crude tert-butyl 6-chloro-1-{2-[4-(2-{[1-(2,6-dioxopiperidin-3-yl)-3-methyl-2-oxo-2,3-dihydro-1H-1,3-benzodiazol-4-yl]oxy}acetyl)piperazin-1-yl]ethyl}1-3-{3-[(6-fluoronaphthalen-1-yl)oxy]propyl}-7-(1,3,5-trimethyl-1H-pyrazol-4-yl)-1H-indole-2-carboxylate (41.8 mg) was dissolved in dry DCM (0.500 mL) under argon atmosphere and TFA (0.500 mL, 6.529 mmol) was added. Reaction was stirred under argon for 16 h. After complete consumption of the starting material the solution was concentrated under reduced pressure and the resulting residue purified with preparative HPLC (C18, H₂O:MeCN+0.1% FA) to give 6-chloro-1-(2-(4-(2-((1-(2,6-dioxopiperidin-3-yl)-3-methyl-2-oxo-2,3-dihydro-1H-benzo[d]imidazol-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (13.9 mg, 0.015 mmol, 50% over 2 steps) as white solid.

LCMS (ESI+): 933.2 m/z [M+H]⁺.

### Step I

To a solution of 6-chloro-1-(2-(4-(2-((1-(2,6-dioxopiperidin-3-yl)-3-methyl-2-oxo-2,3-dihydro-1H-benzo[d]imidazol-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (17.7 mg, 0.019 mmol) in dry MeCN (0.95 mL) was added Et₃N (0.013 mL, 0.095 mmol) followed by chloromethyl pivalate (0.030 mL, 0.021 mmol). The reaction was stirred at room temperature for 3 h. Additional portion of chloromethyl pivalate (0.027 mL, 0.019 mmol) was added and the mixture was stirred at 60° C. for 16 h. Then again chloromethyl pivalate (0.014 mL, 0.010 mmol) was added and the reaction was continued for additional 16 h. After complete consumption of the starting material the mixture was concentrated to dryness under reduced pressure. The resulting residue was diluted with DCM and washed with water. The organic layer was dried over anhydrous MgSO₄, filtered and concentrated. The resulting solid was freeze-dried to give (pivaloyloxy)methyl 6-chloro-1-(2-(4-(2-((1-(2,6-dioxopiperidin-3-yl)-3-methyl-2-oxo-2,3-dihydro-1H-benzo[d]imidazol-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (15.4 mg, 0.015 mmol, 77%) as white solid.

LCMS (ESI): 1047.25 m/z [M+H]⁺.

¹H NMR (500 MHz, DMSO, 353 K) δ 10.77 (s, 1H), 8.21 (dd, J=9.2, 5.9 Hz, 1H), 7.77 (d, J=8.6 Hz, 1H), 7.59 (dd, J=10.4, 2.6 Hz, 1H), 7.45-7.39 (m, 2H), 7.32 (ddd, J=9.2, 8.6, 2.6 Hz, 1H), 7.26 (d, J=8.5 Hz, 1H), 6.93 (t, J=8.2 Hz, 1H), 6.87 (dd, J=5.9, 2.7 Hz, 1H), 6.75 (dd, J=8.0, 0.8 Hz, 1H), 6.72 (dd, J=8.4, 0.8 Hz, 1H), 5.99 (s, 2H), 5.27 (dd, J=12.4, 5.5 Hz, 1H), 4.84 (s, 2H), 4.31-4.19 (m, 3H), 4.19-4.09 (m, 1H), 3.75 (s, 3H), 3.57 (s, 3H), 3.40-3.30 (m, 4H), 3.30-3.21 (m, 2H), 2.95-2.81 (m, 1H), 2.74-2.61 (m, 2H), 2.26-2.18 (m, 2H), 2.14-2.01 (m, 7H), 2.01 (s, 3H), 1.87 (s, 3H), 1.15 (s, 9H).

### Example 18: Acetoxymethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 41)

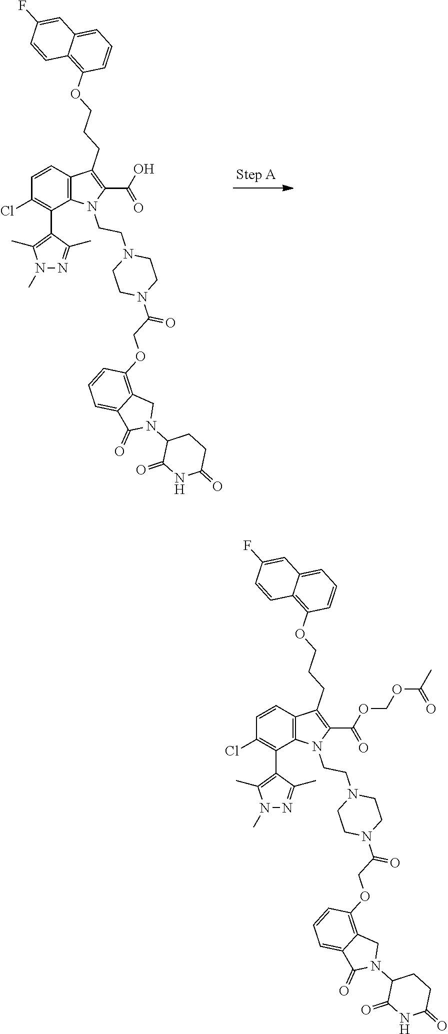 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; CC(=O)OCOC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1]

### Step A

To a solution of 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (10.0 mg, 0.011 mmol) in DMF (0.31 mL) was added DIPEA (0.009 mL, 0.054 mmol) followed by chloromethyl acetate (0.001 mL, 0.016 mmol). The reaction was stirred for 16 h at 55° C. After completion the solution was evaporated. The resulting residue was dissolved in DMSO and purified by reversed phase preparative HPLC (C18, H₂O:MeCN+0.1% FA) to give acetoxymethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (6.6 mg, 0.007 mmol, 61%) as white solid.

LCMS (ESI): 990.25 m/z [M+H]⁺.

¹H NMR (500 MHz, DMSO, 353 K) δ 10.66 (s, 1H), 8.23 (dd, J=9.2, 5.8 Hz, 1H), 7.77 (d, J=8.6 Hz, 1H), 7.59 (dd, J=10.4, 2.6 Hz, 1H), 7.50-7.38 (m, 3H), 7.38-7.29 (m, 2H), 7.26 (d, J=8.6 Hz, 1H), 7.14 (d, J=8.1 Hz, 1H), 6.88 (dd, J=5.6, 3.0 Hz, 1H), 5.96 (d, J=6.0 Hz, 1H), 5.95 (d, J=6.1 Hz, 1H), 5.05 (dd, J=13.0, 5.2 Hz, 1H), 4.89 (s, 2H), 4.40 (d, J=17.1 Hz, 1H), 4.31 (dd, J=17.2, 1.4 Hz, 1H), 4.28-4.19 (m, 3H), 4.19-4.10 (m, 1H), 3.76 (d, J=1.8 Hz, 3H), 3.36-3.30 (m, 4H), 3.30-3.24 (m, 2H), 2.88 (ddd, J=17.3, 13.5, 5.5 Hz, 1H), 2.66-2.59 (m, 1H), 2.48-2.39 (m, 1H), 2.26-2.18 (m, 2H), 2.15-2.02 (m, 10H), 2.01 (s, 3H), 1.88 (s, 3H).

### Example 19: 1-Acetoxyethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (Compound 75)

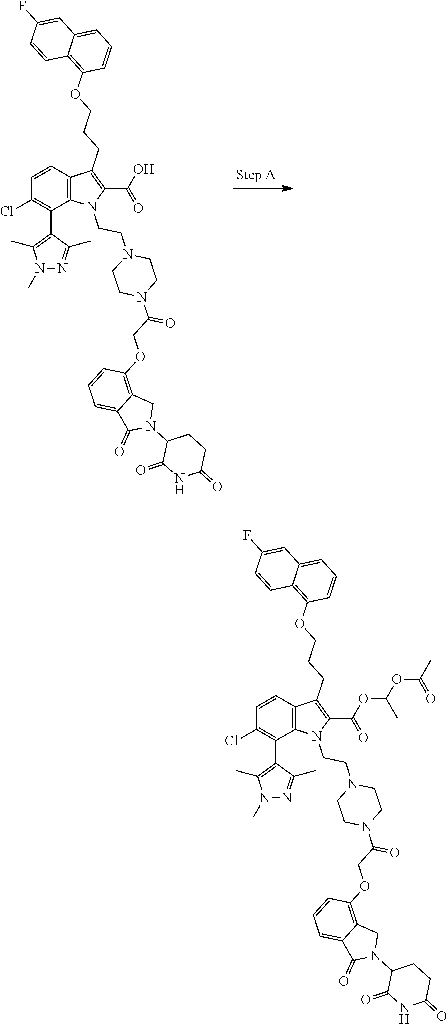 [SMILES: Cc1nn(C)c(C)c1-c1c(Cl)ccc2c(CCCOc3cccc4cc(F)ccc34)c(C(=O)O)n(CCN3CCN(C(=O)COc4cccc5c4CN(C4CCC(=O)NC4=O)C5=O)CC3)c12 ; CC(=O)OC(C)OC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1]

### Step A

6-Chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylic acid (20.0 mg, 0.022 mmol) was dissolved in dry DMF (0.73 mL) and DIPEA (0.019 mL, 0.109 mmol), followed by 1-bromoethyl acetate (4.4 mg, 0.026 mmol). The reaction was stirred at 55° C. for 24 h under argon atmosphere. Second portion of 1-bromoethyl acetate (3.6 mg, 0.022 mmol) was added and the reaction was continued for the next 48 h at 55° C. under argon. After completion, the solution was concentrated under reduced pressure, diluted with DMSO and purified with reversed phase preparative HPLC (C18, H₂O:MeCN+0.1% FA) to give 1-acetoxyethyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1,3,5-trimethyl-1H-pyrazol-4-yl)-1H-indole-2-carboxylate (10.9 mg, 0.011 mmol, 49%) as white solid.

LCMS (ESI): 1004.1 m/z [M+H]⁺.

¹H NMR (500 MHz, DMSO, 353K) δ 10.66 (s, 1H), 8.24 (dd, J=9.2, 5.8 Hz, 1H), 7.77 (d, J=8.6 Hz, 1H), 7.59 (dd, J=10.4, 2.6 Hz, 1H), 7.47-7.38 (m, 3H), 7.37-7.29 (m, 2H), 7.26 (d, J=8.6 Hz, 1H), 7.14 (d, J=8.1 Hz, 1H), 7.08-7.00 (m, 1H), 6.90 (dd, J=5.9, 2.7 Hz, 1H), 5.05 (dd, J=13.1, 5.2 Hz, 1H), 4.89 (s, 2H), 4.40 (d, J=17.0 Hz, 1H), 4.32 (dd, J=17.1, 1.6 Hz, 1H), 4.27 (t, J=6.3 Hz, 2H), 4.25-4.20 (m, 1H), 4.20-4.11 (m, 1H), 3.76 (d, J=2.1 Hz, 3H), 3.37-3.32 (m, 4H), 3.32-3.24 (m, 2H), 2.88 (ddd, J=17.4, 13.5, 5.5 Hz, 1H), 2.67-2.58 (m, 1H), 2.48-2.40 (m, 1H), 2.27-2.20 (m, 2H), 2.17-2.04 (m, 7H), 2.03 (d, J=1.5 Hz, 3H), 2.01 (d, J=1.1 Hz, 3H), 1.88 (s, 3H), 1.59 (dd, J=5.5, 2.1 Hz, 3H).

### Example 20: (Pivaloyloxy)methyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (Compound 149)

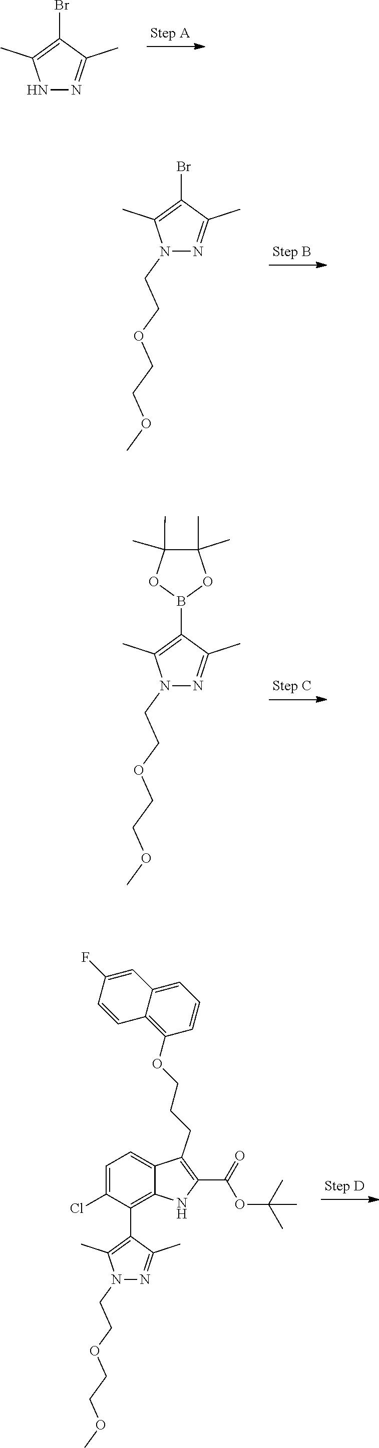

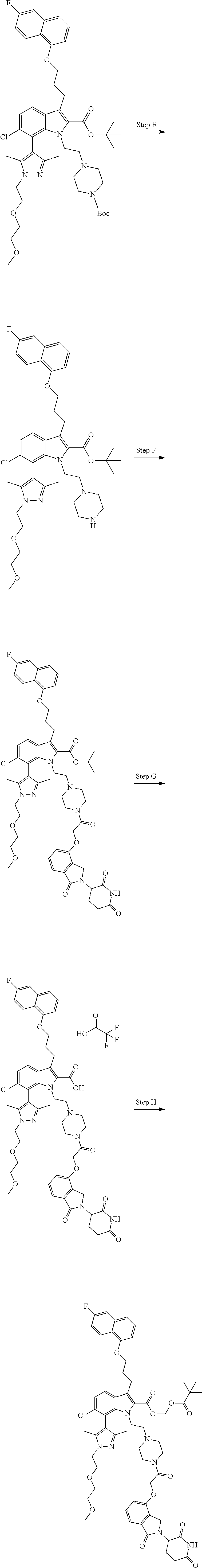

### Step A

To a solution of 4-bromo-3,5-dimethyl-1H-pyrazole (5 g, 28.57 mmol) in DMF (50 mL) was added 1-bromo-2-(2-methoxyethoxy)ethane (5.07 ml, 37.14 mmol) followed by Cs₂CO₃ (18.57 g, 57.14 mmol) at room temperature under nitrogen. The reaction mixture was stirred at room temperature for 2 h. After complete consumption of the starting materials the reaction mixture was diluted with ethyl acetate and washed with water and brine. The organic layer was dried over Na₂SO₄ and concentrated under reduced pressure. The residue was triturated with diethyl ether and n-pentane to get 4.8 g (17.33 mmol, 60%) of 4-bromo-1-(2-(2-methoxyethoxy)ethyl)-3,5-dimethyl-1H-pyrazole as off white solid.

LCMS (ESI): 276.8 m/z [M+H]⁺.

### Step B

To a solution of 4-bromo-1-(2-(2-methoxyethoxy)ethyl)-3,5-dimethyl-1H-pyrazole (1 g, 3.61 mmol) and 2-isopropoxy-4,4,5,5-tetramethyl-1,3,2-dioxaborolane (3.70 ml, 18.12 mmol) in THF (20 mL) was added n-butyllithium (2M in hexanes, 4.5 mL, 9.058 mmol) at −78° C. under nitrogen. The reaction mixture was stirred at the same temperature for 2 h. After full conversion the excess of n-butyllithium was quenched by saturated NH₄Cl solution at −78° C. and the reaction was extracted with ethyl acetate. The combined organic fractions were washed with brine, dried over Na₂SO₄ and concentrated under reduced pressure. Crude 1-(2-(2-methoxyethoxy)ethyl)-3,5-dimethyl-4-(4,4,5,5-tetramethyl-1,3,2-dioxaboroan-2-yl)-1H-pyrazole (1 g) was used in the next step without further purification.

LCMS (ESI): 325.0 m/z [M+H]⁺.

### Step C

To a solution of tert-butyl 7-bromo-6-chloro-3-(3-((6-fluoronaphthalen-1-yl)oxy)propyl)-1H-indole-2-carboxylate (300 mg, 0.57 mmol) in dioxane (4 mL) and water (1 mL) were added crude 1-(2-(2-methoxyethoxy)ethyl)-3,5-dimethyl-4-(4,4,5,5-tetramethyl-1,3,2-dioxaborolan-2-yl)-1H-pyrazole (734 mg) and Cs₂CO₃ (552 mg, 1.70 mmol). The mixture was deoxygenated with argon and PdCl₂(dtbpf) (36 mg, 0.056 mmol) was added under nitrogen atmosphere. The reaction mixture was stirred at 100° C. for 1 h until complete consumption of the starting materials. The reaction mixture was diluted with ethyl acetate and washed successively with water and brine. The organic layer was dried over Na₂SO₄ and concentrated under reduced pressure. tert-Butyl 6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (240 mg, 0.37 mmol, 65%) was purified by column chromatography (SiO₂, 20% EtOAc in hexane).

LCMS (ESI): 650.4 m/z [M+H]⁺.

### Step D

To a solution of tert-butyl 6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (240 mg, 0.37 mmol) in DMF (5 mL) were added tert-butyl 4-(2-chloroethyl)piperazine-1-carboxylate (275 mg, 1.11 mmol) and Cs₂CO₃ (360 mg, 1.11 mmol) at room temperature. The reaction mixture was stirred at 90° C. for 16 h under nitrogen. After complete consumption of the starting materials the reaction mixture was diluted with ethyl acetate and washed with water and brine. The organic layer was dried over Na₂SO₄ and concentrated under reduced pressure. The crude was purified by column chromatography (SiO₂, 30% EtOAc in hexane) to afford 240 mg (0.28 mmol, 75%) of tert-butyl 1-(2-(4-(tert-butoxycarbonyl)piperazin-1-yl)ethyl)-6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate as white solid.

LCMS (ESI): 862.8 m/z [M+H]⁺.

### Step E

To a solution of tert-butyl 1-(2-(4-(tert-butoxycarbonyl)piperazin-1-yl)ethyl)-6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (240 mg, 0.28 mmol) in dioxane (3 mL) was added 6 mL of 4M HCl in dioxane at 0° C. under nitrogen. The reaction mixture was stirred with cooling to 0° C. for 2 h under nitrogen. After complete consumption of the starting material (monitored by TLC and LCMS), the reaction mixture was poured into cold 1M NaOH solution and extracted with dichloromethane. The combined organic fractions were dried over Na₂SO₄ and concentrated in vacuo. Crude compound was purified by trituration with diethyl ether and n-pentane to get 200 mg (0.26 mmol, 94%) of tert-butyl 6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1-(2-(piperazin-1-yl)ethyl)-1H-indole-2-carboxylate as white solid.

LCMS (ESI): 762.6 m/z [M+H]⁺.

### Step F

To a solution of tert-butyl 6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1-(2-(piperazin-1-yl)ethyl)-1H-indole-2-carboxylate (180 mg, 0.24 mmol) in DMF (5 mL) were added 2-((2-(2,6-dioxopiperidin-3-yl)-1-oxoisoindolin-4-yl)oxy)acetic acid (75 mg, 0.24 mmol), HATU (134 mg, 0.36 mmol) and DIPEA (0.082 mL, 0.47 mmol) at room temperature under nitrogen. The mixture was stirred at room temperature for 1 h under nitrogen.

After completion of the reaction, the mixture was poured into ice-cold water and extracted with ethyl acetate. The organic layer was washed with brine, dried over anhydrous Na₂SO₄ and concentrated under reduced pressure. The crude compound was purified by column chromatography (SiO₂, 10% MeOH in DCM) to afford 100 mg (0.094 mmol, 39%) of tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate as off white solid.

LCMS (ESI): 1062.4 m/z [M+H]⁺.

### Step G

To a solution of tert-butyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (100 mg, 0.094 mmol) in DCM (5 mL) was added TFA (1 mL) dropwise at 0° C. under nitrogen. The mixture was stirred at room temperature for 16 h under nitrogen. After complete consumption of the starting material (monitored by TLC and LCMS), the volatiles were evaporated. Crude compound was purified by trituration with diethyl ether and n-pentane to get 93 mg (0.083 mmol, 83%) of 1-(2-(2-carboxy-6-chloro-3-(3-((6-fluoronaphthalen-1-yl)oxy)propyl)-7-(1-(2-(2-methoxyethoxy)ethyl)-3,5-dimethyl-1H-pyrazol-4-yl)-1H-indol-1-yl)ethyl)-4-(2-((2-(2,6-dioxopiperidin-3-yl)-1-oxoisoindolin-4-yl)oxy)acetyl)piperazin-1-ium trifluoroacetate as white solid.

LCMS (ESI): 1006.6 m/z [M+H]⁺.

### Step H

To a solution of 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylic acid trifluoroacetate (16.8 mg, 0.015 mmol) in DMF (75 μL) and DIPEA (16 μL, 0.089 mmol) was added chloromethyl 2,2-dimethylpropanoate (4 μL, 0.030 mmol). The reaction was stirred for 24 h at room temperature. Additional portion of chloromethyl 2,2-dimethylpropanoate (4 μL, 0.030 mmol) was added and the reaction mixture was stirred at 55° C. for 48 h. The solvents were removed under reduced pressure and the product was purified using flash chromatography (SiO₂, 5-10% MeOH in DCM) to get (pivaloyloxy)methyl 6-chloro-1-(2-(4-(2-((2-(2,6-dioxopiperidin-3-yl)-1-oxoisoindolin-4-yl)oxy)acetyl)piperazin-1-yl)ethyl)-3-(3-((6-fluoronaphthalen-1-yl)oxy)propyl)-7-(1-(2-(2-methoxyethoxy)ethyl)-3,5-dimethyl-1H-pyrazol-4-yl)-1H-indole-2-carboxylate (2.1 mg, 0.002 mmol, 12%) as a white solid.

LCMS (ESI): 1120.5 m/z [M+H]⁺.

¹H NMR (500 MHz, DMSO, 353K) δ 10.66 (s, 1H), 8.21 (dd, J=9.3, 5.9, 1H), 7.77 (d, J=8.6, 1H), 7.59 (dd, J=10.3, 2.6, 1H), 7.46-7.40 (m, 3H), 7.35-7.29 (m, 2H), 7.26 (d, J=8.6, 1H), 7.13 (dd, J=8.1, 3.0, 1H), 6.87 (dd, J=6.1, 2.6, 1H), 5.99 (s, 2H), 5.15-5.03 (m, 2H), 4.88 (s, 2H), 4.40 (dd, J=17.1, 4.5, 1H), 4.31 (d, J=17.1, 1H), 4.26-4.17 (m, 6H), 3.81-3.78 (m, 2H), 3.54 (dd, J=5.8, 4.1, 2H), 3.46-3.43 (m, 2H), 3.33-3.29 (m, 4H), 3.28-3.23 (m, 5H), 2.88 (ddd, J=18.5, 13.3, 5.5, 1H), 2.46-2.38 (m, 1H), 2.25-2.19 (m, 2H), 2.12-2.02 (m, 7H), 2.01 (s, 3H), 1.91 (s, 3H), 1.15 (s, 9H).

### Example 21: Cell Viability Assays MV4-11, ARH-77 Cells

The effect of various compounds of the invention on the viability of MV4-11 and ARH-77 cell lines was investigated, using the CTG protocol described below.

MV4-11 cell lines are derived from acute myeloid leukaemia and it is described in the literature as a MCL-1 dependent cell line, as opposed to lymphoblast-like ARH-77 cell line, which is described as independent of MCL-1 (Tron A E et al. Nat Commun. 2018; 9: 5341; Caenepeel S et al. Cancer Discov. 2018 December; 8(12): 1582-1597).

Cells (MV4-11 or ARH-77) were seeded on 348-well plates in a growth medium with 1% FBS. Next, compound stocks were pre-diluted in DMSO and added directly to the cell culture media (final DMSO concentration: 0.5%). Compound concentrations used in this assay: 1/3 dilutions starting at 30 μM (9 to 12 points). After 24 hours of incubation, cell viability was evaluated using CellTiter-Gob® Luminescent Cell Viability Assay (Promega, cat #G7572). The luminescence signal was measured using a CLARIOstar Plus Multi-Mode Microplate Reader (574-590 nm).

Results were normalized to DMSO control. Collected data were analyzed using GraphPad Prism to calculate pCC₅₀ values (i.e. the negative log of the CC₅₀ value). CC₅₀ (50% cytotoxic concentration) is the effective concentration of a cytotoxic compound, which produces 50% of the maximum possible cell death (i.e. the concentration of the compound which is required in order to reduce cell viability by 50%). CC₅₀ is often used rather than IC₅₀ in cell toxicity assays.

TABLE 9
The effect of various compounds of the invention
on the viability of MV4-11 and ARH-77 cell lines

| | Average pCC₅₀ | Average pCC₅₀ |
|---|---|---|
| Compound ID | by molecule MV4-11 | by molecule ARH-77 |
| 41 | >9.0 | <5.0 |
| 43 | >8.5 | <5.5 |
| 55 | >7.0 | <5.0 |
| 57 | >8.0 | <5.0 |
| 69 | >8.0 | <4.5 |
| 70 | >8.0 | <4.5 |
| 71 | >7.0 | <5.0 |
| 73 | >8.5 | <6.0 |
| 74 | >8.5 | <6.0 |
| 75 | >8.0 | <4.5 |
| 99 | >7.5 | <4.5 |
| 100 | >7.5 | <4.5 |
| 101 | >7.5 | <4.5 |
| 124 | >6.0 | <4.5 |
| dMCL-1-2 (Papatzimas | >6.0 | <4.5 |
| et al., 2019) | | |

As illustrated in Table 9, the compounds of the present invention have the capability to decrease the viability of acute myeloid leukaemia cells, and thus can be useful in the treatment of cancer. Simultaneously, the compounds of the present invention have no/little impact on the viability of ARH-77, which further supports a specific, MCL-1 dependent activity of said compounds.

dMCL-1-2 is MCL-1 degrader described in Papatzimas et al., J. Med. Chem. 2019, 62, 5522-5540.

### Example 22: MV4-11 Cells: MCL-1 Protein Degradation, Western Blot

The effect of various compounds of the invention on MCL-1 protein degradation in the MV4-11 cell line was investigated, using the degradation assay protocol below.

MV4-11 cells were maintained in IMDM medium, supplemented with penicillin/streptomycin and 10% Fetal Bovine Serum (FBS). MV4-11 cells were collected, centrifuged (250 rcf, 5 min), resuspended in a growth medium containing 10% FBS and counted. The cell suspension was then adjusted to 1×10⁶ cells/ml using a growth medium containing 10% FBS. Afterwards, 0.5×10⁶ cells were seeded on 24-well plates in 0.5 mL of medium per well. Immediately after seeding, cells were pre-treated for 30 minutes with DMSO or pan-caspase inhibitor—Q-VD-Oph. Next, compounds from stocks pre-diluted in DMSO were added directly to the cell culture medium in appropriate concentrations (the final DMSO concentration was 0.5%). Following the 6 hours treatment with the compounds, cells were collected from the plates and suspended in a lysis buffer (2% SDS, 50 mM Tris, pH 8.0, 100 U/ml Pierce Universal Nuclease for Cell Lysis, complete EDTA-free Protease Inhibitor Cocktail) and incubated on ice for 45 min. The amount of protein was determined via BCA assay, and an equal quantity of each sample was loaded on the precast gel for protein separation. After the antibody staining for MCL-1, α-tubulin, and cleaved forms of PARP and caspase 3, the membranes were washed and signals developed.

Densitometric values for MCL-1 were normalized to total protein level (stain-free membrane). [%] of protein reduction is calculated in comparison to the cells treated with DMSO, which stand for 0%.

TABLE 10
MCL-1 protein degradation results in MV4-11 cells upon exemplary
compound treatment. The table shows densitometric values normalized
to the loading control and calculated as a % of DMSO control.

| Compound | | | |
|---|---|---|---|
| ID/ Conc. | MCL-1 protein abundance | | |
| [nM] | 1 | 10 | 100 |
| Compound 69 | C | B | A |
| Compound 69 + | C | B | B |
| Q-VD-Oph | | | |
| Compound 41 + | B | A | A |
| Q-VD-Oph | | | |

A - Amount of MCL-1 protein ≤25% calculated as % of DMSO control
B - Amount of MCL-1 protein >25% and ≤50% calculated as % of DMSO control
C - Amount of MCL-1 protein >50% and <75% calculated as % of DMSO control
D - Amount of MCL-1 protein ≥75% calculated as % of DMSO control

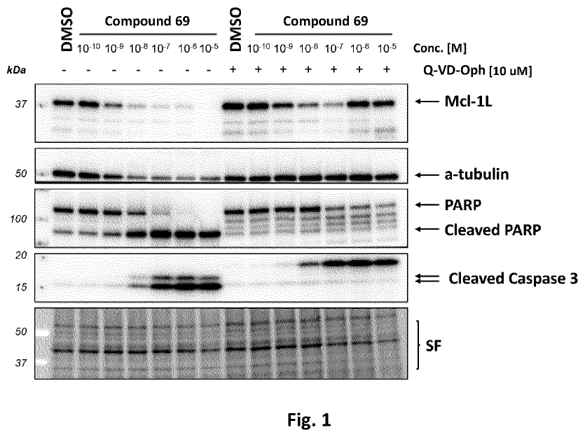

As presented in Table 10 and FIG. 1, the exemplary compound of the present invention induced dose-dependent degradation of MCL-1 protein in MV4-11 cells, resulting in strong apoptosis induction. In consequence, complete degradation of the MCL-1 protein was observed at the highest tested dose. When apoptosis was blocked, the exemplary compound induced expected, incomplete MCL-1 degradation, which could potentially save MCL-1-independent cells (e.g. normal cells) from death. This hypothesis is supported by the results from ARH-77 cells presented in Table 9, which show lack of cytotoxic activity of the exemplary compound in MCL-1-independent leukaemia cells.

### Example 23: Determination of the Cell Permeability and Target Engagement

### NanoBRET—In Cell CRBN Target Engagement Assay

Commercially available, NanoBRET In-cell CRBN Target Engagement Assay (NanoBRET CRBN TE Assay), which uses NanoLuc CRBN HEK293 cells and NanoBRET In-cell CRBN Tracer was used (N2910, N2912, PROMEGA) to determine the cell membrane permeability of prodrugs of MCL-1 degraders.

The assay was run in 96-well plate format (cat. 3600, Corning). NanoLuc CRBN HEK293 cells were treated with CRBN Tracer and compounds. Briefly, prepared was cell suspension (0.17×10⁶cells/ml) in Opti-MEM without phenol red (Life Technologies). The cells were treated with CRBN Tracer (20× concentrated, 5 μl/well. For the live mode: 0.8 μM and for the lytic: 0.17 μM final concentration) or DMSO vehicle control (20× concentrated, 5 μl/well). Such prepared cells were seeded into the wells of 96-well plate on top of compounds (100× concentrated, 1 μl/well; 11-point 2-fold dilution) pre-plated on them with the help of Echo 555 acoustic dispenser (Labcyte). For the lytic mode assay, Digitonin (0.05 mg/ml, 10× concentrated, 10 μl/well) was added additionally. In the live mode assay, the plate was incubated at 37° C., 5% CO₂ for 2 hours, and then at room temperature for 15 minutes to cool down. In the lytic mode assay, there was no incubation. The 3×Complete NanoBRET reagent (50 μl/well) was prepared according to the manufacturer's recommendations, appropriately for live and lytic mode. The plate was incubated for 2 minutes at room temperature and the luminescence and fluorescence readings followed using CLARIOstar Multimode Plate Reader (donor emission RLU—450 nm; acceptor emission RFU—610 nm). RBA value is an indicator of cell membrane permeability (the lower, the better) is calculated from absolute pIC₅₀ (the negative log of the absolute IC50 value when converted to molar) of the live mode assay (pIC₅₀ Live) and the lytic mode assay (pIC₅₀ Lytic).

$$RBA xi = 10 \hat{} ( - Absolute {pIC}_{50} Live xi ) / 10 \hat{} ( - Absolute {pIC}_{50} Lytic xi )$$

TABLE 11
NanoBRET pIC₅₀ [M] values for tested bifunctional
compounds in intact and lysed cells.

| Compound | pIC₅₀ (live) [M] | pIC₅₀ (lytic) [M] | RBA |
|---|---|---|---|
| 41 | 8.1 | 6.3 | 0.014 |
| 43 | 7.4 | 7.1 | 0.52 |
| 69 | 7.4 | 5.2 | 0.006 |
| 70 | 7.6 | 5.1 | 0.003 |
| 73 | 8.2 | 6.4 | 0.018 |
| 74 | 7.9 | 6.5 | 0.038 |
| 101 | 7.2 | 6.1 | 0.077 |
| 129 | 6.5 | 6.45 | 0.9 |
| 133 | 6.4 | 6.6 | 1.7 |
| dMCL-1-2 | 5.3 | 6.2 | 7.9 |
| (Papatzimas et | | | |
| al., 2019) | | | |

Based on the evidence, presented herein prodrug of MCL-1 degraders are cell membrane permeable. Importantly, the permeability for prodrug-modified degraders is 4-2600 times higher than for a non-modified dMCL-1-2 degrader indicating an enhanced cell membrane permeability for prodrugs.

### Abbreviations and Definitions

A list of the abbreviations used in the present application is shown below:

| Abbreviation | Meaning |
|---|---|
| CTG | CellTiter-Glo ® |
| BCA | bicinchoninic acid |
| TFA | trifluoroacetic acid |
| EDTA | ethylenediaminetetraacetic acid |
| FA | formic acid |
| FBS | fetal bovine serum |
| IMDM | Iscove's Modified Dulbecco's Medium |
| Tris | Tris(hydroxymethyl)aminomethane |
| DMSO | dimethylsulfoxide |
| THF | tetrahydrofuran |
| DMF | dimethylformamide |
| DMA | dimethylacetamide |
| DMAP | 4-dimethylaminopyridine |
| EDC | 1-ethyl-3-(3-dimethylaminopropyl)carbodiimide |
| HPLC | High performance liquid chromatography |
| NCS | N-chlorosuccinimide |
| DIPEA | N,N-diisopropylethylamine |
| PARP | Poly (ADP-ribose) polymerase |
| HATU | Hexafluorophosphate Azabenzotriazole Tetramethyl |
| | Uronium (1-[Bis(dimethylamino)methylene]-1H-1,2,3- |
| | triazol[4,5-b]pyridinium-2-oxide hexafluorophosphate) |
| Pd(dppf)Cl₂ | [1,1′-bis(diphenylphosphino)ferrocene]dichloro- |
| CH₂Cl₂ | palladium(II), complex with dichloromethane |
| RT | Room temperature |

As used herein, the term “room temperature” means a temperature of between 20° C. and 25° C.

The invention is further described with reference to the following clauses:
1. A compound of formula (I)

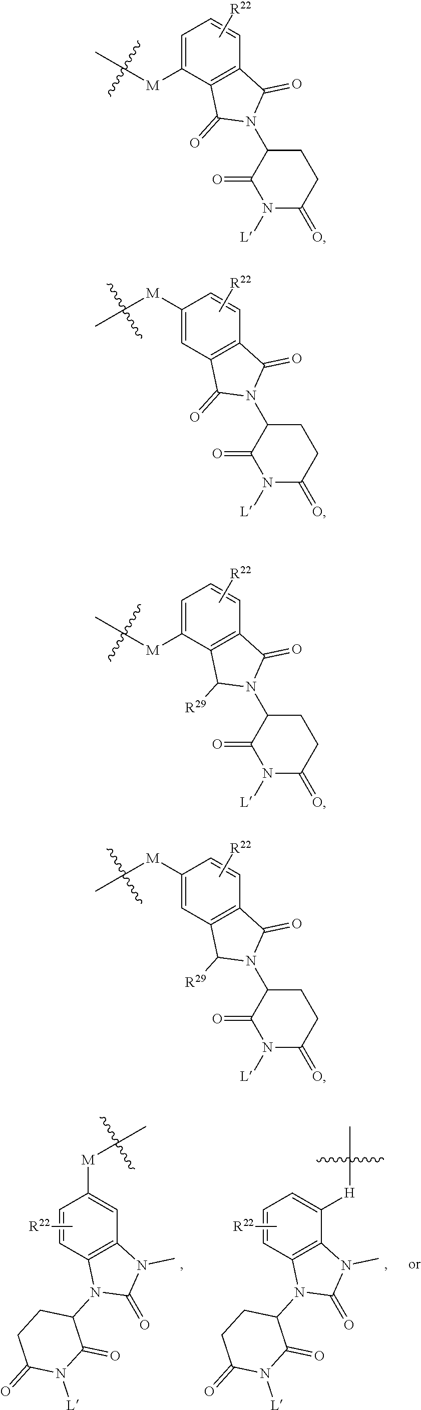

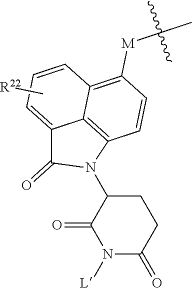

or a salt, solvate, hydrate or isomer thereof,
wherein [ligase ligand moiety] is:

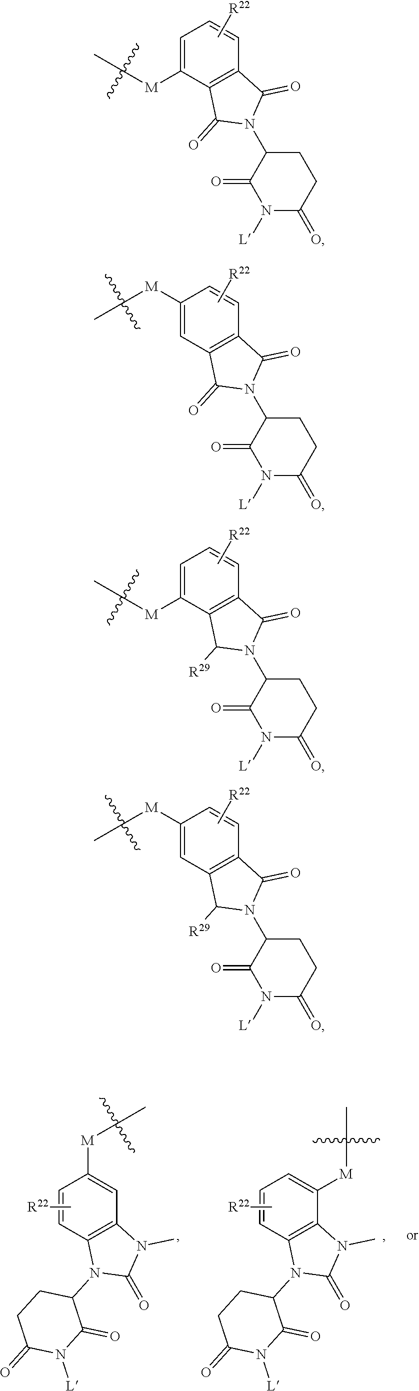

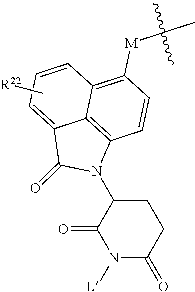

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   R²⁹ is hydrogen or Me
   L′ is hydrogen, alkyl, benzyl, acetyl or pivaloyl;
wherein [MCL-1 ligand prodrug moiety] is

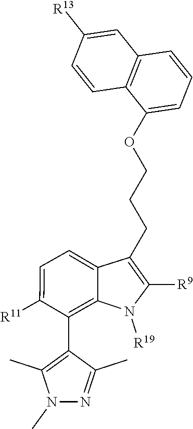

wherein
   R¹¹ is halogen;
   R¹³ is halogen;
   R¹⁹ is a bond connected to R¹⁸ of the linker;
and
   (i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
   wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, —C(O)NMe₂;
   each R^{p} is independently —(CH₂)ₘOC(O)(unsubstituted C₁-C₆alkyl), and
   each m is independently 1, 2, 3 or 4;
   or
   (ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me wherein each p is independently 2, 3, 4, 5, or 6;
and wherein [linker] has the following formula

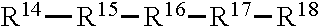

wherein
   R¹⁴ is —C₁-C₆ alkyl, —C₂₋₆ alkenyl, —C₂₋₆ alkynyl, —C₁₋₆ alkyl-N(C₁₋₆ alkyl)-, —C(O)—, —SO₂— or is absent
   R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁-C₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(C₂H₄—O)ₓ, —(C₃H₆—O)ₓ, or is absent
   x is 1-10
   y is 2-10
   R¹⁸ is —C₁₋₆ alkyl, cycloalkyl, —CH₂—NH—C(O)—, heterocycloalkyl, or is absent
   wherein at least one of R¹⁴—R¹⁸ is present.
2. The compound of clause 1, wherein [ligase ligand moiety] is:

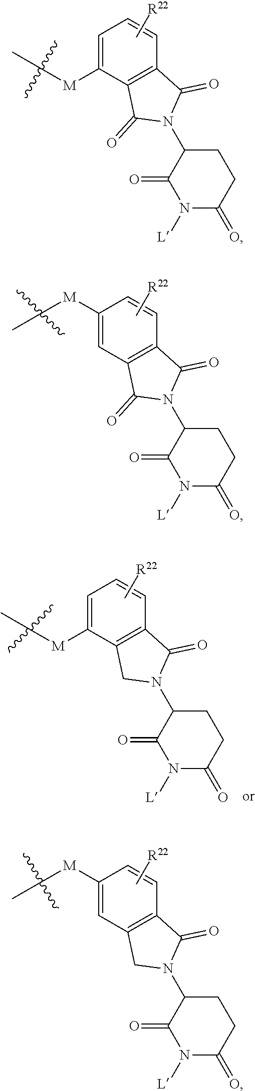

3. The compound of clause 1, wherein [ligase ligand moiety] is:

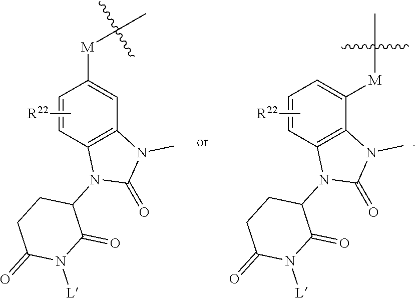

4. The compound of any preceding clause, wherein R²² is hydrogen.
5. The compound of any preceding clause, wherein L′ is hydrogen.
6. The compound of any preceding clause, wherein M is O or NH, or is absent.
7. The compound of any one of clauses 1-2 and 4-6, wherein [ligase ligand moiety] is:

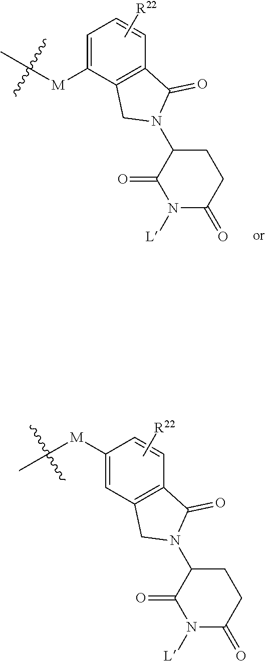

8. The compound of clause 7, wherein [ligase ligand moiety] is

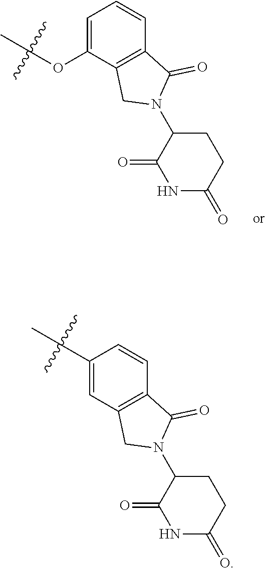 [SMILES: *Oc1cccc2c1CN(C1CCC(=O)NC1=O)C2=O ; "or" ; *c1ccc2c(c1)CN(C1CCC(=O)NC1=O)C2=O]

9. The compound of any preceding clause, wherein R¹⁴ is —C₁₋₆alkyl, —C₂₋₆alkenyl, —C₂₋₆alkynyl, —C(O)—, —SO₂— or is absent.
10. The compound of clause 9, wherein R¹⁴ is —C₁₋₆alkyl.
11. The compound of any preceding clause, wherein R¹⁵ is cycloalkyl, heterocycloalkyl, aryl, heteroaryl, —C₁₋₆ alkyl-NH—, -cycloalkyl-NH— or is absent.
12. The compound of clause 11, wherein R¹⁵ is heterocycloalkyl.
13. The compound of clause 12, wherein R¹⁵ is piperazine, bridged piperazine, piperazine N-oxide, piperazine cation,

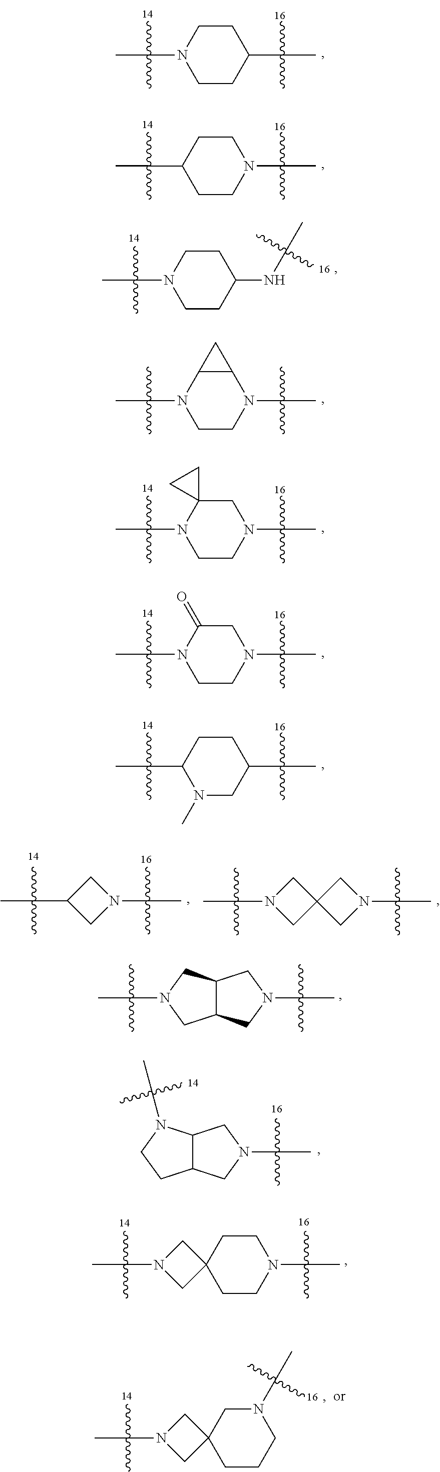

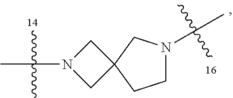 [SMILES: *N1CCC2(C1)CN(*)C2]

wherein [symbol] indicates attachment to R¹⁴ and [symbol] indicates attachment to R¹⁶.
14. The compound of clause 13, wherein R¹⁵ is piperazine or

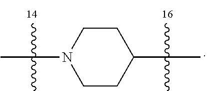 [SMILES: *C1CCN(*)CC1]

15. The compound of any preceding clause, wherein R¹⁶ is —C₁-C₆ alkyl, —CH₂—C(O)—NH— or —C(O)—.
16. The compound of any preceding clause, wherein R¹⁶ is —C₁-C₆ alkyl or —C(O)—.
17. The compound of any preceding clause, wherein R¹¹ is absent.
18. The compound of any preceding clause, wherein R¹⁸ is —C₁₋₆ alkyl, heterocycloalkyl, or is absent
19. The compound of clause 18, wherein R¹⁸ is —C₁₋₆ alkyl or heterocycloalkyl.
20. The compound of clause 19, wherein R¹⁸ is —C₁₋₆ alkyl or piperazine.
21. The compound of any preceding clause, wherein [linker] is selected from

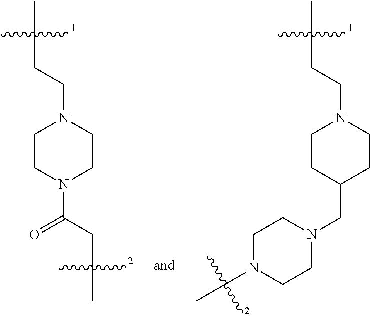 [SMILES: *CCN1CCN(C(=O)C*)CC1 ; *CCN1CCC(CN2CCN(*)CC2)CC1]

wherein
   [symbol] indicates attachment to [MCL-1 ligand prodrug moiety] and
   [symbol] indicates attachment to [ligase ligand moiety].
22. The compound of any preceding clause, wherein R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolyl cation.
23. The compound of clause 22, wherein R⁹ is —C(O)O(C₁-C₆alkyl), —C(O)Ocycloalkyl, —C(O)Oheterocycloalkyl, —C(O)Oaryl, or —P(O)(OR^{p})₂, wherein said C₁-C₆alkyl is substituted by one or more groups selected from —OC(O)Me, —OC(O)Et, —OC(O)ⁱPr, —OC(O)C(CH₃)₃, —OC(O)CH₂OH, —OC(O)CH(CH₃)OH, —OC(O)C(CH₃)₂OH, —OC(O)CH₂CH₂OH, —OC(O)CH(OH)CHMe₂, —OC(O)CH(OH)CH₂CHMe₂, —OC(O)CH₂CH(OH)Me, —OC(O)CH₂C(OH)Me₂, —OC(O)CH(CH₂OH)(NHC(O)Me), —OC(O)CH(NHC(O)Me)(isobutyl), —OC(O)CH(OH)aryl, —OC(O)CH(OH)CH₂aryl, —OC(O)heterocycloalkyl, —OC(O)cycloalkyl, —OC(O)aryl, —OC(O)heteroaryl, —OC(O)CH₂CH₂aryl, —OC(O)CH(NH₂)ⁱPr, —OC(O)CH(ⁱPr)NHC(O)O^{t}Bu, —OC(O)CH₂OCH₂CH₂OMe, —OC(O)CH₂(OCH₂CH₂)₂OMe, —OC(O)OMe, —OC(O)OEt, —OC(O)OⁱPr, —OC(O)Ocycloalkyl, —OC(O)OCH₂CH₂OEt, —OC(O)O(CH₂CH₂O)₂Et, —OC(O)O(CH₂CH₂O)₃Et, —OC(O)O(CH₂CH₂O)₃CH₂CH₂OP(O)(OH)₂, —OC(O)OCH₂CH₂SMe, —OC(O)OCH₂CH₂S(O)Me, —OC(O)OCH₂CH₂S(O)₂Me, —OC(O)OCH₂heteroaryl, —OC(O)OCH₂heterocycloalkyl, —OC(O)OCH₂CH₂OCH₂aryl —OC(O)OCH₂CH₂C(O)NMe₂, —OC(O)O(CH₂)₆NH₂, —OC(O)O(CH₂)₆NMe₂, —OC(O)OCH(CH₂OEt)₂, —OC(O)NHCH₂CH₂OMe, —OC(O)NMeCH₂CH₂OMe, —SMe, —S(O)Me, —P(O)(OH)₂, —OP(O)(OH)₂, —C(O)NMe₂, —C(O)heterocycloalkyl, heteroaryl, heterocycloalkyl, —N⁺(Me)₃, —N⁺(Me)₂cycloalkyl, N-methyl morpholinium cation, N-methyl piperidinium cation and N-methyl imidazolium cation.
24. The compound of clause 23, wherein R⁹ is —C(O)O(C₁-C₆alkyl) wherein said C₁-C₆alkyl is substituted by one or more groups selected from —OC(O)C(CH₃)₃, —OC(O)CH₂CH₂OH, —OC(O)C(CH₃)₂OH, —OC(O)heterocycloalkyl, —OC(O)CH₂OCH₂CH₂OMe, —OC(O)OiPr, —OC(O)Ocycloalkyl, —OC(O)O(CH₂CH₂O)₂Et, —OP(O)(OH)₂, heteroaryl or heterocycloalkyl.
25. The compound of clause 24, wherein R⁹ is —C(O)OCH₂OC(O)C(CH₃)₃, —C(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂OMe, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, —C(O)OCH₂CH₂CH₂piperazine.
26. The compound of any one of clauses 1-21, wherein R⁹ is —C(O)O(CH₂)ₚNMe₂, —C(O)O(CH₂)ₚNHMe, —C(O)OCH₂CH(OH)CH₂OH, C(O)OCH₂CH₂CMe₂OH, or —C(O)OCH₂CH₂SO₂Me wherein each p is independently 2, 3, 4, 5, or 6.
27. The compound of clause 26, wherein R⁹ is —C(O)OCH₂CH₂NMe₂, —C(O)OCH₂CH(OH)CH₂OH or —C(O)OCH₂CH₂SO₂Me.
28. The compound of any preceding clause, wherein R¹¹ is Cl.
29. The compound of any preceding clause, wherein R¹³ is F.
30. The compound of clause 1, which is selected from:

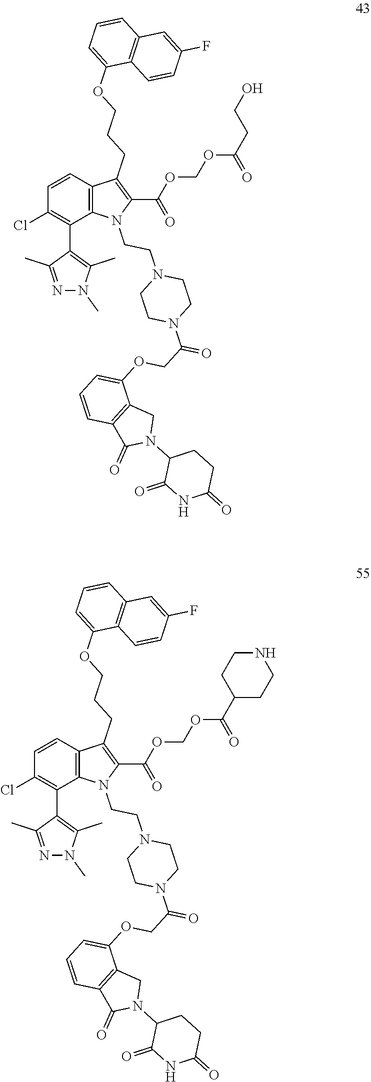 [SMILES: Cc1nn(C)c(C)c1-c1c(Cl)ccc2c(CCCOc3cccc4cc(F)ccc34)c(C(=O)OCOC(=O)CCO)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C3CCNCC3)n(CCN3CCN(C(=O)COc4cccc5c4CN(C4CCC(=O)NC4=O)C5=O)CC3)c12]

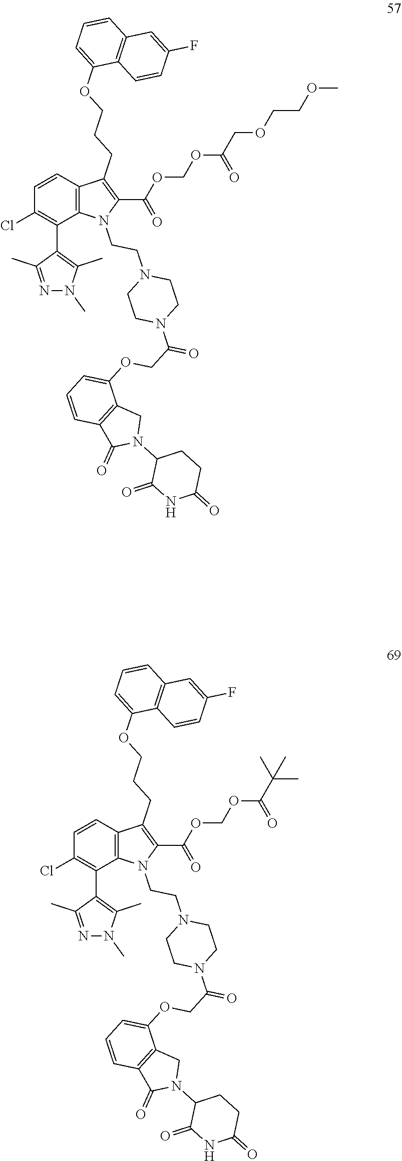

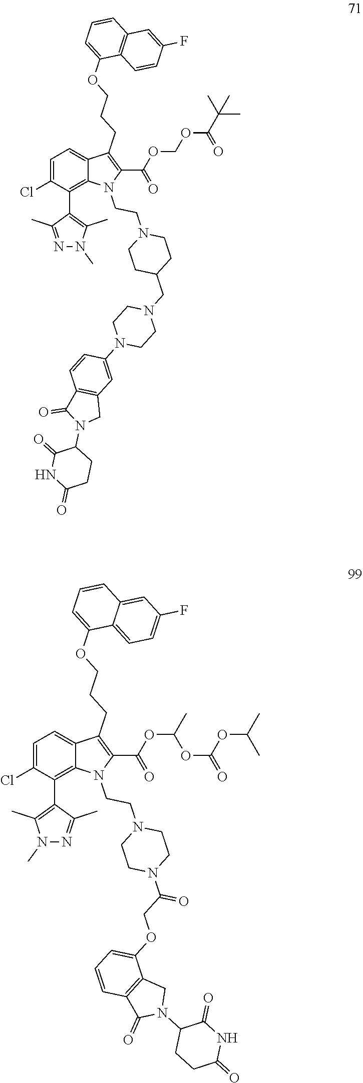

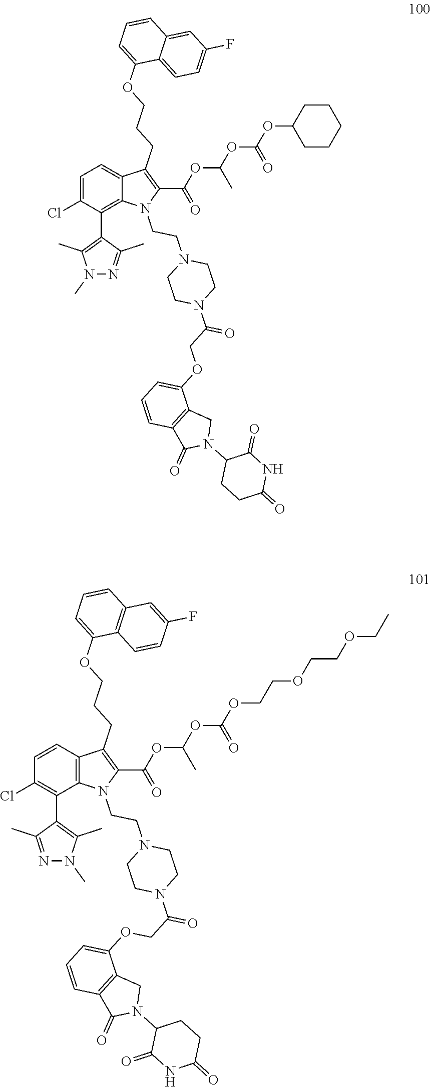 [SMILES: Cc1nn(C)c(C)c1-c1c(Cl)ccc2c(CCCOc3cccc4cc(F)ccc34)c(C(=O)OC(C)OC(=O)OC3CCCCC3)n(CCN3CCN(C(=O)COc4cccc5c4CN(C4CCC(=O)NC4=O)C5=O)CC3)c12 ; CCOCCOCCOC(=O)OC(C)OC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1]

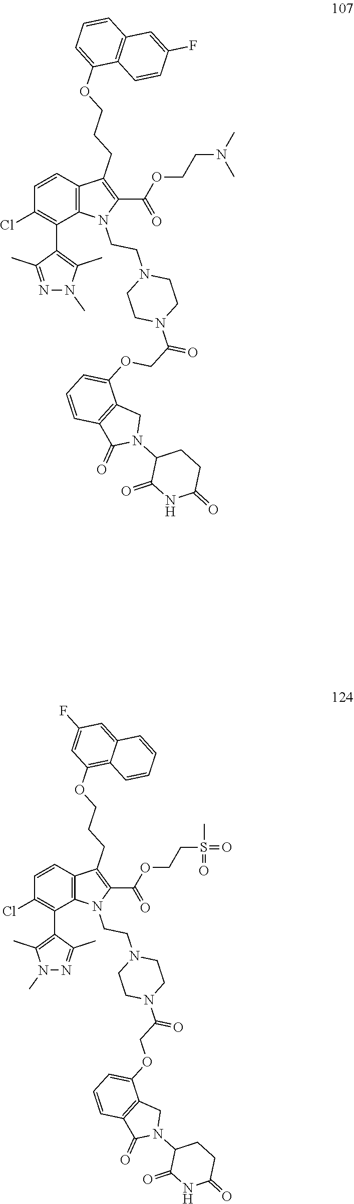

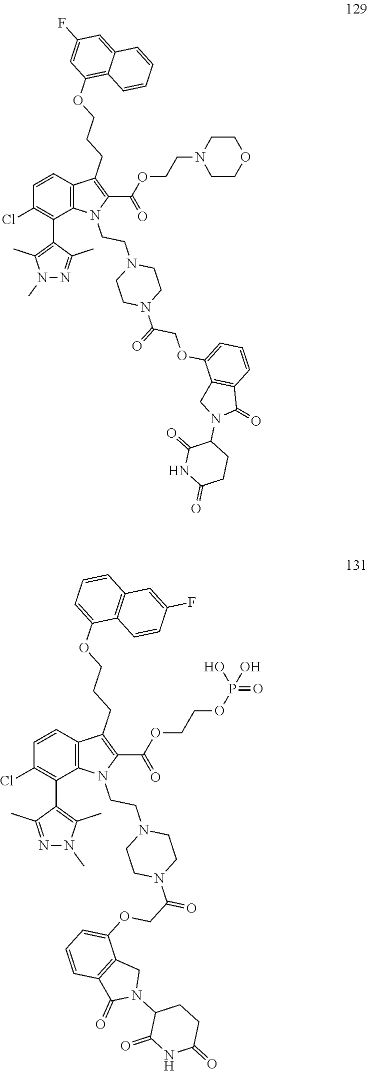 [SMILES: Cc1nn(C)c(C)c1-c1c(Cl)ccc2c(CCCOc3cc(F)cc4ccccc34)c(C(=O)OCCN3CCOCC3)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCOP(=O)(O)O)n(CCN3CCN(C(=O)COc4cccc5c4CN(C4CCC(=O)NC4=O)C5=O)CC3)c12]

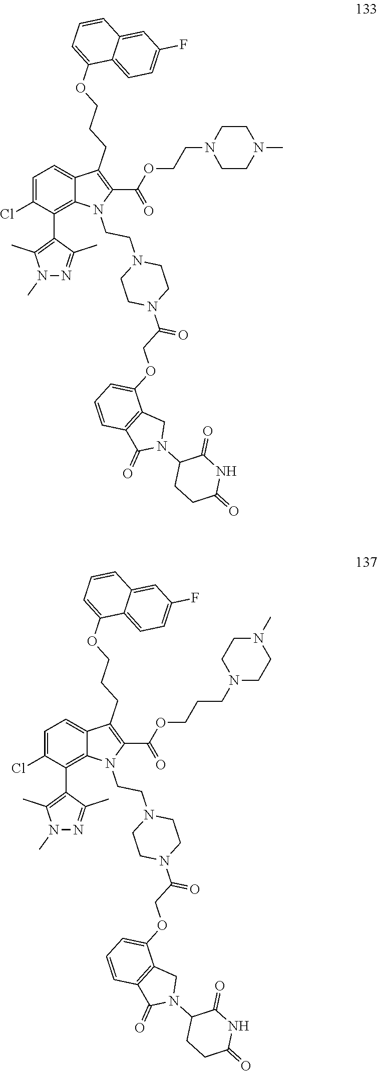

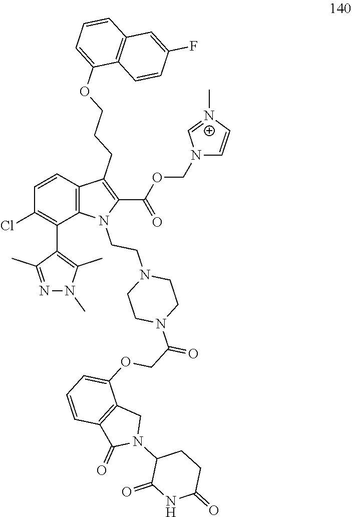 [SMILES: Cc1nn(C)c(C)c1-c1c(Cl)ccc2c(CCCOc3cccc4cc(F)ccc34)c(C(=O)OCn3cc[n+](C)c3)n(CCN3CCN(C(=O)COc4cccc5c4CN(C4CCC(=O)NC4=O)C5=O)CC3)c12]

31. The compound of clause 30, which is selected from:

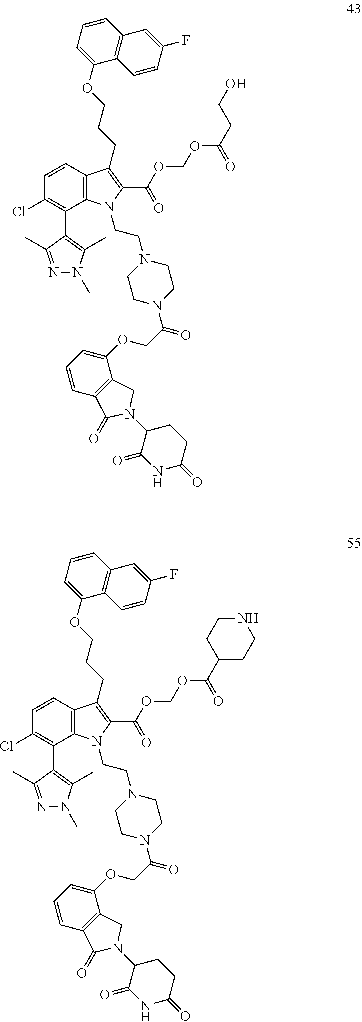 [SMILES: Cc1nn(C)c(C)c1-c1c(Cl)ccc2c(CCCOc3cccc4cc(F)ccc34)c(C(=O)OCOC(=O)CCO)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C3CCNCC3)n(CCN3CCN(C(=O)COc4cccc5c4CN(C4CCC(=O)NC4=O)C5=O)CC3)c12]

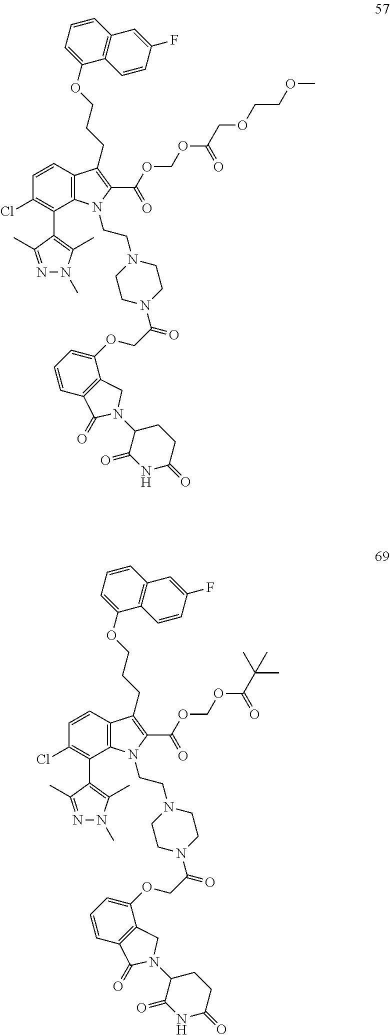 [SMILES: COCCOCC(=O)OCOC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1 ; Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4cccc5c4CN(C4CCC(=O)NC4=O)C5=O)CC3)c12]

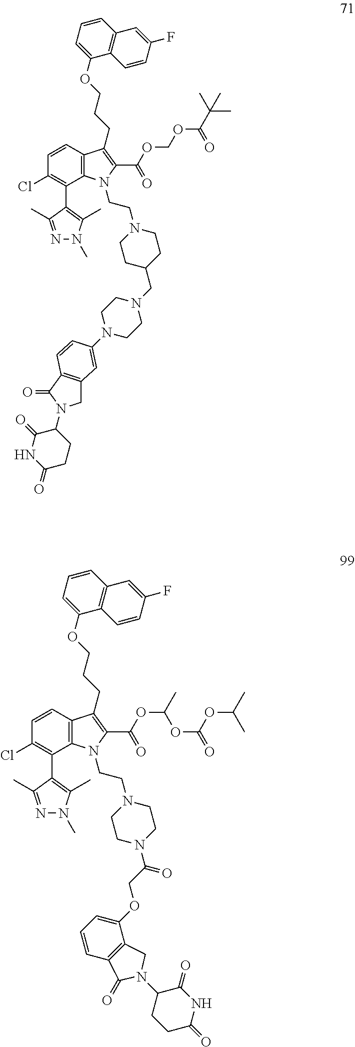 [SMILES: Cc1nn(C)c(C)c1-c1c(Cl)ccc2c(CCCOc3cccc4cc(F)ccc34)c(C(=O)OCOC(=O)C(C)(C)C)n(CCN3CCC(CN4CCN(c5ccc6c(c5)CN(C5CCC(=O)NC5=O)C6=O)CC4)CC3)c12 ; Cc1nn(C)c(C)c1-c1c(Cl)ccc2c(CCCOc3cccc4cc(F)ccc34)c(C(=O)OC(C)OC(=O)OC(C)C)n(CCN3CCN(C(=O)COc4cccc5c4CN(C4CCC(=O)NC4=O)C5=O)CC3)c12]

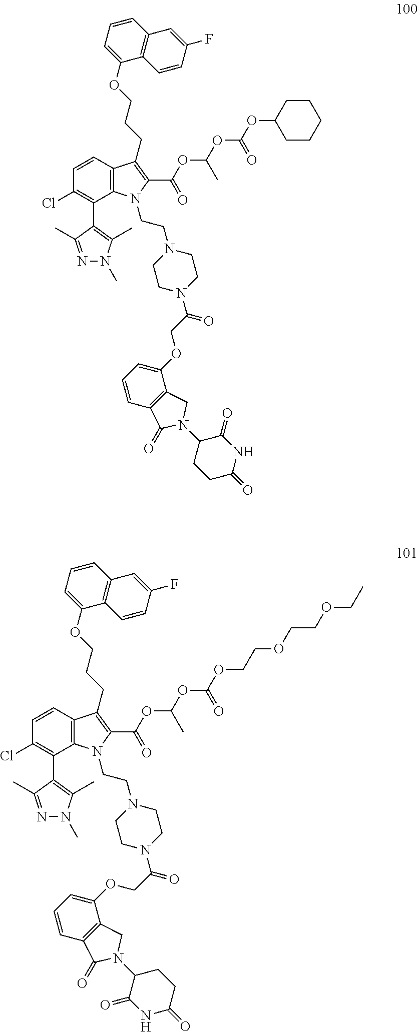 [SMILES: Cc1nn(C)c(C)c1-c1c(Cl)ccc2c(CCCOc3cccc4cc(F)ccc34)c(C(=O)OC(C)OC(=O)OC3CCCCC3)n(CCN3CCN(C(=O)COc4cccc5c4CN(C4CCC(=O)NC4=O)C5=O)CC3)c12 ; CCOCCOCCOC(=O)OC(C)OC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1]

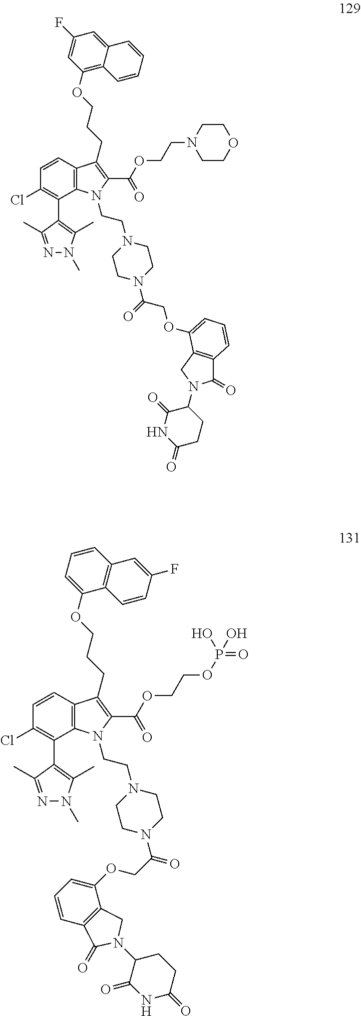 [SMILES: Cc1nn(C)c(C)c1-c1c(Cl)ccc2c(CCCOc3cc(F)cc4ccccc34)c(C(=O)OCCN3CCOCC3)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCOP(=O)(O)O)n(CCN3CCN(C(=O)COc4cccc5c4CN(C4CCC(=O)NC4=O)C5=O)CC3)c12]

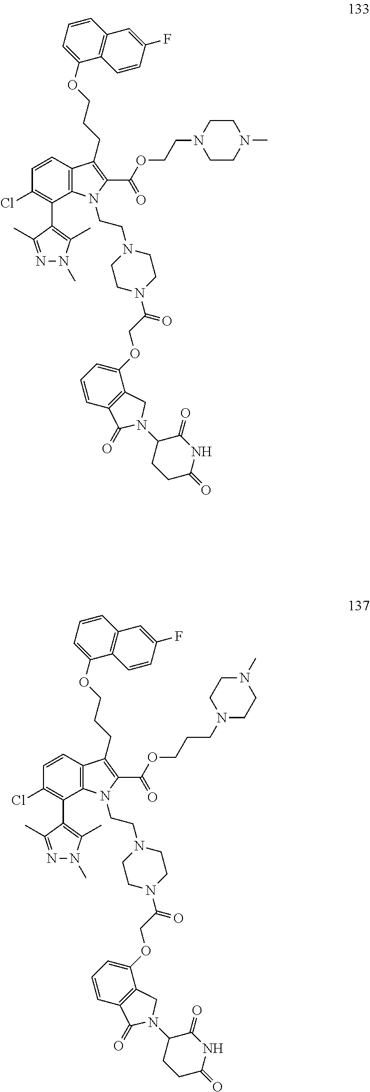 [SMILES: Cc1nn(C)c(C)c1-c1c(Cl)ccc2c(CCCOc3cccc4cc(F)ccc34)c(C(=O)OCCN3CCN(C)CC3)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCCN3CCN(C)CC3)n(CCN3CCN(C(=O)COc4cccc5c4CN(C4CCC(=O)NC4=O)C5=O)CC3)c12]

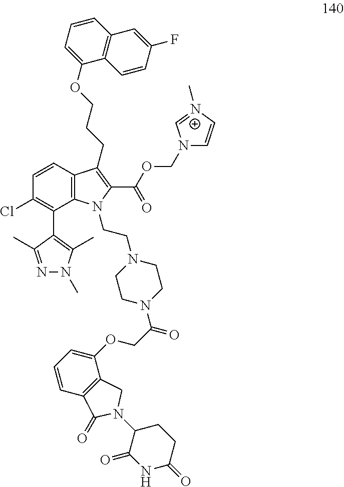 [SMILES: Cc1nn(C)c(C)c1-c1c(Cl)ccc2c(CCCOc3cccc4cc(F)ccc34)c(C(=O)OCn3cc[n+](C)c3)n(CCN3CCN(C(=O)COc4cccc5c4CN(C4CCC(=O)NC4=O)C5=O)CC3)c12]

32. The compound of clause 30, which is selected from:

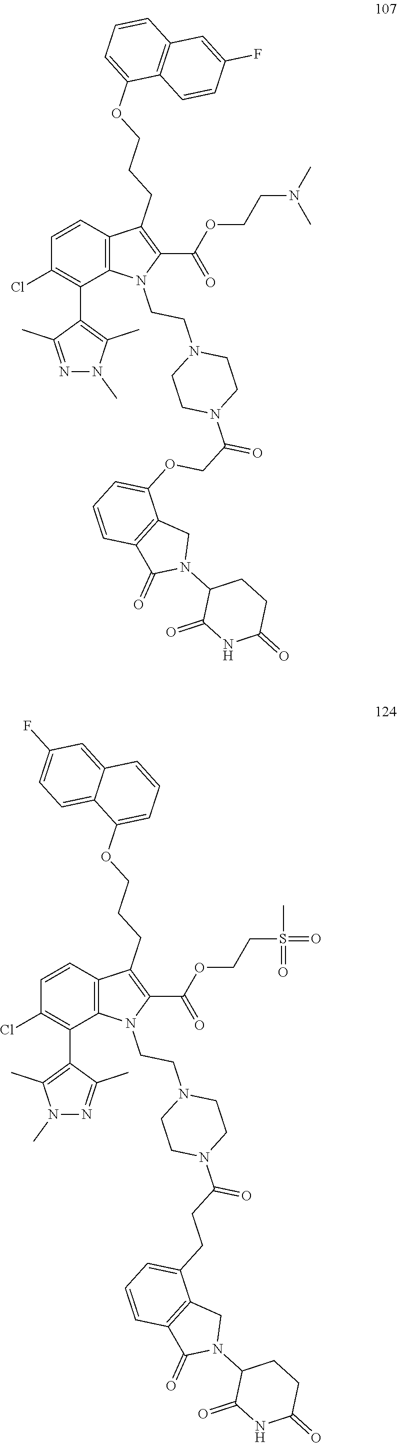

33. The compound of clause 30, which is selected from:

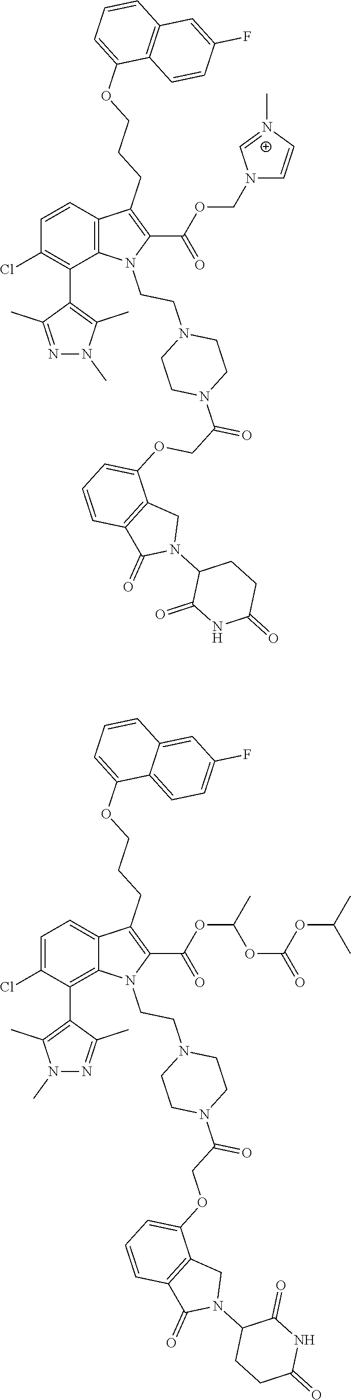 [SMILES: Cc1nn(C)c(C)c1-c1c(Cl)ccc2c(CCCOc3cccc4cc(F)ccc34)c(C(=O)OCn3cc[n+](C)c3)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OC(C)OC(=O)OC(C)C)n(CCN3CCN(C(=O)COc4cccc5c4CN(C4CCC(=O)NC4=O)C5=O)CC3)c12]

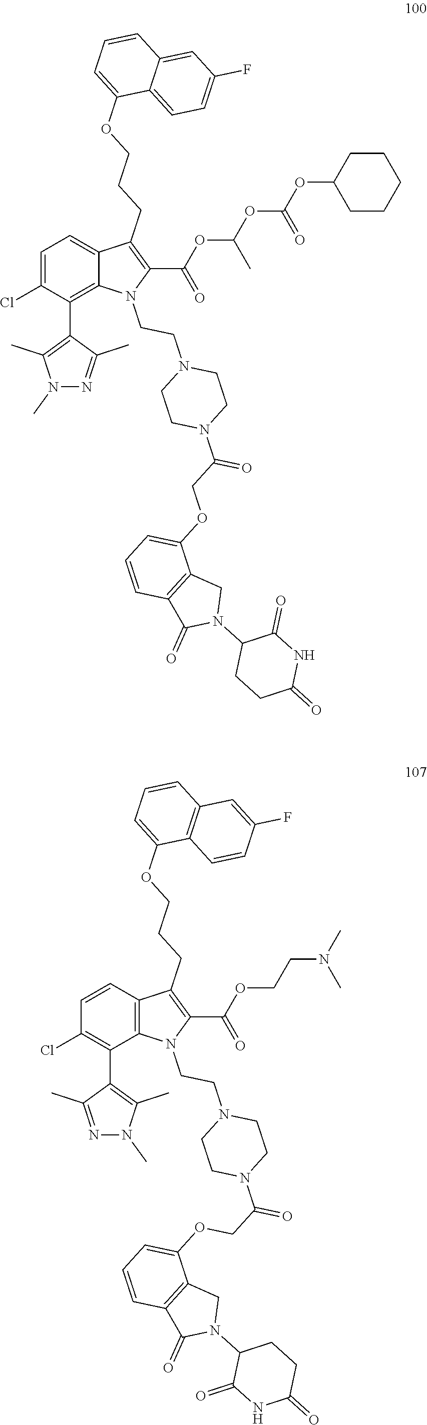

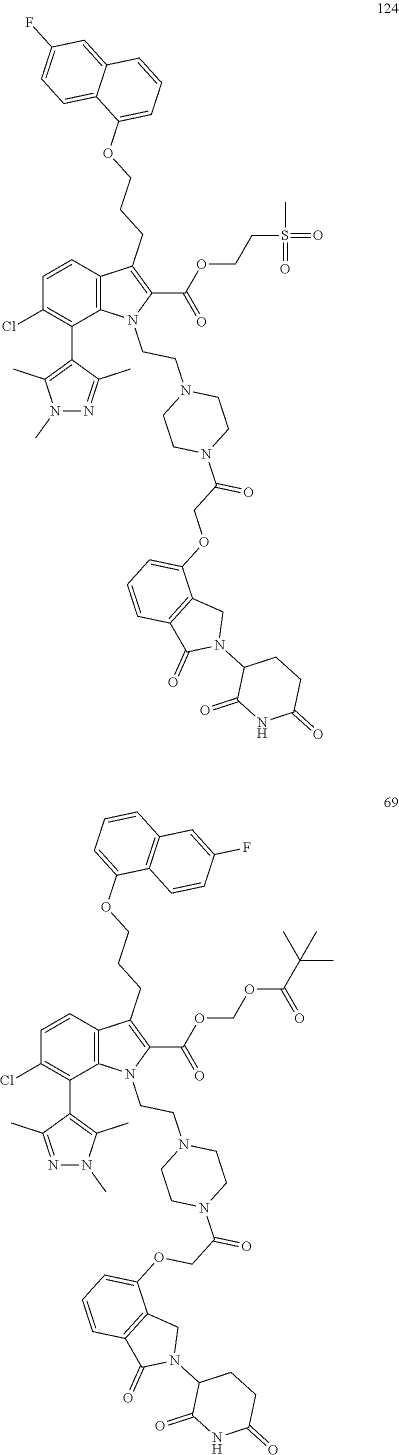 [SMILES: Cc1nn(C)c(C)c1-c1c(Cl)ccc2c(CCCOc3cccc4cc(F)ccc34)c(C(=O)OCCS(C)(=O)=O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4cccc5c4CN(C4CCC(=O)NC4=O)C5=O)CC3)c12]

34. The compound of any preceding clause, wherein unless otherwise specified each alkyl, alkenyl, alkynyl, aryl, heteroaryl and benzyl is unsubstituted.
35. A compound of formula (I)

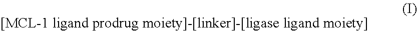

or a salt, solvate, hydrate or isomer thereof,
wherein [ligase ligand moiety] is:
   (a) Formula (IV)

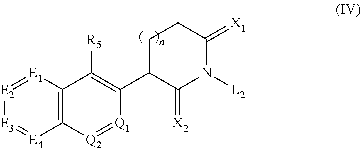

wherein:
each of X₁ and X₂ is independently O or S;
each of Q₁ and Q₂ is independently N or CRS, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
each Rⁱ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl; wherein R²¹ is a bond connected to R¹⁸ of the linker, and wherein Formula (IV) contains a single R²¹;
(b) Formula (VIIa), (VIIb), or (VIIc):

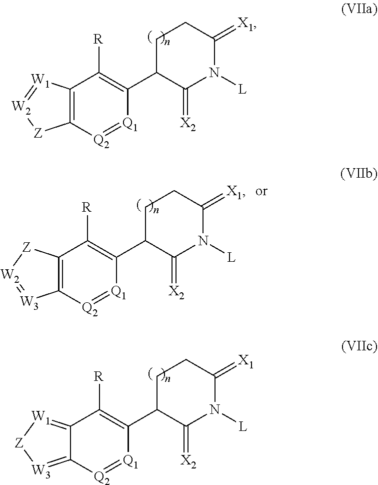

wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR*;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹;
each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII):

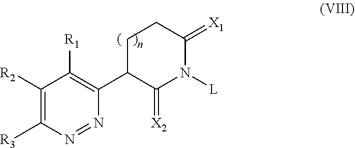

wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX):

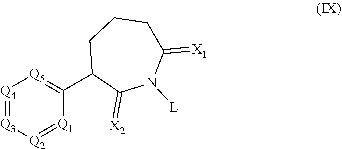

wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single R²¹;
wherein [MCL-1 ligand prodrug moiety] is

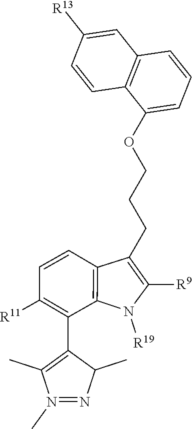

wherein
   R¹¹ is halogen;
   R¹³ is halogen;
   R¹⁹ is a bond connected to R¹⁴ of the linker;
and
   (i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
   wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, C(O)NMe₂;
   each R^{p} is independently —(CH₂)ₘOC(O)(unsubstituted C₁-C₆alkyl), and
   each m is independently 1, 2, 3 or 4;
   or
   (ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me, wherein each p is independently 2, 3, 4, 5, or 6;
and wherein [linker] has the following formula

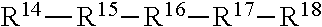

wherein
   R¹⁴ is —C₁₋₆ alkyl, —C₂₋₆ alkenyl, —C₂₋₆ alkynyl, —C₁₋₆ alkyl-N(C₁₋₆ alkyl)-, —C(O)—, —SO₂— or is absent
   R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁₋₆alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(C₂H₄—O)ₓ, —(C₃H₆—O)ₓ, or is absent
   x is 1-10
   y is 2-10
   R¹⁸ is —C₁₋₆ alkyl, cycloalkyl, —CH₂—NH—C(O)—, heterocycloalkyl, or is absent
wherein at least one of R¹⁴—R¹⁸ is present.
36. The compound of clause 35, wherein unless otherwise specified each alkyl, alkenyl, alkynyl, aryl, heteroaryl and benzyl groups is unsubstituted.
37. The compound of clause 35 or 36, wherein each Rⁱ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹.
38. The compound of clause 37, wherein each R^{b} is independently hydrogen, alkyl, cycloalkyl, or aryl.
39. The compound of clause 38, wherein the aryl is substituted with one or more groups selected from halogen, alkyl and O-haloalkyl, optionally wherein the halogen is Cl, the alkyl is methyl and the O-haloalkyl is O—CF₃.
40. The compound of any one of clauses 35-39, wherein one of E₁, E₂, E₃ and E₄ is N, and the remaining three of E₁, E₂, E₃ and E₄ are each CR′.
41. The compound of clause 40, wherein:
   (a) E₁ is N, and E₂, E₃ and E₄ are CR′;
   (b) E₂ is N, and E₁, E₃ and E₄ are CR′;
   (c) E₃ is N, and E₁, E₂ and E₄ are CR′; OR
   (d) E₄ is N, and E₁, E₂ and E₃ are CR′.
42. The compound of any one of clauses 35-39, wherein E₁, E₂, E₃ and E₄ are each CR′, optionally wherein E₁, E₂, E₃ and E₄ are each CH.
43. The compound of clause 42, wherein three of E₁, E₂, E₃ and E₄ are CH, and one of E₁, E₂, E₃ and E₄ is C-halogen, C-alkyl, C-alkenyl, C-alkynyl, C-aryl, C-heteroaryl, C-benzyl, C-haloalkyl, C-haloalkenyl, C-NH₂, C—NHR^{b}, C—NR^{b}₂, C—NR^{b}C(O)R^{b}, C—NR^{b}C(O)OR^{b}, C—NO₂, C—CN, C—C(O)R^{b}, C—C(O)OR^{b}, C—C(O)NH₂, C—C(O)NHR^{b}, C—C(O)NR^{b}₂, C—C(O)NHCHR^{b}₂, C—CHR^{b}NHC(O)NHR^{b}, C—CHR^{b}NHC(O)C(halogen)₂R^{b}, C—OR^{b}, C—OC(O)R^{b}, C—OC(O)OR^{b}, C—OC(O)NH₂, C—OC(O)NHR^{b}, C—OC(O)NR^{b}₂, C—SR^{b}, C—S(O)₂R^{b}, C—S(O)₂OR^{b}, C—S(O)₂NH₂, C—S(O)₂NHR^{b}, C—S(O)₂NR^{b}₂, C—NHS(O)₂R^{b}, —R², —O—R²¹, —NH—R²¹, —C(O)—R²¹, —C(O)—NH—R²¹, or —CH₂—NH—C(O)—R²¹.
44. The compound of clause 43, wherein:
   (a) E₂, E₃ and E₄ are each CH;
   (b) E₁, E₃ and E₄ are each CH; or
   (c) E₁, E₂ and E₄ are each CH.
45. The compound of any one of clauses 35-39, wherein two of E₁, E₂, E₃ and E₄ are N, and the remaining two of E₁, E₂, E₃ and E₄ are each CR′.
46. The compound of any one of clauses 35-39, wherein three of E₁, E₂, E₃ and E₄ are N, and the remaining one of E₁, E₂, E₃ and E₄ is CR′.
47. The compound of any one of clauses 35-46, wherein one of W₁, W₂ and W₃ is N, and the other of W₁, W₂ and W₃ is CR^{a}, optionally wherein R^{a} is H.
48. The compound of any one of clauses 35-46, wherein W₁, W₂ and W₃ are each CR^{a}.
49. The compound of any one of clauses 35-48, wherein W₁ is C—NH₂, C—NHR^{b} or C—NR^{b}₂; optionally C-NH₂.
50. The compound of any one of clauses 35-46, wherein W₁, W₂ and W₃ are each N.
51. The compound of any one of clauses 35-50, wherein Z is NH or N-alkyl, optionally NH or N-Me.
52. The compound of any one of clauses 35-51, wherein in Formula (IV), Formula (VIIa), Formula (VIIb) and Formula (VIIc):
   (a) Q₁ is N and Q₂ is CR;
   (b) Q₁ is N and Q₂ is N; or
   (c) Q₁ is CR and Q₂ is N, optionally wherein Q₁ is C—H or C-alkyl, further optionally wherein Q₁ is C—H or C-Me.
53. The compound of any one of clauses 35-52, wherein in Formula (IX) one of Q₁, Q₂, Q₃, Q₄ and Q₅ is N, and the remaining four of Q₁, Q₂, Q₃, Q₄ and Q₅ are each CR.
54. The compound of clause 53, wherein:
   (a) Q₁ is N;
   (b) Q₂ is N; or
   (c) Q₃ is N.
55. The compound of any one of clauses 35-52, wherein in Formula (IX) two of Q₁, Q₂, Q₃, Q₄ and Q₅ are N, and the remaining three of Q₁, Q₂, Q₃, Q₄ and Q₅ are each CR.
56. The compound of clause 55, wherein:
   (a) Q₁ and Q₂ are N, and Q₃, Q₄ and Q₅ are each CR;
   (b) Q₂ and Q₃ are N, and Q₁, Q₄ and Q₅ are each CR;
   (c) Q₁ and Q₃ are N, and Q₂, Q₄ and Q₅ are each CR;
   (d) Q₂ and Q₄ are N, and Q₁, Q₃ and Q₅ are each CR; or
   (e) Q₁ and Q₄ are N, and Q₂, Q₃ and Q₅ are each CR.
57. The compound of any one of clauses 35-52, wherein in Formula (IX) three of Q₁, Q₂, Q₃, Q₄ and Q₅ are N, and the remaining two of Q₁, Q₂, Q₃, Q₄ and Q₅ are each CR.
58. A compound of formula (I)

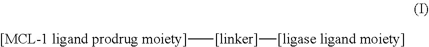

or a salt, solvate, hydrate or isomer thereof,
wherein [ligase ligand moiety] is:
   (a) Formula (Va) or (Vb):

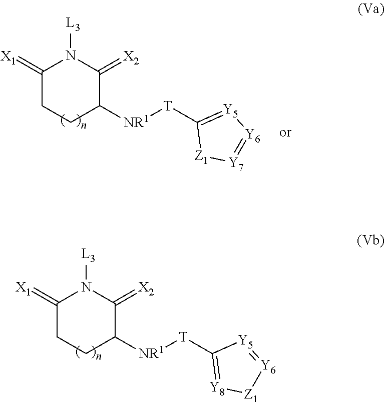

or a pharmaceutically acceptable salt or tautomer thereof,
   wherein
   each of X₁ and X₂ is independently O or S;
   Z₁ is O, S or NR⁶;
   T is is C═O or SO₂;
   R¹ is hydrogen, alkyl, cycloalkyl, alkenyl, cycloalkenyl, aryl, heteroaryl, or benzyl;
   each of Y₅, Y₆, Y₇, and Y₈ is independently N or CR⁷,
   wherein at least one of Y₅, Y₆ and Y₇ in Formula (Va) is CR⁷, and at least one of Y₅, Y₅ and Y₈ in Formula (Vb) is CR⁷;
   n is 0, 1 or 2;
   L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″2, —OR″″, —NR″″₂, or —S(O)₂R″″;
   each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
   each R″″ is independently hydrogen, alkyl, cycloalkyl, alkenyl, cycloalkenyl, aryl, heteroaryl, or benzyl;
   R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
   wherein R²¹ is a bond connected to R¹⁸ of the linker, and wherein formula (Va) and formula (Vb) each contain a single R²¹;
   wherein when Z₁ is O, then Y₆ is CR⁷ and
wherein when the compound is of Formula (Va), then
   (i) when each of Y₅, Y₆ and Y₇ is CR⁷, then at least one of R⁷ is not H;
   (ii) when Z₁ is NR⁶, then Y₆ and Y₇ are CR⁷;
   (iii) when Z₁ is S, then Y₅ is not C—OMe and Y₆ is not C—OMe;
   (iv) when Z₁ is S and Y₅ is C—NHCOMe, then Y₇ is not C—CH₂NR″″C(O)OR″″;
   (v) when Z₁ is S and Y₅ is N, then Y₆ is not C—H, C-aryl or C—C(O)OR″″; and
   (vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
and when the compound is of Formula (Vb), then:
   (vii) when each of Y₅, Y₆ and Y₈ is CR⁷, then at least one of R⁷ is not H;
   (viii) when Z₁ is S, then Y₅ is not C—COOH or C—NHC(O)Me, and Y₈ is not C—Br;
   (ix) when Z₁ is S and Y₆ is C—Br, then Y₈ is C—OR″″
   (x) when Z₁ is S, Y₅ is N and Y₆ is C—H or C—NH₂, then Y₈ is not C—H
   (xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷.
or
   (b) Formula (IIa) or (IIb):

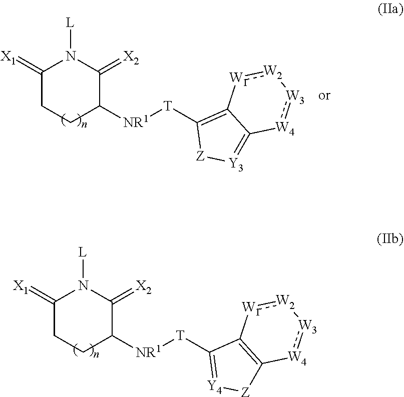

wherein
each of X₁ and X₂ is independently O or S;
Z is O, S or NR²;
T is C═O or SO₂;
Y₃ is N or CR;
Y₄ is N or CR;
[symbol] indicates a single or double bond, wherein
   when each [symbol] is a double bond, each of W₁, W₂, W₃ and W₄ is independently N or CR^{a}, wherein at least one of W₁, W₂, W₃ and W₄ is N, and
   when each [symbol] is a single bond, W₁, W₂, W₃ and W₄ are each CR^{a}₂ and Y₄ is CR;
n is 0, 1 or 2; [symbol]
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
wherein [MCL-1 ligand prodrug moiety] is

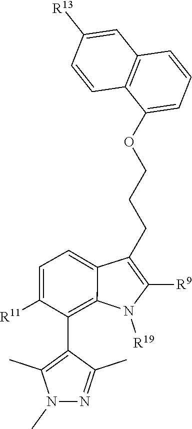

wherein
   R¹¹ is halogen;
   R¹³ is halogen;
   R¹⁹ is a bond connected to R¹⁴ of the linker;
and
   (i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
   wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, —C(O)NMe₂;
   each R^{p} is independently —(CH₂)ₘOC(O)(unsubstituted C₁-C₆alkyl), and
   each m is independently 1, 2, 3 or 4;
   or
   (ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me wherein each p is independently 2, 3, 4, 5, or 6;
and wherein [linker] has the following formula

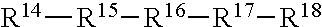

wherein
   R¹⁴ is —C₁-C₆ alkyl, —C₂₋₆ alkenyl, —C₂₋₆ alkynyl, —C₁₋₆ alkyl-N(C₁₋₆ alkyl)-, —C(O)—, —SO₂— or is absent
   R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁-C₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(C₂H₄—O)ₓ, —(C₃H₆—O)ₓ, or is absent
   x is 1-10
   y is 2-10
   R¹⁸ is —C₁₋₆ alkyl, cycloalkyl, —CH₂—NH—C(O)—, heterocycloalkyl, or is absent
wherein at least one of R¹⁴—R¹¹ is present.
59. The compound of clause 58, wherein unless otherwise specified each alkyl, alkenyl, alkynyl, aryl, heteroaryl and benzyl groups is unsubstituted.
60. The compound of any one of clauses 58-59, wherein when Z₁ is S in Formula (Vb), then Y₅ is not C— NHC(O)R″″ or —C(O)OR″″.
61. The compound of any one of clauses 58-60, wherein Z₁ is NR⁶.
62. The compound of any one of clauses 58-61, wherein each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —NR″″C(O)CH(OH)R″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —O—R²¹, —NH—R¹, —C(O)—NH—R¹, or —CH₂—NH—C(O)—R¹.
63. The compound of any one of clauses 58-62, wherein R¹ is hydrogen.
64. The compound of any one of clauses 58-63, wherein R⁶ is hydrogen.
65. The compound of any one of clauses 58-64, wherein [ligase ligand moiety] is of Formula (Va) and Y₅, Y₆ and Y₇ are each CR⁷.
66. The compound of clause 65, wherein
   Y₅ is —C—NHC(O)R″″,
   Y₆ is CH, and
   Y₇ is CH or CCl.
67. The compound of clause 66, wherein:
   L₃ is hydrogen;
   Z₁ is S;
   R¹ is hydrogen;
   T is C═O; and
   Y₇ is CH.
68. The compound of any one of clauses 58-64, wherein the compound is of Formula (Vb) and Y₅, Y₆ and Y₈ are each CR⁷.
69. The compound of clause 68, wherein:
   L₃ is hydrogen;
   Z₁ is S;
   R¹ is H;
   T is C═O;
   Y₅ is CH, C—OR″″, CCI, C—CN, or C—NHC(O)R″″;
   Y₆ is CH, CCI, C-alkyl, C-cycloalkyl, or C-haloalkyl; and
   Y₈ is CH, C—OR″″, C—NHC(O)R″″, C—NHC(O)OR″″, C—NHR″″, C—NH₂, or C—NHSO₂R″″;
   wherein, when Y₅ is CCI, then Y₆ is CH, C-alkyl, C-cycloalkyl, or C-haloalkyl; optionally wherein each R″″ is independently alkyl, cycloalkyl, aryl or benzyl.
70. The compound of clause 69, wherein:
   Y₅ is CH;
   Y₆ is CH or CCl; and
   Y₈ is C—OR″″ or C—NH₂, optionally C—OMe or C—NH₂.
71. The compound of any one of clauses 58-70, wherein:
   (a) Z is NR²; or
   (b) Z is S.
72. The compound of any one of clauses 58-71, wherein each [symbol] is a double bond.
73. The compound of any one of clauses 58-72, wherein L is hydrogen.
74. The compound of clause 72 or 73, wherein one of W₁, W₂, W₃ and W₄ is N, and the remaining three of W₁, W₂, W₃ and W₄ are each CR^{a}; optionally wherein W₄ is CR^{a}.
75. The compound of any one of clauses 58-72, wherein two of W₁, W₂, W₃ and W₄ is N, and the remaining two of W₁, W₂, W₃ and W₄ are each CR^{a}.
76. The compound of any one of clauses 58-72, wherein one of W₁, W₂, W₃ and W₄ is CR^{a}, and the remaining three of W₁, W₂, W₃ and W₄ are each N.
77. The compound of any one of clauses 58-76, wherein each R is independently hydrogen, halogen or —NR^{h}C(O)R^{h}.
78. A compound of formula (I)

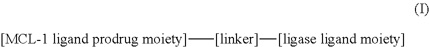

or a salt, solvate, hydrate or isomer thereof,
wherein [ligase ligand moiety] is:
   (a) Formula (VIa) or (Vib):

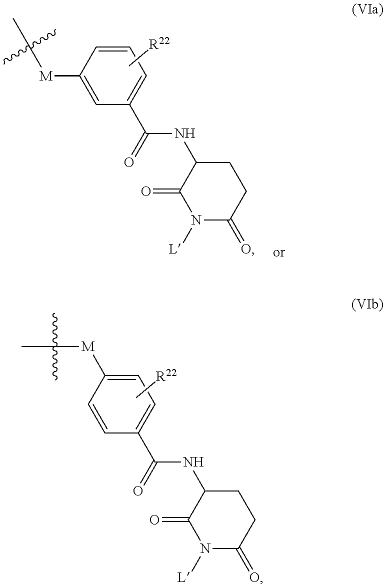

wherein
   M is O, S or NH, or is absent;
   [symbol] indicates attachment to R¹⁸ of the linker;
   R²² is hydrogen, halogen, —OMe, an amino group, heterocycloalkyl, or unsubstituted C₁-C₆ alkyl; and
   L′ is hydrogen, alkyl, benzyl, acetyl or pivaloyl
   (b) Formula (II):

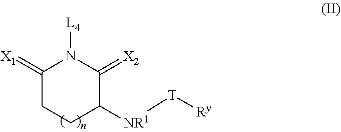

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{γ} is selected from

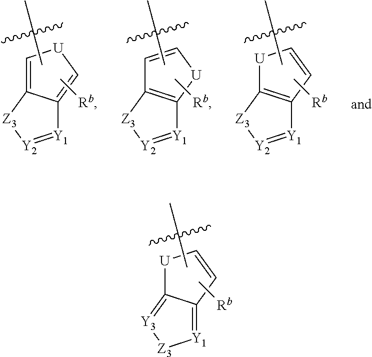

wherein [symbol] indicates attachment to T,
   Z₃ is O, S or NR³;
   U is O, S, NR^{b} or CRⁱ₂;
   each of Y₁, Y₂ and Y₃ is independently N or CR^{d};
   each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
   each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
   each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
   each R^{b} is independently alkyl, cycloalkyl, alkenyl, cycloalkenyl, aryl, heteroaryl, or benzyl;
   R²¹ is a bond connected to R¹⁸ of the linker, wherein Formula (II) contains a single R²¹;
   wherein,
   (i) when R^{γ}

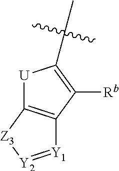

is 2 then Y₂ is CR^{d}; and
(ii) when R^{γ} is

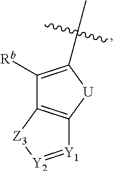

then Rⁱ in CRⁱ₂ is not hydrogen
or
   (c) Formula (III):

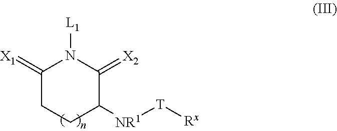

wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from

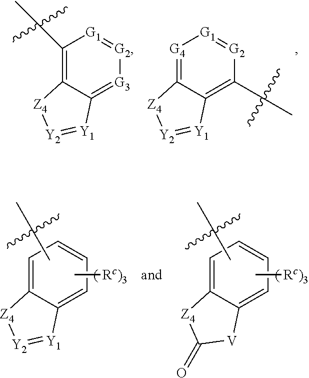

wherein [symbol]
indicates attachment to T,
   Z₄ is O, S or NR⁴;
   V is CR^{f}₂, NR⁴ or S;
   each of G₁, G₂, G₃ and G₄ is independently N or CR^{c},
   each of Y₁ and Y₂ is independently N or CR^{f},
   each R^{f} is independently hydrogen, halogen, alkyl, cycloalkyl, alkenyl, cycloalkenyl, alkynyl, aryl,
   fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f}then each R^{f}, together with the carbon atom to which it is attached, forms a 5- or 6-membered ring;
   each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
   each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
   each R^{b} is independently alkyl, cycloalkyl, alkenyl, cycloalkenyl, aryl, heteroaryl, or benzyl;
   R²¹ is a bond connected to R¹⁸ of the linker, wherein Formula (III) contains a single R²¹;
   wherein, when n=2, each R^{c} is hydrogen, and each of G₁, G₂, G₃ and G₄ is CR^{c}, then C=X₁ may be replaced by CH;
and wherein:
   (i) when R^{x} is

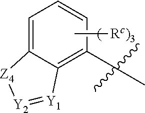

and Z₄ is NH, then L₁ is hydrogen, —CH₂C(O)OR^{b}, or —OR^{b};
(ii) when R^{x} is

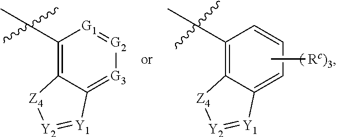

Z₄ is NR⁴, Y₁ is CR^{f}, and Y₂ is N, then R⁴ is not alkyl and at least one of R² and R is not H;
(iii) when R^{x} is

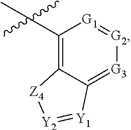

Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not

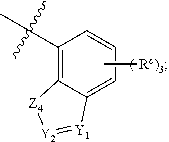

(v) when R^{x} is

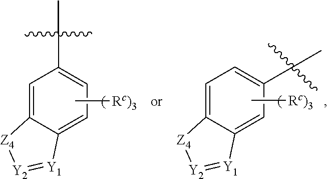

Z₄ is NR⁴, and Y₁ or Y₂ is N, then R⁴ is not alkyl;
(vi) when R^{x} is

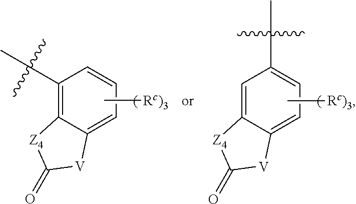

then n=1 or 2; and
(vii) when R^{x} is

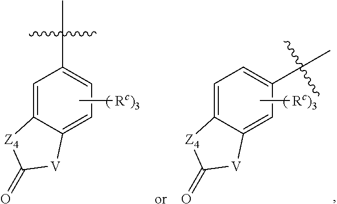

then Z₄=O or S
wherein [MCL-1 ligand prodrug moiety] is

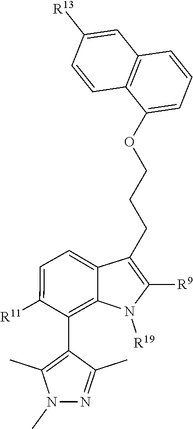

wherein
   R¹¹ is halogen;
   R¹³ is halogen;
   R¹⁹ is a bond connected to R¹⁴ of the linker;
and
   (i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
   wherein each R¹⁰ is independently —OH, —O(unsubstituted C₁-C₆alkyl), —Obn, —NH₂, —Nme₂, —NHC(O)(unsubstituted C₁-C₆alkyl), —NHC(O)O(unsubstituted C₁-C₆alkyl), heterocycloalkyl, heteroaryl, aryl, —C(O)Nme₂;
   each R^{p} is independently —(CH₂)ₘOC(O)(unsubstituted C₁-C₆alkyl), and
   each m is independently 1, 2, 3 or 4;
   or
   (ii) R⁹ is —C(O)O(CH₂)ₚNme₂, —C(O)O(CH₂)ₚNHMe, —C(O)OCH₂CH(OH)CH₂OH, —C(O)OCH₂CH₂CMe₂OH, or —C(O)OCH₂CH₂SO₂Me, wherein each p is independently 2, 3, 4, 5, or 6;
and wherein [linker] has the following formula

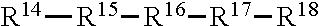

wherein
   R¹⁴ is —C₁-C₆ alkyl, —C₂₋₆ alkenyl, —C₂₋₆ alkynyl, C₁₋₆ alkyl-N(C₁₋₆ alkyl)-, —C(O)—, —SO₂— or is absent
   R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
   R¹⁶ is —C₁-C₆ alkyl, —C(O)—, —C(O)—NH—, —C(O)O—, —CH₂—C(O)—, —CH₂—C(O)—NH—, —CH₂—C(O)O— or is absent
   R¹⁷ is —CH₂(C₂H₄—O)_{y}, —(C₂H₄—O)ₓ, —(C₃H₆—O)ₓ, or is absent
   x is 1-10
   y is 2-10
   R¹⁸ is —C₁₋₆ alkyl, cycloalkyl, —CH₂—NH—C(O)—, heterocycloalkyl, or is absent
   wherein at least one of R¹⁴—R¹⁸ is present
79. The compound of clause 78, wherein unless otherwise specified each alkyl, alkenyl, alkynyl, aryl, heteroaryl and benzyl is unsubstituted.
80. The compound of any one of clauses 78-79, wherein in Formula (III):
   each of X₁ and X₂ is O;
   T is C═O;
   R¹ is hydrogen,
   L₁ is hydrogen,
   R^{x} is

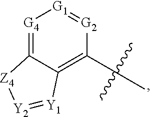

Z₄ is NR⁴;
each of G₁, G₂ and G₄ is CR^{c},
Y₁ is N, and
Y₂ is CR^{f}, wherein R^{f} is not hydrogen.
81. The compound of any one of clauses 78-80 wherein [ligase ligand moiety] is Formula (III):

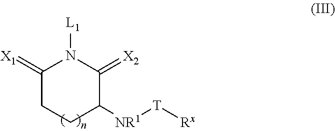

82. The compound of any one of clauses 78-81, wherein:
   (a) one of R^{c} is —O—R²¹, —NH—R²¹, —C(O)—NH—R²¹, or —CH₂—NH—C(O)—R²¹;
   (b) G₁ is C—O—R²¹, C—NH—R²¹, C—C(O)—NH—R²¹, or C—CH₂—NH—C(O)—R²¹;
   (c) G₂ is C—O—R²¹, C—NH—R²¹, C—C(O)—NH—R²¹, or C—CH₂—NH—C(O)—R²¹;
   (d) R⁴ is R²¹, —C(O)—NH—R²¹, or —CH₂—NH—C(O)—R²¹; or
   (e) one of R^{f} is —R²¹, —O—R²¹, —NH—R²¹, —C(O)—NH—R²¹, or —CH₂—NH—C(O)—R²¹.
83. The compound of any one of clauses 78-81, wherein Y₂ is C—R²¹, CO—R²¹, C—NH—R²¹, C—C(O)—NH—R²¹, or C—CH₂—NH—C(O)—R²¹.
84. The compound of any one of clauses 78-83, wherein [ligase ligand moiety] is selected from

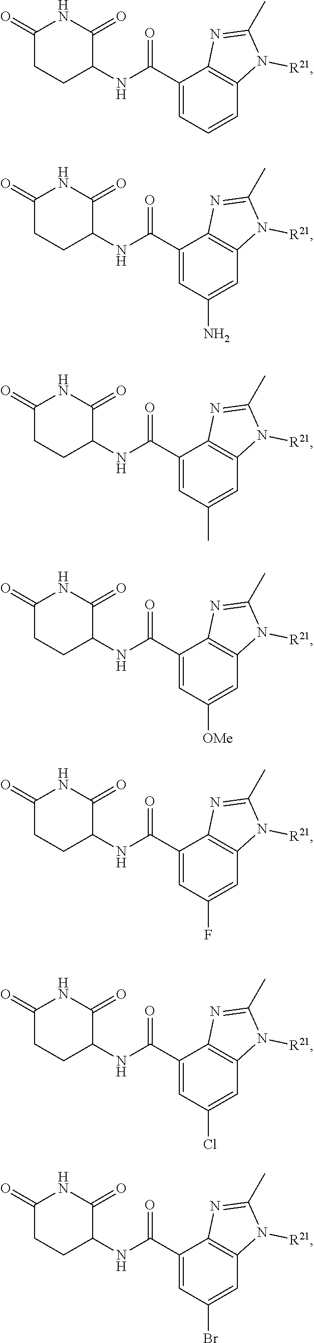

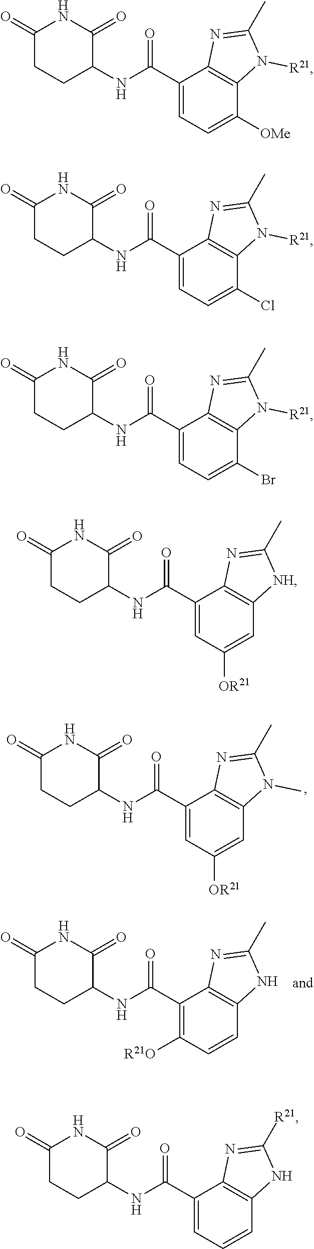

85. The compound of clause 84, wherein [ligase ligand moiety] is

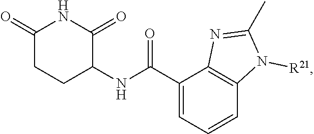

86. The compound of any one of clauses 35-85, wherein R¹⁴ is —C₁₋₆alkyl, —C₂₋₆alkenyl, —C₂₋₆alkynyl, —C(O)—, —SO₂— or is absent
87. The compound of clause 86, wherein R¹⁴ is —C₁-C₆alkyl.
88. The compound of any one of clauses 35-87, wherein R¹⁵ is cycloalkyl, heterocycloalkyl, aryl, heteroaryl, —C₁₋₆ alkyl-NH—, -cycloalkyl-NH— or is absent.
89. The compound of clause 88, wherein R¹⁵ is heterocycloalkyl.
90. The compound of clause 89, wherein R¹⁵ is piperazine, bridged piperazine, piperazine N-oxide, piperazine cation,

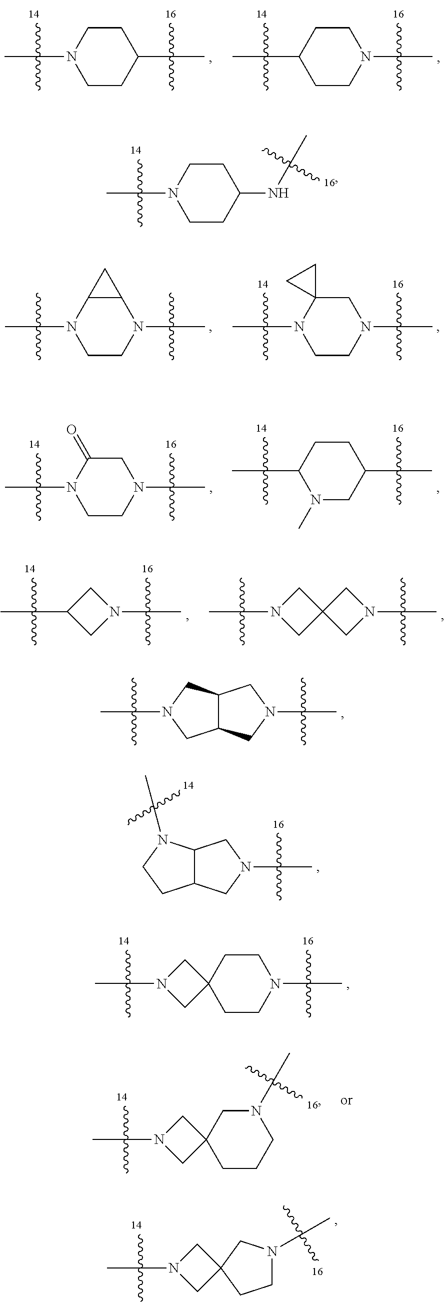

wherein [symbol] indicates attachment to R¹⁴ and [symbol] indicates attachment to R¹⁶.
91. The compound of clause 90, wherein R¹⁵ is piperazine or

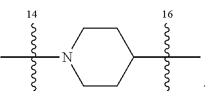 [SMILES: *C1CCN(*)CC1]

92. The compound of any one of clauses 35-91, wherein R⁶ is —C₁₋₆ alkyl, —CH₂—C(O)—NH— or —C(O)—.
93. The compound of clause 92, wherein R¹⁶ is —C₁alkyl or —C(O)—.
94. The compound of any one of clauses 35-93, wherein R¹¹ is absent.
95. The compound of any one of clauses 35-94, wherein R¹⁸ is —C₁₋₆ alkyl, heterocycloalkyl, or is absent.
96. The compound of clause 95, wherein R¹⁸ is —C₁₋₆ alkyl or heterocycloalkyl.
97. The compound of clause 96, wherein R¹⁸ is —C₁₋₆ alkyl or piperazine.
98. The compound of any one of clauses 35-97, wherein [linker] is selected from

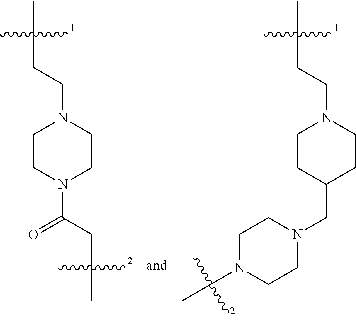 [SMILES: *CCN1CCN(C(=O)C*)CC1 ; *CCN1CCC(CN2CCN(*)CC2)CC1]

wherein
   [symbol] indicates attachment to [MCL-1 ligand prodrug moiety] and
   [symbol] indicates attachment to [ligase ligand moiety].
99. The compound of any one of clauses 35-98, wherein R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), -oC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolyl cation.
100. The compound of clause 99, wherein R⁹ is —C(O)O(C₁-C₆alkyl), —C(O)Ocycloalkyl, —C(O)Oheterocycloalkyl, —C(O)Oaryl, or —P(O)(OR^{p})₂, wherein said C₁-C₆alkyl is substituted by one or more groups selected from —OC(O)Me, —OC(O)Et, —OC(O)ⁱPr, —OC(O)C(CH₃)₃, —OC(O)CH₂OH, —OC(O)CH(CH₃)OH, —OC(O)C(CH₃)₂OH, —OC(O)CH₂CH₂OH, —OC(O)CH(OH)CHMe₂, —OC(O)CH(OH)CH₂CHMe₂, —OC(O)CH₂CH(OH)Me, —OC(O)CH₂C(OH)Me₂, —OC(O)CH(CH₂OH)(NHC(O)Me), —OC(O)CH(NHC(O)Me)(isobutyl), —OC(O)CH(OH)aryl, —OC(O)CH(OH)CH₂aryl, —OC(O)heterocycloalkyl, —OC(O)cycloalkyl, —OC(O)aryl, —OC(O)heteroaryl, —OC(O)CH₂CH₂aryl, —OC(O)CH(NH₂)ⁱPr, —OC(O)CH(ⁱPr)NHC(O)O^{t}Bu, —OC(O)CH₂OCH₂CH₂OMe, —OC(O)CH₂(OCH₂CH₂)₂OMe, —OC(O)OMe, —OC(O)OEt, —OC(O)OiPr, —OC(O)Ocycloalkyl, —OC(O)OCH₂CH₂OEt, —OC(O)O(CH₂CH₂O)₂Et, —OC(O)O(CH₂CH₂O)₃Et, —OC(O)O(CH₂CH₂O)₃CH₂CH₂OP(O)(OH)₂, —OC(O)OCh₂CH₂SMe, —OC(O)OCH₂CH₂S(O)Me, —OC(O)OCH₂CH₂S(O)₂Me, —OC(O)OCH₂heteroaryl, —OC(O)OCH₂heterocycloalkyl, —OC(O)OCH₂CH₂OCH₂aryl —OC(O)OCH₂CH₂C(O)NMe₂, —OC(O)O(CH₂)₆NH₂, —OC(O)O(CH₂)₆NMe₂, —OC(O)OCH(CH₂OEt)₂, —OC(O)NHCH₂CH₂OMe, -oC(O)NMeCH₂CH₂Ome, —SMe, —S(O)Me, —P(O)(OH)₂, —OP(O)(OH)₂, —C(O)NMe₂, —C(O)heterocycloalkyl, heteroaryl, heterocycloalkyl, —N⁺(Me)₃, —N⁺(Me)₂cycloalkyl, N-methyl morpholinium cation, N-methyl piperidinium cation and N-methyl imidazolium cation.
101. The compound of clause 100, wherein R⁹ is —C(O)O(C₁-C₆alkyl) wherein said C₁-C₆alkyl is substituted by one or more groups selected from —OC(O)C(CH₃)₃, —OC(O)CH₂CH₂OH, —OC(O)C(CH₃)₂OH, —OC(O)heterocycloalkyl, —OC(O)CH₂OCH₂CH₂OMe, —OC(O)OiPr, —OC(O)Ocycloalkyl, —OC(O)O(CH₂CH₂O)₂Et, —OP(O)(OH)₂, heteroaryl or heterocycloalkyl.
102. The compound of clause 101, wherein R⁹ is —C(O)O(C₁-C₆alkyl) wherein said C₁-C₆alkyl is substituted by —OC(O)C(CH₃)₃
103. The compound of clause 101, wherein R⁹ is —C(O)OCH₂OC(O)C(CH₃)₃, —C(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂OMe, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, —C(O)OCH₂CH₂CH₂piperazine.
104. The compound of clause 103, wherein R⁹ is —C(O)OCH₂OC(O)C(CH₃)₃.
105. The compound of any one of clauses 35-104, wherein [MCL-1 ligand prodrug moiety] is:

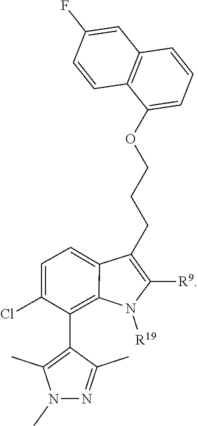

106. The compound of clause 78, wherein the compound is:

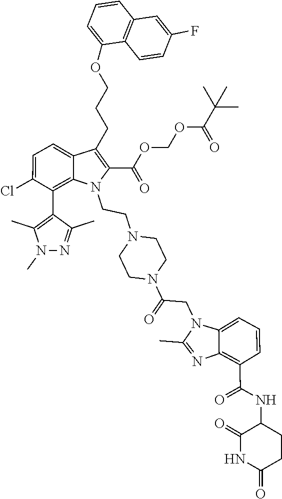 [SMILES: Cc1nn(C)c(C)c1-c1c(Cl)ccc2c(CCCOc3cccc4cc(F)ccc34)c(C(=O)OCOC(=O)C(C)(C)C)n(CCN3CCN(C(=O)Cn4c(C)nc5c(C(=O)NC6CCC(=O)NC6=O)cccc54)CC3)c12]

107. The compound of any preceding clause, wherein T is C═O.
108. The compound of any preceding clause, wherein:
   (a) X₁ and X₂ are O;
   (b) X₁ is O and X₂ is S;
   (c) X₁ is Sand X₂ is O; or
   (d) X₁ and X₂ are S.
109. The compound of any preceding clause, wherein n is 0.
110. The compound of any one of clauses 1-108, wherein n is 1 or 2.
111. The compound of clause 110, wherein n is 1.
112. The compound of clause 110, wherein n is 2.
113. A pharmaceutical composition comprising a compound of any one of clauses 1-112.
114. The compound of any one of clauses 1-112 or the pharmaceutical composition of clause 113, for use in medicine.
115. The compound of any one of clauses 1-112 or the pharmaceutical composition of clause 113, for use in the treatment of cancer.
116. The compound or composition for use of clause 115, wherein the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer.
117. The compound or composition for use of clause 116, wherein the cancer is multiple myeloma or acute myeloid leukaemia.
118. A method of treating cancer in a subject in need thereof, the method comprising administering to the subject an effective amount of a compound according to any one of clauses 1-112, or a pharmaceutical composition according to clause 113.
119. The method of clause 118, wherein the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer.
120. The method of clause 119, wherein the cancer is multiple myeloma acute myeloid leukaemia.
121. The method of any one of clauses 118-120, wherein the administration does not result in cytotoxicity in cardiomyocytes in the subject.
122. The method of any one of clauses 118-121, further comprising administering at least one additional active agent to the subject.
123. The method of clause 122, wherein the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.
124. The compound of any one of clauses 1-112 or the pharmaceutical composition of clause 112, for use in reversing resistance to chemotherapy or targeted cancer therapies.
125. A method of reversing resistance to chemotherapy or targeted cancer therapies in a subject in need thereof, the method comprising administering to the subject an effective amount of a compound according to any one of clauses 1-112, or a pharmaceutical composition according to clause 113.
126. A combined preparation of a compound of any one of clauses 1-112 and at least one additional active agent, for simultaneous, separate or sequential use in therapy.
127. The combined preparation of clause 126, wherein the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.
128. The combined preparation of any one of clauses 126-127 wherein the therapy is the treatment of cancer.
129. A method of reducing the cardiac cytotoxicity of an MCL-1 inhibitor, comprising coupling a cereblon binding moiety to the MCL-1 inhibitor, wherein the cereblon binding moiety is a [ligase ligand moiety] as defined in any one of clauses 1-112 and the MCL-1 inhibitor is an [MCL-1 ligand prodrug moiety] as defined in any one of clauses 1-112.

1. A compound of formula (I) 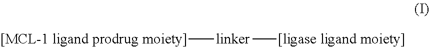
or a salt, solvate, hydrate or isomer thereof,
wherein [ligase ligand moiety] is: 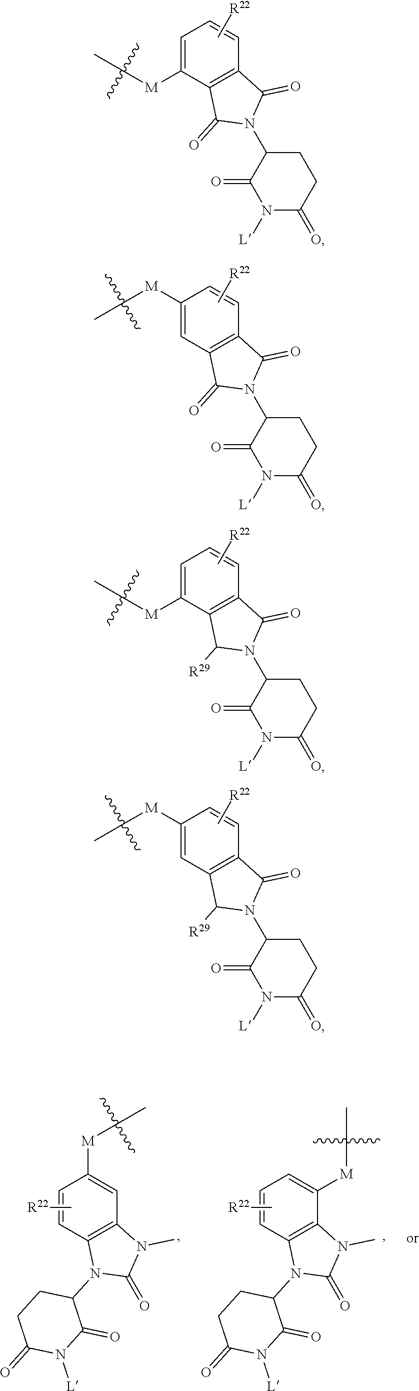 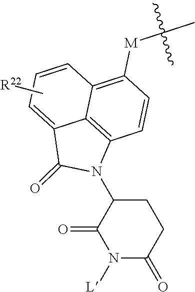
wherein
M is O, S or NH, or is absent; [symbol]
indicates attachment to R¹⁸ of the linker;
R²² is hydrogen, halogen, —OMe, an amino group, heterocycloalkyl, or unsubstituted C₁-C₆ alkyl; and
R²⁹ is hydrogen or Me
L′ is hydrogen, alkyl, benzyl, acetyl or pivaloyl;
wherein [MCL-1 ligand prodrug moiety] is a compound of Formula (A1), (A2), (A3) or (A4): 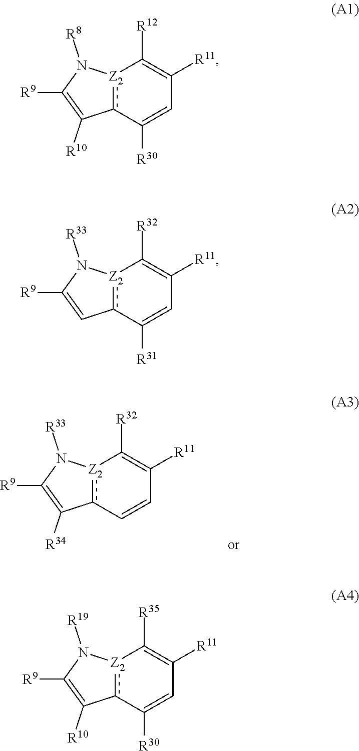
wherein [symbol]
is a single bond or a double bond;
each Z₂ is independently N or C, wherein when Z₂ is N, then [symbol] is a single bond; and when Z₂ is C, then [symbol] is a double bond;
each R¹¹ is interpedently H, halogen or C₁-C₆ alkyl,
R⁸ is C₁-C₆ alkyl substituted with a piperazine;
in each of Formula (A1) and Formula (A4) one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹³, wherein R¹³ is phenyl, napthyl or tetraline, wherein the phenyl, naphthyl or tetraline is optionally substituted with at least one substitutent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is 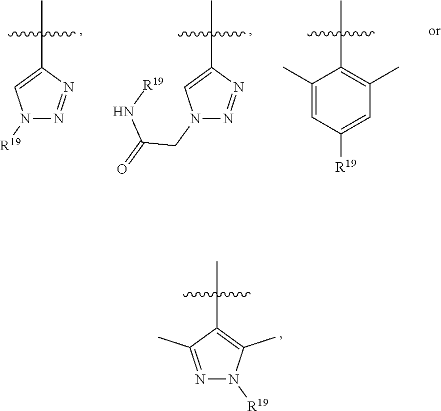
R³¹ is —C₂₋₅alkyl-O—R¹³, —O—C₂₋₅alkyl-R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, 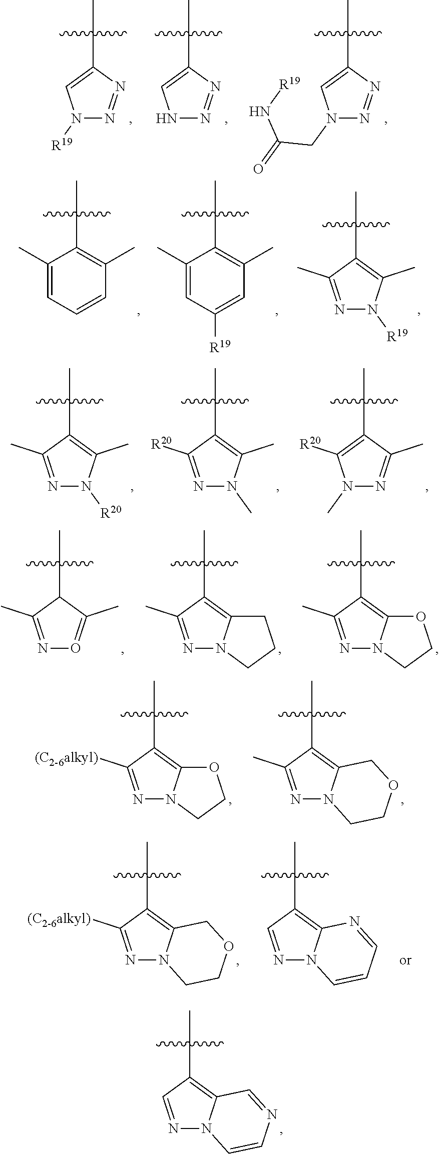
each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R¹³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is 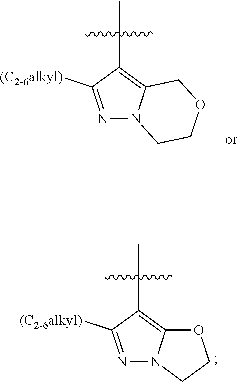
R¹⁹ is a bond connected to R¹⁴ of the linker; wherein each of Formula (A1), Formula (A2), Formula (A3) and Formula (A4) contains a single R¹⁹; and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, —C(O)NMe₂;
each R^{p} is independently —(CH₂)ₘOC(O)(unsubstituted C₁-C₆alkyl), and
each m is independently 1, 2, 3 or 4;
or
(ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me wherein each p is independently 2, 3, 4, 5, or 6;
and wherein [linker] has the following formula 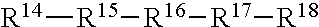
wherein
R¹⁴ is —C₁-C₆ alkyl, —C₂₋₆ alkenyl, —C₂₋₆ alkynyl, —C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)ₓ, —(C₃H₆—O)ₓ, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
wherein at least one of R¹⁴—R¹⁸ is present.

2. The compound of claim 1, wherein [ligase ligand moiety] is:

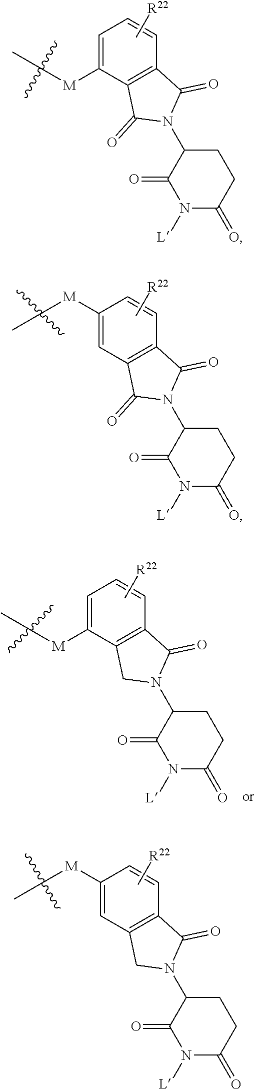

3. The compound of claim 1, wherein [ligase ligand moiety] is:

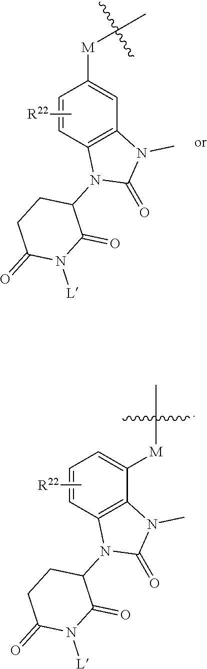

4. The compound of claim 3, wherein [ligase ligand moiety] is:

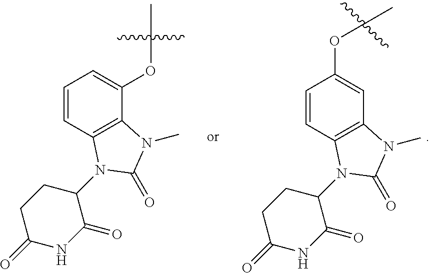 [SMILES: *Oc1cccc2c1n(C)c(=O)n2C1CCC(=O)NC1=O ; *Oc1ccc2c(c1)n(C)c(=O)n2C1CCC(=O)NC1=O]

5. The compound of any preceding claim, wherein R²² is hydrogen.

6. The compound of any preceding claim, wherein L′ is hydrogen.

7. The compound of any preceding claim, wherein M is O or NH, or is absent.

8. The compound of any one of claims 1-2 and 4-7, wherein [ligase ligand moiety] is

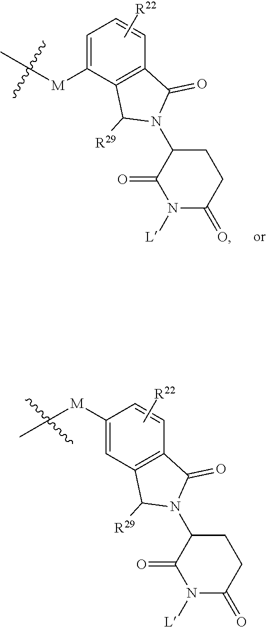

9. The compound of claim 8, wherein [ligase ligand moiety] is:

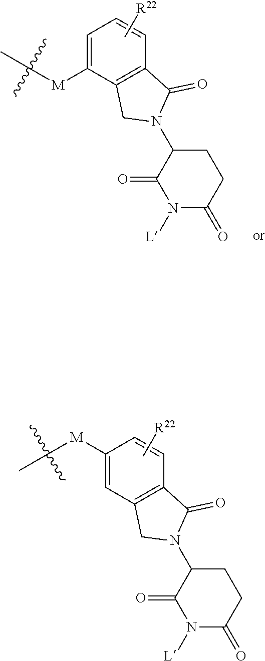

10. The compound of any preceding claim, wherein:
(a) [ligase ligand moiety] is: 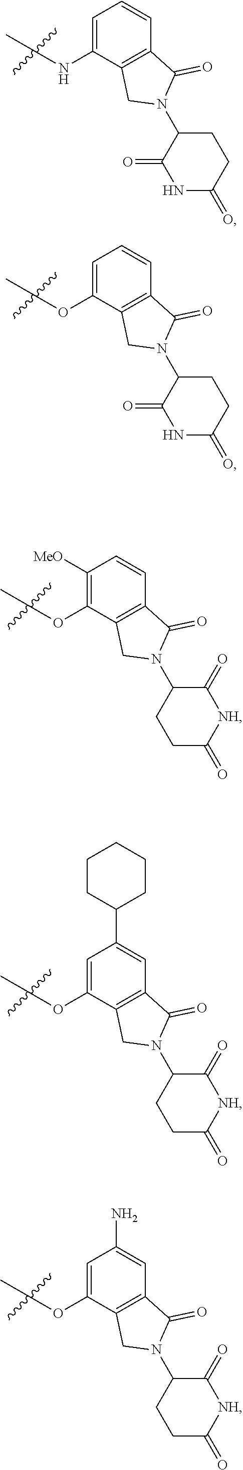 [SMILES: *Nc1cccc2c1CN(C1CCC(=O)NC1=O)C2=O ; *Oc1cccc2c1CN(C1CCC(=O)NC1=O)C2=O ; *Oc1c(OC)ccc2c1CN(C1CCC(=O)NC1=O)C2=O ; *Oc1cc(C2CCCCC2)cc2c1CN(C1CCC(=O)NC1=O)C2=O ; *Oc1cc(N)cc2c1CN(C1CCC(=O)NC1=O)C2=O] 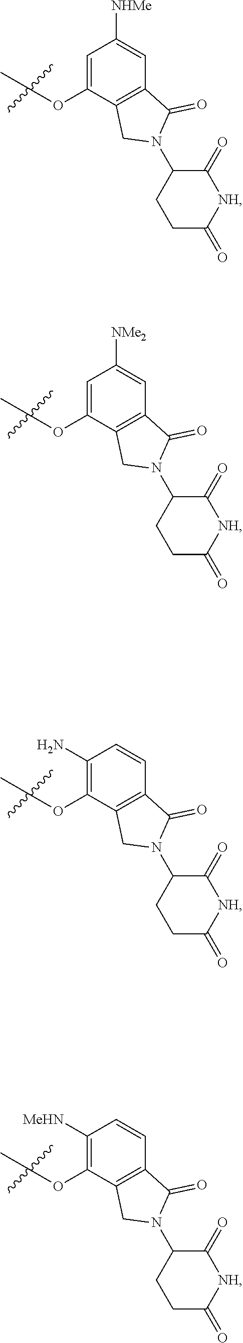 [SMILES: *Oc1cc(NC)cc2c1CN(C1CCC(=O)NC1=O)C2=O ; *Oc1cc(N(C)C)cc2c1CN(C1CCC(=O)NC1=O)C2=O ; *Oc1c(N)ccc2c1CN(C1CCC(=O)NC1=O)C2=O ; *Oc1c(NC)ccc2c1CN(C1CCC(=O)NC1=O)C2=O] 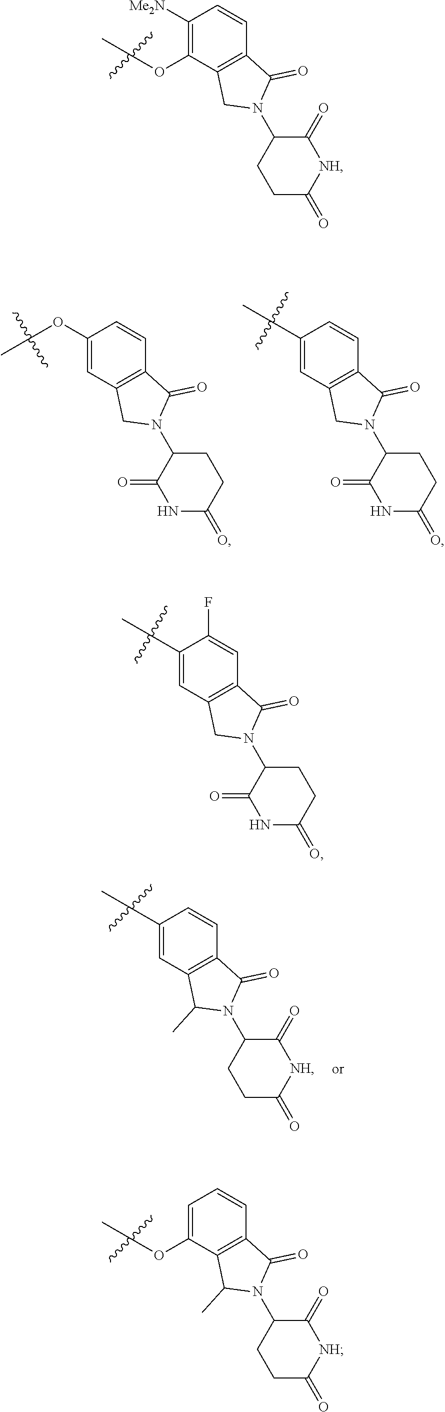 [SMILES: *Oc1c(N(C)C)ccc2c1CN(C1CCC(=O)NC1=O)C2=O ; *Oc1ccc2c(c1)CN(C1CCC(=O)NC1=O)C2=O ; *c1ccc2c(c1)CN(C1CCC(=O)NC1=O)C2=O ; *c1cc2c(cc1F)C(=O)N(C1CCC(=O)NC1=O)C2 ; *c1ccc2c(c1)C(C)N(C1CCC(=O)NC1=O)C2=O ; *Oc1cccc2c1C(C)N(C1CCC(=O)NC1=O)C2=O]
(b) [ligase ligand moiety] is: 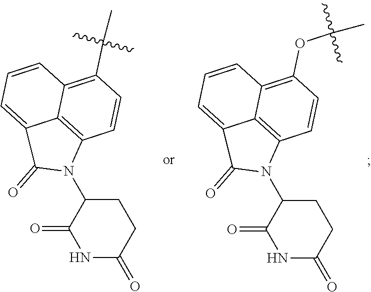 [SMILES: *c1ccc2c3c(cccc13)C(=O)N2C1CCC(=O)NC1=O ; *Oc1ccc2c3c(cccc13)C(=O)N2C1CCC(=O)NC1=O]
or
(c) [ligase ligand moiety] is:

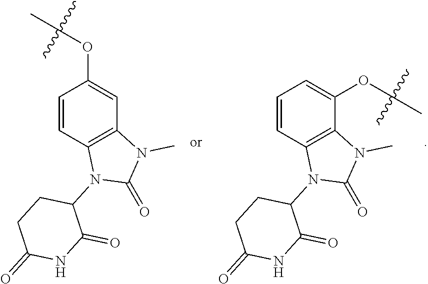 [SMILES: *Oc1ccc2c(c1)n(C)c(=O)n2C1CCC(=O)NC1=O ; *Oc1cccc2c1n(C)c(=O)n2C1CCC(=O)NC1=O]

11. The compound of any preceding claim, wherein:
(a) [ligase ligand moiety] is 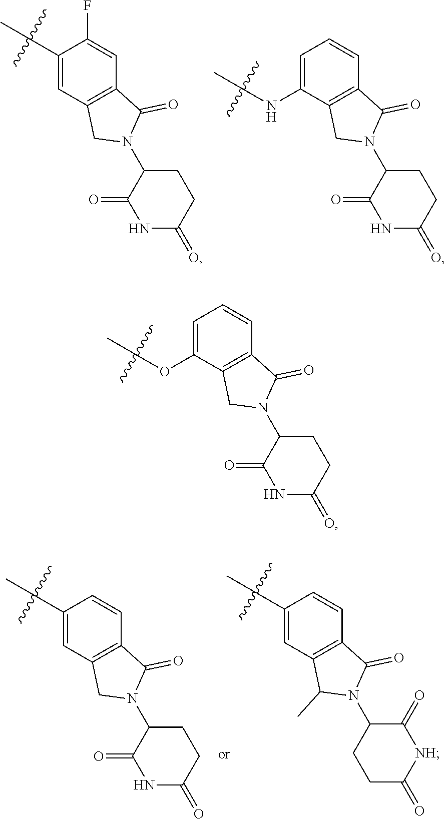 [SMILES: *c1cc2c(cc1F)C(=O)N(C1CCC(=O)NC1=O)C2 ; *Nc1cccc2c1CN(C1CCC(=O)NC1=O)C2=O ; *Oc1cccc2c1CN(C1CCC(=O)NC1=O)C2=O ; *c1ccc2c(c1)CN(C1CCC(=O)NC1=O)C2=O ; *c1ccc2c(c1)C(C)N(C1CCC(=O)NC1=O)C2=O]
or
(b) [ligase ligand moiety] is:

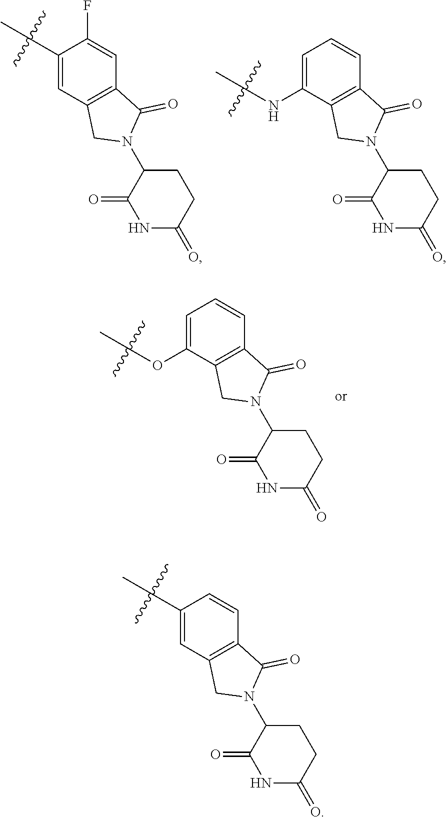 [SMILES: *c1cc2c(cc1F)C(=O)N(C1CCC(=O)NC1=O)C2 ; *Nc1cccc2c1CN(C1CCC(=O)NC1=O)C2=O ; *Oc1cccc2c1CN(C1CCC(=O)NC1=O)C2=O ; *c1ccc2c(c1)CN(C1CCC(=O)NC1=O)C2=O]

12. The compound of any preceding claim, wherein:
R¹⁶ is —C₁-C₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(C₂H₄—O)ₓ, —(C₃H₆—O)ₓ, or is absent
R¹⁸ is —C₁₋₆ alkyl, cycloalkyl, —CH₂—NH—C(O)—, heterocycloalkyl, or is absent.

13. The compound of any preceding claim, wherein R¹⁴ is —C₁₋₆alkyl, —C₂₋₆alkenyl, —C₂₋₆alkynyl, —C(O)—, —SO₂— or is absent.

14. The compound of claim 13, wherein R¹⁴ is —C₁-C₆ alkyl.

15. The compound of any preceding claim, wherein R¹⁵ is cycloalkyl, heterocycloalkyl, aryl, heteroaryl, —C₁₋₆ alkyl-NH—, -cycloalkyl-NH— or is absent.

16. The compound of claim 15, wherein R¹⁵ is heterocycloalkyl.

17. The compound of claim 16, wherein R¹⁵ is piperazine, bridged piperazine, piperazine N-oxide, piperazine cation, 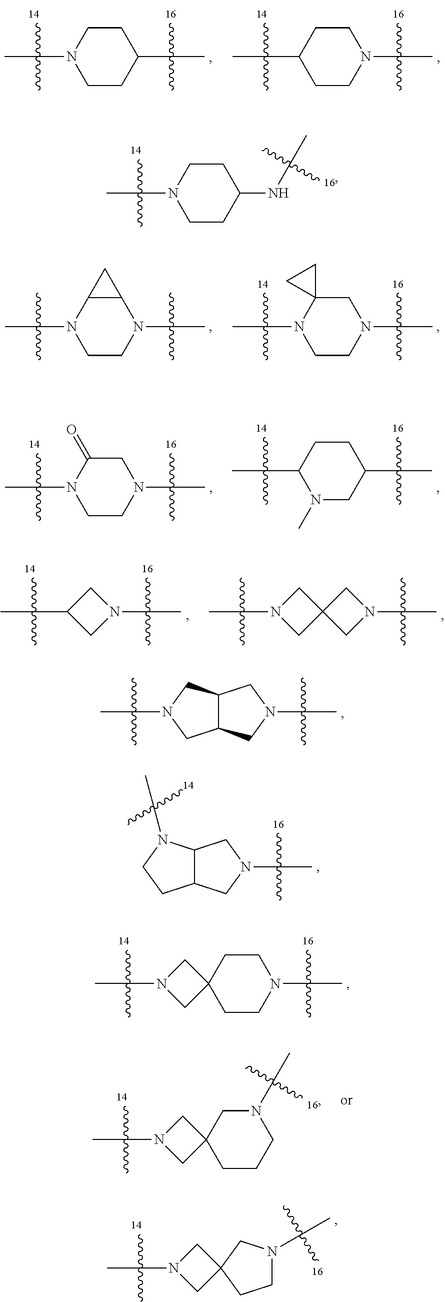
wherein [symbol] indicates attachment to R¹⁴ and [symbol] indicates attachment to R¹⁶.

18. The compound of claim 17, wherein R¹⁵ is piperazine or

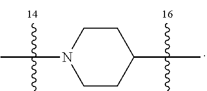 [SMILES: *C1CCN(*)CC1]

19. The compound of claim 15, wherein R¹⁵ is absent.

20. The compound of any preceding claim, wherein R¹⁶ is —C₁₋₆alkyl, —CH₂—C(O)—NH— or —C(O)—.

21. The compound of claim 20, wherein R¹⁶ is —C₁-C₆ alkyl or —C(O)—.

22. The compound of any preceding claim, wherein R¹¹ is absent.

23. The compound of any preceding claim, wherein R¹⁸ is —C₁₋₆ alkyl, heterocycloalkyl, or is absent

24. The compound of claim 23, wherein R¹⁸ is —C₁₋₆ alkyl or heterocycloalkyl; optionally —C₁₋₆ alkyl or piperazine.

25. The compound of claim 23, wherein R¹⁸ is absent.

26. The compound of any preceding claim, wherein [linker] is selected from 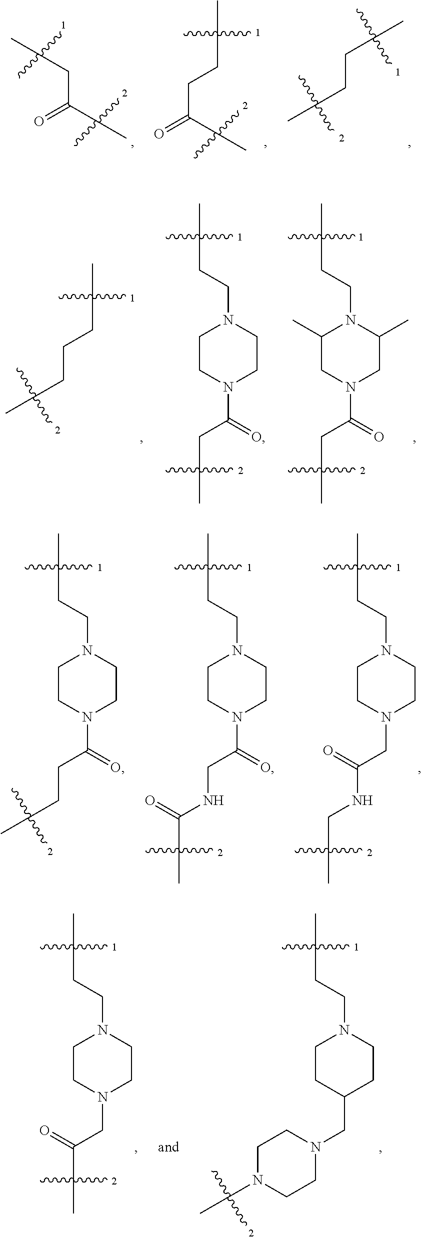
wherein [symbol]
indicates attachment to [MCL-1ligand prodrug moiety] and [symbol]
indicates attachment to [ligase ligand moiety];
optionally wherein [linker] is selected from 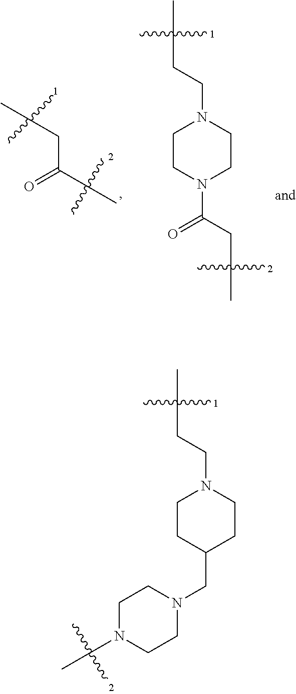 [SMILES: *CC(*)=O ; *CCN1CCN(C(=O)C*)CC1 ; *CCN1CCC(CN2CCN(*)CC2)CC1]
wherein [symbol]
indicates attachment to [MCL-1 ligand prodrug moiety] and [symbol]
indicates attachment to [ligase ligand moiety].

27. The compound of any preceding claim wherein in each of Formula (A1) and Formula (A4), one of R¹⁰ and R³⁰ is H and the other of R¹⁰ and R³⁰ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

28. The compound of any preceding claim, wherein in Formula (A1) R¹² is

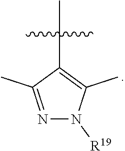

29. The compound of any preceding claim, wherein in Formula (A4) R³⁵ is

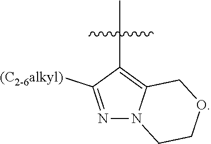

30. The compound of any preceding claim, wherein in Formula (A2) R³¹ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

31. The compound of any preceding claim, wherein in Formula (A3) R³⁴ is C₂₋₅alkyl-O—R¹³ or —O—C₂₋₅alkyl-R³, wherein R¹³ is phenyl or naphthyl, wherein the phenyl or naphthyl is optionally substituted with at least one substituent selected from halogen and C₁-C₆ alkyl.

32. The compound of any preceding claim, wherein Z₂ is C and [symbol] is a double bond.

33. The compound of any preceding claim, wherein [MCL-1 ligand prodrug moiety] is: 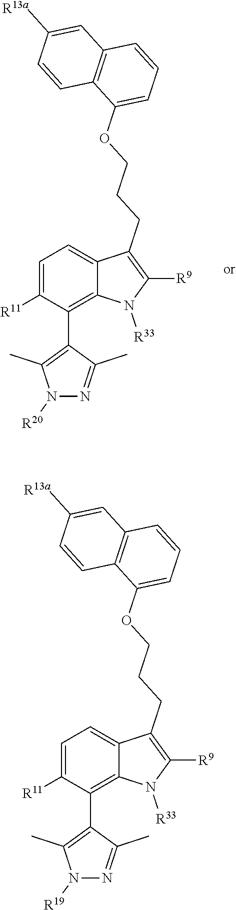
wherein
R¹¹ is halogen;
R¹³, is halogen; and
R²⁰ is Me, —CH₂—OMe, or —(CH₂CH₂O)ₚ(C₁-C₆ alkyl), wherein p is 1-5.

34. The compound of any preceding claim, wherein R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolyl cation.

35. The compound of claim 34, wherein R⁹ is —C(O)O(C₁-C₆alkyl), —C(O)Ocycloalkyl, —C(O)Oheterocycloalkyl, —C(O)Oaryl, or —P(O)(OR^{p})₂, wherein said C₁-C₆alkyl is substituted by one or more groups selected from —OC(O)Me, —OC(O)Et, —OC(O)ⁱPr, —OC(O)C(CH₃)₃, —OC(O)CH₂OH, —OC(O)CH(CH₃)OH, —OC(O)C(CH₃)₂OH, —OC(O)CH₂CH₂OH, —OC(O)CH(OH)CHMe₂, —OC(O)CH(OH)CH₂CHMe₂, —OC(O)CH₂CH(OH)Me, —OC(O)CH₂C(OH)Me₂, —OC(O)CH(CH₂OH)(NHC(O)Me), —OC(O)CH(NHC(O)Me)(isobutyl), —OC(O)CH(OH)aryl, —OC(O)CH(OH)CH₂aryl, —OC(O)heterocycloalkyl, —OC(O)cycloalkyl, —OC(O)aryl, —OC(O)heteroaryl, —OC(O)CH₂CH₂aryl, —OC(O)CH(NH₂)ⁱPr, —OC(O)CH(ⁱPr)NHC(O)O^{t}Bu, —OC(O)CH₂OCH₂CH₂OMe, —OC(O)CH₂(OCH₂CH₂)₂OMe, —OC(O)OMe, —OC(O)OEt, —OC(O)OⁱPr, —OC(O)Ocycloalkyl, —OC(O)OCH₂CH₂OEt, —OC(O)O(CH₂CH₂O)₂Et, —OC(O)O(CH₂CH₂O)₃Et, —OC(O)O(CH₂CH₂O)₃CH₂CH₂OP(O)(OH)₂, —OC(O)OCH₂CH₂SMe, —OC(O)OCH₂CH₂S(O)Me, —OC(O)OCH₂CH₂S(O)₂Me, —OC(O)OCH₂heteroaryl, —OC(O)OCH₂heterocycloalkyl, —OC(O)OCH₂CH₂OCH₂aryl —OC(O)OCH₂CH₂C(O)NMe₂, —OC(O)O(CH₂)₆NH₂, —OC(O)O(CH₂)₆NMe₂, —OC(O)OCH(CH₂OEt)₂, —OC(O)NHCH₂CH₂OMe, —OC(O)NMeCH₂CH₂OMe, —SMe, —S(O)Me, —P(O)(OH)₂, —OP(O)(OH)₂, —C(O)NMe₂, —C(O)heterocycloalkyl, heteroaryl, heterocycloalkyl, —N⁺(Me)₃, —N⁺(Me)₂cycloalkyl, N-methyl morpholinium cation, N-methyl piperidinium cation and N-methyl imidazolium cation.

36. The compound of claim 35, wherein R⁹ is —C(O)O(C₁-C₆alkyl) wherein said C₁-C₆alkyl is substituted by one or more groups selected from —OC(O)C(CH₃)₃, —OC(O)CH₂CH₂OH, —OC(O)C(CH₃)₂OH, —OC(O)heterocycloalkyl, —OC(O)CH₂OCH₂CH₂OMe, —OC(O)OiPr, —OC(O)Ocycloalkyl, —OC(O)O(CH₂CH₂O)₂Et, —OP(O)(OH)₂, heteroaryl or heterocycloalkyl.

37. The compound of claim 35, wherein R⁹ is —C(O)OCH₂OC(O)C(CH₃)₃, —C(O)OCH₂OC(O)Me, —C(O)OCH(Me)OC(O)Me, —C(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂OMe, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, —C(O)OCH₂CH₂CH₂piperazine, or —P(O)(OCH₂OC(O)C(CH₃)₃)₂:
optionally wherein R⁹ is —C(O)OCH₂OC(O)C(CH₃)₃, —C(O)OCH₂OC(O)Me, —C(O)OCH(Me)OC(O)Me, —C(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂Ome, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, or —C(O)OCH₂CH₂CH₂piperazine.

38. The compound of any one of claims 1-33, wherein R⁹ is —C(O)O(CH₂)ₚNMe₂, —C(O)O(CH₂)ₚNHMe, —C(O)OCH₂CH(OH)CH₂OH, C(O)OCH₂CH₂CMe₂OH, or —C(O)OCH₂CH₂SO₂Me wherein each p is independently 2, 3, 4, 5, or 6.

39. The compound of claim 38, wherein R⁹ is —C(O)OCH₂CH₂NMe₂, —C(O)OCH₂CH(OH)CH₂OH or —C(O)OCH₂CH₂SO₂Me.

40. The compound of any preceding claim, wherein R²⁰ is Me or —(CH₂CH₂O)₂Me.

41. The compound of any preceding claim, wherein R³³ is R¹⁹.

42. The compound of any one of claims 1-40, wherein R³³ is C₁-C₆ alkyl substituted with morpholine or a piperazine.

43. The compound of any preceding claim, wherein the C₁-C₆ alkyl substituted with morpholine or a piperazine is

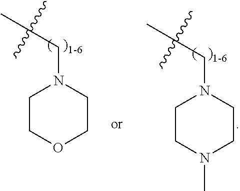

44. The compound of any preceding claim, wherein R¹¹ is Cl.

45. The compound of any preceding claim, wherein R^{13a} is F.

46. The compound of any preceding claim, wherein:
(a) [MCL-1 ligand prodrug moiety] is: 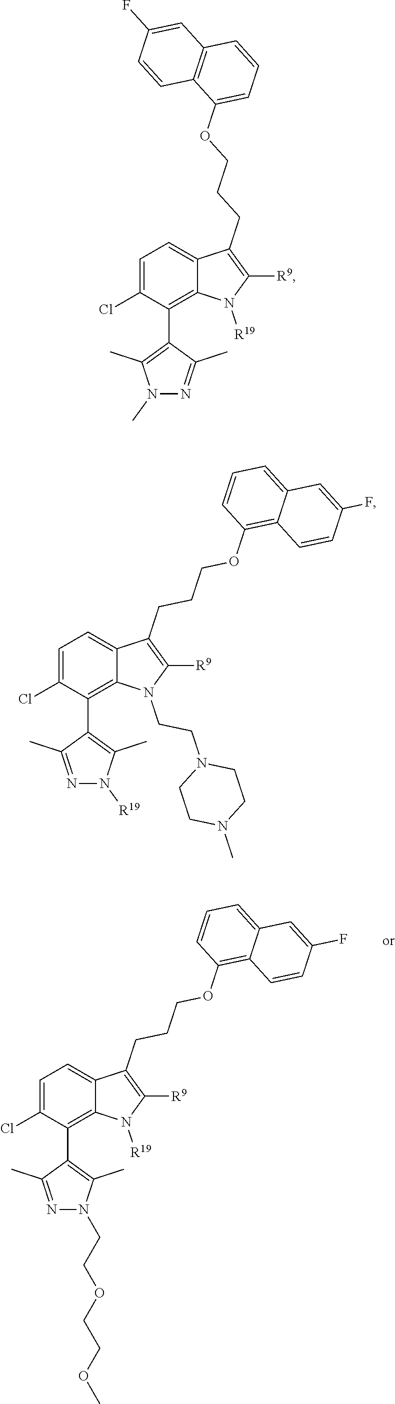 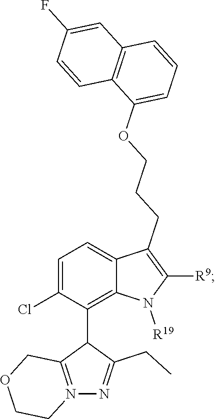
or
(b) [MCL-1 ligand prodrug moiety] is:

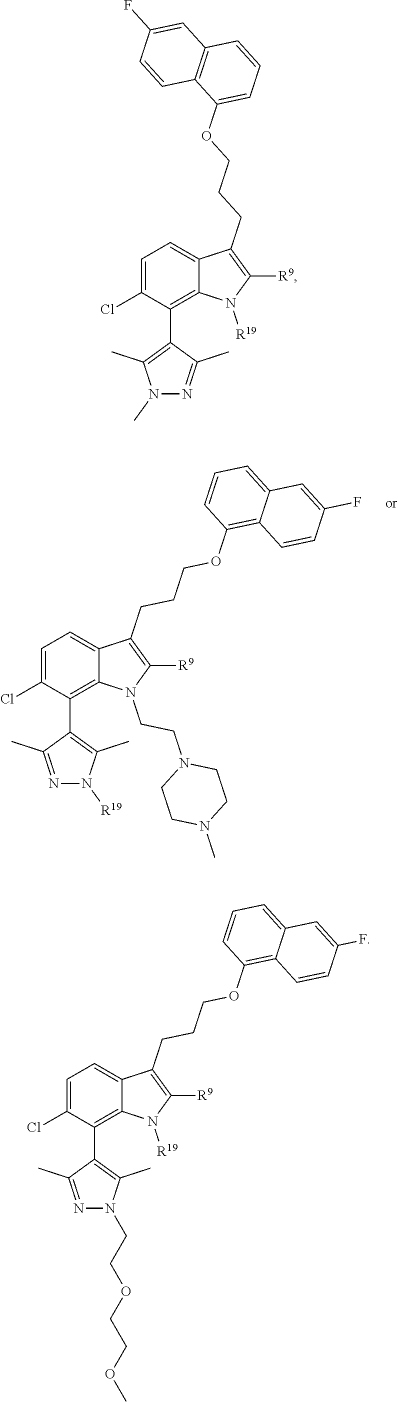

47. The compound of claim 26, wherein:
(a)
[linker] is 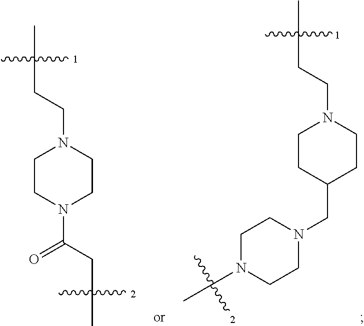 [SMILES: *CCN1CCN(C(=O)C*)CC1 ; *CCN1CCC(CN2CCN(*)CC2)CC1]
[ligase ligand moiety] is 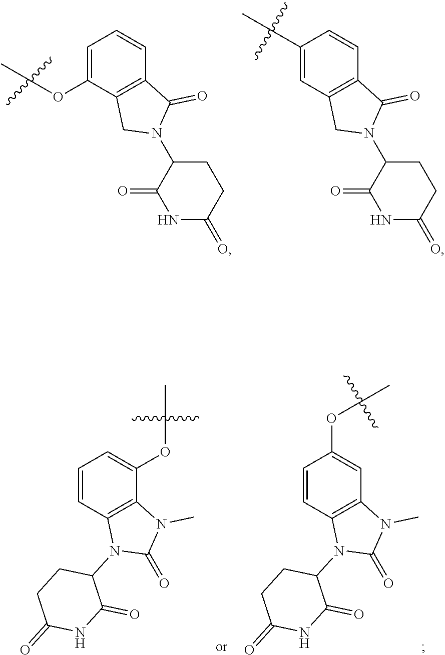 [SMILES: *Oc1cccc2c1CN(C1CCC(=O)NC1=O)C2=O ; *c1ccc2c(c1)CN(C1CCC(=O)NC1=O)C2=O ; *Oc1cccc2c1n(C)c(=O)n2C1CCC(=O)NC1=O ; *Oc1ccc2c(c1)n(C)c(=O)n2C1CCC(=O)NC1=O]
and
[MCL-1 ligand prodrug moiety] is a compound of Formula (A3), wherein
R³² is 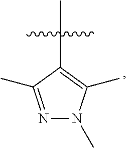
and
R⁹ is C(O)O(linear C₁-C₆alkyl)OC(O)C(CH₃)₃, C(O)O(linear C₁-C₆alkyl)OC(O)heterocyloalkyl, C(O)O(linear C₁-C₅alkyl)OC(O)CH₂CH₂OH, C(O)O(linear C₁-C₆alkyl)OC(O)CH₂OCH₂CH₂OMe, C(O)O(linear C₁-C₆alkyl)OC(O)Me, C(O)O(branched C₁-C₆alkyl)OC(O)OiPr, C(O)O(branched C₁-C₆alkyl)OC(O)Ocycloalkyl, C(O)O(branched C₁-C₆alkyl)OC(O)O(CH₂CH₂O)₂Et, or C(O)O(branched C₁-C₆alkyl)OC(O)Me.
or
(b)
[linker] is 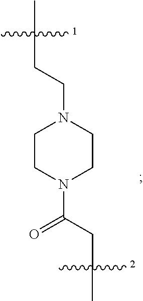 [SMILES: *CCN1CCN(C(=O)C*)CC1]
[ligase ligand moiety] is 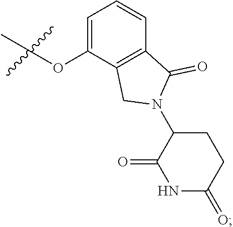 [SMILES: *Oc1cccc2c1CN(C1CCC(=O)NC1=O)C2=O]
and
[MCL-1 ligand prodrug moiety] is a compound of Formula (A3),
wherein
R³² is 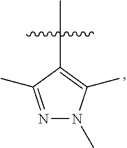
and
R⁹ is C(O)O(linear C₁-C₆alkyl)OC(O)C(CH₃)₃, C(O)O(branched C₁-C₆alkyl)OC(O)OiPr, C(O)O(branched C₁-C₆alkyl)OC(O)Ocycloalkyl, C(O)O(branched C₁-C₆alkyl)OC(O)O(CH₂CH₂O)₂Et, or C(O)O(branched C₁-C₆alkyl)OC(O)Me.
or
(c)
[linker] is 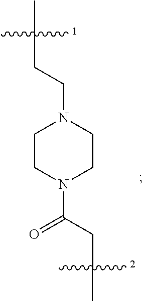 [SMILES: *CCN1CCN(C(=O)C*)CC1]
[ligase ligand moiety] is 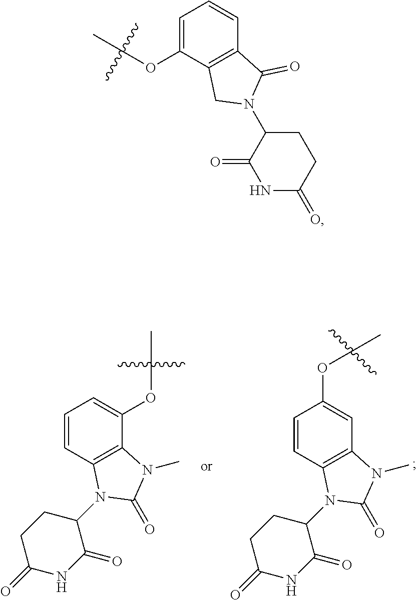 [SMILES: *Oc1cccc2c1CN(C1CCC(=O)NC1=O)C2=O ; *Oc1cccc2c1n(C)c(=O)n2C1CCC(=O)NC1=O ; *Oc1ccc2c(c1)n(C)c(=O)n2C1CCC(=O)NC1=O]
and
[MCL-1 ligand prodrug moiety] is a compound of Formula (A3), wherein
R³² is 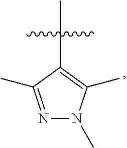
and
R⁹ is C(O)O(linear C₁-C₆alkyl)OC(O)C(CH₃)₃, C(O)O(linear C₁-C₅alkyl)OC(O)CH₂CH₂OH, C(O)O(linear C₁-C₆alkyl)OC(O)Me, C(O)OCH₂CH₂heterocycloalkyl, or C(O)O(branched C₁-C₆alkyl)OC(O)O(CH₂CH₂O)₂Et;
or
(d)
[linker] is 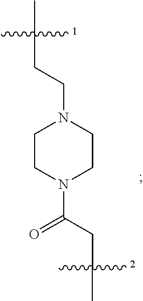 [SMILES: *CCN1CCN(C(=O)C*)CC1]
[ligase ligand moiety] is 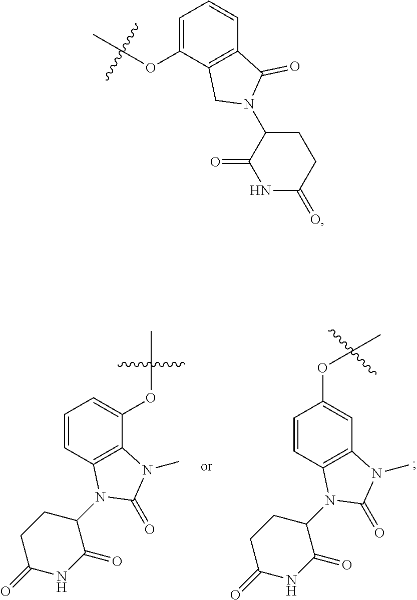 [SMILES: *Oc1cccc2c1CN(C1CCC(=O)NC1=O)C2=O ; *Oc1cccc2c1n(C)c(=O)n2C1CCC(=O)NC1=O ; *Oc1ccc2c(c1)n(C)c(=O)n2C1CCC(=O)NC1=O]
and
[MCL-1 ligand prodrug moiety] is a compound of Formula (A3), wherein
R³² is 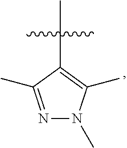
and
R⁹ is C(O)O(linear C₁-C₆alkyl)OC(O)C(CH₃)₃, C(O)O(linear C₁-C₅alkyl)OC(O)CH₂CH₂OH, C(O)O(linear C₁-C₆alkyl)OC(O)Me, or C(O)OCH₂CH₂heterocycloalkyl;
wherein when R⁹ is C(O)O(linear C₁-C₆alkyl)OC(O)C(CH₃)₃ then [ligase ligand moiety] is

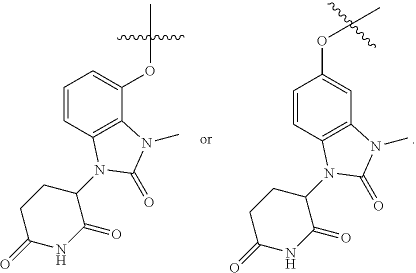 [SMILES: *Oc1cccc2c1n(C)c(=O)n2C1CCC(=O)NC1=O ; *Oc1ccc2c(c1)n(C)c(=O)n2C1CCC(=O)NC1=O]

48. The compound of claim 1, which is selected from:

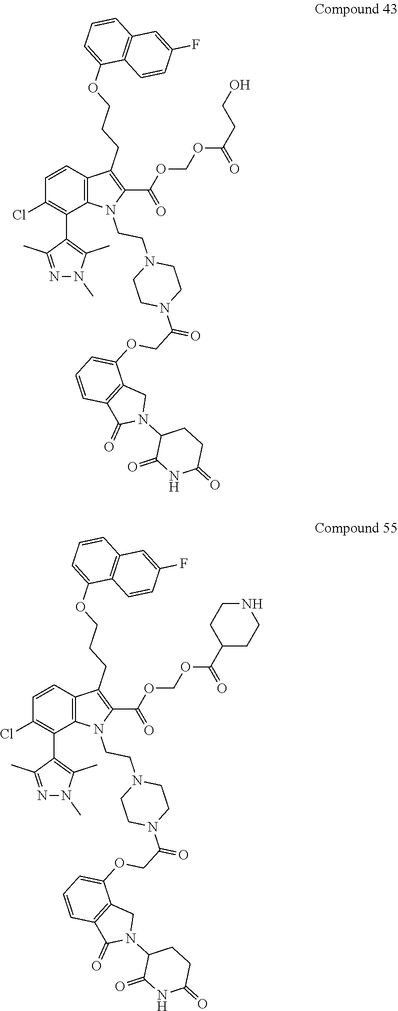 [SMILES: Cc1nn(C)c(C)c1-c1c(Cl)ccc2c(CCCOc3cccc4cc(F)ccc34)c(C(=O)OCOC(=O)CCO)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C3CCNCC3)n(CCN3CCN(C(=O)COc4cccc5c4CN(C4CCC(=O)NC4=O)C5=O)CC3)c12]

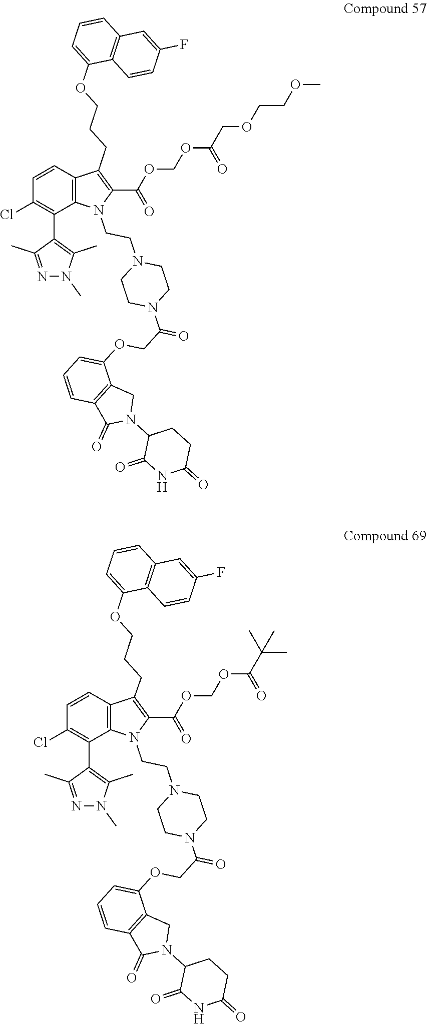

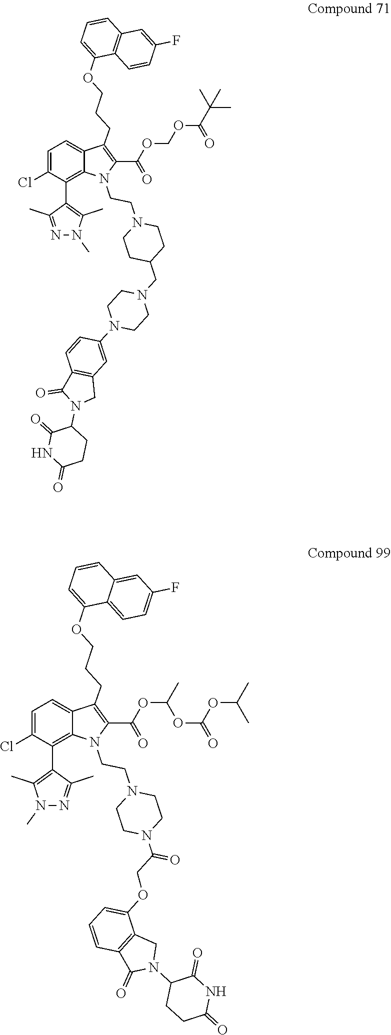

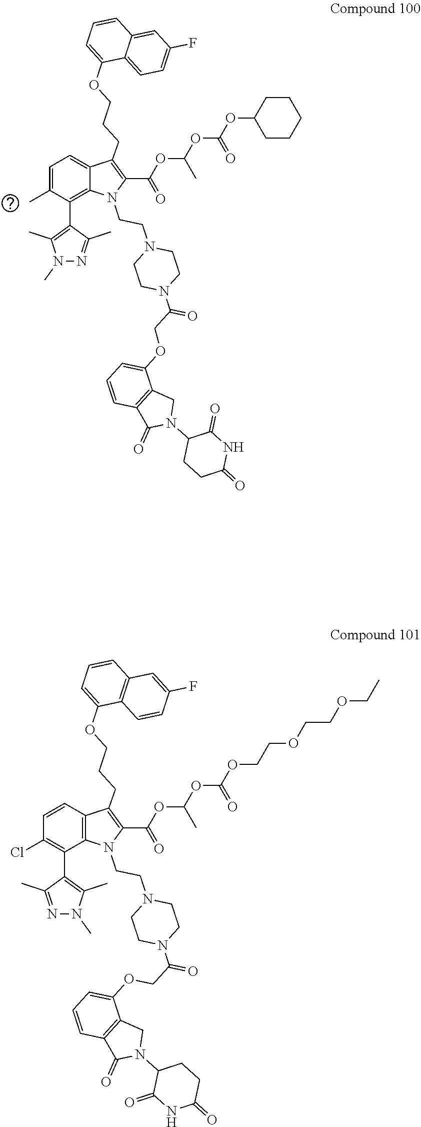 [SMILES: Cc1ccc2c(CCCOc3cccc4cc(F)ccc34)c(C(=O)OC(C)OC(=O)OC3CCCCC3)n(CCN3CCN(C(=O)COc4cccc5c4CN(C4CCC(=O)NC4=O)C5=O)CC3)c2c1-c1c(C)nn(C)c1C ; CCOCCOCCOC(=O)OC(C)OC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1]

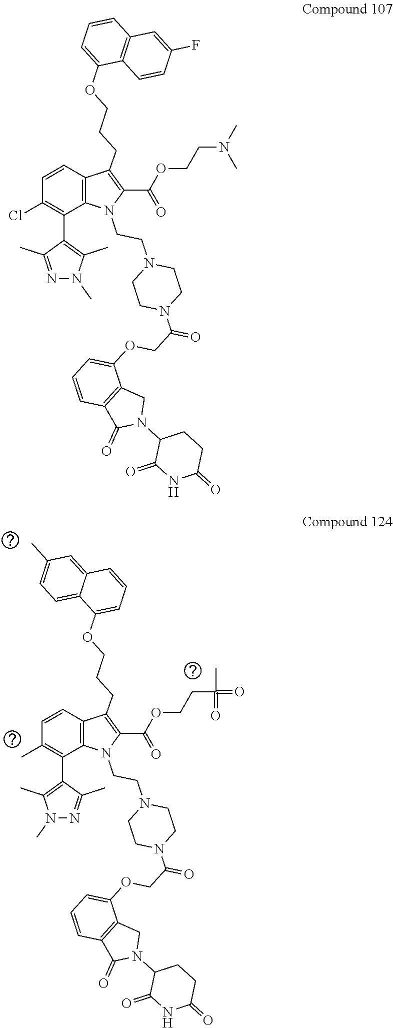

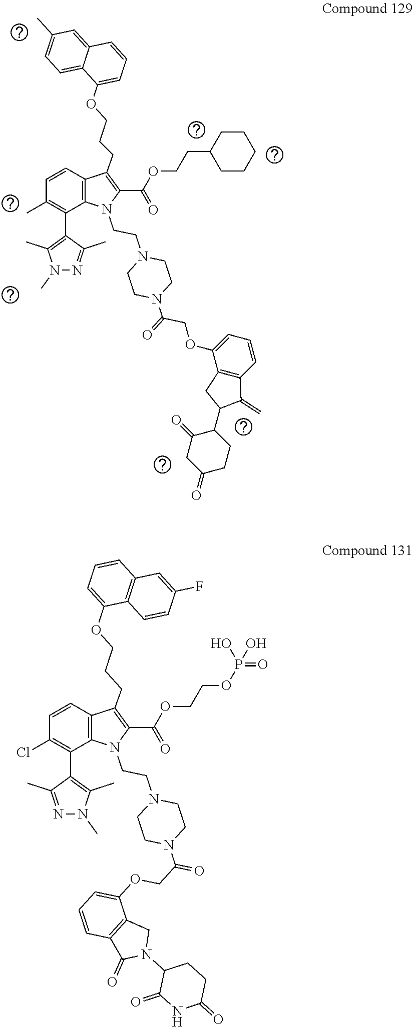

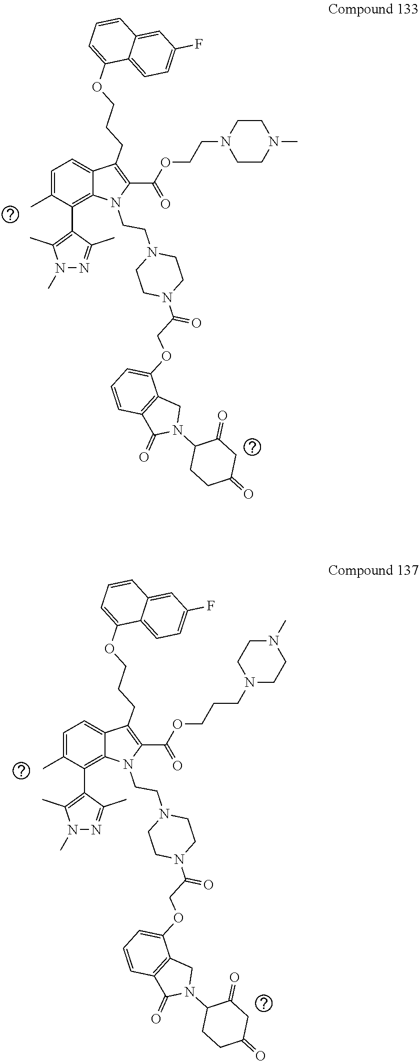

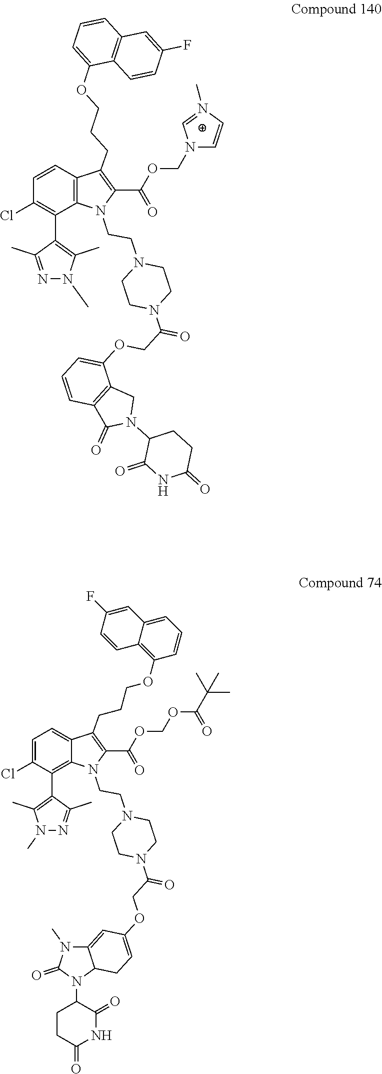

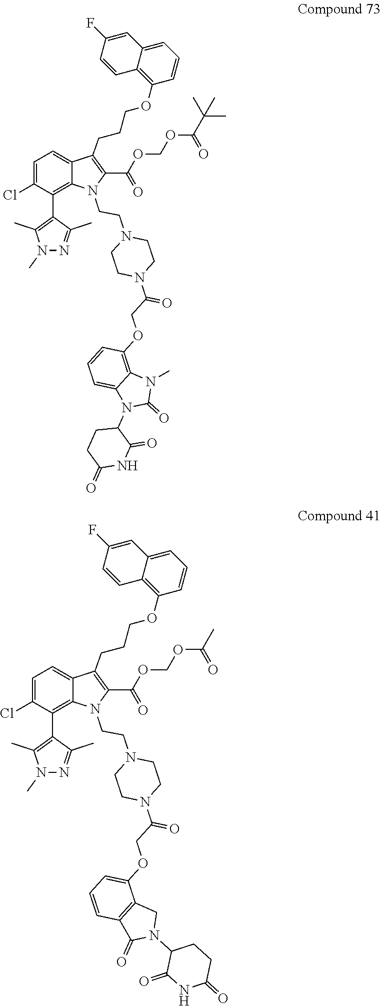

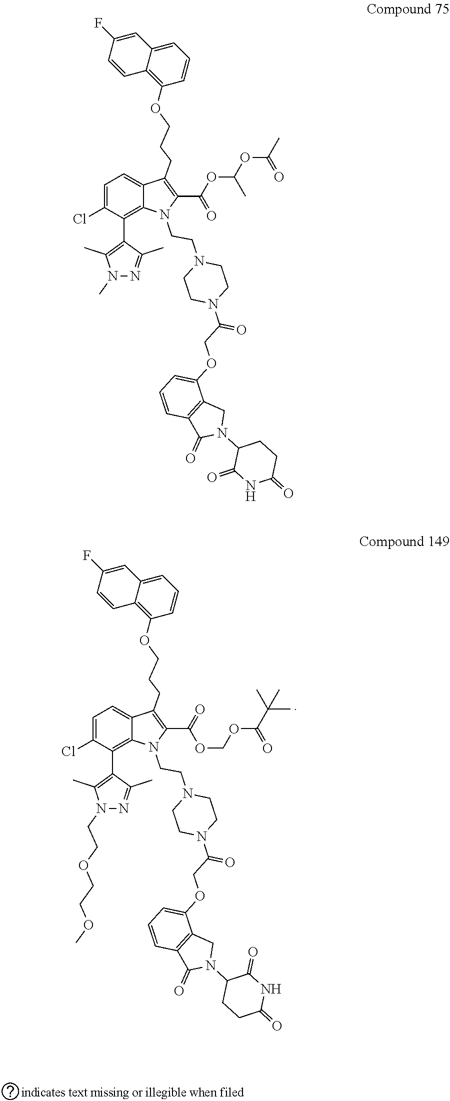 [SMILES: CC(=O)OC(C)OC(=O)c1c(CCCOc2cccc3cc(F)ccc23)c2ccc(Cl)c(-c3c(C)nn(C)c3C)c2n1CCN1CCN(C(=O)COc2cccc3c2CN(C2CCC(=O)NC2=O)C3=O)CC1 ; COCCOCCn1nc(C)c(-c2c(Cl)ccc3c(CCCOc4cccc5cc(F)ccc45)c(C(=O)OCOC(=O)C(C)(C)C)n(CCN4CCN(C(=O)COc5cccc6c5CN(C5CCC(=O)NC5=O)C6=O)CC4)c23)c1C]

49. The compound of claim 48, which is selected from:
(a)

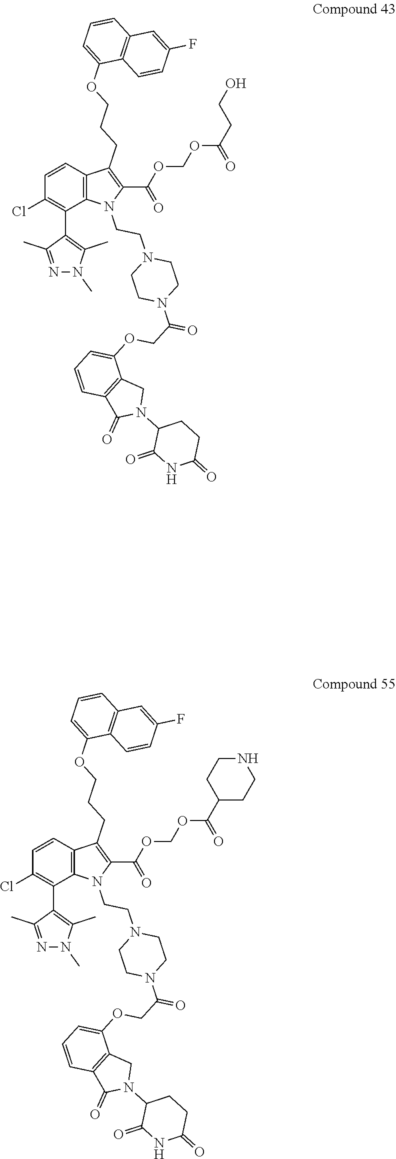 [SMILES: Cc1nn(C)c(C)c1-c1c(Cl)ccc2c(CCCOc3cccc4cc(F)ccc34)c(C(=O)OCOC(=O)CCO)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCOC(=O)C3CCNCC3)n(CCN3CCN(C(=O)COc4cccc5c4CN(C4CCC(=O)NC4=O)C5=O)CC3)c12]

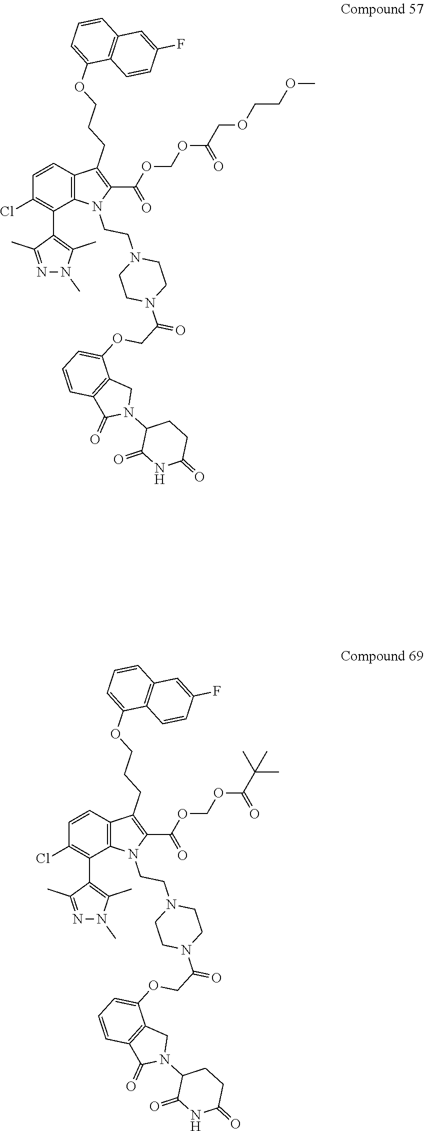

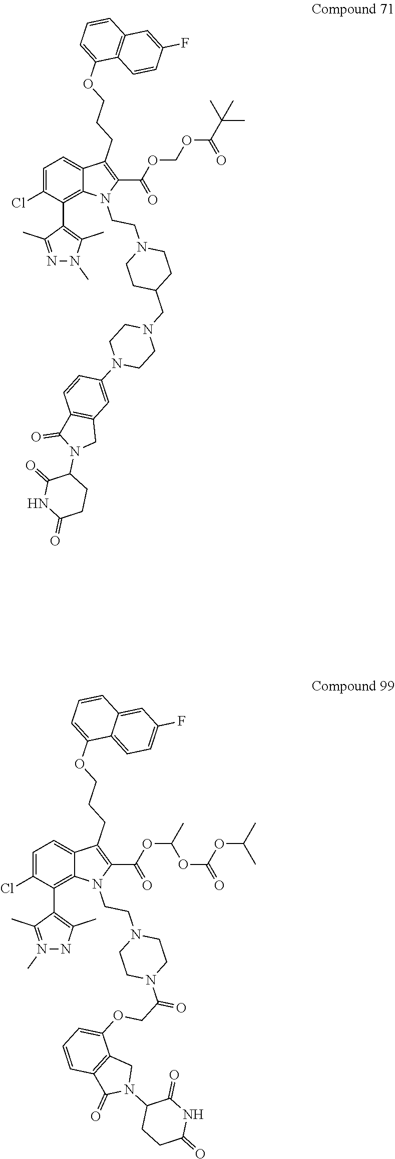

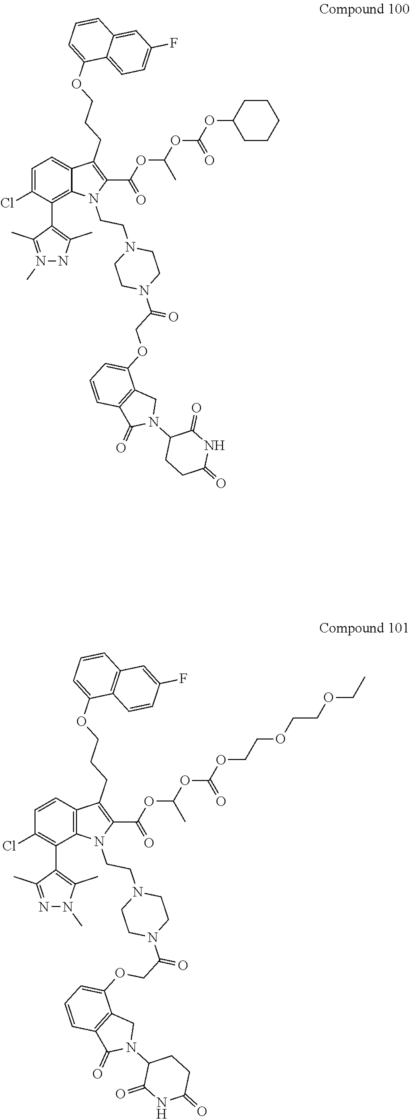

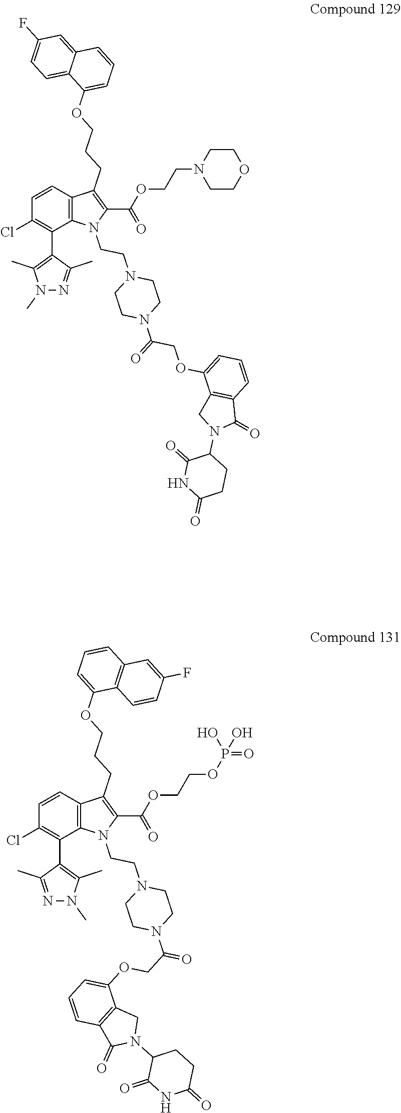 [SMILES: Cc1nn(C)c(C)c1-c1c(Cl)ccc2c(CCCOc3cccc4cc(F)ccc34)c(C(=O)OCCN3CCOCC3)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCCOP(=O)(O)O)n(CCN3CCN(C(=O)COc4cccc5c4CN(C4CCC(=O)NC4=O)C5=O)CC3)c12]

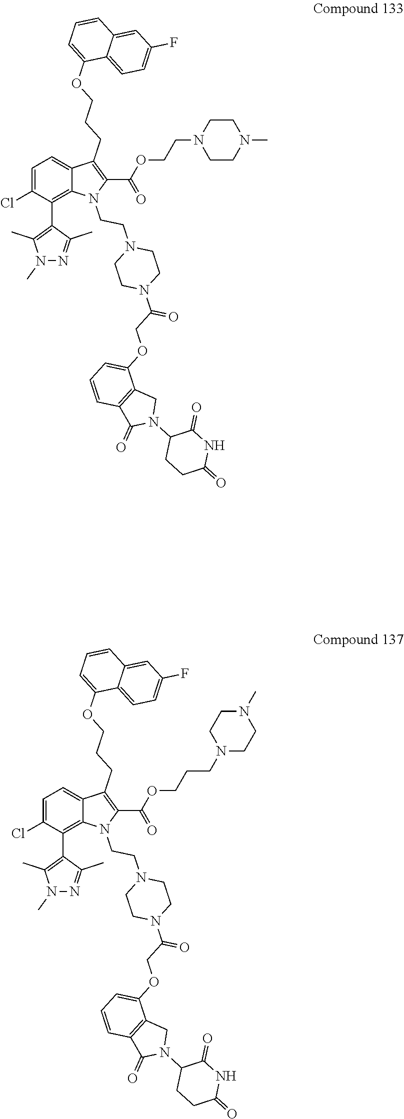

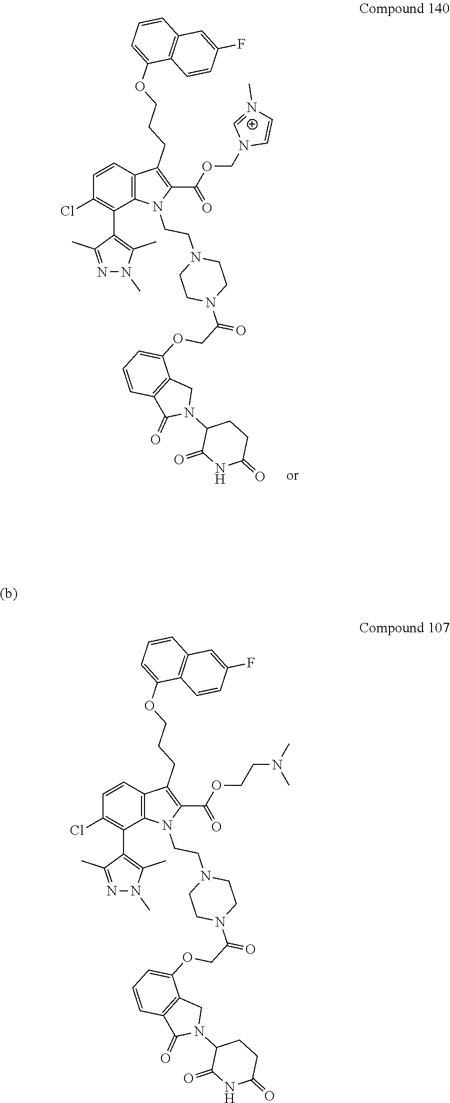

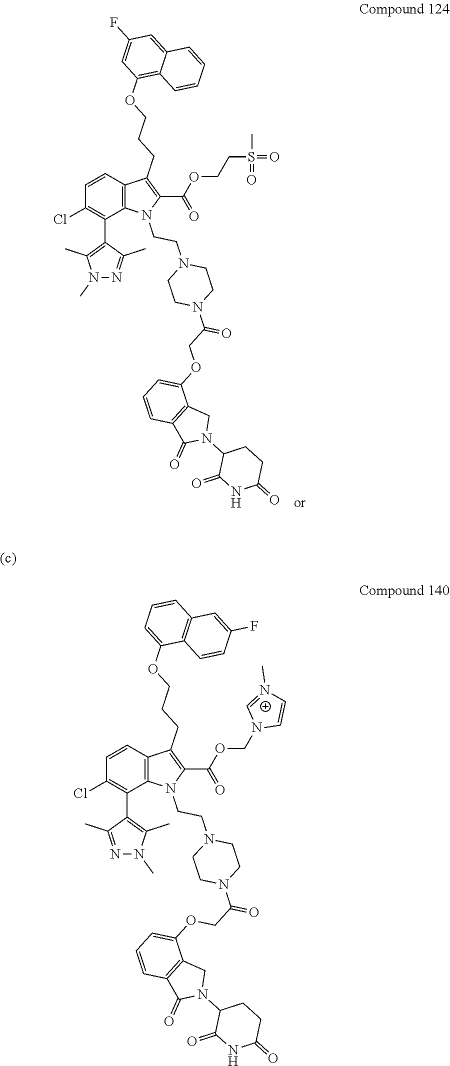 [SMILES: Cc1nn(C)c(C)c1-c1c(Cl)ccc2c(CCCOc3cc(F)cc4ccccc34)c(C(=O)OCCS(C)(=O)=O)n(CCN3CCN(C(=O)COc4cccc5c4CN(C4CCC(=O)NC4=O)C5=O)CC3)c12 ; Cc1nn(C)c(C)c1-c1c(Cl)ccc2c(CCCOc3cccc4cc(F)ccc34)c(C(=O)OCn3cc[n+](C)c3)n(CCN3CCN(C(=O)COc4cccc5c4CN(C4CCC(=O)NC4=O)C5=O)CC3)c12]

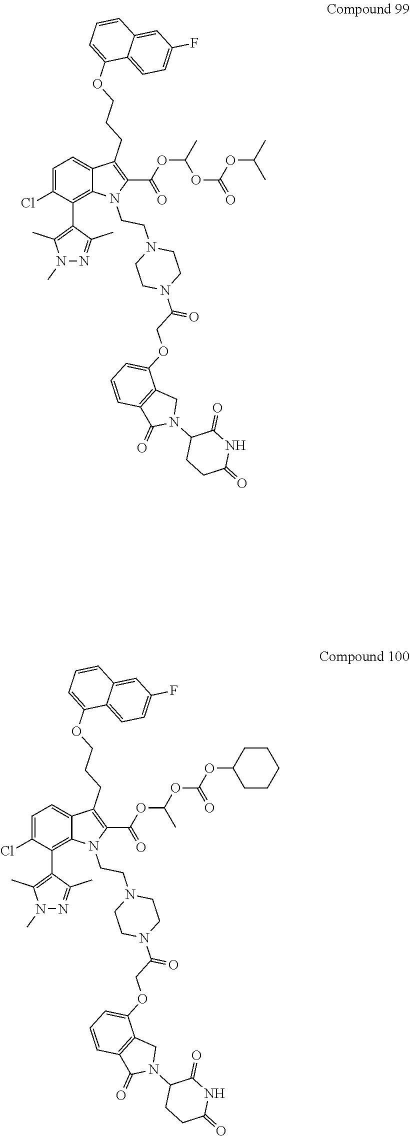

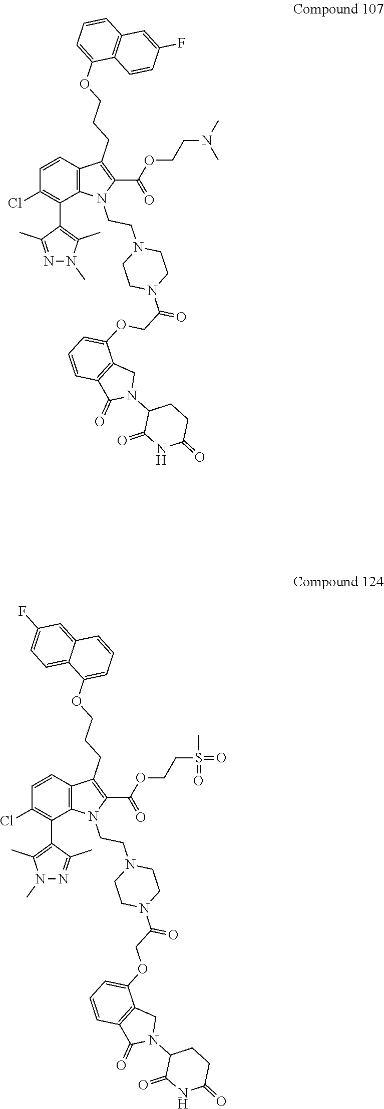

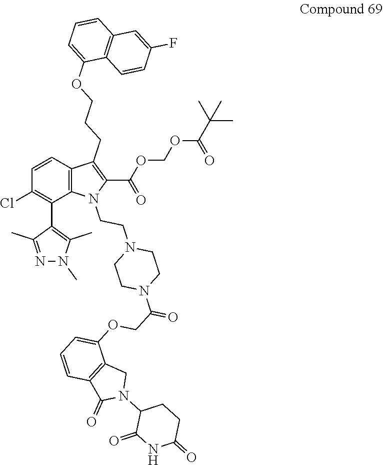 [SMILES: Cc1nn(C)c(C)c1-c1c(Cl)ccc2c(CCCOc3cccc4cc(F)ccc34)c(C(=O)OCOC(=O)C(C)(C)C)n(CCN3CCN(C(=O)COc4cccc5c4CN(C4CCC(=O)NC4=O)C5=O)CC3)c12]

50. The compound of claim 48, which is selected from:
(a) Compounds 41, 43, 55, 57, 69, 71, 73, 74, 75, 99, 100, 101 and 124; or
(b) Compounds 41, 43, 55, 57, 69, 71, 73, 74, 75, 99, 100 and 101; or
(c) Compounds 69, 75, 99, 100, 101 and 124; or
(d) Compounds 69, 75, 99, 100 and 101.

51. The compound of claim 48, which is selected from:
(a) Compounds 41, 43, 69, 73, 74, 101, 129 and 133; or
(b) Compounds 41, 43, 73, 74, 129 and 133.

52. The compound of any preceding claim, wherein unless otherwise specified each alkyl, alkenyl, alkynyl, aryl, heteroaryl and benzyl is unsubstituted.

53. A compound of formula (I) 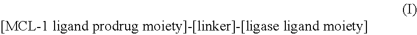
or a salt, solvate, hydrate or isomer thereof,
wherein [ligase ligand moiety] is:
(a) Formula (IV) or (IVa) 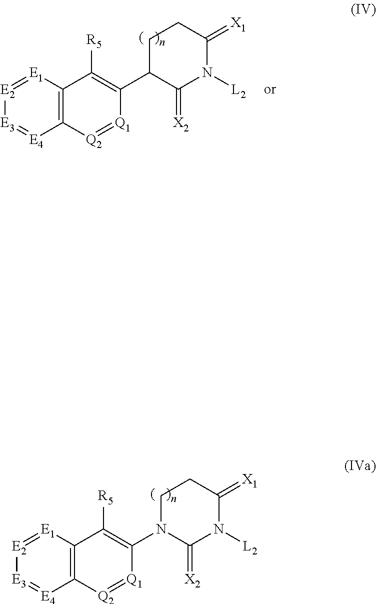
wherein:
each of X₁ and X₂ is independently O or S;
each of Q₁ and Q₂ is independently N or CRS, wherein at least one of Q₁ and Q₂ is N;
each of E₁, E₂, E₃ and E₄ is independently N or CR′;
n is 0, 1 or 2;
L₂ is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R⁵ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂; —O—R²¹, —NH—R²¹, —C(O)—NH—R²¹, or —CH₂—NH—C(O)—R²¹;
each R′ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —C(O)NHCHR^{b}₂, —CHR^{b}NHC(O)NHR^{b}, —CHR^{b}NHC(O)C(halogen)₂R^{b}, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —NHS(O)₂R^{b}, —R²¹, —O—R²¹, —NH—R²¹, —C(O)—R²¹, —C(O)—NH—R²¹, or —CH₂—NH—C(O)—R²¹;
and
each R^{b} is independently hydrogen, alkyl, alkenyl, aryl, heteroaryl, or benzyl;
wherein R²¹ is a bond connected to R¹⁸ of the linker, and wherein Formula (IV) contains a single R²¹;
(b) Formula (VIIa), (VIIb), (VIIc) or (VIId): 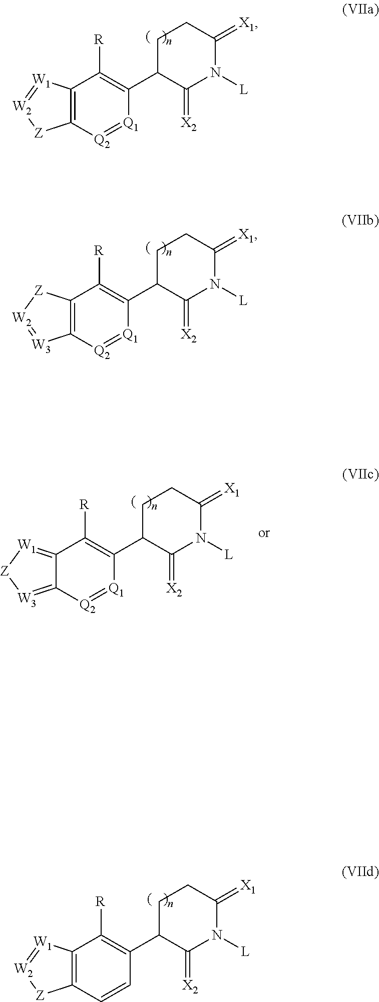
wherein:
each of X₁ and X₂ is independently O or S;
each of Q₁ and Q₂ is independently N or CR, wherein at least one of Q₁ and Q₂ is N;
each of W₁, W₂ and W₃ is independently N or CR^{a};
Z is O, S, or NR*;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹;
each R^{a} is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, —S(O)₂NR^{b}₂, —R²¹, —O—R²¹, —NH—R²¹, —C(O)—R²¹, —C(O)—NH—R²¹, or —CH₂—NH—C(O)—R²¹; and;
each R^{e} is independently hydrogen, alkyl, alkenyl, aryl, heteroaryl, benzyl, —R²¹, —O—R²¹, —NH—R²¹, —C(O)—R²¹, —C(O)—NH—R²¹, or —CH₂—NH—C(O)—R²¹;
each R^{b} is independently hydrogen, alkyl, alkenyl, aryl, heteroaryl, or benzyl;
wherein R²¹ is a bond connected to R¹⁸ of the linker, and wherein each of Formula (VIIa), Formula (VIIb) and Formula (VIIc) contains a single R²¹;
(c) Formula (VIII): 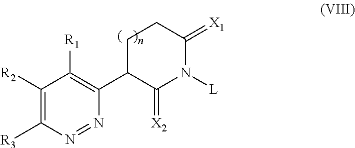
wherein
each of X₁ and X₂ is independently O or S;
n is 0, 1 or 2;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of R₁, R₂ and R₃ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, S(O)₂R^{b}, —S(O)₂OR^{b}, —S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (VIII) contains a single R²¹; or
(d) Formula (IX): 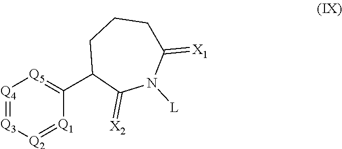
wherein
each of X₁ and X₂ is independently O or S;
L is hydrogen, alkyl, alkenyl, aryl, heteroaryl, benzyl, haloalkyl, haloalkenyl, —C(O)R^{b}, —C(O)OR^{b}, —C(O)NH₂, —C(O)NHR^{b}, —C(O)NR^{b}₂, —OR^{b}, —NR^{b}₂, or —S(O)₂R^{b};
each of Q₁, Q₂, Q₃, Q₄ and Q₅ is independently N or CR, wherein at least one of Q₁, Q₂, Q₃, Q₄ and Q₅ is N;
each R is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, —CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, —S(O)₂NH₂, —S(O)₂NHR^{b}, or —S(O)₂NR^{b}₂, —R²¹, —O—R²¹, —NH—R²¹, —C(O)—R²¹, —C(O)—NH—R²¹, or —CH₂—NH—C(O)—R²¹; and
each R^{b} is independently hydrogen, alkyl, alkenyl, aryl, heteroaryl, or benzyl;
wherein R²¹ is a bond connected to R¹⁸ of the linker, and wherein Formula (IX) contains a single R²¹;
wherein [MCL-1 ligand prodrug moiety] is a compound of Formula (A1), (A2), (A3) or (A4): 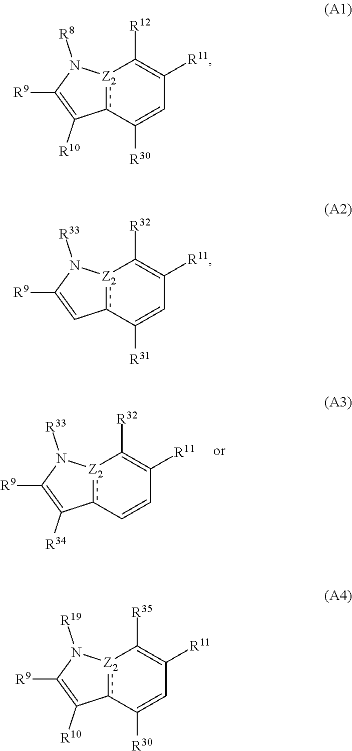
wherein [symbol]
is a single bond or a double bond;
each Z₂ is independently N or C, wherein when Z₂ is N, then [symbol] is a single bond; and when Z₂ is C, then [symbol] is a double bond;
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is 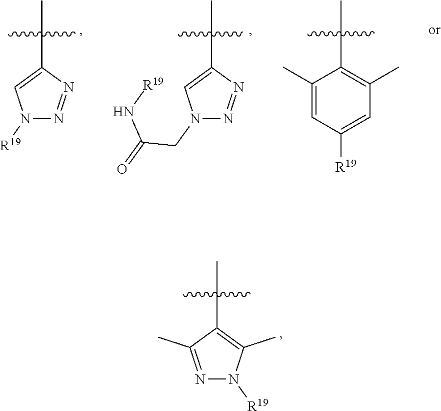
R³¹ is —C₂₋₅alkyl-O—R¹³, —O—C₂₋₅alkyl-R¹³ or —C₂₋₅alkyl-NMe-R⁷³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, 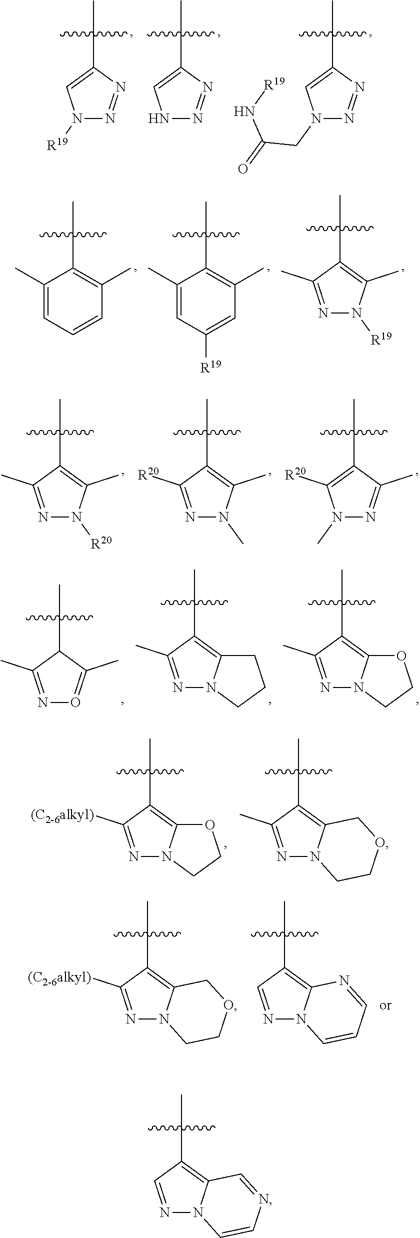
each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is 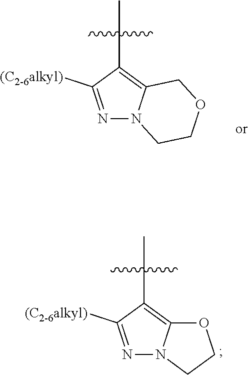
R¹⁹ is a bond connected to R¹⁴ of the linker; wherein each of Formula (A1), Formula (A2), Formula (A3) and Formula (A4) contains a single R¹⁹; and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, C(O)NMe₂;
each R^{p} is independently —(CH₂)ₘOC(O)(unsubstituted C₁-C₆alkyl), and
each m is independently 1, 2, 3 or 4;
or
(ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me, wherein each p is independently 2, 3, 4, 5, or 6;
and wherein [linker] has the following formula 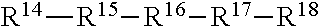
wherein
R¹⁴ is —C₁-C₆ alkyl, —C₂₋₆ alkenyl, —C₂₋₆ alkynyl, —C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)ₓ, —(C₃H₆—O)ₓ, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
wherein at least one of R¹⁴—R¹⁸ is present.

54. The compound of claim 53, wherein unless otherwise specified each alkyl, alkenyl, alkynyl, aryl, heteroaryl and benzyl groups is unsubstituted.

55. The compound of claim 53 or 54, wherein each Rⁱ is independently hydrogen, halogen, alkyl, alkenyl, alkynyl, aryl, heteroaryl, benzyl, haloalkyl, haloalkenyl, —NH₂, —NHR^{b}, —NR^{b}₂, —NR^{b}C(O)R^{b}, —NR^{b}C(O)OR^{b}, —NO₂, CN, —C(O)R^{b}, —C(O)OR^{b}, —C(O)NH₂, —C(O)NHR^{b}, —C(O)NR^{b}₂, —OR^{b}, —OC(O)R^{b}, —OC(O)OR^{b}, —OC(O)NH₂, —OC(O)NHR^{b}, —OC(O)NR^{b}₂, —SR^{b}, —S(O)₂R^{b}, —S(O)₂OR^{b}, S(O)₂NH₂, —S(O)₂NHR^{b}, —S(O)₂NR^{b}₂, —R², —O—R²¹, —NH—R²¹, —C(O)—R²¹, —C(O)—NH—R²¹, or —CH₂—NH—C(O)—R²¹.

56. The compound of claim 55, wherein each R^{b} is independently hydrogen, alkyl, cycloalkyl, or aryl.

57. The compound of claim 56, wherein the aryl is substituted with one or more groups selected from halogen, alkyl and O-haloalkyl, optionally wherein the halogen is Cl, the alkyl is methyl and the O-haloalkyl is O—CF₃.

58. The compound of any one of claims 53-57, wherein one of E₁, E₂, E₃ and E₄ is N, and the remaining three of E₁, E₂, E₃ and E₄ are each CR′.

59. The compound of claim 58, wherein:
(a) E₁ is N, and E₂, E₃ and E₄ are CR′; or
(b) E₂ is N, and E₁, E₃ and E₄ are CR′; or
(c) E₃ is N, and E₁, E₂ and E₄ are CR′; or
(d) E₄ is N, and E₁, E₂ and E₃ are CR′.

60. The compound of any one of claims 53-57, wherein E₁, E₂, E₃ and E₄ are each CR′, optionally wherein E₁, E₂, E₃ and E₄ are each CH.

61. The compound of claim 60, wherein three of E₁, E₂, E₃ and E₄ are CH, and one of E₁, E₂, E₃ and E₄ is C-halogen, C-alkyl, C-alkenyl, C-alkynyl, C-aryl, C-heteroaryl, C-benzyl, C-haloalkyl, C-haloalkenyl, C—NH₂, C—NHR^{b}, C—NR^{b}₂, C—NR^{b}C(O)R^{b}, C—NR^{b}C(O)OR^{b}, C—NO₂, C—CN, C—C(O)R^{b}, C—C(O)OR^{b}, C—C(O)NH₂, C—C(O)NHR^{b}, C—C(O)NR^{b}₂, C—C(O)NHCHR^{b}₂, C—CHR^{b}NHC(O)NHR^{b}, C—CHR^{b}NHC(O)C(halogen)₂R^{b}, C—OR^{b}, C—OC(O)R^{b}, C—OC(O)OR^{b}, C—OC(O)NH₂, C—OC(O)NHR^{b}, C—OC(O)NR^{b}₂, C—SR^{b}, C—S(O)₂R^{b}, C—S(O)₂OR^{b}, C—S(O)₂NH₂, C—S(O)₂NHR^{b}, C—S(O)₂NR^{b}₂, C—NHS(O)₂R^{b}, —R²¹, —O—R²¹, —NH—R²¹, —C(O)—R²¹, —C(O)—NH—R²¹, or —CH₂—NH—C(O)—R²¹.

62. The compound of claim 61, wherein:
(a) E₂, E₃ and E₄ are each CH; or
(b) E₁, E₃ and E₄ are each CH; or
(c) E₁, E₂ and E₄ are each CH.

63. The compound of any one of claims 53-57, wherein two of E₁, E₂, E₃ and E₄ are N, and the remaining two of E₁, E₂, E₃ and E₄ are each CR′.

64. The compound of any one of claims 53-57, wherein three of E₁, E₂, E₃ and E₄ are N, and the remaining one of E₁, E₂, E₃ and E₄ is CR′.

65. The compound of any one of claims 53-64, wherein one of W₁, W₂ and W₃ is N, and the other of W₁, W₂ and W₃ is CR^{a}, optionally wherein R^{a} is H.

66. The compound of any one of claims 53-64, wherein W₁, W₂ and W₃ are each CR^{a}.

67. The compound of any one of claims 53-66, wherein W₁ is C—NH₂, C—NHR^{b} or C—NR^{b}₂; optionally C—NH₂.

68. The compound of any one of claims 53-64, wherein W₁, W₂ and W₃ are each N.

69. The compound of any one of claims 53-68, wherein Z is NH or N-alkyl, optionally NH or N-Me.

70. The compound of any one of claims 53-69, wherein in Formula (IV), Formula (VIIa), Formula (VIIb) and Formula (VIIc):
(a) Q₁ is N and Q₂ is CR; or
(b) Q₁ is N and Q₂ is N; or
(c) Q₁ is CR and Q₂ is N, optionally wherein Q₁ is C—H or C-alkyl, further optionally wherein Q₁ is C—H or C-Me.

71. The compound of any one of claims 53-70, wherein in Formula (IX) one of Q₁, Q₂, Q₃, Q₄ and Q₅ is N, and the remaining four of Q₁, Q₂, Q₃, Q₄ and Q₅ are each CR.

72. The compound of claim 71, wherein:
(a) Q₁ is N; or
(b) Q₂ is N; or
(c) Q₃ is N.

73. The compound of any one of claims 53-70, wherein in Formula (IX) two of Q₁, Q₂, Q₃, Q₄ and Q₅ are N, and the remaining three of Q₁, Q₂, Q₃, Q₄ and Q₅ are each CR.

74. The compound of claim 73, wherein:
(a) Q₁ and Q₂ are N, and Q₃, Q₄ and Q₅ are each CR; or
(b) Q₂ and Q₃ are N, and Q₁, Q₄ and Q₅ are each CR; or
(c) Q₁ and Q₃ are N, and Q₂, Q₄ and Q₅ are each CR; or
(d) Q₂ and Q₄ are N, and Q₁, Q₃ and Q₅ are each CR; or
(e) Q₁ and Q₄ are N, and Q₂, Q₃ and Q₅ are each CR.

75. The compound of any one of claims 53-70, wherein in Formula (IX) three of Q₁, Q₂, Q₃, Q₄ and Q₅ are N, and the remaining two of Q₁, Q₂, Q₃, Q₄ and Q₅ are each CR.

76. The compound of any one of claims 53-70, wherein [ligase ligand moiety] is:

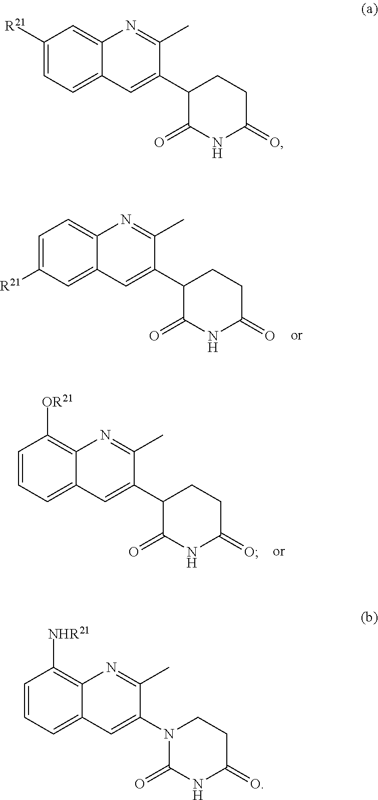

77. The compound of any one of claims 53-70, wherein [ligase ligand moiety] is:

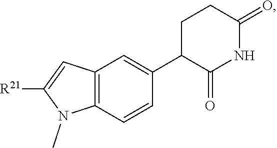

78. A compound of formula (I) 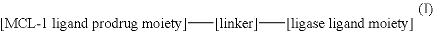
or a salt, solvate, hydrate or isomer thereof,
wherein [ligase ligand moiety] is:
(a) Formula (Va) or (Vb): 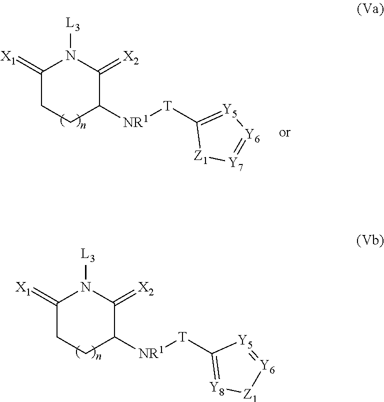
or a pharmaceutically acceptable salt or tautomer thereof,
wherein
each of X₁ and X₂ is independently O or S;
Z₁ is O, S or NR⁶;
T is is C═O or SO₂;
R¹ is hydrogen, alkyl, cycloalkyl, alkenyl, cycloalkenyl, aryl, heteroaryl, or benzyl;
each of Y₅, Y₆, Y₇, and Y₈ is independently N or CR⁷,
wherein at least one of Y₅, Y₆ and Y₇ in Formula (Va) is CR⁷, and at least one of Y₅, Y₅ and Y₈ in Formula (Vb) is CR⁷;
n is 0, 1 or 2;
L₃ is hydrogen, alkyl, alkenyl, aryl, heteroaryl, benzyl, haloalkyl, haloalkenyl, —C(O)R″″, —CH₂C(O)OR″″, —C(O)OR″″, —C(O)NH₂, —C(O)NHR″″, —C(O)NR″″2, —OR″″, —NR″″₂, or —S(O)₂R″″;
each R⁷ is independently hydrogen, halogen, alkyl, cycloalkyl, heterocycloalkyl, alkenyl, cycloalkenyl, alkynyl, aryl, heteroaryl, benzyl, haloalkyl, haloalkenyl, —NH₂, —NHR″″, —NR″″₂, —CH₂NR″″₂, —NR″″C(O)R″″, —NR″″C(O)CH₂NR″″₂, —NR″″C(O)CH₂-heterocycloalkyl, —NR″″C(O)CH(OH)R″″, —CH₂NR″″C(O)OR″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —NHC(S)NHR″″, SR″″, or —S(O)₂R″″, —S(O)₂OR″″, —S(O)₂NH₂, —S(O)₂NHR″″, —S(O)₂NR″″₂, —O—R²¹, —NH—R²¹, —C(O)—NH—R²¹, or —CH₂—NH—C(O)—R²¹;
each R″″ is independently hydrogen, alkyl, cycloalkyl, alkenyl, cycloalkenyl, aryl, heteroaryl, or benzyl;
R⁶ is hydrogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —N[C(O)R″″]₂, —NR″″C(O)OR″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —R²¹, —C(O)—NH—R²¹, or —CH₂—NH—C(O)—R²¹;
wherein R²¹ is a bond connected to R¹⁸ of the linker, and wherein formula (Va) and formula (Vb) each contain a single R²¹;
wherein when Z₁ is O, then Y₆ is CR⁷ and
wherein when the compound is of Formula (Va), then
(i) when each of Y₅, Y₆ and Y₇ is CR⁷, then at least one of R⁷ is not H;
(ii) when Z₁ is NR⁶, then Y₆ and Y₇ are CR⁷;
(iii) when Z₁ is S, then Y₅ is not C—OMe and Y₆ is not C—OMe;
(iv) when Z₁ is S and Y₅ is C—NHCOMe, then Y₇ is not C—CH₂NR″″C(O)OR″″;
(v) when Z₁ is S and Y₅ is N, then Y₆ is not C—H, C-aryl or C—C(O)OR″″; and
(vi) when Z₁ is S and Y₆ is N, then Y₇ is C—NH₂, C—NHR″″, C—NR″″₂, C—NR″″C(O)OR″″, C—CH₂NR″″C(O)OR″″, C-haloalkyl, C-^{t}Butyl, C—OR″″, C—COOR″″ or C—SR″″; wherein when Y₇ is C—NH₂, C—NHR″″ or C—NR″″₂, then Y₅ is C—H;
and when the compound is of Formula (Vb), then:
(vii) when each of Y₅, Y₆ and Y₈ is CR⁷, then at least one of R⁷ is not H;
(viii) when Z₁ is S, then Y₅ is not C—COOH or C—NHC(O)Me, and Y₈ is not C—Br;
(ix) when Z₁ is S and Y₆ is C—Br, then Y₈ is C—OR″″
(x) when Z₁ is S, Y₅ is N and Y₆ is C—H or C—NH₂, then Y₈ is not C—H
(xi) when Z₁ is S and Y₅ is N, then Y₆ is not C-halogen, C-alkyl, C-cycloalkyl, C-aryl, C-heteroaryl, C—CH₂NH₂, C—COOalkyl, or C—NHC(O)alkyl; (xii) when Z₁ is NR⁶, then Y₅, Y₆ and Y₈ are CR⁷.
or
(b) Formula (IIa) or (IIb): 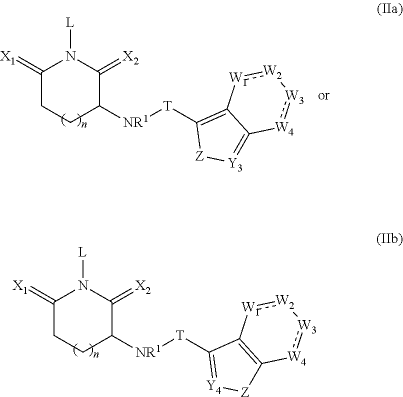
wherein
each of X₁ and X₂ is independently O or S;
Z is O, S or NR²;
T is C═O or SO₂;
Y₃ is N or CR;
Y₄ is N or CR; [symbol]
indicates a single or double bond, wherein
when each [symbol] is a double bond, each of W₁, W₂, W₃ and W₄ is independently N or CR^{a}, wherein at least one of W₁, W₂, W₃ and W₄ is N, and
when each [symbol] is a single bond, W₁, W₂, W₃ and W₄ are each CR^{a}₂ and Y₄ is CR;
n is 0, 1 or 2;
L is hydrogen, alkyl, alkenyl, aryl, heteroaryl, benzyl, haloalkyl, haloalkenyl, —C(O)R^{h}, —C(O)OR^{h}, —C(O)NH₂, —C(O)NHR^{h}, —C(O)NR^{h}₂, —OR^{h}, —NR^{h}₂, or —S(O)₂R^{h};
each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH₂R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, or —S(O)₂R^{h}, —S(O)₂OR^{h}, —S(O)₂NH₂, —S(O)₂NHR^{h}, or —S(O)₂NR^{h}₂;
each R^{a} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —NR^{h}C(O)CH(OH)R^{h}, —NR^{h}C(O)OR^{h}, —NR^{h}SO₂R^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, —S(O)₂NR^{h}₂, —O—R²¹, —NH—R²¹, —C(O)—NH—R²¹, or —CH₂—NH—C(O)—R²¹;
each R^{h} is independently hydrogen, alkyl, cycloalkyl, heterocycloalkyl, alkenyl, cycloalkenyl, aryl, heteroaryl, or benzyl;
R² is hydrogen, alkyl, cycloalkyl, alkenyl, cycloalkenyl, alkynyl, aryl, heteroaryl, benzyl, haloalkyl, haloalkenyl, —NH₂, —NHR^{h}, —NR^{h}₂, —NR^{h}C(O)R^{h}, —N[C(O)R^{h}]₂, —NR^{h}C(O)OR^{h}, —NO₂, —CN, —C(O)R^{h}, —C(O)OR^{h}, —C(O)NH₂, —C(O)NHR^{h}, —C(O)NR^{h}₂, —OR^{h}, —OC(O)R^{h}, —OC(O)OR^{h}, —OC(O)NH₂, —OC(O)NHR^{h}, —OC(O)NR^{h}₂, —SR^{h}, —S(O)₂R^{h}, —S(O)₂OR^{h}, —S(O)₂NH₂, —S(O)₂NHR^{h}, or —S(O)₂NR^{h}₂; and
R¹ is hydrogen, alkyl, cycloalkyl, alkenyl, cycloalkenyl, aryl, heteroaryl, or benzyl;
R²¹ is a bond connected to R¹⁸ of the linker, and wherein formula (IIa) and formula (IIb) each contain a single R²¹;
wherein when each [symbol] is a double bond, Z is NR², R² is hydrogen, and each R^{a} is hydrogen, then W₄ is CR^{a};
wherein [MCL-1 ligand prodrug moiety] is a compound of Formula (A1), (A2), (A3) or (A4): 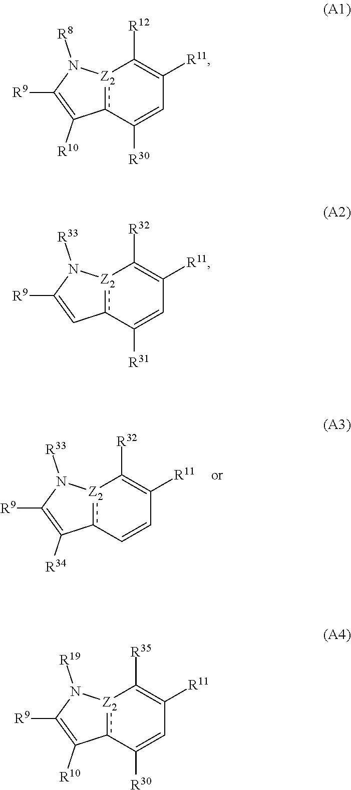
wherein [symbol]
is a single bond or a double bond;
each Z₂ is independently N or C, wherein when Z₂ is N, then [symbol] is a single bond; and when Z₂ is C, then [symbol] is a double bond;
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is 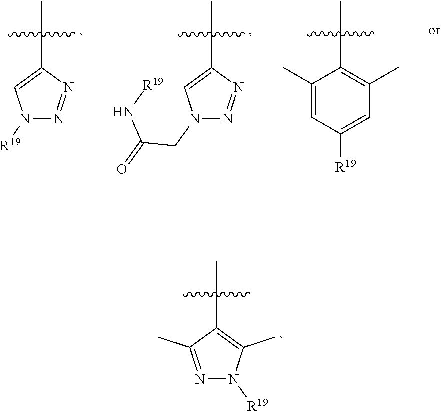
R³¹ is —C₂₋₅alkyl-O—R¹³, —O—C₂₋₅alkyl-R¹³ or —C₂₋₅alkyl-NMe-R⁷³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, 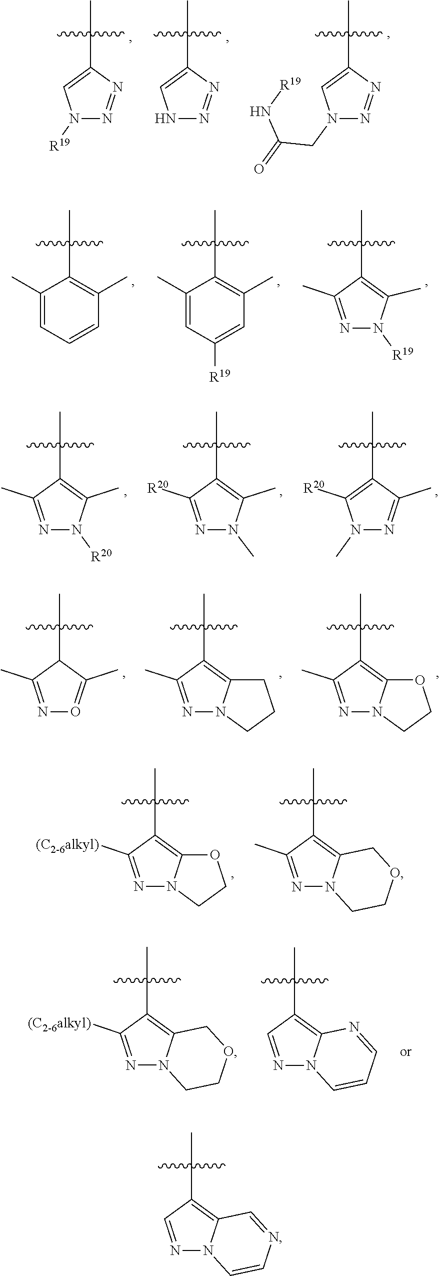
each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is 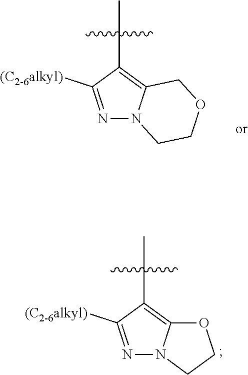
R¹⁹ is a bond connected to R¹⁴ of the linker; wherein each of Formula (A1), Formula (A2), Formula (A3) and Formula (A4) contains a single R¹⁹; and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
wherein each R¹⁰ is independently —OH, —O(unsubstituted C₁-C₆alkyl), —OBn, —NH₂, —NMe₂, —NHC(O)(unsubstituted C₁-C₆alkyl), —NHC(O)O(unsubstituted C₁-C₆alkyl), heterocycloalkyl, heteroaryl, aryl, —C(O)NMe₂;
each R^{p} is independently —(CH₂)ₘOC(O)(unsubstituted C₁-C₆alkyl), and
each m is independently 1, 2, 3 or 4;
or
(ii) R⁹ is —C(O)O(CH₂)ₚNMe₂, —C(O)O(CH₂)ₚNHMe, —C(O)OCH₂CH(OH)CH₂OH, —C(O)OCH₂CH₂CMe₂OH, or —C(O)OCH₂CH₂SO₂Me wherein each p is independently 2, 3, 4, 5, or 6;
and wherein [linker] has the following formula 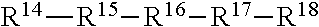
wherein
R¹⁴ is —C₁₋₆ alkyl, —C₂₋₆ alkenyl, —C₂₋₆ alkynyl, —C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)ₓ, —(C₃H₆—O)ₓ, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
wherein at least one of R¹⁴—R¹⁸ is present.

79. The compound of claim 78, wherein unless otherwise specified each alkyl, alkenyl, alkynyl, aryl, heteroaryl and benzyl groups is unsubstituted.

80. The compound of any one of claims 78-79, wherein when Z₁ is S in Formula (Vb), then Y₁ is not C—NHC(O)R″″ or —C(O)OR″″.

81. The compound of any one of claims 78-80, wherein Z₁ is NR⁶.

82. The compound of any one of claims 78-81, wherein each R is independently hydrogen, halogen, alkyl, cycloalkyl, alkenyl, cycloalkenyl, alkynyl, aryl, heteroaryl, benzyl, haloalkyl, haloalkenyl, —NH₂, —NHR″″, —NR″″₂, —NR″″C(O)R″″, —NR″″C(O)CH(OH)R″″, —NR″″C(O)OR″″, —NR″″SO₂R″″, —NO₂, —CN, —C(O)R″″, —C(O)OR″″, —C(O)NH₂, —C(O)NHR″″, —C(O)NR″″₂, —OR″″, —OC(O)R″″, —OC(O)OR″″, —OC(O)NH₂, —OC(O)NHR″″, —OC(O)NR″″₂, —SR″″, or —S(O)₂R″″, —S(O)₂OR″″, —S(O)₂NH₂, —S(O)₂NHR″″, —S(O)₂NR″″₂, —O—R²¹, —NH—R¹, —C(O)—NH—R¹, or —CH₂—NH—C(O)—R¹.

83. The compound of any one of claims 78-82, wherein R¹ is hydrogen.

84. The compound of any one of claims 78-83, wherein R⁶ is hydrogen.

85. The compound of any one of claims 78-84, wherein [ligase ligand moiety] is of Formula (Va) and Y₅, Y₆ and Y₇ are each CR⁷.

86. The compound of claim 85, wherein
Y₅ is —C—NHC(O)R″″,
Y₆ is CH, and
Y₇ is CH or CCl.

87. The compound of claim 86, wherein:
L₃ is hydrogen;
Z₁ is S;
R¹ is hydrogen;
T is C═O; and
Y₇ is CH.

88. The compound of any one of claims 78-84, wherein the compound is of Formula (Vb) and Y₅, Y₆ and Y₈ are each CR⁷.

89. The compound of claim 88, wherein:
L₃ is hydrogen;
Z₁ is S;
R¹ is H;
T is C═O;
Y₅ is CH, C—OR″″, CCI, C—CN, or C—NHC(O)R″″;
Y₆ is CH, CCI, C-alkyl, C-cycloalkyl, or C-haloalkyl; and
Y₈ is CH, C—OR″″, C—NHC(O)R″″, C—NHC(O)OR″″, C—NHR″″, C—NH₂, or C—NHSO₂R″″;
wherein, when Y₅ is CCI, then Y₆ is CH, C-alkyl, C-cycloalkyl, or C-haloalkyl;
optionally wherein each R″″ is independently alkyl, cycloalkyl, aryl or benzyl.

90. The compound of claim 89, wherein:
Y₅ is CH;
Y₆ is CH or CCl; and
Y₈ is C—OR″″ or C—NH₂, optionally C—OMe or C—NH₂.

91. The compound of any one of claims 78-90, wherein:
(a) Z is NR²; or
(b) Z is S.

92. The compound of any one of claims 78-91, wherein each [symbol] is a double bond.

93. The compound of any one of claims 78-92, wherein L is hydrogen.

94. The compound of claim 92 or 93, wherein one of W₁, W₂, W₃ and W₄ is N, and the remaining three of W₁, W₂, W₃ and W₄ are each CR^{a}; optionally wherein W₄ is CR^{a}.

95. The compound of any one of claims 78-92, wherein two of W₁, W₂, W₃ and W₄ is N, and the remaining two of W₁, W₂, W₃ and W₄ are each CR^{a}.

96. The compound of any one of claims 78-92, wherein one of W₁, W₂, W₃ and W₄ is CR^{a}, and the remaining three of W₁, W₂, W₃ and W₄ are each N.

97. The compound of any one of claims 78-96, wherein each R is independently hydrogen, halogen or —NR^{h}C(O)R^{h}.

98. A compound of formula (I) 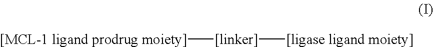
or a salt, solvate, hydrate or isomer thereof,
wherein [ligase ligand moiety] is:
(a) Formula (Via) or (VIb): 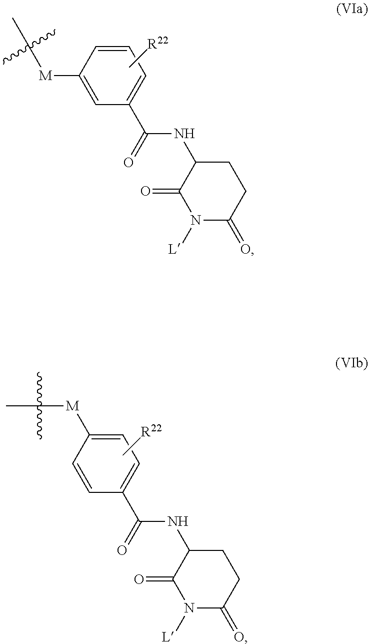
wherein
M is O, S or NH, or is absent; [symbol]
indicates attachment to R¹⁸ of the linker;
R²² is hydrogen, halogen, —OMe, an amino group, heterocycloalkyl, or unsubstituted C₁-C₆ alkyl;
and
L′ is hydrogen, alkyl, benzyl, acetyl or pivaloyl
(b) Formula (II): 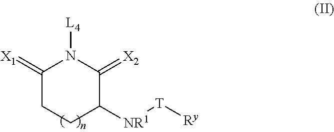
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₄ is hydrogen, alkyl, alkenyl, aryl, heteroaryl, benzyl, haloalkyl, haloalkenyl, —C(O)H, —C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{y} is selected from 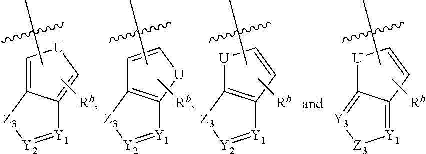
wherein [symbol]
indicates attachment to T,
Z₃ is O, S or NR³;
U is O, S, NR^{b} or CRⁱ₂;
each of Y₁, Y₂ and Y₃ is independently N or CR^{d};
each R^{d} is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each Rⁱ is independently hydrogen, halogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R_{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, or —S(O)₂NR^{b}₂;
each R³ is independently hydrogen, alkyl, cycloalkyl, alkenyl, cycloalkenyl, alkynyl, aryl, heteroaryl, benzyl, haloalkyl, haloalkenyl, —NH₂, —NHR^{b}, —NR^{b}₂, —NHC(O)R^{b}, —NR^{b}C(O)R^{b}, NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —C(O)—NH—R²¹, or —CH₂—NH—C(O)—R²¹;
each R^{b} is independently alkyl, cycloalkyl, alkenyl, cycloalkenyl, aryl, heteroaryl, or benzyl;
R²¹ is a bond connected to R¹⁸ of the linker, wherein Formula (II) contains a single R²¹;
wherein,
(i) when R^{y} is 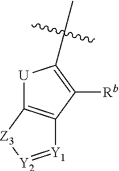
then Y₂ is CR^{d}; and
(ii) when R^{y} is 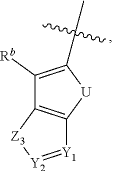
then Rⁱ in CRⁱ₂ is not hydrogen
or
(c) Formula (III): 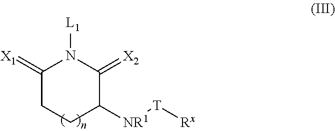
wherein:
each of X₁ and X₂ is independently O or S;
T is C═O or SO₂;
R¹ is hydrogen, alkyl, cycloalkyl, alkenyl, cycloalkenyl, aryl, heteroaryl, or benzyl;
n is 0, 1 or 2;
L₁ is hydrogen, alkenyl, aryl, heteroaryl, benzyl, haloalkyl, haloalkenyl, —C(O)H, —C(O)R^{b}, —C(O)OH, —C(O)OR^{b}, —CH₂C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H or —S(O)₂R^{b};
R^{x} is selected from 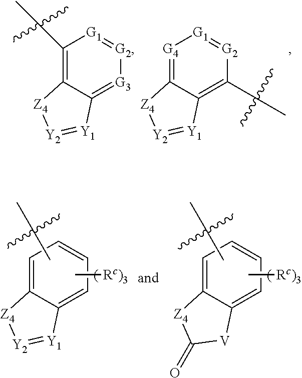
wherein [symbol] indicates attachment to T,
Z₄ is O, S or NR⁴;
V is CR^{f}₂, NR⁴ or S;
each of G₁, G₂, G₃ and G₄ is independently N or CR^{c},
each of Y₁ and Y₂ is independently N or CR^{f},
each R^{f} is independently hydrogen, halogen, alkyl, cycloalkyl, alkenyl, cycloalkenyl, alkynyl, aryl, fused aryl-cycloalkyl, fused aryl-heterocycloalkyl, heteroaryl, heteroaryl substituted with at least one aryl group, benzyl, haloalkyl, haloalkenyl, —NH₂, —NHR^{b}, —NR^{b}₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}₂, —NO₂, —CN, —C(O)H, —C(O)R^{b}, —C(O)OH, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —R²¹, —O—R²¹, —NH—R²¹, —C(O)—NH—R²¹, or —CH₂—NH—C(O)—R²¹; or when Y₁ and Y₂ are CR^{f}then each R^{f}, together with the carbon atom to which it is attached, forms a 5- or 6-membered ring;
each R^{c} is independently hydrogen, halogen, alkyl, cycloalkyl, alkenyl, cycloalkenyl, alkynyl, aryl, aryl substituted with at least one —OR^{b}, heteroaryl, benzyl, haloalkyl, haloalkenyl, —NH₂, —NHR^{b}, —NR^{b}₂, —CH₂NH₂, —NHC(O)R^{b}, —NR^{b}C(O)R^{b}, —NHC(O)CH(OH)R^{b}, —NR^{b}C(O)CH(OH)R^{b}, —NHC(O)OR^{b}, —NR^{b}C(O)OR^{b}, —NHSO₂R^{b}, —NR^{b}SO₂R^{b}, —NO₂, —CN, —C(O)H, —C(O)R^{b}, —C(O)OR^{b}, —C(O)NH₂, —C(O)NHR^{b}, —C(O)NR^{b}₂, —OH, —OR^{b}, —OC(O)H, —OC(O)R^{b}, —OC(O)OH, —OC(O)OR^{b}, —OC(O)NH₂, —OC(O)NHR^{b}, —OC(O)NR^{b}₂, —SH, —SR^{b}, —S(O)₂H, —S(O)₂R^{b}, —S(O)₂OH, —S(O)₂OR^{b}, —S(O)₂NH₂, —S(O)₂NHR^{b}, —S(O)₂NR^{b}₂, —O—R²¹, —NH—R²¹, —C(O)—NH—R²¹, or —CH₂—NH—C(O)—R²¹;
each R⁴ is independently hydrogen, halogen, alkyl, cycloalkyl, alkenyl, cycloalkenyl, alkynyl, aryl, heteroaryl, benzyl, haloalkyl, haloalkenyl, —C(O)H, C(O)R^{b}, —C(O)OH, —C(O)OR^{b}, —C(O)NH₂, —C(O)NHR^{b}, —C(O)NR^{b}₂, —OH, —OR^{b}, —NH₂, —NHR^{b}, —NR^{b}₂, —S(O)₂H, —S(O)₂R^{b}, —R²¹, —C(O)—NH—R²¹, or —CH₂—NH—C(O)—R²¹; and
each R^{b} is independently alkyl, cycloalkyl, alkenyl, cycloalkenyl, aryl, heteroaryl, or benzyl;
R²¹ is a bond connected to R¹⁸ of the linker, wherein Formula (III) contains a single R²¹;
wherein, when n=2, each R^{c} is hydrogen, and each of G₁, G₂, G₃ and G₄ is CR^{c}, then C=X₁ may be replaced by CH;
and wherein:
(i) when R^{x} is 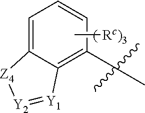
and Z₄ is NH, then L₁ is hydrogen, —CH₂C(O)OR^{b}, or —OR^{b};
(ii) when R^{x} is 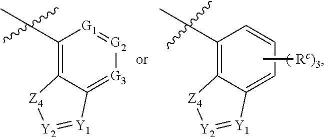
Z₄ is NR⁴, Y₁ is CR^{f}, and Y₂ is N, then R⁴ is not alkyl and at least one of R² and R is not H;
(iii) when R^{x} is 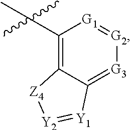
Z₄ is NR⁴, and Y₁ and Y₂ are CR^{f}, then at least one of G₁, G₂ and G₃ is N;
(iv) when Z₄ is NR⁴, and Y₁ and Y₂ are CR^{f}, then R^{x} is not 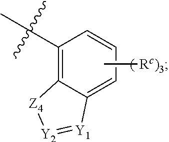
(v) when R^{x} is 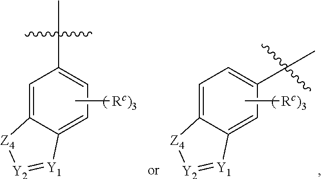
Z₄ is NR⁴, and Y₁ or Y₂ is N, then R⁴ is not alkyl;
(vi) when R^{x} is 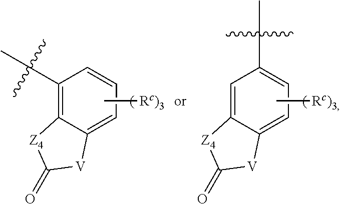
then n=1 or 2; and
(vii) when R^{x} is 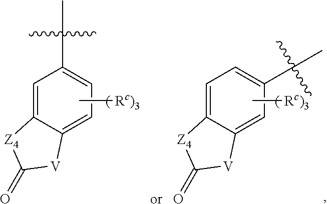
then Z₄=O or S
wherein [MCL-1 ligand prodrug moiety] is a compound of Formula (A1), (A2), (A3) or (A4): 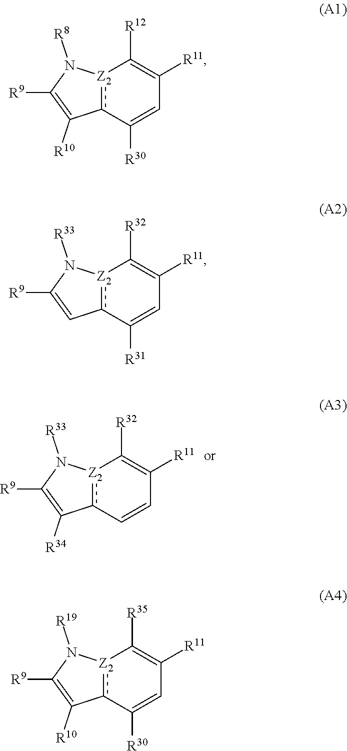
wherein [symbol]
is a single bond or a double bond;
each Z₂ is independently N or C, wherein when Z₂ is N, then [symbol] is a single bond; and when Z₂ is C, then [symbol] is a double bond;
each R¹¹ is independently H, halogen or C₁-C₆ alkyl,
R⁸ is C₁-C₆ alkyl substituted with a piperazine;
in each of Formula (A1) and Formula (A4), one of R¹⁰ and R³⁰ is H and the other of R¹⁰ and R³⁰ is —C₂₋₅alkyl-O—R¹³, —O—C₂₋₅alkyl-R¹³ or —C₂₋₅alkyl-NMe-R¹, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—,
R¹² is 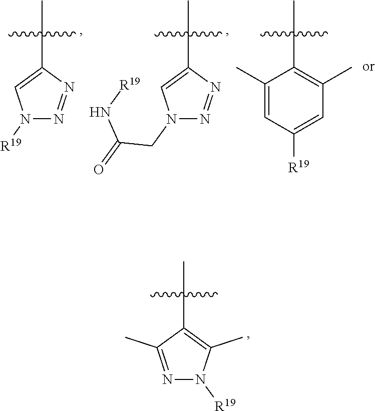
R³¹ is —C₂₋₅alkyl-O—R¹³, —O—C₂₋₅alkyl-R¹³ or —C₂₋₅alkyl-NMe-R¹³ wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
each R³² is independently H, 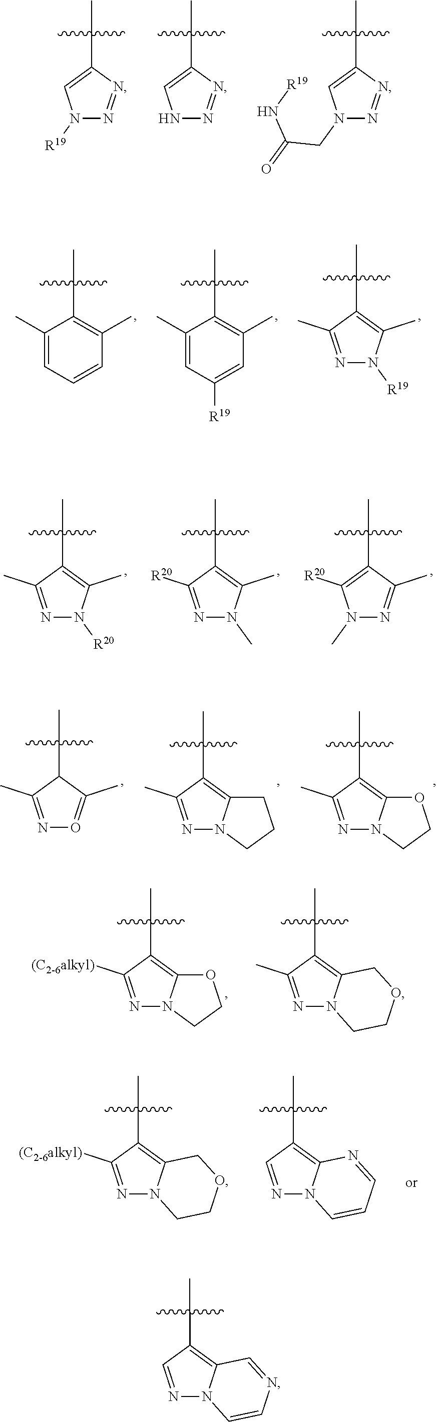
each R³³ is independently R¹⁹ or C₁-C₆ alkyl, wherein the C₁-C₆ alkyl is optionally substituted with morpholine or a piperazine;
R²⁰ is Me, —CH₂—OMe, —(CH₂CH₂O)ₚ(C₁-C₆ alkyl), or —CH₂—O-bromobenzaldehyde, wherein p is 1-5;
R³⁴ is C₂₋₅alkyl-O—R¹³ or —O—C₂₋₅alkyl-R³, wherein R¹³ is phenyl, naphthyl or tetraline, wherein the phenyl, naphthyl or tetraline is optionally substituted with at least one substituent selected from halogen, C₁-C₆ alkyl and —O(C₁-C₆ alkyl); or wherein the tetraline is optionally substituted with a bridging —CH₂— group; or wherein the naphthyl is optionally substituted with —O— or —S—;
R³⁵ is 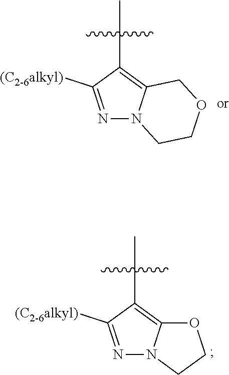
R¹⁹ is a bond connected to R¹⁴ of the linker; wherein each of Formula (A1), Formula (A2), Formula (A3) and Formula (A4) contains a single R¹⁹; and
(i) R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), —OC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolium cation;
wherein each R¹⁰ is independently —OH, —O(unsubstituted C₁-C₆alkyl), —Obn, —NH₂, —Nme₂, —NHC(O)(unsubstituted C₁-C₆alkyl), —NHC(O)O(unsubstituted C₁-C₆alkyl), heterocycloalkyl, heteroaryl, aryl, —C(O)Nme₂;
each R^{p} is independently —(CH₂)ₘOC(O)(unsubstituted C₁-C₆alkyl), and
each m is independently 1, 2, 3 or 4;
or
(ii) R⁹ is —C(O)O(CH₂)ₚNme₂, —C(O)O(CH₂)ₚNHMe, —C(O)OCH₂CH(OH)CH₂OH, —C(O)OCH₂CH₂CMe₂OH, or —C(O)OCH₂CH₂SO₂Me, wherein each p is independently 2, 3, 4, 5, or 6;
and wherein [linker] has the following formula 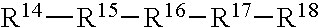
wherein
R¹⁴ is —C₁-C₆ alkyl, —C₂₋₆ alkenyl, —C₂₋₆ alkynyl, C₁₋₆ alkyl-N(C₁₋₆ alkyl)-, —C(O)—, —SO₂— or is absent
R¹⁵ is cycloalkyl, heterocycloalkyl, aryl, heteroaryl, —C₁₋₆ alkyl-NH—, —C₁₋₆ alkyl-N(C₁₋₆ alkyl)-, -cycloalkyl-NH—, -heterocycloalkyl-NH— or is absent
R¹⁶ is —C₁₋₆alkyl, —C(O)—, —C(O)—C₁₋₆alkyl-, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(CH₂O)ₓ, —(C₂H₄—O)ₓ, —(C₃H₆—O)ₓ, or is absent
x is 1-10
y is 2-10
R¹⁸ is —C₁₋₆ alkyl, —C₁₋₆ alkyl-C(O)—, cycloalkyl, —CH₂—NH—C(O)—, heterocycloalkyl, or is absent
wherein at least one of R¹⁴—R¹⁸ is present

99. The compound of claim 98, wherein unless otherwise specified each alkyl, alkenyl, alkynyl, aryl, heteroaryl and benzyl is unsubstituted.

100. The compound of any one of claims 98-99, wherein in Formula (III):
each of X₁ and X₂ is O;
T is C═O;
R¹ is hydrogen,
L₁ is hydrogen,
R^{x} is 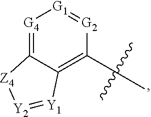
Z₄ is NR⁴;
each of G₁, G₂ and G₄ is CR^{c},
Y₁ is N, and
Y₂ is CR^{f}, wherein R^{f} is not hydrogen.

101. The compound of any one of claims 98-100 wherein [ligase ligand moiety] is Formula (III):

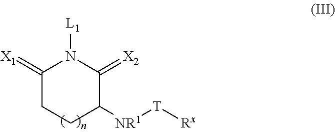

102. The compound of any one of claims 98-101, wherein:
(a) one of R^{c} is —O—R²¹, —NH—R²¹, —C(O)—NH—R²¹, or —CH₂—NH—C(O)—R²¹;
(b) G₁ is C—O—R²¹, C—NH—R²¹, C—C(O)—NH—R²¹, or C—CH₂—NH—C(O)—R²¹;
(c) G₂ is C—O—R²¹, C—NH—R²¹, C—C(O)—NH—R²¹, or C—CH₂—NH—C(O)—R²¹;
(d) R⁴ is R²¹, —C(O)—NH—R²¹, or —CH₂—NH—C(O)—R²¹; or
(e) one of R^{f} is —R²¹, —O—R²¹, —NH—R²¹, —C(O)—NH—R²¹, or —CH₂—NH—C(O)—R²¹.

103. The compound of any one of claims 98-102, wherein Y₂ is C—R²¹, CO—R²¹, C—NH—R²¹, C—C(O)—NH—R²¹, or C—CH₂—NH—C(O)—R²¹.

104. The compound of any one of claims 98-103, wherein:
(a) [ligase ligand moiety] is selected from: 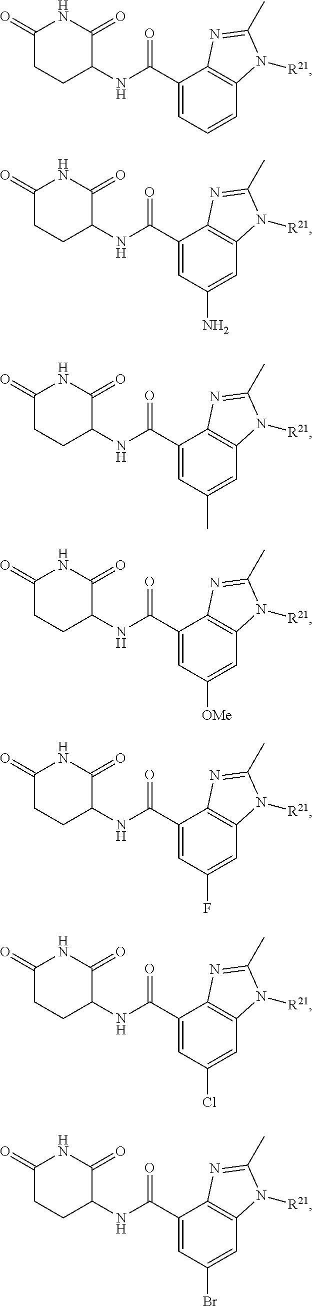 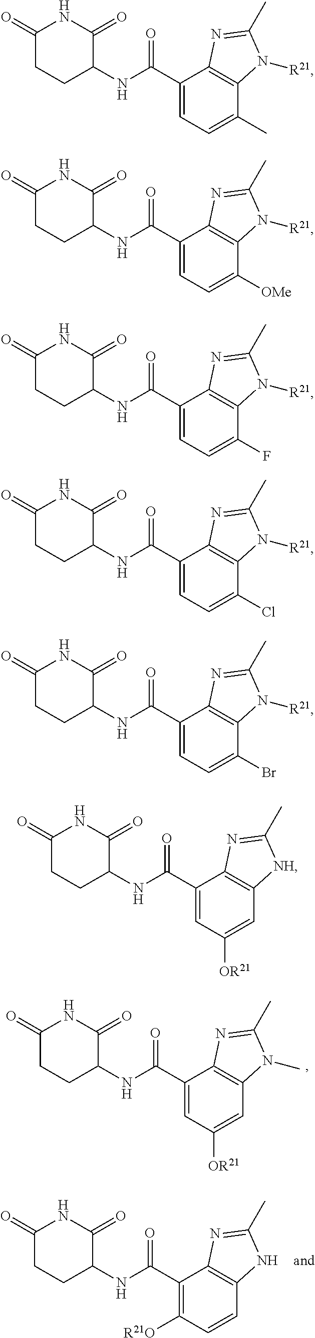 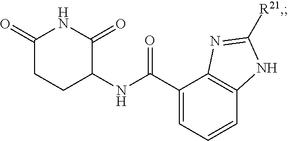
or
(b) [ligase ligand moiety] is selected from:

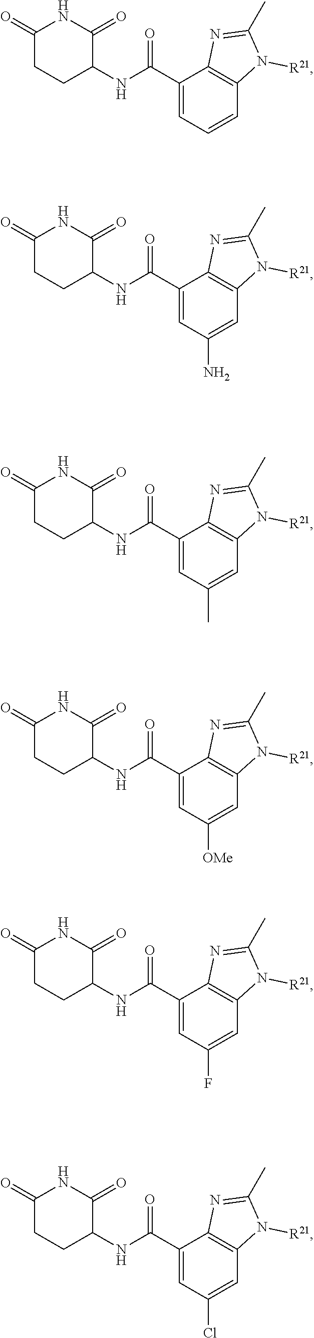

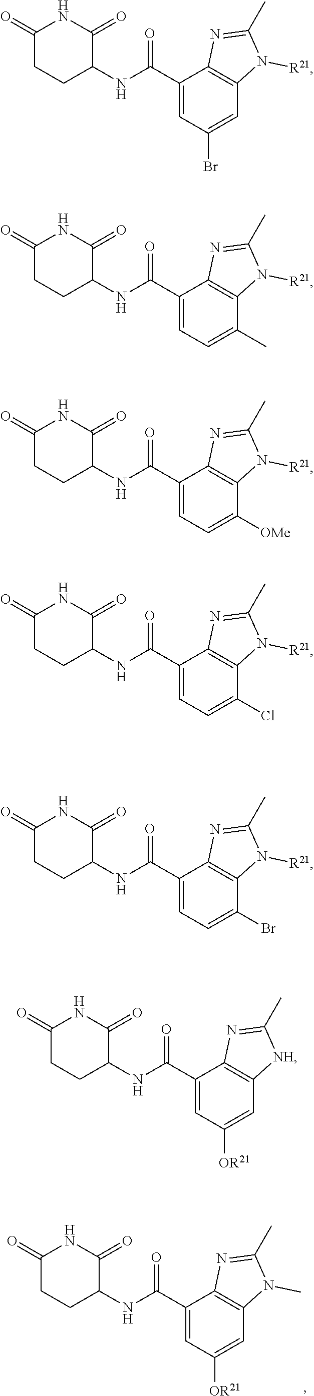

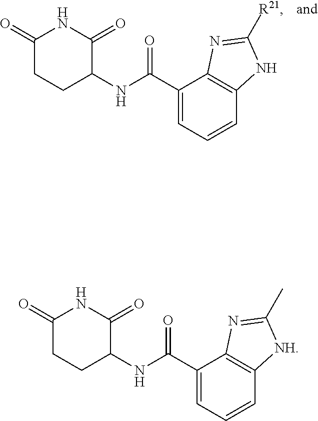

105. The compound of claim 104, wherein [ligase ligand moiety] is

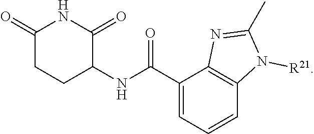

106. The compound of any one of claims 98-99, wherein [ligase ligand moiety] is

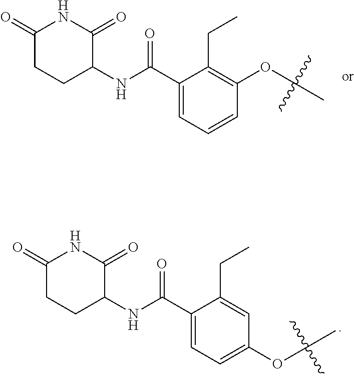 [SMILES: *Oc1cccc(C(=O)NC2CCC(=O)NC2=O)c1CC ; *Oc1ccc(C(=O)NC2CCC(=O)NC2=O)c(CC)c1]

107. The compound of any one of claims 53-106, wherein:
R¹⁶ is —C₁-C₆ alkyl, —C(O)—, —C(O)—NH—, —C(O)O—, —CH₂—C(O)—, —CH₂—C(O)—NH—, —CH₂—C(O)O— or is absent
R¹⁷ is —CH₂(C₂H₄—O)_{y}, —(C₂H₄—O)ₓ, —(C₃H₆—O)ₓ, or is absent
R¹⁸ is —C₁₋₆ alkyl, cycloalkyl, —CH₂—NH—C(O)—, heterocycloalkyl, or is absent.

108. The compound of any one of claims 53-107, wherein R¹⁴ is —C₁₋₆alkyl, —C₂₋₆alkenyl, —C₂₋₆alkynyl, —C(O)—, —SO₂— or is absent

109. The compound of claim 108, wherein R¹⁴ is —C₁₋₆alkyl.

110. The compound of any one of claims 53-109, wherein R¹⁵ is cycloalkyl, heterocycloalkyl, aryl, heteroaryl, —C₁₋₆ alkyl-NH—, -cycloalkyl-NH— or is absent.

111. The compound of claim 110, wherein R¹⁵ is heterocycloalkyl.

112. The compound of claim 111, wherein R¹⁵ is piperazine, bridged piperazine, piperazine N-oxide, piperazine cation, 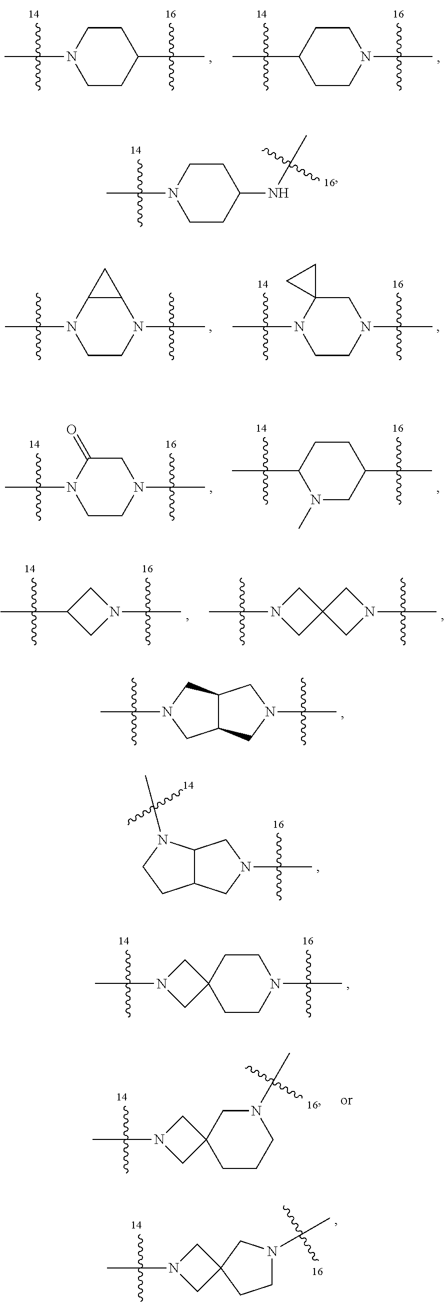
wherein [symbol] indicates attachment to R¹⁴ and [symbol] indicates attachment to R¹⁶.

113. The compound of claim 112, wherein R¹⁵ is piperazine or

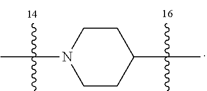 [SMILES: *C1CCN(*)CC1]

114. The compound of claim 110, wherein R¹⁵ is absent.

115. The compound of any one of claims 53-114, wherein R¹⁶ is —C₁₋₆alkyl, —CH₂—C(O)—NH— or —C(O)—.

116. The compound of claim 115, wherein R¹⁶ is —C₁₋₆alkyl or —C(O)—.

117. The compound of any one of claims 53-116, wherein R¹¹ is absent.

118. The compound of any one of claims 53-117, wherein R¹⁸ is —C₁₋₆ alkyl, heterocycloalkyl, or is absent

119. The compound of claim 118, wherein R¹⁸ is —C₁₋₆ alkyl or heterocycloalkyl.

120. The compound of claim 119, wherein R¹⁸ is —C₁₋₆ alkyl or piperazine.

121. The compound of claim 118, wherein R¹⁸ is absent.

122. The compound of any one of claims 53-121, wherein [linker] is selected from 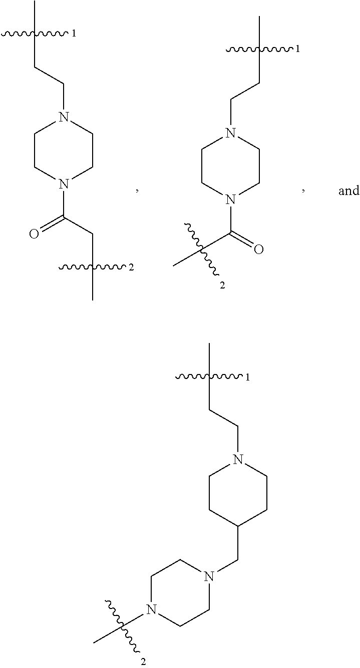 [SMILES: *CCN1CCN(C(=O)C*)CC1 ; *CCN1CCN(C(*)=O)CC1 ; *CCN1CCC(CN2CCN(*)CC2)CC1]
wherein [symbol]
indicates attachment to [MCL-1 ligand prodrug moiety] and [symbol]
indicates attachment to [ligase ligand moiety].

123. The compound of any one of claims 53-122, wherein in each of Formula (A1) and Formula (A4), one of R¹⁰ and R³⁰ is H and the other of R¹⁰ and R³⁰ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

124. The compound of any one of claims 53-123, wherein in Formula (A1) R¹² is

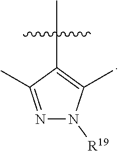

125. The compound of any one of claims 53-124, wherein in Formula (A4) R³⁵ is

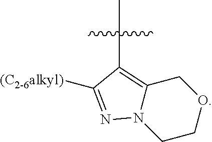

126. The compound of one of claims 53-125, wherein in Formula (A2) R³¹ is —C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

127. The compound of any one of claims 53-126, wherein in Formula (A3) R³⁴ is C₂₋₅alkyl-O—R¹³ or —O—C₂₋₅alkyl-R¹³, wherein R¹³ is phenyl or naphthyl, wherein the phenyl or naphthyl is optionally substituted with at least one substituent selected from halogen and C₁-C₆ alkyl.

128. The compound of any one of claims 53-127, wherein Z₂ is C and [symbol] is a double bond.

129. The compound of any one of claims 53-128, wherein [MCL-1 ligand prodrug moiety] is: 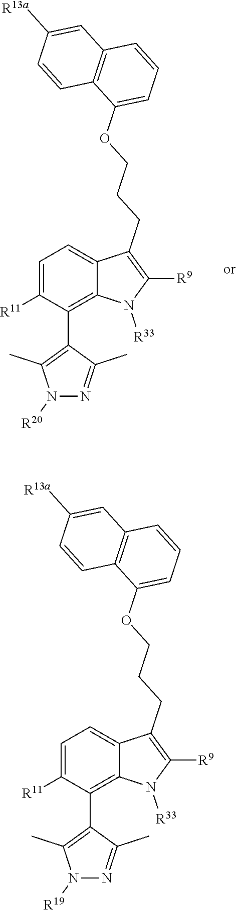
wherein
R¹¹ is halogen;
R^{13a} is halogen; and
R²⁰ is Me, —CH₂—OMe, or —(CH₂CH₂O)ₚ(C₁-C₆ alkyl), wherein p is 1-5.

130. The compound of any one of claims 53-129, wherein R⁹ is —C(O)O(C₁-C₆alkyl), —C(O)Ocycloalkyl, —C(O)Oheterocycloalkyl, —C(O)Oaryl or —P(O)(OR^{p})₂, wherein said C₁-C₆alkyl is substituted by one or more groups selected from —OC(O)(unsubstituted C₁-C₆alkyl), —OC(O)(C₁-C₆alkyl substituted with at least one R¹⁰), —OC(O)cycloalkyl, —OC(O)heterocycloalkyl, —OC(O)aryl, —OC(O)heteroaryl, —OC(O)(CH₂)ₘO(CH₂)ₘO(unsubstituted C₁-C₆alkyl), —OC(O)(CH₂)ₘO(CH₂CH₂O)ₘ(unsubstituted C₁-C₆alkyl), —OC(O)O(unsubstituted C₁-C₆alkyl), —OC(O)O(C₁-C₆alkyl substituted with at least one R¹⁰), —OC(O)Ocycloalkyl, —OC(O)O(CH₂CH₂O)ₘ(unsubstituted C₁-C₆alkyl), —OC(O)O(CH₂CH₂O)ₘ(C₁-C₆alkyl substituted with OP(O)(OH)₂), —OC(O)O(CH₂CH₂S)ₘ(unsubstituted C₁-C₆alkyl), —OC(O)O(CH₂)ₘS(O)(unsubstituted C₁-C₆alkyl), —OC(O)O(CH₂)ₘS(O)₂(unsubstituted C₁-C₆alkyl), —OC(O)NH(C₁-C₆alkyl substituted with OMe), -oC(O)NMe(C₁-C₆alkyl substituted with OMe), —P(O)(OH)₂, —OP(O)(OH)₂, —C(O)heterocycloalkyl, —C(O)N(unsubstituted C₁-C₆alkyl)₂, heteroaryl, heterocycloalkyl, —S(unsubstituted C₁-C₆alkyl), —S(O)(unsubstituted C₁-C₆alkyl), —N⁺(unsubstituted C₁-C₆alkyl)₃, —N⁺(unsubstituted C₁-C₆alkyl)₂cycloalkyl, N-(unsubstituted C₁-C₆alkyl)piperidinium cation, N-(unsubstituted C₁-C₆alkyl)morpholinium cation, and N-(unsubstituted C₁-C₆alkyl)imidazolyl cation.

131. The compound of claim 130, wherein R⁹ is —C(O)O(C₁-C₆alkyl), —C(O)Ocycloalkyl, —C(O)Oheterocycloalkyl, —C(O)Oaryl, or —P(O)(OR^{p})₂, wherein said C₁-C₆alkyl is substituted by one or more groups selected from —OC(O)Me, —OC(O)Et, —OC(O)ⁱPr, —OC(O)C(CH₃)₃, —OC(O)CH₂OH, —OC(O)CH(CH₃)OH, —OC(O)C(CH₃)₂OH, —OC(O)CH₂CH₂OH, —OC(O)CH(OH)CHMe₂, —OC(O)CH(OH)CH₂CHMe₂, —OC(O)CH₂CH(OH)Me, —OC(O)CH₂C(OH)Me₂, —OC(O)CH(CH₂OH)(NHC(O)Me), —OC(O)CH(NHC(O)Me)(isobutyl), —OC(O)CH(OH)aryl, —OC(O)CH(OH)CH₂aryl, —OC(O)heterocycloalkyl, —OC(O)cycloalkyl, —OC(O)aryl, —OC(O)heteroaryl, —OC(O)CH₂CH₂aryl, —OC(O)CH(NH₂)ⁱPr, —OC(O)CH(ⁱPr)NHC(O)O^{t}Bu, —OC(O)CH₂OCH₂CH₂OMe, —OC(O)CH₂(OCH₂CH₂)₂OMe, —OC(O)OMe, —OC(O)OEt, —OC(O)OiPr, —OC(O)Ocycloalkyl, —OC(O)OCH₂CH₂OEt, —OC(O)O(CH₂CH₂O)₂Et, —OC(O)O(CH₂CH₂O)₃Et, —OC(O)O(CH₂CH₂O)₃CH₂CH₂OP(O)(OH)₂, —OC(O)OCh₂CH₂SMe, —OC(O)OCH₂CH₂S(O)Me, —OC(O)OCH₂CH₂S(O)₂Me, —OC(O)OCH₂heteroaryl, —OC(O)OCH₂heterocycloalkyl, —OC(O)OCH₂CH₂OCH₂aryl —OC(O)OCH₂CH₂C(O)NMe₂, —OC(O)O(CH₂)₆NH₂, —OC(O)O(CH₂)₆NMe₂, —OC(O)OCH(CH₂OEt)₂, —OC(O)NHCH₂CH₂OMe, -oC(O)NMeCH₂CH₂OMe, —SMe, —S(O)Me, —P(O)(OH)₂, —OP(O)(OH)₂, —C(O)NMe₂, —C(O)heterocycloalkyl, heteroaryl, heterocycloalkyl, —N⁺(Me)₃, —N⁺(Me)₂cycloalkyl, N-methyl morpholinium cation, N-methyl piperidinium cation and N-methyl imidazolium cation.

132. The compound of claim 131, wherein R⁹ is —C(O)O(C₁-C₆alkyl) wherein said C₁-C₆alkyl is substituted by one or more groups selected from —OC(O)C(CH₃)₃, —OC(O)CH₂CH₂OH, —OC(O)C(CH₃)₂OH, —OC(O)heterocycloalkyl, —OC(O)CH₂OCH₂CH₂OMe, —OC(O)OiPr, —OC(O)Ocycloalkyl, —OC(O)O(CH₂CH₂O)₂Et, —OP(O)(OH)₂, heteroaryl or heterocycloalkyl.

133. The compound of claim 132, wherein R⁹ is —C(O)O(C₁-C₆alkyl) wherein said C₁-C₆alkyl is substituted by —OC(O)C(CH₃)₃

134. The compound of claim 133, wherein R⁹ is —C(O)OCH₂OC(O)C(CH₃)₃.

135. The compound of claim 131, wherein R⁹ is —C(O)OCH₂OC(O)C(CH₃)₃, —C(O)OCH₂OC(O)Me, —C(O)OCH(Me)OC(O)Me, —C(O)OCH₂OC(O)CH₂CH₂OH, —C(O)OCH₂OC(O)C(CH₃)₂OH, —C(O)OCH₂OC(O)piperidine, —C(O)OCH₂OC(O)CH₂OCH₂CH₂OMe, —C(O)OCH₂OC(O)OiPr, —C(O)OCHMeOC(O)OiPr —C(O)OCHMeOC(O)Ocyclohexyl, —C(O)OCHMeOC(O)O(CH₂CH₂O)₂Et, —C(O)OCH₂CH₂OP(O)(OH)₂, —C(O)OCH₂(N-methyl imidazolium cation), —C(O)OCH₂(N-methyl piperidinium cation), —C(O)OCH₂dioxolane, —C(O)OCH₂CH₂morpholine, —C(O)OCH₂CH₂piperazine, —C(O)OCH₂CH₂CH₂piperazine or —P(O)(OCH₂OC(O)C(CH₃)₃)₂.

136. The compound of any one of claims 53-135, wherein R²⁰ is Me or —(CH₂CH₂O)₂Me.

137. The compound of any one of claims 53-136, wherein R³³ is R¹⁹.

138. The compound of any one of claims 53-136, wherein R³³ is C₁-C₆ alkyl substituted with morpholine or a piperazine.

139. The compound of any one of claims 53-138, wherein the C₁-C₆ alkyl substituted with morpholine or a piperazine is

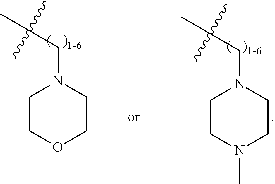

140. The compound of any one of claims 53-139, wherein:
(a) [MCL-1 ligand prodrug moiety] is: 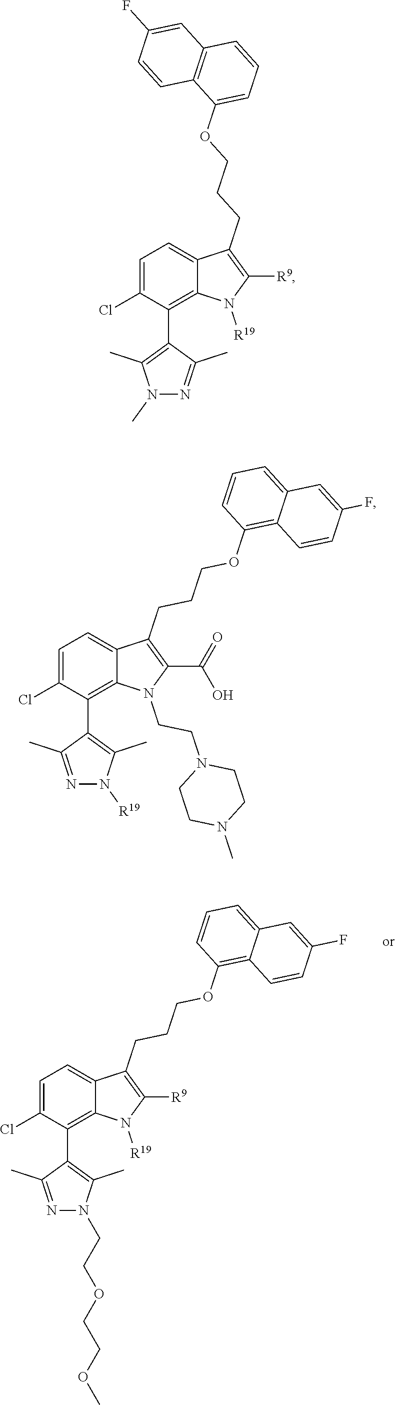 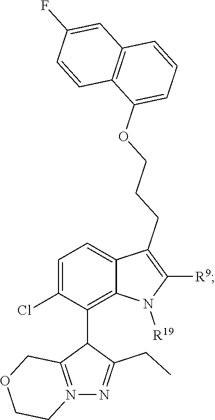
or
(b) [MCL-1 ligand prodrug moiety] is:

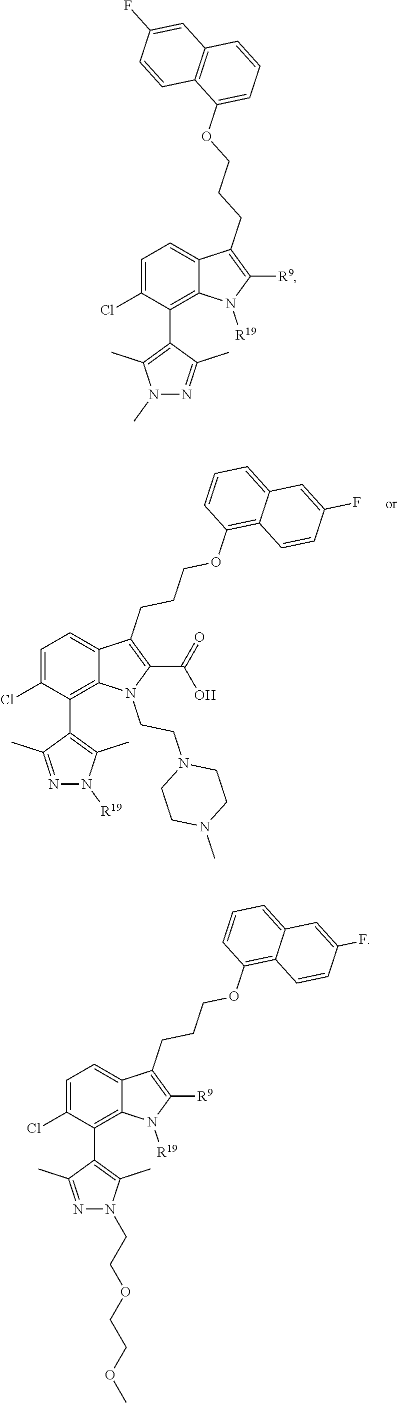

141. The compound of claim 98, wherein the compound is:

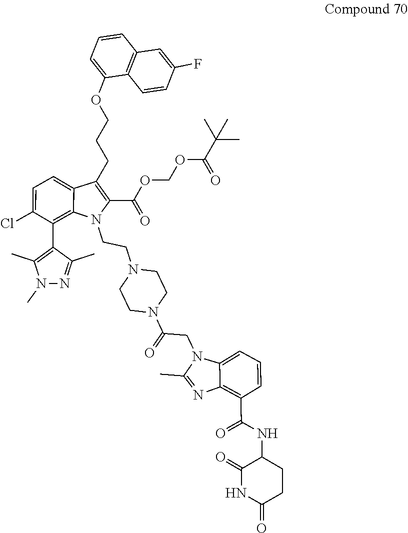 [SMILES: Cc1nn(C)c(C)c1-c1c(Cl)ccc2c(CCCOc3cccc4cc(F)ccc34)c(C(=O)OCOC(=O)C(C)(C)C)n(CCN3CCN(C(=O)Cn4c(C)nc5c(C(=O)NC6CCC(=O)NC6=O)cccc54)CC3)c12]

142. The compound of any preceding claim, wherein T is C═O.

143. The compound of any preceding claim, wherein:
(a) X₁ and X₂ are O;
(b) X₁ is O and X₂ is S;
(c) X₁ is S and X₂ is O; or
(d) X₁ and X₂ are S.

144. The compound of any preceding claim, wherein n is 0.

145. The compound of any one of claims 1-143127, wherein n is 1 or 2.

146. The compound of claim 145, wherein n is 1.

147. The compound of claim 145, wherein n is 2.

148. A pharmaceutical composition comprising a compound of any one of claims 1-147.

149. The compound of any one of claims 1-147 or the pharmaceutical composition of claim 148, for use in medicine.

150. The compound of any one of claims 1-147 or the pharmaceutical composition of claim 148, for use in the treatment of cancer.

151. The compound or composition for use of claim 150, wherein the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer.

152. The compound or composition for use of claim 151, wherein the cancer is multiple myeloma or acute myeloid leukaemia.

153. A method of treating cancer in a subject in need thereof, the method comprising administering to the subject an effective amount of a compound according to any one of claims 1-147, or a pharmaceutical composition according to claim 148.

154. The method of claim 153, wherein the cancer is selected from breast cancer, triple negative breast cancer, colorectal cancer, pancreatic cancer, skin cancer, melanoma, ovarian cancer, kidney cancer, lung cancer, small-cell lung cancer, non-small-cell lung cancer, lymphoma, non-Hodgkin's lymphoma, multiple myeloma, cervical cancer, leukaemia, chronic lymphocytic leukaemia (CLL), acute myeloid leukaemia (AML), chronic myelogenous leukaemia (CML), acute lymphoblastic leukaemia (ALL), bladder cancer, and prostate cancer.

155. The method of claim 154, wherein the cancer is multiple myeloma acute myeloid leukaemia.

156. The method of any one of claims 153-155, wherein the administration does not result in cytotoxicity in cardiomyocytes in the subject.

157. The method of any one of claims 153-156, further comprising administering at least one additional active agent to the subject.

158. The method of claim 157, wherein the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

159. The compound of any one of claims 1-147 or the pharmaceutical composition of claim 148, for use in reversing resistance to chemotherapy or targeted cancer therapies.

160. A method of reversing resistance to chemotherapy or targeted cancer therapies in a subject in need thereof, the method comprising administering to the subject an effective amount of a compound according to any one of claims 1-147, or a pharmaceutical composition according to claim 148.

161. A combined preparation of a compound of any one of claims 1-147 and at least one additional active agent, for simultaneous, separate or sequential use in therapy.

162. The combined preparation of claim 161, wherein the at least one additional active agent is an anti-cancer agent selected from eribulin; fulvestrant; midostaurin; an immune checkpoint inhibitor selected from anti-pd-1 antibody, anti-pd-11 antibody, and anti pd-1/pd-11 interaction inhibitor; nivolumab; pembrolizumab; atezolizumab; pidilizumab; carfilzomib; venetoclax; cytarabine; anthracyclines; a taxane compound; and hypomethylating agents.

163. The combined preparation of any one of claims 161-162 wherein the therapy is the treatment of cancer.

164. A method of reducing the cardiac cytotoxicity of an MCL-1 inhibitor, comprising coupling a cereblon binding moiety to the MCL-1 inhibitor, wherein the cereblon binding moiety is a [ligase ligand moiety] as defined in any one of claims 1-147 and the MCL-1 inhibitor is an [MCL-1 ligand prodrug moiety] as defined in any one of claims 1-147.
